(19) United States
(12) Patent Application Publication
(10) Pub. No.: US 2009/0182020 A1
(43) Pub. Date: Jul. 16, 2009

(54) SUBSTITUTED BIS(HETERO)AROMATIC N--ETHYLPROPIOLAMIDES AND USE THEREOF FOR PRODUCTION OF MEDICAMENTS

(75) Inventors: Michael Haurand, Aachen (DE); Klaus Schiene, Juchen (DE); Sven Kuhnert, Duren (DE); Stefan Oberborsch, Aachen (DE)

Correspondence Address: NORRIS, MCLAUGHLIN & MARCUS, PA, 875 THIRD AVENUE, 18TH FLOOR, NEW YORK, NY, 10022, US

(73) Assignee: GRUNENTHAL GMBH, Aachen (DE)

(21) Appl. No.: 12/146,700

(22) Filed: Jun. 26, 2008

Related U.S. Application Data
(63) Continuation of application No. PCT/EP2006/012480, filed on Dec. 22, 2006.

(30) Foreign Application Priority Data
Dec. 28, 2005 (DE) 10 2005 062 985.7

(51) Int. Cl. A61K 31/165; C07C 233/02; A61K 31/44; C07D 211/70; C07D 333/22; A61K 31/38; A61K 31/40; C07D 209/18

(52) U.S. Cl. 514/357; 564/182; 514/617; 546/337; 549/77; 514/438; 514/419; 548/495

(57) ABSTRACT

The present invention relates to substituted bis(hetero)aromatic N-ethylpropiolamides, methods for the production thereof, medicaments containing these compounds and the use thereof for the production of medicaments.

The present invention relates to substituted bis(hetero)aromatic N-ethylpropiolamides, methods for the production thereof, medicaments containing these compounds and the use thereof for the production of medicaments.

Pain is one of the basic symptoms in clinics. There is a worldwide need for effective pain treatments. The urgency of the requirement for providing tailored and targeted treatment of chronic and non-chronic pain, this being taken to mean pain treatment which is effective and satisfactory from the patient's standpoint, is also evident from the large number of scientific papers relating to applied analgesia and to basic nociception research which have appeared in recent times.

Traditional opioids, such as morphine, are effective in the treatment of severe to very severe pain, but often lead to undesired side effects such as respiratory depression, vomiting, sedation, constipation or development of tolerance. Moreover, they are often not sufficiently effective in the case of neuropathic pain, from which tumour patients in particular often suffer.

One object of the present invention was therefore to provide new compounds which are particularly suitable as active pharmaceutical substances in medicaments, preferably in medicaments for the treatment of pain.

It was surprisingly found that the substituted bis(hetero)aromatic N-ethylpropiolamides of the general formula I indicated below are suitable for mGluR5 receptor regulation and can therefore be used in particular as active pharmaceutical substances in medicaments for the prevention and/or treatment of disorders or illnesses connected to these receptors or processes.

One subject matter of the present invention is therefore substituted bis(hetero)aromatic N-ethylpropiolamides of the general formula I,

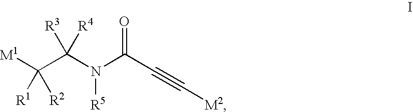

in which

### I.)

M¹ denotes phenyl, which can be unsubstituted or substituted with 1, 2, 3, 4 or 5 substituents mutually independently selected from the group comprising F, Cl, Br, I, —CN, —NO₂, —OH, —SH, —NH₂, —C(═O)—OH, methyl, ethyl, n-propyl, isopropyl, n-butyl, isobutyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, —S(═O)₂—C₁₋₅-alkyl, —S(═O)—C₁₋₅-alkyl, —NH—C₁₋₅-alkyl, —N(C₁₋₅-alkyl)₂, —C(═O)—O—C₁₋₅-alkyl, —C(═O)—O-phenyl, —C(═O)—H; —C(═O)—C₁₋₅-alkyl, —CH₂—O—C(═O)-phenyl, —O—C(═O)-phenyl, —O—C(═O)—C₁₋₅-alkyl, —NH—C(═O)—C₁₋₅-alkyl, —C(═O)—NH₂, —C(═O)—NH—C₁₋₅-alkyl, —C(═O)—N(C₁₋₅-alkyl)₂, —C(═O)—N(C₁₋₅-alkyl)(phenyl), —C(═O)—NH-phenyl, cyclopropyl, cyclobutyl, cyclopentyl, cyclohexyl, pyrazolyl, phenyl, furyl (furanyl), thiazolyl, thiadiazolyl, thiophenyl (thienyl), benzyl and phenethyl, whereby the above-mentioned C₁₋₅-alkyl residues can in each case be linear or branched and the cyclic substituents or the cyclic residues of these substituents themselves can be substituted with optionally 1, 2, 3, 4 or 5 substituents mutually independently selected from the group comprising F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O—phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂ and —S—CH₂F;
and M² denotes phenyl, which is substituted with 1, 2, 3, 4 or 5 substituents mutually independently selected from the group comprising F, Cl, Br, I, —CN, —NO₂, —OH, —SH, —NH₂, —C(═O)—OH, methyl, ethyl, n-propyl, isopropyl, n-butyl, isobutyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, —S(═O)₂—C₁₋₅-alkyl, —S(═O)—C₁₋₅-alkyl, —NH—C₁₋₅-alkyl, —N(C₁₋₅alkyl)₂, —C(═O)—O—C₁₋₅-alkyl, —C(═O)—O-phenyl, —C(═O)—H; —C(═O)—C₁₋₅-alkyl, —CH₂—O—C(═O)-phenyl, —O—C(═O)-phenyl, —O—C(═O)—C₁₋₅-alkyl, —NH—C(═O)—C₁₋₅-alkyl, —C(═O)—NH₂, —C(═O)—NH—C₁₋₅-alkyl, —C(═O)—N(C₁₋₅-alkyl)₂, —C(═O)—N(C₁₋₅-alkyl)(phenyl), —C(═O)—NH-phenyl, cyclopropyl, cyclobutyl, cyclopentyl, cyclohexyl, pyrazolyl, phenyl, furyl (furanyl), thiazolyl, thiadiazolyl, thiophenyl (thienyl), benzyl and phenethyl, whereby the above-mentioned C₁₋₅-alkyl residues can in each case be linear or branched and the cyclic substituents or the cyclic residues of these substituents themselves can be substituted with optionally 1, 2, 3, 4 or 5 substituents mutually independently selected from the group comprising F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂ and —S—CH₂F;
or M² denotes unsubstituted or substituted heteroaryl or unsubstituted or substituted naphthyl or denotes an an unsubstituted or substituted phenyl residue, which can be condensed (annelated) with unsubstituted or substituted 5- to 7-membered heterocycloalkyl or with unsubstituted or substituted C₅₋₇-cycloalkyl;

### or II.)

M¹ denotes unsubstituted or substituted heteroaryl or unsubstituted or substituted naphthyl or an unsubstituted or substituted phenyl residue, which can be condensed (annelated) with unsubstituted or substituted 5- to 7-membered heterocycloalkyl or with unsubstituted or substituted C₅₋₇-cycloalkyl;
and M² denotes unsubstituted or substituted heteroaryl or unsubstituted or substituted naphthyl or an unsubstituted or substituted phenyl residue, which can be condensed (annelated) with unsubstituted or substituted 5- to 7-membered heterocycloalkyl or with unsubstituted or substituted C₅₋₇-cycloalkyl;
or M² denotes phenyl, which can be unsubstituted or substituted with 1, 2, 3, 4 or 5 substituents mutually independently selected from the group comprising F, Cl, Br, I, —CN, —NO₂, —OH, —SH, —NH₂, —C(═O)—OH, methyl, ethyl, n-propyl, isopropyl, n-butyl, isobutyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, —S(═O)₂—C₁₋₅-alkyl, —S(═O)—C₁₋₅-alkyl, —NH—C₁₋₅-alkyl, —N(C₁₋₅alkyl)₂, —C(═O)—O—C₁₋₅-alkyl, —C(═O)—O-phenyl, —C(═O)—H; —C(═O)—C₁₋₅-alkyl, —CH₂—O—C(═O)-phenyl, —O—C(═O)-phenyl, —O—C(═O)—C₁₋₅-alkyl, —NH—C(═O)—C₁₋₅-alkyl, —C(═O)—NH₂, —C(═O)—NH—C₁₋₅-alkyl, —C(═O)—N(C₁₋₅-alkyl)₂, —C(═O)—N(C₁₋₅-alkyl)(phenyl), —C(═O)—NH-phenyl, cyclopropyl, cyclobutyl, cyclopentyl, cyclohexyl, pyrazolyl, phenyl, furyl (furanyl), thiazolyl, thiadiazolyl, thiophenyl (thienyl), benzyl and phenethyl, whereby the above-mentioned C₁₋₅-alkyl residues can in each case be linear or branched and the cyclic substituents or the cyclic residues of these substituents themselves can be substituted with optionally 1, 2, 3, 4 or 5 substituents mutually independently selected from the group comprising F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂ and —S—CH₂F;
and in each case
R¹ and R², mutually independently, in each case denote H; F; Cl; Br; I; —NO₂; —CN; —NH₂; —OH; —SH; —O—R⁶; —S—R⁷; —NH—R⁸; —NR⁹R¹⁰; unsubstituted or substituted alkyl, alkenyl or alkynyl; unsubstituted or substituted heteroalkyl, heteroalkenyl or heteroalkynyl; unsubstituted or substituted cycloalkyl or cycloalkenyl; unsubstituted or substituted heterocycloalkyl or heterocycloalkenyl; unsubstituted or substituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or substituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; or unsubstituted or substituted -(heteroalkylene)-heterocycloalkyl, -(heteroalkenylene)-heterocycloalkyl, -(heteroalkylene)-heterocycloalkenyl or -(heteroalkenylene)-heterocycloalkenyl or aryl;
or R¹ and R² jointly denote an oxo group (═O);
R³ and R⁴, mutually independently, in each case denote H; F; Cl; Br; I; —NO₂; —CN; —NH₂; —OH; —SH; —C(═O)—OH; —C(═O)—H; —NH—C(═O)—H; —O—R⁶; —S—R⁷; —NH—R⁸; —NR⁹R¹⁰; —C(═O)—R¹¹; —C(═O)—O—R¹²; —O—C(═O)—R¹³; —NH—C(═O)—R¹⁴; —NR¹⁵—C(═O)—R¹⁶; —C(═O)—NH₂; —C(═O)—NH—R¹⁷; —C(═O)—NR¹⁸R¹⁹; —S(═O)—R²⁰; —S(═O)₂—R²¹; —NH—S(═O)₂—R²²; —NR²³—S(═O)₂—R²⁴; unsubstituted or substituted alkyl, alkenyl or alkynyl; unsubstituted or substituted heteroalkyl, heteroalkenyl or heteroalkynyl; unsubstituted or substituted cycloalkyl or cycloalkenyl; unsubstituted or substituted heterocycloalkyl or heterocycloalkenyl; unsubstituted or substituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or substituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; or unsubstituted or substituted -(heteroalkylene)-heterocycloalkyl, -(heteroalkenylene)-heterocycloalkyl, -(heteroalkylene)-heterocycloalkenyl or -(heteroalkenylene)-heterocycloalkenyl;
R⁵ denotes H; —C(═O)—O—R¹²; —C(═O)—NH₂; —C(═O)—NH—R¹⁷; —C(═O)—NR¹⁸R¹⁹; —S(═O)—R²⁰; —S(═O)₂—R²¹; unsubstituted or substituted alkyl, alkenyl or alkynyl; unsubstituted or substituted heteroalkyl, heteroalkenyl or heteroalkynyl; unsubstituted or substituted cycloalkyl or cycloalkenyl; unsubstituted or substituted heterocycloalkyl or heterocycloalkenyl; unsubstituted or substituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or substituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; unsubstituted or substituted -(heteroalkylene)-heterocycloalkyl, -(heteroalkenylene)-heterocycloalkyl, -(heteroalkylene)-heterocycloalkenyl or -(heteroalkenylene)-heterocycloalkenyl; unsubstituted or substituted aryl; unsubstituted or substituted heteroaryl; unsubstituted or substituted -(alkylene)-aryl, whereby aryl can be unsubstituted or substituted with 1, 2, 3, 4 or 5 substituents mutually independently selected from the group comprising F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, —S(═O)₂—C₁₋₅-alkyl, —S(═O)—C₁₋₅-alkyl, —NH—C₁₋₅-alkyl, N(C₁₋₅alkyl)₂, —C(═O)—O—C₁₋₅-alkyl, —C(═O)—O-phenyl, —C(═O)—H, —C(═O)—C₁₋₅-alkyl, —CH₂—O—C(═O)-phenyl, —O—C(═O)-phenyl, —O—C(═O)—C₁₋₅-alkyl, —NH—C(═O)—C₁₋₅-alkyl, —C(═O)—NH₂, —C(═O)—NH—C₁₋₅-alkyl, —C(═O)—N(C₁₋₅-alkyl)₂, —C(═O)—N(C₁₋₅-alkyl)(phenyl), —C(═O)—NH-phenyl, cyclopropyl, cyclobutyl, cyclopentyl, cyclohexyl, pyrazolyl, phenyl, furyl (furanyl), thiazolyl, thiadiazolyl, thiophenyl (thienyl), benzyl and phenethyl; -(alkenylene)-aryl, -(alkynylene)-aryl, -(heteroalkylene)-aryl or -(heteroalkenylene)-aryl; or unsubstituted or substituted -(alkylene)-heteroaryl, -(alkenylene)-heteroaryl, -(alkynylene)-heteroaryl, -(heteroalkylene)-heteroaryl or -(heteroalkenylene)-heteroaryl;
and, provided that M² denotes unsubstituted or substituted heteroaryl or unsubstituted or substituted naphthyl or an unsubstituted or substituted phenyl residue, which can be condensed (annelated) with unsubstituted or substituted 5- to 7-membered heterocycloalkyl or with unsubstituted or substituted C₅₋₇-cycloalkyl, R⁵ additionally can denote —C(═O)—R¹¹;
and R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³ and R²⁴, mutually independently, in each case denote unsubstituted or substituted alkyl, alkenyl or alkynyl; unsubstituted or substituted heteroalkyl, heteroalkenyl or heteroalkynyl; unsubstituted or substituted cycloalkyl or cycloalkenyl; unsubstituted or substituted heterocycloalkyl or heterocycloalkenyl; unsubstituted or substituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or substituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; unsubstituted or substituted -(heteroalkylene)-heterocycloalkyl, -(heteroalkenylene)-heterocycloalkyl, -(heteroalkylene)-heterocycloalkenyl; or -(heteroalkenylene)-heterocycloalkenyl; unsubstituted or substituted aryl; unsubstituted or substituted heteroaryl; unsubstituted or substituted -(alkylene)-aryl, -(alkenylene)-aryl, -(alkynylene)-aryl, -(heteroalkylene)-aryl or -(heteroalkenylene)-aryl; or unsubstituted or substituted -(alkylene)-heteroaryl, -(alkenylene)-heteroaryl, -(alkynylene)-heteroaryl, -(heteroalkylene)-heteroaryl or -(heteroalkenylene)-heteroaryl;
in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

The following compounds are preferably excluded
a. 2-(N-(2-methoxy-2-oxoethyl)-3-(naphthalene-2-yl)propiolamido)-3-phenylpropanic acid methyl ester,
b. Disodium salt of 2-(N-(carboxymethyl)-3-(naphthalene-2-yl)propiolamido)-3-phenylpropanic acid,
c. 2-(N-(2-methoxy-2-oxoethyl)-3-(naphthalene-2-yl)propiolamido)-3-p-tolylpropanic acid methyl ester,
d. Disodium salt of 2-(N-(carboxymethyl)-3-(naphthalene-2-yl)propiolamido)-3-p-tolylpropanic acid,
e. 3-(4-ethylphenyl)-2-(N-(2-methoxy-2-oxoethyl)-3-(naphthalene-2-yl)propiolamido)propanic acid methyl ester,
f. Disodium salt of 2-(N-(carboxymethyl)-3-(naphthalene-2-yl)propiolamido)-3-(4-ethylphenyl)propanic acid,
g. 3-(4-isopropylphenyl)-2-(N-(2-methoxy-2-oxoethyl)-3-(naphthalene-2-yl)propiolamido)propanic acid methyl ester,
h. Disodium salt of 2-(N-(carboxymethyl)-3-(naphthalene-2-yl)propiolamido)-3-(4-isopropylphenyl)propanic acid,
i. 3-(biphenyl-4-yl)-2-(N-(2-methoxy-2-oxoethyl)-3-(naphthalene-2-yl)propiolamido)propanic acid methyl ester,
j. Disodium salt of 3-(biphenyl-4-yl)-2-(N-(carboxymethyl)-3-(naphthalene-2-yl)propiolamido)propanic acid,
k. 3-(4-cyclohexylphenyl)-2-(N-(2-methoxy-2-oxoethyl)-3-(naphthalene-2-yl)propiolamido)propanic acid methyl ester,
l. Disodium salt of 2-(N-(carboxymethyl)-3-(naphthalene-2-yl)propiolamido)-3-(4-cyclohexylphenyl)propanic acid,
m. 2-(N-(2-methoxy-2-oxoethyl)-3-(naphthalene-2-yl)propiolamido)-3-(naphthalene-2-yl)propanic acid methyl ester and
n. Disodium salt of 2-(N-(carboxymethyl)-3-(naphthalene-2-yl)propiolamido)-3-(naphthalene-2-yl)propanic acid.

An alkyl residue in the position of the substituent R⁵ is likewise preferably unsubstituted or substituted with 1, 2 or 3 substituents mutually independently selected from the group comprising F, Cl, Br, I, —NO₂, —CN, —OH, —SH, —NH₂, —N(CH₃)₂, —N(C₂H₅)₂ and —N(CH₃)(C₂H₅).

The term “alkyl” encompasses, within the meaning of the present invention, acyclic saturated hydrocarbon residues which can be branched or straight-chained and unsubstituted or at least monosubstituted with, as in the case of C₁₋₁₂-alkyl, 1 to 12 (i.e. 1, 2, 3, 4, 5, 6, 7, 8, 9, 10, 11 or 12) C-atoms or with, as in the case of C₁₋₆-alkyl, 1 to 6 (i.e. 1, 2, 3, 4, 5 or 6) C-atoms. Provided that one or more of the substituents denote an alkyl residue or have an alkyl residue, which is monosubstituted or multiply substituted, this residue can preferably be substituted with optionally 1, 2, 3, 4 or 5, particularly preferably with 1, 2 or 3 substituents mutually independently selected from the group comprising F, Cl, Br, I, —NO₂, —CN, —OH, —SH, —NH₂, —N(C₁₋₅-alkyl)₂, —N(C₁₋₅-alkyl)(phenyl), —N(C₁₋₅-alkyl)(CH₂-phenyl), —N(C₁₋₅-alkyl)(CH₂—CH₂-phenyl), —C(═O)—C₁₋₅-alkyl, —C(═O)-phenyl, —C(═O)-phenyl, —C(═S)—C₁₋₅-alkyl, —C(═S)-phenyl, —C(═O)—OH, —C(═O)—O—C₁₋₅-alkyl, —C(═O)—O-phenyl, —C(═O)—NH₂, —C(═O)—NH—C₁₋₅-alkyl, —C(═O)—N(C₁₋₅-alkyl)₂, —S(═O)—C₁₋₅-alkyl, —S(═O)-phenyl, —S(═O)₂—C₁₋₅-alkyl, —S(═O)₂-phenyl, —S(═O)₂—NH₂ and —SO₃H, whereby the above-mentioned C₁₋₅-alkyl residues can in each case be linear or branched and the above-mentioned phenyl residues can be substituted preferably with 1, 2, 3, 4 or 5 substituents mutually independently selected from the group comprising F, Cl, Br, I, —CN, —CF₃, —OH, —NH₂, —O—CF₃, —SH, —O—CH₃, —O—C₂H₅, —O—C₃H₇, methyl, ethyl, n-propyl, isopropyl, n-butyl, 2-butyl, isobutyl and tert-butyl. Particularly preferred substituents can be mutually independently selected from the group comprising F, Cl, Br, I, —NO₂, —CN, —OH, —SH, —NH₂, —N(CH₃)₂, —N(C₂H₅)₂ and —N(CH₃)(C₂H₅).

By way of example, methyl, ethyl, n-propyl, isopropyl, n-butyl, isobutyl, 2-butyl, tert-butyl, n-pentyl, 2-pentyl, 3-pentyl, iso-pentyl, neo-pentyl, n-hexyl, 2-hexyl, 3-hexyl, n-heptyl, n-octyl, —C(H)(C₂H₅)₂, —C(H)(n-C₃H₇)₂ and —CH₂—CH₂—C(H)(CH₃)—(CH₂)₃—CH₃ are cited as suitable C₁₋₁₂-alkyl residues which can be unsubstituted or monosubstituted or multiply substituted. By way of example, methyl, ethyl, n-propyl, isopropyl, n-butyl, isobutyl, 2-butyl, tert-butyl, n-pentyl, 2-pentyl, 3-pentyl, iso-pentyl, neo-pentyl, n-hexyl, 2-hexyl and 3-hexyl are cited as suitable C₁₋₆-alkyl residues.

Multiply substituted alkyl residues refer to such alkyl residues which are multiply substituted, preferably twice or three times, either at different or at the same C-atoms, for example, three times at the same C-atom as in the case of —CF₃ or at various points as in the case of —(CHCl)—(CH₂F). The multiple substitution can be performed with the same or with different substituents. By way of example, —CF₃, —CF₂H, —CFH₂, —(CH₂)—OH, —(CH₂)—NH₂, —(CH₂)—CN, —(CH₂)—(CF₃), —(CH₂)—(CHF₂), —(CH₂)—(CH₂F), —(CH₂)—(CH₂)—OH, —(CH₂)—(CH₂)—NH₂, —(CH₂)—(CH₂)—CN, —(CF₂)—(CF₃), —(CH₂)—(CH₂)—(CF₃) and —(CH₂)—(CH₂)—(CH₂)—OH are cited as suitable substituted alkyl residues.

The term “alkenyl” encompasses, within the meaning of the present invention, acyclic unsaturated hydrocarbon residues which can be branched or straight-chained and unsubstituted or at least monosubstituted and have at least one double-bond, preferably 1, 2 or 3 double-bonds, with as in the case of C₂₋₁₂-alkenyl 2 to 12 (i.e. 2, 3, 4, 5, 6, 7, 8, 9, 10, 11 or 12) C-atoms or with as in the case of C₂₋₆-alkenyl 2 to 6 (i.e. 2, 3, 4, 5 or 6) C-atoms. Provided that one or more of the substituents denote an alkenyl residue or have an alkenyl residue which is monosubstituted or multiply substituted, this residue can preferably be substituted with optionally 1, 2, 3, 4 or 5, particularly preferably with 1, 2 or 3 substituents mutually independently selected from the group comprising F, Cl, Br, I, —NO₂, —CN, —OH, —SH, —NH₂, —N(C₁₋₅-alkyl)₂, —N(C₁₋₅-alkyl)(phenyl), —N(C₁₋₅-alkyl)(CH₂-phenyl), —N(C₁₋₅-alkyl)(CH₂—CH₂-phenyl), —C(═O)—H, —C(═O)—C₁₋₅-alkyl, —C(═O)-phenyl, —C(═S)—C₁₋₅-alkyl, —C(═S)-phenyl, —C(═O)—OH, —C(═O)—O—C₁₋₅-alkyl, —C(═O)—O-phenyl, —C(═O)—NH₂, —C(═O)—NH—C₁₋₅-alkyl, —C(═O)—N(C₁₋₅-alkyl)₂, —S(═O)—C₁₋₅-alkyl, —S(═O)-phenyl, —S(═O)₂—C₁₋₅-alkyl, —S(═O)₂-phenyl, —S(═O)₂—NH₂ and —SO₃H, whereby the above-mentioned C₁₋₅-alkyl residues can in each case be linear or branched and the above-mentioned phenyl residues can preferably be substituted with 1, 2, 3, 4 or 5 substituents mutually independently selected from the group comprising F, Cl, Br, I, —CN, —CF₃, —OH, —NH₂, —O—CF₃, —SH, —O—CH₃, —O—C₂H₅, —O—C₃H₇, methyl, ethyl, n-propyl, isopropyl, n-butyl, 2-butyl, isobutyl and tert-butyl. Particularly preferred substituents can be selected mutually independently from the group comprising F, Cl, Br, I, —NO₂, —CN, —OH, —SH, —NH₂, —N(CH₃)₂, —N(C₂H₅)₂ and —N(CH₃)(C₂H₅).

By way of example, ethenyl, 1-propenyl, 2-propenyl, 1-butenyl, 2-butenyl, 3-butenyl, 1-pentenyl, 2-pentenyl, 3-pentenyl, 4-pentenyl, hexenyl, —CH═CH—CH═CH—CH₃ and —CH₂—CH₂—CH═CH₂ are cited as suitable C₂₋₁₂-alkenyl residues.

Multiply substituted alkenyl residues refer to such alkenyl residues which are multiply substituted, preferably twice, at different or at the same C-atoms, for example, twice at the same C-atom as in the case of —CH═CCl₂ or at various points as in the case of —CCl═CH—(CH₂)—NH₂. The multiple substitution can be performed with the same or with different substituents. By way of example, —CH═CH—(CH₂)—OH, —CH═CH—(CH₂)—NH₂ and —CH═CH—CN are cited as suitable substituted alkenyl residues.

The term “alkynyl” encompasses, within the meaning of the present invention, acyclic unsaturated hydrocarbon residues which can be branched or straight-chained and unsubstituted or at least monosubstituted and have at least one triple-bond, preferably 1 or 2 triple-bonds, with as in the case of C₂₋₁₂-alkynyl 2 to 12 (i.e. 2, 3, 4, 5, 6, 7, 8, 9, 10, 11 or 12) C-atoms or with as in the case of C₂₋₆-alkynyl 2 to 6 (i.e. 2, 3, 4, 5 or 6) C-atoms. Provided that one or more of the substituents denote an alkynyl residue or have an alkynyl residue which is monosubstituted or multiply substituted, this residue can preferably be substituted with optionally 1, 2, 3, 4 or 5, particularly preferably with optionally 1 or 2 substituents mutually independently selected from the group comprising F, Cl, Br, I, —NO₂, —CN, —OH, —SH, —NH₂, —N(C₁₋₅-alkyl)₂, —N(C₁₋₅-alkyl)(phenyl), —N(C₁₋₅-alkyl)(CH₂-phenyl), —N(C₁₋₅-alkyl)(CH₂—CH₂-phenyl), —C(═O)—H, —C(═O)—C₁₋₅-alkyl, —C(═O)-phenyl, —C(═S)—C₁₋₅-alkyl, —C(═S)-phenyl, —C(═O)—OH, —C(═O)—O—C₁₋₅-alkyl, —C(═O)—O-phenyl, —C(═O)—NH₂, —C(═O)—NH—C₁₋₅-alkyl, —C(═O)—N(C₁₋₅-alkyl)₂, —S(═O)—C₁₋₅-alkyl, —S(═O)-phenyl, —S(═O)₂—C₁₋₅-alkyl, —S(═O)₂-phenyl, —S(═O)₂—NH₂ and —SO₃H, whereby the above-mentioned C₁₋₅-alkyl residues can in each case be linear or branched and the above-mentioned phenyl residues can preferably be substituted with 1, 2, 3, 4 or 5 substituents mutually independently selected from the group comprising F, Cl, Br, I, —CN, —CF₃, —OH, —NH₂, —O—CF₃, —SH, —O—CH₃, —O—C₂H₅, —O—C₃H₇, methyl, ethyl, n-propyl, isopropyl, n-butyl, 2-butyl, isobutyl and tert-butyl. Particularly preferred substituents can be selected mutually independently from the group comprising F, Cl, Br, I, —NO₂, —CN, —OH, —SH, —NH₂, —N(CH₃)₂, —N(C₂H₅)₂ and —N(CH₃)(C₂H₅).

By way of example, ethynyl, 1-propynyl, 2-propynyl, 1-butynyl, 2-butynyl, 3-butynyl, 1-pentynyl, 2-pentynyl, 3-pentynyl, 4-pentynyl and hexynyl are cited as suitable C₂₋₁₂-alkynyl residues.

Multiply substituted alkynyl residues refer to those alkynyl residues which are either multiply substituted at different C-atoms, for example, twice at different C-atoms as in the case of —CHCl—C≡CCl. By way of example, —C≡C—F, —C≡C—Cl and —C≡C—I are cited as suitable substituted alkynyl residues.

The term “heteroalkyl” denotes an alkyl residue as described above in which one or more C-atoms have been replaced in each case by a heteroatom mutually independently selected from the group comprising oxygen, sulphur and nitrogen (NH). Heteroalkyl residues can preferably have 1, 2 or 3 heteroatom(s), mutually independently, selected from the group comprising oxygen, sulphur and nitrogen (NH) as the chain member(s). Heteroalkyl residues can preferably be 2- to 12-membered, particularly preferably 2- to 6-membered.

By way of example, —CH₂—O—CH₃, —CH₂—O—C₂H₅, —CH₂—O—CH(CH₃)₂, —CH₂—O—C(CH₃)₃, —CH₂—S—CH₃, —CH₂—S—C₂H₅, —CH₂—S—CH(CH₃)₂, —CH₂—S—C(CH₃)₃, —CH₂—NH—CH₃, —CH₂—NH—C₂H₅, —CH₂—NH—CH(CH₃)₂, —CH₂—NH—C(CH₃)₃, —CH₂—CH₂—O—CH₃, —CH₂—CH₂—O—C₂H₅, —CH₂—CH₂—O—CH(CH₃)₂, —CH₂—CH₂—O—C(CH₃)₃, —CH₂—CH₂—S—CH₃, —CH₂—CH₂—S—C₂H₅, —CH₂—CH₂—S—CH(CH₃)₂, —CH₂—CH₂—S—C(CH₃)₃, —CH₂—CH₂—NH—CH₃, —CH₂—CH₂—NH—C₂H₅, —CH₂—CH₂—NH—CH(CH₃)₂, —CH₂—CH₂—NH—C(CH₃)₃, —CH₂—S—CH₂—O—CH₃, —CH₂—O—CH₂—O—C₂H₅, —CH₂—O—CH₂—O—CH(CH₃)₂, —CH₂—S—CH₂—O—C(CH₃)₃, —CH₂—O—CH₂—S—CH₃, —CH₂—O—CH₂—S—C₂H₅, —CH₂—O—CH₂—S—CH(CH₃)₂, —CH₂—NH—CH₂—S—C(CH₃)₃, —CH₂—O—CH₂—NH—CH₃, —CH₂—O—CH₂—NH—C₂H₅, —CH₂—O—CH₂—NH—CH(CH₃)₂, —CH₂—S—CH₂—NH—C(CH₃)₃ and —CH₂—CH₂—C(H)(CH₃)—(CH₂)₃—CH₃ are cited as suitable heteroalkyl residues which can be unsubstituted or monosubstituted or multiply substituted.

By way of example, —(CH₂)—O—(CF₃), —(CH₂)—O—(CHF₂), —(CH₂)—O—(CH₂F), —(CH₂)—S—(CF₃), —(CH₂)—S—(CHF₂), —(CH₂)—S—(CH₂F), —(CH₂)—(CH₂)—O—(CF₃), —(CF₂)—O—(CF₃), —(CH₂)—(CH₂)—S—(CF₃) and —(CH₂)—(CH₂)—(CH₂)—O—(CF₃) are cited as suitable substituted heteroalkyl residues.

The term “heteroalkenyl” denotes an alkenyl residue as described above in which one or more C-atoms have been replaced in each case by a heteroatom mutually independently selected from the group comprising oxygen, sulphur and nitrogen (NH). Heteroalkenyl residues can preferably have 1, 2 or 3 heteroatom(s), mutually independently, selected from the group comprising oxygen, sulphur and nitrogen (NH) as the chain member(s). Heteroalkenyl residues can preferably be 2- to 12-membered, particularly preferably 2- to 6-membered.

By way of example, —CH₂—O—CH═CH₂, —CH═CH—O—CH═CH—CH₃, —CH₂—CH₂—O—CH═CH₂, —CH₂—S—CH═CH₂, —CH═CH—S—CH═CH—CH₃, —CH₂—CH₂—S—CH═CH₂, —CH₂—NH—CH═CH₂, —CH═CH—NH—CH═CH—CH₃ and —CH₂—CH₂—NH—CH═CH₂ are cited as suitable heteroalkenyl residues.

By way of example, —CH₂—O—CH═CH—(CH₂)—OH, —CH₂—S—CH═CH—(CH₂)—NH₂ and —CH₂—NH—CH═CH—CN are cited as suitable substituted heteroalkenyl residues.

The term “heteroalkynyl” denotes an alkynyl residue as described above in which one or more C-atoms have been replaced in each case by a heteroatom mutually independently selected from the group comprising oxygen, sulphur and nitrogen (NH). Heteroalkynyl residues can preferably have 1, 2 or 3 heteroatom(s), mutually independently, selected from the group comprising oxygen, sulphur and nitrogen (NH) as the chain member(s). Heteroalkynyl residues can preferably be 2- to 12-membered, particularly preferably 2- to 6-membered.

By way of example, —CH₂—O—C≡CH, —CH₂—CH₂—O—C≡CH, —CH₂—O—C≡C—CH₃, —CH₂—CH₂—O—C≡C—CH₃, —CH₂—S—C≡CH, —CH₂—CH₂—S—C≡CH, —CH₂—S—C≡C—CH₃, —CH₂—CH₂—S—C≡C—CH₃ are cited as suitable heteroalkynyl residues.

By way of example, —CH₂—O—C═C≡C—Cl, —CH₂—CH₂—O—C≡C—I, —CHF—O—C≡C—CH₃, —CHF—CH₂—O—C≡C—CH₃, —CH₂—S—C≡C—Cl, —CH₂—CH₂—S—C≡C—Cl, —CHF—S—C≡C—CH₃, —CHF—CH₂—S—C≡C—CH₃ are cited as suitable substituted heteroalkynyl residues.

The term “cycloalkyl” means, in terms of the present invention, a cyclic saturated hydrocarbon residue with preferably 3, 4, 5, 6, 7, 8 or 9 C-atoms, particularly preferably with 3, 4, 5, 6 or 7 C-atoms, very particularly preferably with 5 or 6 C-atoms, whereby the residue can be unsubstituted or monosubstituted or multiply identically or differently substituted.

By way of example, cyclopropyl, cyclobutyl, cyclopentyl, cyclohexyl, cycloheptyl, cyclooctyl and cyclononyl are cited as suitable C₃₋₉-cycloalkyl residues which can be unsubstituted or monosubstituted or multiply substituted. Cyclopropyl, cyclobutyl, cyclopentyl, cyclohexyl and cycloheptyl are cited as suitable C₃₋₇-cycloalkyl residues.

The term “cycloalkenyl” means, in terms of the present invention, a cyclic unsaturated hydrocarbon residue with preferably 3, 4, 5, 6, 7, 8 or 9 C-atoms, particularly preferably with 3, 4, 5, 6 or 7 C-atoms, very particularly preferably with 5 or 6 C-atoms, which has at least one double-bond, preferably one double-bond, and can be unsubstituted or monosubstituted or multiply identically or differently substituted.

Cyclobutenyl, cyclopentenyl, cyclohexenyl, cycloheptenyl, cyclononenyl and cyclooctenyl are cited as suitable C₃₋₉-cycloalkenyl residues which can be unsubstituted or monosubstituted or multiply substituted. Cyclopentenyl and cyclohexenyl are cited as suitable C₅₋₆-cycloalkenyl residues.

The term “heterocycloalkyl” means, in terms of the present invention, a cyclic saturated hydrocarbon residue with preferably 3, 4, 5, 6, 7, 8 or 9 C-atoms, particularly preferably with 3, 4, 5, 6 or 7 C-atoms, very particularly preferably with 5 or 6 C-atoms, in which one or more C-atoms have been replaced in each case by a heteroatom mutually independently selected from the group comprising oxygen, sulphur and nitrogen (NH). Heterocycloalkyl residues can preferably have 1, 2 or 3 heteroatom(s), mutually independently, selected from the group comprising oxygen, sulphur and nitrogen (NH) as the ring member(s). A heterocycloalkyl residue can be unsubstituted or monosubstituted or multiply identically or differently substituted. Heterocycloalkyl residues can preferably be 3- to 9-membered, particularly preferably 3- to 7-membered, very particularly preferably 5- to 7-membered.

By way of example, imidazolidinyl, tetrahydrofuranyl, tetrahydrothiophenyl, pyrrolidinyl, piperidinyl, morpholinyl, piperazinyl, thiomorpholinyl, tetrahydropyranyl, oxetanyl, azepanyl, azocanyl, diazepanyl, dithiolanyl, (1,3)-dioxolan-2-yl, isoxazolidinyl, isothioazolidinyl, pyrazolidinyl, oxazolidinyl, (1,2,4)-oxadiazolidinyl, (1,2,4)-thiadiazolidinyl, (1,2,4)-triazolidin-3-yl, (1,3,4)-thiadiazolidin-2-yl, (1,3,4)-triazolidin-1-yl, (1,3,4)-triazoldidin-2-yl, tetrahydropyridazinyl, tetrahydropyrimidinyl, tetrahydropyrazinyl, (1,3,5)-tetrahydrotriazinyl, (1,2,4)-tetrahydrotriazin-1-yl, (1,3)-dithian-2-yl and (1,3)-thiazolidinyl are cited as suitable 3- to 9-membered heterocycloalkyl residues which can be unsubstituted or monosubstituted or multiply substituted. By way of example, imidazolidinyl, tetrahydrofuranyl, tetrahydrothiophenyl, pyrrolidinyl, piperidinyl, morpholinyl, piperazinyl, thiomorpholinyl, tetrahydropyranyl, oxetanyl, azepanyl, diazepanyl and (1,3)-dioxolan-2-yl are cited as suitable 5- to 7-membered heterocycloalkyl residues.

The term “heterocycloalkenyl” means, in terms of the present invention, a cyclic unsaturated hydrocarbon residue with preferably 4, 5, 6, 7, 8 or 9 C-atoms, particularly preferably with 4, 5, 6 or 7 C-atoms, very particularly preferably with 5 or 6 C-atoms, which has at least one double-bond, preferably one double-bond, and in which one or more C-atoms have been replaced in each case by a heteroatom mutually independently selected from the group comprising oxygen, sulphur and nitrogen (NH). Heterocycloalkenyl residues can preferably have 1, 2 or 3 heteroatom(s), mutually independently, selected from the group comprising oxygen, sulphur and nitrogen (NH) as the ring member(s). A heterocycloalkenyl residue can be unsubstituted or monosubstituted or multiply identically or differently substituted. Heterocycloalkenyl residues can preferably be 4- to 9-membered, particularly preferably 4- to 7-membered, very particularly preferably 5- to 7-membered.

By way of example, (2,3)-dihydrofuranyl, (2,5)-dihydrofuranyl, (2,3)-dihydrothienyl, (2,5)-dihydrothienyl, (2,3)-dihydropyrrolyl, (2,5)-dihydropyrrolyl, (2,3)-dihydroisoxazolyl, (4,5)-dihydroisoxazolyl, (2,5)-dihydroisothiazolyl, (2,3)-dihydropyrazolyl, (4,5)-dihydropyrazolyl, (2,5)-dihydropyrazolyl, (2,3)-dihydrooxazolyl, (4,5)-dihydrooxazolyl, (2,5)-dihydrooxazolyl, (2,3)-dihydrothiazolyl, (4,5)-dihydrothiazolyl, (2,5)-dihydrothiazolyl, (2,3)-dihydroimidazolyl, (4,5)-dihydroimidazolyl, (2,5)-dihydroimidazolyl, (3,4,5,6)-tetrahydropyridine-2-yl, (1,2,5,6)-tetrahydropyridine-1-yl, (1,2)-dihydropyridine-1-yl, (1,4)-dihydropyridine-1-yl, dihydropyranyl and (1,2,3,4)-tetrahydropyridine-1-yl are cited as suitable heterocycloalkenyl residues or as suitable 5- to 7-membered heterocycloalkenyl residues which can be unsubstituted or monosubstituted or multiply substituted.

Cycloalkyl residue, heterocycloalkyl residue, cycloalkenyl residue or heterocyclalkenyl residue can, within the meaning of the present invention, be condensed (annelated) with an unsubstituted or at least monosubstituted mono- or bicyclic ring system. A mono- or bicyclic ring system refers, in the context of the present invention, to mono- or bicyclic hydrocarbon residues which can be saturated, unsaturated or aromatic and can optionally have one or more heteroatoms as ring members. The rings of the above-mentioned mono- or bicyclic ring systems are preferably respectively 4-, 5- or 6-membered and can have in each case preferably optionally 0, 1, 2, 3, 4 or 5 heteroatom(s), particularly preferably optionally 0, 1 or 2 heteroatom(s) as the ring member(s), which are mutually independently selected from the group comprising oxygen, nitrogen and sulphur. Provided that one bicyclic ring system is present, the different rings can, in each case mutually independently, have a different degree of saturation, i.e. be saturated, unsaturated or aromatic.

Provided that one or more of the substituents have a monocyclic or bicyclic ring system which is monosubstituted or multiply substituted, this ring system can be preferably substituted with optionally 1, 2, 3, 4 or 5, particularly preferably with optionally 1, 2 or 3 substituents, which can be mutually independently selected from the group comprising F, Cl, Br, I, —CN, —NO₂, —OH, —SH, —NH₂, oxo (═O), thioxo (═S), —C(═O)—OH, C₁₋₅-alkyl, —C₂₋₅-alkenyl, —C₂₋₅-alkynyl, —C≡C—Si(CH₃)₃, —C≡C—Si(C₂H₅)₃, —(CH₂)—O—C₁₋₅-alkyl, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, —S(═O)₂—C₁₋₅-alkyl, —S(═O)—C₁₋₅-alkyl, —NH—C₁₋₅-alkyl, —N(C₁₋₅-alkyl)(C₁₋₅-alkyl), —C(═O)—O—C₁₋₅-alkyl, —C(═O)—H, —C(═O)—C₁₋₅-alkyl, —CH₂—O—C(═O)-phenyl, —O—C(═O)-phenyl, —NH—S(═O)₂—C₁₋₅-alkyl, —NH—C(═O)—C₁₋₅-alkyl, —C(═O)—NH₂, —C(═O)—NH—C₁₋₅-alkyl, —C(═O)—N(C₁₋₅-alkyl)₂, pyrazolyl, phenyl, furyl (furanyl), thiadiazolyl, thiophenyl (thienyl) and benzyl, whereby the above-mentioned C₁₋₅-alkyl residues can in each case be linear or branched and the cyclic substituents or the cyclic residues of these substituents themselves can in each case be substituted with optionally 1, 2, 3, 4 or 5, preferably with optionally 1, 2, 3 or 4 substituents mutually independently selected from the group comprising F, Cl, Br, I, —CN, —CF₃, —OH, —NH₂, —O—CF₃, —SH, —O—C₁₋₅-alkyl, —O-phenyl, —O—CH₂-phenyl, —(CH₂)—O—C₁₋₅-alkyl, —S—C₁₋₅-alkyl, —S-phenyl, —S—CH₂-phenyl, —C₁₋₅-alkyl, —C₂₋₅-alkenyl, —C₂₋₅-alkynyl, —CδC—Si(CH₃)₃, —C≡C—Si(C₂H₅)₃, —C(═O)—O—C₁₋₅-alkyl and —C(═O)—CF₃.

The substituents can be particularly preferably, in each case mutually independently, selected from the group comprising F, Cl, Br, I, —CN, —NO₂, —OH, —SH, methyl, ethyl, n-propyl, isopropyl, n-butyl, isobutyl, 2-butyl, tert-butyl, n-pentyl, neo-pentyl, ethenyl, allyl, ethynyl, propynyl, —C≡C—Si(CH₃)₃, —C≡C—Si(C₂H₅)₃, —C≡C—Si(CH₃)₃, —C≡C—Si(C₂H₅)₃, —CH₂—O—CH₃, —CH₂—O—C₂H₅, —OH, —SH, —NH₂, oxo (═O), —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, pyrazolyl, phenyl, —N(CH₃)₂, —N(C₂H₅)₂, —NH—CH₃, —NH—C₂H₅, —CH₂—O—C(═O)-phenyl, —NH—S(═O)₂—CH₃, —C(═O)—O—CH₃, —C(═O)—O—C₂H₅, —C(═O)—O—C(CH₃)₃, —C(═O)—H, —C(═O)—CH₃, —C(═O)—C₂H₅, —NH—C(═O)—CH₃, —NH—C(═O)—C₂H₅, —O—C(═O)-phenyl, —C(═O)—NH₂, —C(═O)—NH—CH₃, —C(═O)—N(CH₃)₂, phenyl, furyl (furanyl), thiadiazolyl, thiophenyl (thienyl) and benzyl, whereby the cyclic substituents or the cyclic residues of these substituents themselves can be substituted with optionally 1, 2, 3, 4 or 5, preferably with 1, 2, 3 or 4 substituents mutually independently selected from the group comprising F, Cl, Br, I, —CN, —CF₃, —OH, —NH₂, —O—CF₃, —SH, —O—CH₃, —O—C₂H₅, —O—C₃H₇, methyl, ethyl, n-propyl, isopropyl, n-butyl, 2-butyl, isobutyl, tert-butyl, ethenyl, allyl, ethynyl, propynyl, —C≡C—Si(CH₃)₃, —C≡C—Si(C₂H₅)₃, —C(═O)—O—C₁₋₅-alkyl and —C(═O)—CF₃.

By way of example, (1,2,3,4)-tetrahydroquinolinyl, (1,2,3,4)-tetrahydroisoquinolinyl, (2,3)-dihydro-1H-isoindolyl, (1,2,3,4)-tetrahydronaphthyl, (2,3)-dihydro-benzo[1,4]dioxinyl, benzo[1,3]dioxolyl, (3,4)-dihydro-2H-benzo[1,4]oxazinyl and octahydro-pyrrolo[3,4-c]pyrrolyl are cited as a suitable cycloalkyl residue, heterocycloalkyl residue, cycloalkenyl residue or heterocyclalkenyl residue which can be unsubstituted or monosubstituted or multiply substituted and are condensed with a mono- or bicyclic ring system.

Provided that one or more of the substituents denote a cycloalkyl residue, heterocycloalkyl residue, cycloalkenyl residue or heterocycloalkenyl residue or have such a residue which is monosubstituted or multiply substituted, this residue can preferably be substituted with optionally 1, 2, 3, 4 or 5, particularly preferably with optionally 1, 2 or 3 substituents mutually independently selected from the group comprising F, Cl, Br, I, —CN, —CF₃, —OH, —NH₂, —O—CF₃, —SH, —O—C₁₋₅-alkyl, —O-phenyl, —O—CH₂-phenyl, —(CH₂)—O—C₁₋₅-alkyl, —S—C₁₋₅-alkyl, —S-phenyl, —S—CH₂-phenyl, —C₁₋₅-alkyl, —C₂₋₅-alkenyl, —C₂₋₅-alkynyl, —C≡C—Si(CH₃)₃, —C≡C—Si(C₂H₅)₃, —C(═O)—O—C₁₋₅-alkyl, —C(═O)—CF₃, —S(═O)₂—C₁₋₅-alkyl, —S(═O)—C₁₋₁₅-alkyl, —S(═O)₂-phenyl, oxo (═O), thioxo (═S), —N(C₁₋₅-alkyl)₂, —N(H)(C₁₋₅-alkyl), —NO₂, —S—CF₃, —C(═O)—OH, —NH—S(═O)₂—C₁₋₅-alkyl, —NH—C(═O)—C₁₋₅-alkyl, —C(═O)—H, —C(═O)—C₁₋₅-alkyl, —C(═O)—NH₂, —C(═O)—N(C₁₋₅-alkyl)₂, —C(═O)—N(H)(C₁₋₅-alkyl) and phenyl, whereby the above-mentioned C₁₋₅-alkyl residues can in each case be linear or branched and the phenyl residues can be respectively unsubstituted or substituted with 1, 2, 3, 4 or 5, preferably with 1, 2, 3 or 4 substituents mutually independently selected from the group comprising F, Cl, Br, I, —CN, —CF₃, —OH, —NH₂, —O—CF₃, —SH, —O—C₁₋₅-alkyl, —O-phenyl, —O—CH₂-phenyl, —(CH₂)—O—C₁₋₅-alkyl, —S—C₁₋₅-alkyl, —S-phenyl, —S—CH₂-phenyl, —C₁₋₅-alkyl, —C₂₋₅-alkenyl, —C₂₋₅-alkynyl, —C≡C—Si(CH₃)₃, —C≡C—Si(C₂H₅)₃, —C(═O)—O—C₁₋₅-alkyl and —C(═O)—CF₃.

The substituents can be particularly preferably, in each case mutually independently, selected from the group comprising F, Cl, Br, I, —CN, methyl, ethyl, n-propyl, isopropyl, n-butyl, 2-butyl, isobutyl, tert-butyl, ethenyl, allyl, ethynyl, propynyl, —C≡C—Si(CH₃)₃, —C≡C—Si(C₂H₅)₃, —OH, oxo, thioxo, —O—CH₃, —O—C₂H₅, —O—C₃H₇, —(CH₂)—O—CH₃, —(CH₂)—O—C₂H₅, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃, —S—C₂H₅, —S(═O)—CH₃, —S(═O)₂—CH₃, —S(═O)—C₂H₅, —S(═O)₂—C₂H₅, —NH—S(═O)₂—CH₃, —C(═O)—OH, —C(═O)—H; —C(═O)—CH₃, —C(═O)—C₂H₅, —C(═O)—N(CH₃)₂, —C(═O)—NH—CH₃, —C(═O)—NH₂, —NH—C(═O)—CH₃, —NH—C(═O)—C₂H₅, —C(═O)—O—CH₃, —C(═O)—O—C₂H₅, —C(═O)—O—C(CH₃)₃ and phenyl, whereby the phenyl residue can be substituted with 1, 2, 3, 4 or 5, preferably 1, 2 or 3 substituents mutually independently selected from the group comprising F, Cl, Br, I, —CN, —CF₃, —OH, —NH₂, —O—CF₃, —SH, —O—CH₃, —O—C₂H₅, —O—C₃H₇, methyl, ethyl, n-propyl, isopropyl, n-butyl, 2-butyl, isobutyl, tert-butyl, ethenyl, allyl, ethynyl, propynyl, —C≡C—Si(CH₃)₃, —C≡C—Si(C₂H₅)₃, —C(═O)—O—C₁₋₅-alkyl and —C(═O)—CF₃.

The term “aryl” means, in the context of the present invention, a mono- or polycyclic, preferably a mono- or bicyclic, aromatic hydrocarbon residue with preferably 6, 10 or 14 C-atoms. An aryl residue can be unsubstituted or monosubstituted or multiply identically or differently substituted. By way of example, phenyl, 1-naphthyl, 2-naphthyl and anthracenyl are cited as suitable aryl residues. An aryl residue is particularly preferably a phenyl residue.

The term “heteroaryl” means, in the context of the present invention, a monocyclic or polycyclic, preferably a mono-, bi- or tricyclic, aromatic hydrocarbon residue with preferably 5, 6, 7, 8, 9, 10, 11, 12, 13 or 14 C-atoms, particularly preferably with 5, 6, 9, 10, 13 or 14 C-atoms, very particularly preferably with 5 or 6 C-atoms, in which one or more C-atoms have in each case been replaced by a heteroatom mutually independently selected from the group comprising oxygen, sulphur and nitrogen (NH). Heteroaryl residues can preferably have 1, 2, 3, 4 or 5, particularly preferably 1, 2 or 3 heteroatom(s), mutually independently, selected from the group comprising oxygen, sulphur and nitrogen (NH) as the ring member(s). A heteroaryl residue can be unsubstituted or monosubstituted or multiply identically or differently substituted.

By way of example, indolizinyl, benzimidazolyl, tetrazolyl, triazinyl, isoxazolyl, phthalazinyl, carbazolyl, carbolinyl, diaza-naphthyl, thienyl, furyl, pyrrolyl, pyrazolyl, pyrazinyl, pyranyl, triazolyl, pyridinyl, imidazolyl, indolyl, isoindolyl, benzo[b]furanyl, benzo[b]thiophenyl, benzo[d]thiazolyl, benzodiazolyl, benzotriazolyl, benzoxazolyl, benzisoxazolyl, thiazolyl, thiadiazolyl, oxazolyl, oxadiazolyl, isoxazolyl, pyridazinyl, pyrimidinyl, indazolyl, quinoxalinyl, quinazolinyl, quinolinyl, naphthridinyl and isoquinolinyl are cited as suitable heteroaryl residues.

Aryl or heteroaryl residues can, in the context of the present invention, be condensed (annelated) with a mono- or bicyclic ring system.

(1,2,3,4)-tetrahydroquinolinyl, (1,2,3,4)-tetrahydroisoquinolinyl, (2,3)-dihydro-1H-isoindolyl, (1,2,3,4)-tetrahydronaphthyl, (2,3)-dihydro-benzo[1,4]dioxinyl, benzo[1,3]dioxolyl and (3,4)-dihydro-2H-benzo[1,4]oxazinyl are cited by way of example as aryl residues which are condensed with a mono- or bicyclic ring system.

(2,3)-dihydrobenzo[b]thiophenyl, (2,3)-dihydro-1H-indenyl, indolinyl, (2,3)-dihydrobenzofuranyl, (2,3)-dihydrobenzo[d]oxazolyl, benzo[d][1,3]dioxolyl, benzo[d][1,3]oxathiolyl, isoindolinyl, (1,3)-dihydroisobenzofuranyl, (1,3)-dihydrobenzo[c]thiophenyl, (1,2,3,4)-tetrahydronaphthyl, (1,2,3,4)-tetrahydroquinolinyl, chromanyl, thiochromanyl, (1,2,3,4)-tetrahydroisoquinolinyl, (1,2,3,4)-tetrahydroquinoxalinyl, (3,4)-dihydro-2H-benzo[b][1,4]oxazinyl, (3,4)-dihydro-2H-benzo[b][1,4]thiazinyl, (2,3)-dihydrobenzo[b][1,4]dioxinyl, (2,3)-dihydrobenzo[b][1,4]oxathiinyl, (6,7,8,9)-tetrahydro-5H-benzo[7]annulenyl, (2,3,4,5)-tetrahydro-1H-benzo[b]azepinyl and (2,3,4,5)-tetrahydro-1H-benzo[c]azepinyl are cited by way of example as phenyl residues which are condensed (annelated) with unsubstituted or substituted 5- to 7-membered heterocycloalkyl or with unsubstituted or substituted C₅₋₇-cycloalkyl.

Unless indicated otherwise, provided that one or more of the substituents denote an aryl or heteroaryl residue or have an aryl or heteroaryl residue which is monosubstituted or multiply substituted, these aryl or heteroaryl residues can preferably be substituted with optionally 1, 2, 3, 4 or 5, particularly preferably with optionally 1, 2 or 3 substituents mutually independently selected from the group comprising F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, —S(═O)₂—C₁₋₅-alkyl, —S(═O)—C₁₋₅-alkyl, —NH—C₁₋₅-alkyl, N(C₁₋₅-alkyl)₂, —C(═O)—O—C₁₋₅-alkyl, —C(═O)—O-phenyl, —C(═O)—H; —C(═O)—C₁₋₅-alkyl, —CH₂—O—C(═O)-phenyl, —O—C(═O)—C₁₋₅-alkyl, —O—C(═O)-phenyl, —NH—S(═O)₂—C₁₋₅-alkyl, —NH—C(═O)—C₁₋₅-alkyl, —C(═O)—NH₂, —C(═O)—NH—C₁₋₅-alkyl, —C(═O)—N(C₁₋₅-alkyl)₂, —C(═O)—N(C₁₋₅-alkyl)(phenyl), —C(═O)—NH-phenyl, cyclopropyl, cyclobutyl, cyclopentyl, cyclohexyl, pyrazolyl, phenyl, furyl (furanyl), thiazolyl, thiadiazolyl, thiophenyl (thienyl), benzyl and phenethyl, whereby the above-mentioned C₁₋₅-alkyl residues can in each case be linear or branched and the cyclic substituents or the cyclic residues of these substituents themselves can be substituted with optionally 1, 2, 3, 4 or 5, preferably with optionally 1, 2, 3 or 4 substituents mutually independently selected from the group comprising F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂ and —S—CH₂F.

The substituents can be particularly preferably, in each case mutually independently selected from the group comprising F, Cl, Br, I, —CN, —NO₂, —OH, —SH, methyl, ethyl, n-propyl, isopropyl, n-butyl, isobutyl, 2-butyl, tert-butyl, n-pentyl, neo-pentyl, ethenyl, allyl, ethynyl, propynyl, —C≡C—Si(CH₃)₃, —C≡C—Si(C₂H₅)₃, —CH₂—O—CH₃, —CH₂—O—C₂H₅, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —CH₃, —OG₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, pyrazolyl, phenyl, —N(CH₃)₂, —N(C₂H₅)₂, —NH—CH₃, —NH—C₂H₅, —CH₂—O—C(═O)-phenyl, —NH—S(═O)₂—CH₃, —C(═O)—O—CH₃, —C(═O)—O—C₂H₅, —C(═O)—O—C(CH₃)₃, —C(═O)—H, —C(═O)—CH₃, —C(═O)—C₂H₅, —NH—C(═O)—CH₃, —NH—C(═O)—C₂H₅, —O—C(═O)-phenyl, —C(═O)—NH₂, —C(═O)—NH—CH₃, —C(═O)—N(CH₃)₂, —C(═O)—O—CH(CH₃)₂, —C(═O)—O—(CH₂)₃—CH₃, —C(═O)—O-phenyl, —O—C(═O)—C₂H₃, —O—C(═O)—C₂H₅, —C(═O)—NH—C₂H₅, —C(═O)—NH—C(CH₃)₃, —C(═O)—N(C₂H₅)₂, —C(═O)—NH-phenyl, —C(═O)—N(CH₃)-phenyl, —C(═O)—N(C₂H₅)-phenyl, cyclopropyl, cyclobutyl, cyclopentyl, cyclohexyl, phenyl, furyl (furanyl), thiadiazolyl, thiophenyl (thienyl) and benzyl, whereby the cyclic substituents or the cyclic residues of these substituents themselves can in each case be substituted with optionally 1, 2, 3, 4, or 5, preferably with optionally 1, 2, 3 or 4 substituents mutually independently selected from the group comprising F, Cl, Br, I, —CN, —NO₂, —OH, —SH, —NH₂, —C(═O)—OH, methyl, ethyl, n-propyl, isopropyl, n-butyl, isobutyl, 2-butyl, tert-butyl, n-pentyl, neo-pentyl, ethenyl, allyl, ethynyl, propynyl, —C≡C—Si(CH₃)₃, —C≡C—Si(C₂H₅)₃, —CH₂—O—CH₃, —CH₂—O—C₂H₅, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂ and —S—CH₂F.

A substituted aryl residue can very particularly preferably be selected from the group comprising 2-methyl-phenyl, 3-methyl-phenyl, 4-methyl-phenyl, 2-fluoro-phenyl, 3-fluoro-phenyl, 4-fluoro-phenyl, 2-cyano-phenyl, 3-cyano-phenyl, 4-cyano-phenyl, 2-hydroxy-phenyl, 3-hydroxy-phenyl, 4-hydroxy-phenyl, 2-amino-phenyl, 3-amino-phenyl, 4-amino-phenyl, 2-dimethylamino-phenyl, 3-dimethylamino-phenyl, 4-dimethylamino-phenyl, 2-methylamino-phenyl, 3-methylamino-phenyl, 4-methylamino-phenyl, 2-acetyl-phenyl, 3-acetyl-phenyl, 4-acetyl-phenyl, 2-methylsulfinyl-phenyl, 3-methylsulfinyl-phenyl, 4-methylsulfinyl-phenyl, 2-methylsulfonyl-phenyl, 3-methylsulfonyl-phenyl, 4-methylsulfonyl-phenyl, 2-methoxy-phenyl, 3-methoxy-phenyl, 4-methoxy-phenyl, 2-chloro-phenyl, 3-chloro-phenyl, 4-chloro-phenyl, 2-ethoxy-phenyl, 3-ethoxy-phenyl, 4-ethoxyphenyl, 2-trifluoromethyl-phenyl, 3-trifluoromethyl-phenyl, 4-trifluoromethyl-phenyl, 2-difluoromethyl-phenyl, 3-difluoromethyl-phenyl, 4-difluoromethyl-phenyl, 2-fluoromethyl-phenyl, 3-fluoromethyl-phenyl, 4-fluoromethyl-phenyl, 2-nitro-phenyl, 3-nitro-phenyl, 4-nitro-phenyl, 2-ethyl-phenyl, 3-ethyl-phenyl, 4-ethyl-phenyl, 2-propyl-phenyl, 3-propyl-phenyl, 4-propyl-phenyl, 2-isopropyl-phenyl, 3-isopropyl-phenyl, 4-isopropyl-phenyl, 2-tert-butyl-phenyl, 3-tert-butyl-phenyl, 4-tert-butyl-phenyl, 2-carboxyphenyl, 3-carboxy-phenyl, 4-carboxyphenyl, 2-ethenyl-phenyl, 3-ethenyl-phenyl, 4-ethenyl-phenyl, 2-ethynyl-phenyl, 3-ethynyl-phenyl, 4-ethynyl-phenyl, 2-allyl-phenyl, 3-allyl-phenyl, 4-allyl-phenyl, 2-trimethylsilanylethynyl-phenyl, 3-trimethylsilanylethynyl-phenyl, 4-trimethylsilanylethynyl-phenyl, 2-formyl-phenyl, 3-formyl-phenyl, 4-formyl-phenyl, 2-acetamino-phenyl, 3-acetamino-phenyl, 4-acetamino-phenyl, 2-dimethylaminocarbonyl-phenyl, 3-dimethylaminocarbonyl-phenyl, 4-dimethylaminocarbonyl-phenyl, 2-methoxymethyl-phenyl, 3-methoxymethyl-phenyl, 4-methoxymethyl-phenyl, 2-ethoxymethyl-phenyl, 3-ethoxymethyl-phenyl, 4-ethoxymethyl-phenyl, 2-aminocarbonyl-phenyl, 3-aminocarbonyl-phenyl, 4-aminocarbonyl-phenyl, 2-methylaminocarbonyl-phenyl, 3-methylaminocarbonyl-phenyl, 4-methylaminocarbonyl-phenyl, 2-carboxymethylester-phenyl, 3-carboxymethylester-phenyl, 4-carboxymethylester-phenyl, 2-carboxyethylester-phenyl, 3-carboxyethylester-phenyl, 4-carboxyethylester-phenyl, 2-carboxy-tert-butylester-phenyl, 3-carboxy-tert-butylester-phenyl, 4-carboxy-tert-butylester-phenyl, 2-methylmercapto-phenyl, 3-methylmercapto-phenyl, 4-methylmercapto-phenyl, 2-ethylmercapto-phenyl, 3-ethylmercapto-phenyl, 4-ethylmercaptophenyl, 2-biphenyl, 3-biphenyl, 4-biphenyl, 2-bromophenyl, 3-bromophenyl, 4-bromophenyl, 2-iodophenyl, 3-iodophenyl, 4-iodophenyl, 2-trifluoromethoxy-phenyl, 3-trifluoromethoxy-phenyl, 4-trifluoromethoxy-phenyl, 2-fluoro-3-trifluoromethylphenyl, 2-fluoro-4-methyl-phenyl, (2,3)-difluorophenyl, (2,3)-dimethyl-phenyl, (2,3)-dichlorophenyl, 3-fluoro-2-trifluoromethylphenyl, (2,4)-dichloro-phenyl, (2,4)-difluorophenyl, 4-fluoro-2-trifluoromethyl-phenyl, (2,4)-dimethoxyphenyl, 2-chloro-4-fluoro-phenyl, 2-chloro-4-nitro-phenyl, 2-chloro-4-methyl-phenyl, 2-chloro-5-trifluoromethyl-phenyl, 2-chloro-5-methoxy-phenyl, 2-bromo-5-trifluoromethyl-phenyl, 2-bromo-5-methoxy-phenyl, (2,4)-dibromo-phenyl, (2,4)-dimethyl-phenyl, 2-fluoro-4-trifluoromethyl-phenyl, (2,5)-difluoro-phenyl, 2-fluoro-5-trifluoromethyl-phenyl, 5-fluoro-2-trifluoromethyl-phenyl, 5-chloro-2-trifluoromethyl-phenyl, 5-bromo-2-trifluoromethyl-phenyl, (2,5)-dimethoxy-phenyl, (2,5)-bis-trifluoromethyl-phenyl, (2,5)-dichloro-phenyl, (2,5)-dibromo-phenyl, 2-methoxy-5-nitro-phenyl, 2-fluoro-6-trifluoromethyl-phenyl, (2,6)-dimethoxy-phenyl, (2,6)-dimethyl-phenyl, (2,6)-dichloro-phenyl, 2-chloro-6-fluoro-phenyl, 2-bromo-6-chloro-phenyl, 2-bromo-6-fluoro-phenyl, (2,6)-difluoro-phenyl, (2,6)-difluoro-3-methyl-phenyl, (2,6)-dibromo-phenyl, (2,6)-dichlorophenyl, 3-chloro-2-fluoro-phenyl, 3-chloro-5-methyl-phenyl, (3,4)-dichlorophenyl, (3,4)-dimethyl-phenyl, 3-methyl-4-methoxy-phenyl, 4-chloro-3-nitro-phenyl, (3,4)-dimethoxy-phenyl, 4-fluoro-3-trifluoromethylphenyl, 3-fluoro-4-trifluoromethyl-phenyl, (3,4)-difluoro-phenyl, 3-cyano-4-fluoro-phenyl, 3-cyano-4-methyl-phenyl, 3-cyano-4-methoxy-phenyl, 3-bromo-4-fluoro-phenyl, 3-bromo-4-methyl-phenyl, 3-bromo-4-methoxy-phenyl, 4-chloro-2-fluoro-phenyl, 4-chloro-3-trifluoromethyl, 4-bromo-3-methyl-phenyl, 4-bromo-5-methyl-phenyl, 3-chloro-4-fluoro-phenyl, 4-fluoro-3-nitro-phenyl, 4-bromo-3-nitro-phenyl, (3,4)-dibromo-phenyl, 4-chloro-3-methyl-phenyl, 4-bromo-3-methyl-phenyl, 4-fluoro-3-methyl-phenyl, 3-fluoro-4-methyl-phenyl, 3-fluoro-5-methyl-phenyl, 2-fluoro-3-methyl-phenyl, 4-methyl-3-nitro-phenyl, (3,5)-dimethoxy-phenyl, (3,5)-dimethyl-phenyl, (3,5)-bis-trifluoromethyl-phenyl, (3,5)-difluoro-phenyl, (3,5)-dinitro-phenyl, (3,5)-dichloro-phenyl, 3-fluoro-5-trifluoromethyl-phenyl, 5-fluoro-3-trifluoromethyl-phenyl, (3,5)-dibromo-phenyl, 5-chloro-4-fluoro-phenyl, 5-chloro-4-fluoro-phenyl, 5-bromo-4-methyl-phenyl, (2,3,4)-trifluorophenyl, (2,3,4)-trichlorophenyl, (2,3,6)-trifluoro-phenyl, 5-chloro-2-methoxy-phenyl, (2,3)-difluoro-4-methyl, (2,4,5)-trifluoro-phenyl, (2,4,5)-trichloro-phenyl, (2,4)-dichloro-5-fluoro-phenyl, (2,4,6)-trichloro-phenyl, (2,4,6)-trimethylphenyl, (2,4,6)-trifluoro-phenyl, (2,4,6)-trimethoxy-phenyl, (3,4,5)-trimethoxy-phenyl, (2,3,4,5)-tetrafluoro-phenyl, 4-methoxy-(2,3,6)-trimethyl-phenyl, 4-methoxy-(2,3,6)-trimethyl-phenyl, 4-chloro-2,5-dimethyl-phenyl, 2-chloro-6-fluoro-3-methyl-phenyl, 6-chloro-2-fluoro-3-methyl, (2,4,6)-trimethylphenyl and (2,3,4,5,6)-pentafluoro-phenyl.

A substituted heteroaryl residue can very particularly preferably be selected from the group comprising 3-methyl-pyrid-2-yl, 4-methyl-pyrid-2-yl, 5-methyl-pyrid-2-yl, 6-methyl-pyrid-2-yl, 2-methyl-pyrid-3-yl, 4-methyl-pyrid-3-yl, 5-methyl-pyrid-3-yl, 6-methyl-pyrid-3-yl, 2-methyl-pyrid-4-yl, 3-methyl-pyrid-4-yl, 3-fluoro-pyrid-2-yl, 4-fluoro-pyrid-2-yl, 5-fluoro-pyrid-2-yl, 6-fluoro-pyrid-2-yl, 3-chloro-pyrid-2-yl, 4-chloro-pyrid-2-yl, 5-chloro-pyrid-2-yl, 6-chloro-pyrid-2-yl, 3-trifluoromethyl-pyrid-2-yl, 4-trifluoromethyl-pyrid-2-yl, 5-trifluoromethyl-pyrid-2-yl, 6-trifluoromethyl-pyrid-2-yl, 3-methoxy-pyrid-2-yl, 4-methoxy-pyrid-2-yl, 5-methoxy-pyrid-2-yl, 6-methoxy-pyrid-2-yl, 4-methyl-thiazol-2-yl, 5-methyl-thiazol-2-yl, 4-trifluoromethyl-thiazol-2-yl, 5-trifluoromethyl-thiazol-2-yl, 4-chloro-thiazol-2-yl, 5-chloro-thiazol-2-yl, 4-bromo-thiazol-2-yl, 5-bromo-thiazol-2-yl, 4-fluoro-thiazol-2-yl, 5-fluoro-thiazol-2-yl, 4-cyano-thiazol-2-yl, 5-cyano-thiazol-2-yl, 4-methoxy-thiazol-2-yl, 5-methoxy-thiazol-2-yl, 4-methyl-oxazol-2-yl, 5-methyl-oxazol-2-yl, 4-trifluoromethyl-oxazol-2-yl, 5-trifluoromethyl-oxazol-2-yl, 4-chloro-oxazol-2-yl, 5-chloro-oxazol-2-yl, 4-bromo-oxazol-2-yl, 5-bromo-oxazol-2-yl, 4-fluoro-oxazol-2-yl, 5-fluoro-oxazol-2-yl, 4-cyano-oxazol-2-yl, 5-cyano-oxazol-2-yl, 4-methoxy-oxazol-2-yl, 5-methoxy-oxazol-2-yl, 2-methyl-(1,2,4)-thiadiazol-5-yl, 2-trifluoromethyl-(1,2,4)-thiadiazol-5-yl, 2-chloro-(1,2,4)-thiadiazol-5-yl, 2-fluoro-(1,2,4)-thiadiazol-5-yl, 2-methoxy-(1,2,4)-thiadiazol-5-yl, 2-cyano-(1,2,4)-thiadiazol-5-yl, 2-methyl-(1,2,4)-oxadiazol-5-yl, 2-trifluoromethyl-(1,2,4)-oxadiazol-5-yl, 2-chloro-(1,2,4)-oxadiazol-5-yl, 2-fluoro-(1,2,4)-oxadiazol-5-yl, 2-methoxy-(1,2,4)-oxadiazol-5-yl and 2-cyano-(1,2,4)-oxadiazol-5-yl.

The term “alkylene” encompasses, in the context of the present invention, acyclic saturated hydrocarbon chains which connect an aryl, heteroaryl, cycloalkyl, heterocyloalkyl, cycloalkenyl or heterocycloalkenyl residue to the compounds of the general formula I or to another substituent. Alkylene chains can be branched or straight-chained and unsubstituted or at least monosubstituted with as in the case of C₁₋₁₂-alkylene 1 to 12 (i.e. 1, 2, 3, 4, 5, 6, 7, 8, 9, 10, 11 or 12) C-atoms, with as in the case of C₁₋₆-alkylene 1 to 6 (i.e. 1, 2, 3, 4, 5 or 6) C-atoms or with as in the case of C₁₋₃-alkylene 1 to 3 (i.e. 1, 2 or 3) C-atoms. C₁₋₆-alkylene groups such as —(CH₂)—, —(CH₂)₂—, —C(H)(CH₃)—, —(CH₂)₃—, —(CH₂)₄—, —(CH₂)₅—, —C(CH₃)₂—, —C(H)(CH₃)—, —C(H)(C(H)(CH₃)₂)— and C(C₂H₅)(H)— are cited by way of example. —(CH₂)—, —(CH₂)₂— and —(CH₂)₃— are cited by way of example as a suitable C₁₋₃-alkylene group.

The term “alkenylene” encompasses, in the context of the present invention, acyclic unsaturated hydrocarbon chains which connect an aryl, heteroaryl, cycloalkyl, heterocyloalkyl, cycloalkenyl or heterocycloalkenyl residue to the compounds of the general formula I or to another substituent. Alkenylene chains have at least one double-bond, preferably 1, 2 or 3 double-bonds, and can be branched or straight-chained and unsubstituted or at least monosubstituted with as in the case of C₂₋₁₂-alkenylene 2 to 12 (i.e. 2, 3, 4, 5, 6, 7, 8, 9, 10, 11 or 12) C-atoms, with as in the case of C₂₋₆-alkenylene 2 to 6 (i.e. 2, 3, 4, 5 or 6) C-atoms or with as in the case of C₂₋₃-alkenylene 2 to 3 (i.e. 2 or 3) C-atoms. C₂₋₃-alkenylene groups such as —CH═CH— and —CH₂—CH═CH— are cited by way of example.

The term “alkynylene” encompasses, in the context of the present invention, acyclic unsaturated hydrocarbon chains which connect an aryl, heteroaryl, cycloalkyl, heterocyloalkyl, cycloalkenyl or heterocycloalkenyl residue to the compounds of the general formula I or to another substituent. Alkynylene chains have at least one triple-bond, preferably 1 or 2 triple-bonds, and can be branched or straight-chained and unsubstituted or at least monosubstituted with as in the case of C₂₋₁₂-alkynylene 2 to 12 (i.e. 2, 3, 4, 5, 6, 7, 8, 9, 10, 11 or 12) C-atoms, with as in the case of C₂₋₆-alkynylene 2 to 6 (i.e. 2, 3, 4, 5 or 6) C-atoms or with as in the case of C₂₋₃-alkynylene 2 to 3 (i.e. 2 or 3) C-atoms. C₂₋₃-alkynylene groups such as —C≡C— and —CH₂—C≡C— are cited by way of example.

The term “heteroalkylene” denotes an alkylene chain as described above, in which one or more C-atoms have in each case been replaced by a heteroatom mutually independently selected from the group comprising oxygen, sulphur and nitrogen (NH). Heteroalkylene groups can preferably have 1, 2 or 3 heteroatom(s), particularly preferably one heteroatom, selected from the group comprising oxygen, sulphur and nitrogen (NH) as the chain member(s). Heteroalkylene groups can preferably be 2- to 12-membered, particularly preferably 2- to 6-membered, very particularly preferably 2- or 3-membered.

Heteroalkylene groups such as —(CH₂)—O—, —(CH₂)₂—O—, —(CH₂)₃—O—, —(CH₂)₄—O—, —O—(CH₂)—, —O—(CH₂)₂—, —O—(CH₂)₃—, —O—(CH₂)₄—, —C(C₂H₅)(H)—O—, —O—C(C₂H₅)(H)—, —CH₂—O—CH₂—, —CH₂—S—CH₂—, —CH₂—NH—CH₂—, —CH₂—NH— and —CH₂—CH₂—NH—CH₂—CH₂ are cited by way of example.

The term “heteroalkenylene” denotes an alkenylene chain as described above, in which one or more C-atoms have in each case been replaced by a heteroatom mutually independently selected from the group comprising oxygen, sulphur and nitrogen (NH). Heteroalkenylene groups can preferably have 1, 2 or 3 heteroatom(s), particularly preferably 1 heteroatom, selected from the group comprising oxygen, sulphur and nitrogen (NH) as the chain member(s). Heteroalkenylene groups can preferably be 2- to 12-membered, particularly preferably 2- to 6-membered, very particularly preferably 2- or 3-membered. Heteroalkenylene groups such as —CH═CH—NH—, —CH═CH—O— and —CH═CH—S— are cited by way of example.

Provided that one or more of the substituents denote an alkylene, alkenylene, alkynylene, heteroalkylene or heteroalkenylene group or have such a group which is monosubstituted or multiply substituted, this group can preferably be substituted with optionally 1, 2, 3, 4 or 5, particularly preferably with optionally 1, 2 or 3 substituents mutually independently selected from the group comprising phenyl, F, Cl, Br, I, —NO₂, —CN, —OH, —O-phenyl, —O—CH₂-phenyl, —SH, —S-phenyl, —S—CH₂-phenyl, —NH₂, —N(C₁₋₅-alkyl)₂, —NH-phenyl, —N(C₁₋₅-alkyl)(phenyl), —N(C₁₋₅-alkyl)(CH₂-phenyl), —N(C₁₋₅-alkyl)(CH₂—CH₂-phenyl), —C(═O)—H, —C(═O)—C₁₋₅-alkyl, —C(═O)-phenyl, —C(═S)—C₁₋₅-alkyl, —C(═S)-phenyl, —C(═O)—OH, —C(═O)—O—C₁₋₅-alkyl, —C(═O)—O-phenyl, —C(═O)—NH₂, —C(═O)—NH—C₁₋₅-alkyl, —C(═O)—N(C₁₋₅-alkyl)₂, —S(═O)—C₁₋₅-alkyl, —S(═O)-phenyl, —S(═O)₂—C₁₋₅-alkyl, —S(═O)₂-phenyl, —S(═O)₂—NH₂ and —SO₃H, whereby the above-mentioned C₁₋₅-alkyl residues can in each case be linear or branched and the above-mentioned phenyl residues can be substituted with 1, 2, 3, 4 or 5, preferably with 1, 2, 3 or 4 substituents mutually independently selected from the group comprising F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂ and —S—CH₂F.

Alkylene, alkenylene, alkynylene, heteroalkylene or heteroalkenylene groups can particularly preferably be substituted with 1, 2 or 3 substituents mutually independently selected from the group comprising phenyl, F, Cl, Br, I, —NO₂, —CN, —OH, —O-phenyl, —SH, —S-phenyl, —NH₂, —N(CH₃)₂, —N(C₂H₅)₂ and —N(CH₃)(C₂H₅), whereby the phenyl residue can be substituted with 1, 2, 3, 4 or 5 substituents mutually independently selected from the group comprising F, Cl, Br, I, —OH, —SH, —NO₂, —CN, —O—CH₃, —O—CF₃ and —O—C₂H₅.

Substituted bis(hetero)aromatic N-ethylpropiolamides of the above-mentioned general formula I are preferred, in which

### I.)

M¹ denotes phenyl, which can be unsubstituted or substituted with 1, 2, 3, 4 or 5 substituents mutually independently selected from the group comprising F, Cl, Br, I, —CN, —NO₂, —OH, —SH, —NH₂, —C(═O)—OH, methyl, ethyl, n-propyl, isopropyl, n-butyl, isobutyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, —S(═O)₂—C₁₋₅-alkyl, —S(═O)—C₁₋₅-alkyl, —NH—C₁₋₅-alkyl, —N(C₁₋₅-alkyl)₂, —C(═O)—O—C₁₋₅-alkyl, —C(═O)—O-phenyl, —C(═O)—H; —C(═O)—C₁₋₅-alkyl, —CH₂—O—C(═O)-phenyl, —O—C(═O)-phenyl, —O—C(═O)—C₁₋₅-alkyl, —NH—C(═O)—C₁₋₅-alkyl, —C(═O)—NH₂, —C(═O)—NH—C₁₋₅-alkyl, —C(═O)—N(C₁₋₅-alkyl)₂, —C(═O)—N(C₁₋₅-alkyl)(phenyl), —C(═O)—NH-phenyl, cyclopropyl, cyclobutyl, cyclopentyl, cyclohexyl, pyrazolyl, phenyl, furyl (furanyl), thiazolyl, thiadiazolyl, thiophenyl (thienyl), benzyl and phenethyl, whereby the above-mentioned C₁₋₅-alkyl residues can in each case be linear or branched and the cyclic substituents or the cyclic residues of these substituents themselves can be substituted with optionally 1, 2, 3, 4 or 5 substituents mutually independently selected from the group comprising F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂ and —S—CH₂F;
and M² denotes phenyl, which is substituted with 1, 2, 3, 4 or 5 substituents mutually independently selected from the group comprising F, Cl, Br, I, —CN, —NO₂, —OH, —SH, —NH₂, —C(═O)—OH, methyl, ethyl, n-propyl, isopropyl, n-butyl, Isobutyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, —S(═O)₂—C₁₋₅-alkyl, —S(═O)—C₁₋₅-alkyl, —NH—C₁₋₅-alkyl, —N(C₁₋₅alkyl)₂, —C(═O)—O—C₁₋₅-alkyl, —C(═O)—O-phenyl, —C(═O)—H; —C(═O)—C₁₋₅-alkyl, —CH₂—O—C(═O)-phenyl, —O—C(═O)-phenyl, —O—C(═O)—C₁₋₅-alkyl, —NH—C(═O)—C₁₋₅-alkyl, —C(═O)—NH₂, —C(═O)—NH—C₁₋₅-alkyl, —C(═O)—N(C₁₋₅-alkyl)₂, —C(═O)—N(C₁₋₅-alkyl)(phenyl), —C(═O)—NH-phenyl, cyclopropyl, cyclobutyl, cyclopentyl, cyclohexyl, pyrazolyl, phenyl, furyl (furanyl), thiazolyl, thiadiazolyl, thiophenyl (thienyl), benzyl and phenethyl, whereby the above-mentioned C₁₋₅-alkyl residues can in each case be linear or branched and the cyclic substituents or the cyclic residues of these substituents themselves can be substituted with optionally 1, 2, 3, 4 or 5 substituents mutually independently selected from the group comprising F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂ and —S—CH₂F;
or M² denotes unsubstituted or substituted heteroaryl or unsubstituted or substituted naphthyl or denotes an unsubstituted or substituted phenyl residue, which can be condensed (annelated) with unsubstituted or substituted 5- to 7-membered heterocycloalkyl or with unsubstituted or substituted C₅₋₇-cycloalkyl;

### or II.)

M¹ denotes unsubstituted or substituted heteroaryl or unsubstituted or substituted naphthyl or an unsubstituted or substituted phenyl residue, which can be condensed (annelated) with unsubstituted or substituted 5- to 7-membered heterocycloalkyl or with unsubstituted or substituted C₅₋₇-cycloalkyl;
and M² denotes unsubstituted or substituted heteroaryl or unsubstituted or substituted naphthyl or an unsubstituted or substituted phenyl residue, which can be condensed (annelated) with unsubstituted or substituted 5- to 7-membered heterocycloalkyl or with unsubstituted or substituted C₅₋₇-cycloalkyl;
or M² denotes phenyl, which can be unsubstituted or substituted with 1, 2, 3, 4 or 5 substituents mutually independently selected from the group comprising F, Cl, Br, I, —CN, —NO₂, —OH, —SH, —NH₂, —C(═O)—OH, methyl, ethyl, n-propyl, isopropyl, n-butyl, isobutyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, —S(═O)₂—C₁₋₅-alkyl, —S(═O)—C₁₋₅-alkyl, —NH—C₁₋₅-alkyl, —N(C₁₋₅alkyl)₂, —C(═O)—O—C₁₋₅-alkyl, —C(═O)—O-phenyl, —C(═O)—H; —C(═O)—C₁₋₅-alkyl, —CH₂—O—C(═O)-phenyl, —O—C(═O)-phenyl, —O—C(═O)—C₁₋₅-alkyl, —NH—C(═O)—C₁₋₅-alkyl, —C(═O)—NH₂, —C(═O)—NH—C₁₋₅-alkyl, —C(═O)—N(C₁₋₅-alkyl)₂, —C(═O)—N(C₁₋₅-alkyl)(phenyl), —C(═O)—NH-phenyl, cyclopropyl, cyclobutyl, cyclopentyl, cyclohexyl, pyrazolyl, phenyl, furyl (furanyl), thiazolyl, thiadiazolyl, thiophenyl (thienyl), benzyl and phenethyl, whereby the above-mentioned C₁₋₅-alkyl residues can in each case be linear or branched and the cyclic substituents or the cyclic residues of these substituents themselves can be substituted with optionally 1, 2, 3, 4 or 5 substituents mutually independently selected from the group comprising F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂ and —S—CH₂F;
and in each case
R¹ and R², mutually independently, in each case denote H; F; Cl; Br; I; —NO₂; —CN; —NH₂; —OH; —SH; —O—R⁶; —S—R⁷; —NH—R⁸; —NR⁹R¹⁰; unsubstituted or substituted alkyl, alkenyl or alkynyl; unsubstituted or substituted heteroalkyl, heteroalkenyl or heteroalkynyl; unsubstituted or substituted cycloalkyl or cycloalkenyl; unsubstituted or substituted heterocycloalkyl or heterocycloalkenyl; unsubstituted or substituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or substituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; or unsubstituted or substituted -(heteroalkylene)-heterocycloalkyl, -(heteroalkenylene)-heterocycloalkyl, -(heteroalkylene)-heterocycloalkenyl or -(heteroalkenylene)-heterocycloalkenyl or aryl;
or R¹ and R² jointly denote an oxo group (═O);
R³ and R⁴, mutually independently, in each case denote H; F; Cl; Br; I; —NO₂; —CN; —NH₂; —OH; —SH; —C(═O)—OH; —C(═O)—H; —NH—C(═O)—H; —O—R⁶; —S—R⁷; —NH—R⁸; —NR⁹R¹⁰; —C(═O)—R¹¹; —C(═O)—O—R¹²; —O—C(═O)—R¹³; —NH—C(═O)—R¹⁴; —NR¹⁵—C(═O)—R¹⁶; —C(═O)—NH₂; —C(═O)—NH—R¹⁷; —C(═O)—NR-3R¹⁸R¹⁹; —S(═O)—R²⁰; —S(═O)₂—R²¹; —NH—S(═O)₂—R²²; —NR²³—S(═O₂—R²⁴; unsubstituted or substituted alkyl, alkenyl or alkynyl; unsubstituted or substituted heteroalkyl, heteroalkenyl or heteroalkynyl; unsubstituted or substituted cycloalkyl or cycloalkenyl; unsubstituted or substituted heterocycloalkyl or heterocycloalkenyl; unsubstituted or substituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or substituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; or unsubstituted or substituted -(heteroalkylene)-heterocycloalkyl, -(heteroalkenylene)-heterocycloalkyl, -(heteroalkylene)-heterocycloalkenyl or -(heteroalkenylene)-heterocycloalkenyl;
R⁵ denotes H; —C(═O)—O—R¹²; —C(═O)—NH₂; —C(═O)—NH—R¹⁷; —C(═O)—NR¹⁸R¹⁹; —S(═O)—R²⁰; —S(═O)₂—R²¹; unsubstituted or substituted alkyl, alkenyl or alkynyl; unsubstituted or substituted heteroalkyl, heteroalkenyl or heteroalkynyl; unsubstituted or substituted cycloalkyl or cycloalkenyl; unsubstituted or substituted heterocycloalkyl or heterocycloalkenyl; unsubstituted or substituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or substituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; unsubstituted or substituted -(heteroalkylene)-heterocycloalkyl, -(heteroalkenylene)-heterocycloalkyl, -(heteroalkylene)-heterocycloalkenyl or -(heteroalkenylene)-heterocycloalkenyl; unsubstituted or substituted aryl; unsubstituted or substituted heteroaryl; unsubstituted or substituted -(alkylene)-aryl, whereby aryl can be unsubstituted or substituted with 1, 2, 3, 4 or 5 substituents mutually independently selected from the group comprising F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, —S(═O)₂—C₁₋₅-alkyl, —S(═O)—C₁₋₅-alkyl, —NH—C₁₋₅-alkyl, N(C₁₋₅alkyl)₂, —C(═O)—O—C₁₋₅-alkyl, —C(═O)—O-phenyl, —C(═O)—H, —C(═O)—C₁₋₅-alkyl, —CH₂—O—C(═O)-phenyl, —O—C(═O)-phenyl, —O—C(═O)—C₁₋₅-alkyl, —NH—C(═O)—C₁₋₅-alkyl, —C(═O)—NH₂, —C(═O)—NH—C₁₋₅-alkyl, —C(═O)—N(C₁₋₅-alkyl)₂, —C(═O)—N(C₁₋₅-alkyl)(phenyl), —C(═O)—NH-phenyl, cyclopropyl, cyclobutyl, cyclopentyl, cyclohexyl, pyrazolyl, phenyl, furyl (furanyl), thiazolyl, thiadiazolyl, thiophenyl (thienyl), benzyl and phenethyl; -(alkenylene)-aryl, -(alkynylene)-aryl, -(heteroalkylene)-aryl or -(heteroalkenylene)-aryl; or unsubstituted or substituted -(alkylene)-heteroaryl, -(alkenylene)-heteroaryl, -(alkynylene)-heteroaryl, -(heteroalkylene)-heteroaryl or -(heteroalkenylene)-heteroaryl;
and, provided that M² denotes unsubstituted or substituted heteroaryl or unsubstituted or substituted naphthyl or an unsubstituted or substituted phenyl residue, which can be condensed (annelated) with unsubstituted or substituted 5- to 7-membered heterocycloalkyl or with unsubstituted or substituted C₅₋₇-cycloalkyl, R⁵ additionally can denote —C(═O)—R¹¹;
and R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³ and R²⁴, mutually independently, in each case denote unsubstituted or substituted alkyl, alkenyl or alkynyl; unsubstituted or substituted heteroalkyl, heteroalkenyl or heteroalkynyl; unsubstituted or substituted cycloalkyl or cycloalkenyl; unsubstituted or substituted heterocycloalkyl or heterocycloalkenyl; unsubstituted or substituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or substituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; unsubstituted or substituted -(heteroalkylene)-heterocycloalkyl, -(heteroalkenylene)-heterocycloalkyl, -(heteroalkylene)-heterocycloalkenyl; or -(heteroalkenylene)-heterocycloalkenyl; unsubstituted or substituted aryl; unsubstituted or substituted heteroaryl; unsubstituted or substituted -(alkylene)-aryl, -(alkenylene)-aryl, -(alkynylene)-aryl, -(heteroalkylene)-aryl or -(heteroalkenylene)-aryl; or unsubstituted or substituted -(alkylene)-heteroaryl, -(alkenylene)-heteroaryl, -(alkynylene)-heteroaryl, -(heteroalkylene)-heteroaryl or -(heteroalkenylene)-heteroaryl;
whereby
the above-mentioned alkyl residues are in each case branched or straight-chained and have 1, 2, 3, 4, 5, 6, 7, 8, 9, 10, 11 or 12 carbon atoms as chain members;
the above-mentioned alkenyl residues are in each case branched or straight-chained and have 2, 3, 4, 5, 6, 7, 8, 9, 10, 11 or 12 carbon atoms as chain members;
the above-mentioned alkynyl residues are in each case branched or straight-chained and have 2, 3, 4, 5, 6, 7, 8, 9, 10, 11 or 12 carbon atoms as chain members;
the above-mentioned heteroalkyl residues, heteroalkenyl residues and heteroalkynyl residues are in each case 2-, 3-, 4-, 5-, 6-, 7-, 8-, 9-, 10-, 11- or 12-membered;
the above-mentioned heteroalkyl residues, heteroalkenyl residues and heteroalkynyl residues in each case have optionally 1, 2 or 3 heteroatom(s), mutually independently, selected from the group comprising oxygen, sulphur and nitrogen as the chain member(s);
the above-mentioned alkyl residues, alkenyl residues, alkynyl residues, heteroalkyl residues, heteroalkenyl residues and heteroalkynyl residues can in each case be substituted with optionally 1, 2, 3, 4 or 5 substituents mutually independently selected from the group comprising F, Cl, Br, I, —NO₂, —CN, —OH, —SH, —NH₂, —N(C₁₋₅-alkyl)₂, —N(C₁₋₅-alkyl)(phenyl), —N(C₁₋₅-alkyl)(CH₂-phenyl), —N(C₁₋₅-alkyl)(CH₂—CH₂-phenyl), —C(═O)—H, —C(═O)—C₁₋₅-alkyl, —C(═O)-phenyl, —C(═S)—C₁₋₅-alkyl, —C(═S)-phenyl, —C(═O)—OH, —C(═O)—O—C₁₋₅-alkyl, —C(═O)—O-phenyl, —C(═O)—NH₂, —C(═O)—NH—C₁₋₅-alkyl, —C(═O)—N(C₁₋₅-alkyl)₂, —S(═O)—C₁₋₅-alkyl, —S(═O)-phenyl, —S(═O)₂—C₁₋₅-alkyl, —S(═O)₂-phenyl, —S(═O)₂—NH₂ and —SO₃H, whereby the phenyl residues can be substituted with 1, 2, 3, 4 or 5 substituents mutually independently selected from the group comprising F, Cl, Br, I, —CN, —CF₃, —OH, —NH₂, —O—CF₃, —SH, —O—CH₃, —O—C₂H₅, —O—C₃H₇, methyl, ethyl, n-propyl, isopropyl, n-butyl, 2-butyl, isobutyl and tert-butyl; unless indicated otherwise, the above-mentioned cycloalkyl residues in each case have 3, 4, 5, 6, 7, 8 or 9 carbon atoms as ring members;
the above-mentioned cycloalkenyl residues in each case have 3, 4, 5, 6, 7, 8 or 9 carbon atoms as ring members;
unless indicated otherwise, the above-mentioned heterocycloalkyl residues are in each case 3-, 4-, 5-, 6-, 7-, 8- or 9-membered;
the above-mentioned heterocycloalkenyl residues are in each case 4-, 5-, 6-, 7-, 8- or 9-membered;
the above-mentioned heterocycloalkyl residues and heterocycloalkenyl residues in each case have optionally 1, 2 or 3 heteroatom(s), mutually independently, selected from the group comprising oxygen, sulphur and nitrogen (NH) as the ring member(s);
the above-mentioned cycloalkyl residues, heterocycloalkyl residues, cycloalkenyl residues or heterocycloalkenyl residues can in each case be substituted with optionally 1, 2, 3, 4 or 5 substituents mutually independently selected from the group comprising F, Cl, Br, I, —CN, —CF₃, —OH, —NH₂, —O—CF₃, —SH, —O—C₁₋₅-alkyl, —O-phenyl, —O—CH₂-phenyl, —(CH₂)—O—C₁₋₅-alkyl, —S—C₁₋₅-alkyl, —S-phenyl, —S—CH₂-phenyl, —C₁₋₅-alkyl, —C₂₋₅-alkenyl, —C₂₋₅-alkynyl, —C≡C—Si(CH₃)₃, —C≡C—Si(C₂H₅)₃, —C(═O)—O—C₁₋₅-alkyl, —C(═O)—CF₃, —S(═O)₂—C₁₋₅-alkyl, —S(═O)—C₁₋₅-alkyl, —S(═O)₂-phenyl, oxo (═O), thioxo (═S), —N(C₁₋₅-alkyl)₂, —N(H)(C₁₋₅-alkyl), —NO₂, —S—CF₃, —C(═O)—OH, —NH—C(═O)—C₁₋₅-alkyl, —C(═O)—H, —C(═O)—C₁₋₅-alkyl, —C(═O)—NH₂, —C(═O)—N(C₁₋₅-alkyl)₂, —C(═O)—N(H)(C₁₋₅-alkyl) and phenyl, whereby the phenyl residues can respectively be unsubstituted or substituted with 1, 2, 3, 4 or 5 substituents mutually independently selected from the group comprising F, Cl, Br, I, —CN, —CF₃, —OH, —NH₂, —O—CF₃, —SH, —O—C₁₋₅-alkyl, —O-phenyl, —O—CH₂-phenyl, —(CH₂)—O—C₁₋₅-alkyl, —S—C₁₋₅-alkyl, —S-phenyl, —S—CH₂-phenyl, —C₁₋₅-alkyl, —C₂₋₅-alkenyl, —C₂₋₅-alkynyl, —C≡C—Si(CH₃)₃, —C≡C—Si(C₂H₅)₃, —C(═O)—O—C₁₋₅-alkyl and —C(═O)—CF₃, whereby the above-mentioned phenyl residues can preferably be substituted with 1, 2, 3, 4 or 5 substituents mutually independently selected from the group comprising F, Cl, Br, I, —CN, —CF₃, —OH, —NH₂, —O—CF₃, —SH, —O—CH₃, —O—C₂H₅, —O—C₃H₇, methyl, ethyl, n-propyl, isopropyl, n-butyl, 2-butyl, isobutyl and tert-butyl;
the above-mentioned alkylene residues are in each case branched or straight-chained and have 1, 2, 3, 4, 5, 6, 7, 8, 9, 10, 11 or 12 carbon atoms as chain members;
the above-mentioned alkenylene residues are in each case branched or straight-chained and have 2, 3, 4, 5, 6, 7, 8, 9, 10, 11 or 12 carbon atoms as chain members;
the above-mentioned alkynylene residues are in each case branched or straight-chained and have 2, 3, 4, 5, 6, 7, 8, 9, 10, 11 or 12 carbon atoms as chain members;
the above-mentioned heteroalkylene residues and heteroalkenylene residues are in each case 2-, 3-, 4-, 5-, 6-, 7-, 8-, 9-, 10-, 11- or 12-membered;
the above-mentioned heteroalkylene and heteroalkenylene groups have in each case optionally 1, 2 or 3 heteroatom(s), mutually independently, selected from the group comprising oxygen, sulphur and nitrogen (NH) as the chain member(s);
the above-mentioned alkylene, alkenylene, alkynylene, heteroalkylene or heteroalkenylene group can in each case be unsubstituted or substituted with optionally 1, 2, 3, 4 or 5 substituents mutually independently selected from the group comprising phenyl, F, Cl, Br, I, —NO₂, —CN, —OH, —O-phenyl, —O—CH₂-phenyl, —SH, —S-phenyl, —S—CH₂-phenyl, NH₂, —N(C₁₋₅-alkyl)₂, —NH-phenyl, —N(C₁₋₅-alkyl)(phenyl), —N(C₁₋₅-alkyl)(CH₂-phenyl), —N(C₁₋₅-alkyl)(CH₂—CH₂-phenyl), —C(═O)—H, —C(═O)—C₁₋₅-alkyl, —C(═O)-phenyl, —C(═S)—C₁₋₅-alkyl, —C(═S)-phenyl, —C(═O)—OH, —C(═O)—O—C₁₋₅-alkyl, —C(═O)—O-phenyl, —C(═O)—NH₂, —C(═O)—NH—C₁₋₅-alkyl, —C(═O)—N(C₁₋₅-alkyl)₂, —S(═O)—C₁₋₅-alkyl, —S(═O)-phenyl, —S(═O)₂—C₁₋₅-alkyl, —S(═O)₂-phenyl, —S(═O)₂—NH₂ and —SO₃H, whereby the phenyl residues can be substituted with 1, 2, 3, 4 or 5 substituents mutually independently selected from the group comprising F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂ and —S—CH₂F;
the above-mentioned aryl residues are mono- or bicyclic and have 6, 10 or 14 carbon atoms;
the above-mentioned heteroaryl residues are mono-, bi- or tricyclic and 5-, 6-, 7-, 8-, 9-, 10-, 11-, 12-, 13- or 14-membered;
the above-mentioned 5- to 14-membered heteroaryl residues in each case have optionally 1, 2, 3, 4 or 5 heteroatom(s), mutually independently, selected from the group comprising oxygen, sulphur and nitrogen (NH) as the ring member(s);
and, unless indicated otherwise, the above-mentioned naphthyl residues, aryl residues and heteroaryl residues can in each case be substituted with optionally 1, 2, 3, 4 or 5 substituents mutually independently selected from the group comprising F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, —S(═O)₂—C₁₋₅-alkyl, —S(═O)—C₁₋₅-alkyl, —NH—C₁₋₅-alkyl, N(C₁₋₅-alkyl)₂, —C(═O)—O—C₁₋₅-alkyl, —C(═O)—O-phenyl, —C(═O)—H, —C(═O)—C₁₋₅-alkyl, —CH₂—O—C(═O)-phenyl, —O—C(═O)-phenyl, —O—C(═O)—C₁₋₅-alkyl, —NH—C(═O)—C₁₋₅-alkyl, —C(═O)—NH₂, —C(═O)—NH—C₁₋₅-alkyl, —C(═O)—N(C₁₋₅-alkyl)₂, —C(═O)—N(C₁₋₅-alkyl)(phenyl), —C(═O)—NH-phenyl, cyclopropyl, cyclobutyl, cyclopentyl, cyclohexyl, pyrazolyl, phenyl, furyl (furanyl), thiazolyl, thiadiazolyl, thiophenyl (thienyl), benzyl and phenethyl, whereby the cyclic substituents or the cyclic residues of these substituents themselves can be substituted with optionally 1, 2, 3, 4 or 5 substituents mutually independently selected from the group comprising F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂ and —S—CH₂F;
in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Substituted bis(hetero)aromatic N-ethylpropiolamides of the above-mentioned general formula I are also preferred, in which M¹ denotes a phenyl residue, which in each case is unsubstituted or substituted with 1, 2, 3, 4 or 5 substituents mutually independently selected from the group comprising F, Cl, Br, I, —CN, —NO₂, methyl, ethyl, n-propyl, isopropyl, ethenyl, allyl, ethynyl, propynyl, —C≡C—Si(CH₃)₃, —C≡C—Si(C₂H₅)₃, —CH₂—O—CH₃, —CH₂—O—C₂H₅, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, pyrazolyl, phenyl, —N(CH₃)₂, —N(C₂H₅)₂, —NH—CH₃, —NH—C₂H₅, —CH₂—O—C(═O)-phenyl, —C(═O)—O—CH₃, —C(═O)—O—C₂H₅, —C(═O)—O—CH(CH₃)₂, —C(═O)—O—(CH₂)₃—CH₃, —C(═O)—O—C(CH₃)₃, —C(═O)—O-phenyl, —C(═O)—H, —C(═O)—CH₃, —C(═O)—C₂H₅, —NH—C(═O)—CH₃, —NH—C(═O)—C₂H₅, —O—C(═O)-phenyl, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅, —C(═O)—NH—C(CH₃)₃, —C(═O)—N(C₂H₅)₂, —C(═O)—NH-phenyl, —C(═O)—N(CH₃)— phenyl, —C(═O)—N(C₂H₅)-phenyl, cyclopropyl, cyclobutyl, cyclopentyl, cyclohexyl, phenyl, furyl (furanyl), thiadiazolyl, thiophenyl (thienyl) and benzyl;

and M² denotes a phenyl residue, which is substituted with 1, 2, 3, 4 or 5 substituents mutually independently selected from the group comprising F, Cl, Br, I, —CN, —NO₂, methyl, ethyl, n-propyl, isopropyl, n-butyl, tert-butyl, n-pentyl, ethenyl, allyl, ethynyl, propynyl, —C≡C—Si(CH₃)₃, —C≡C—Si(C₂H₅)₃, —CH₂—O—CH₃, —CH₂—O—C₂H₅, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, pyrazolyl, phenyl, —N(CH₃)₂, —N(C₂H₅)₂, —NH—CH₃, —NH—C₂H₅, —CH₂—O—C(═O)-phenyl, —C(═O)—O—CH₃, —C(═O)—O—C₂H₅, —C(═O)—O—CH(CH₃)₂, —C(═O)—O—(CH₂)₃—CH₃, —C(═O)—O—C(CH₃)₃, —C(═O)—O-phenyl, —C(═O)—H, —C(═O)—CH₃, —C(═O)—C₂H₅, —NH—C(═O)—CH₃, —NH—C(═O)—C₂H₅, —O—C(═O)-phenyl, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅, —C(═O)—NH—C(CH₃)₃, —C(═O)—N(C₂H₅)₂, —C(═O)—NH-phenyl, —C(═O)—N(CH₃)-phenyl, —C(═O)—N(C₂H₅)-phenyl, cyclopropyl, cyclobutyl, cyclopentyl, cyclohexyl, phenyl, furyl (furanyl), thiadiazolyl, thiophenyl (thienyl) and benzyl
or M² denotes a residue selected from the group comprising naphthyl, thiophenyl (thienyl), furanyl (furyl), pyrrolyl, pyrazolyl, pyranyl, triazolyl, pyridinyl, imidazolyl, indolyl, isoindolyl, indolizinyl, benzimidazolyl, benzo[b]furanyl, benzo[b]thiophenyl, benzo[d]thiazolyl, benzodiazolyl, benzotriazolyl, benzoxazolyl, benzisoxazolyl, thiazolyl, thiadiazolyl, oxazolyl, oxadiazolyl, tetrazolyl, triazinyl, isoxazolyl, pyridazinyl, pyrazinyl, pyrimidinyl, indazolyl, phthalazinyl, carbazolyl, carbolinyl, diaza-naphthyl, quinoxalinyl, quinazolinyl, quinolinyl, naphthridinyl, isoquinolinyl, indolinyl, (2,3)-dihydrobenzofuranyl, (2,3)-dihydrobenzo[d]oxazolyl, benzo[d][1,3]dioxolyl, isoindolinyl, (1,2,3,4)-tetrahydroquinolinyl and (2,3)-dihydrobenzo[b][1,4]dioxinyl which is unsubstituted or substituted with 1, 2, 3, 4 or 5 substituents mutually independently selected from the group comprising F, Cl, Br, I, —CN, —NO₂, methyl, ethyl, n-propyl, isopropyl, n-butyl, tert-butyl, n-pentyl, ethenyl, allyl, ethynyl, propynyl, —C≡C—Si(CH₃)₃, —C≡C—Si(C₂H₅)₃, —CH₂—O—CH₃, —CH₂—O—C₂H₅, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, pyrazolyl, phenyl, —N(CH₃)₂, —N(C₂H₅)₂, —NH—CH₃, —NH—C₂H₅, —CH₂—O—C(═O)-phenyl, —C(═O)—O—CH₃, —C(═O)—O—C₂H₅, —C(═O)—O—CH(CH₃)₂, —C(═O)—O—(CH₂)₃—CH₃, —C(═O)—O—C(CH₃)₃, —C(═O)—O—phenyl, —C(═O)—H, —C(═O)—CH₃, —C(═O)—C₂H₅, —NH—C(═O)—CH₃, —NH—C(═O)—C₂H₅, —O—C(═O)-phenyl, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅, —C(═O)—NH—C(CH₃)₃, —C(═O)—N(C₂H₅)₂, —C(═O)—NH-phenyl, —C(═O)—N(CH₃)-phenyl, —C(═O)—N(C₂H₅)-phenyl, cyclopropyl, cyclobutyl, cyclopentyl, cyclohexyl, phenyl, furyl (furanyl), thiadiazolyl, thiophenyl (thienyl) and benzyl;
and in each case the remaining residues have the above-mentioned meaning, in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Substituted bis(hetero)aromatic N-ethylpropiolamides of the above-mentioned general formula I are further preferred, in which M¹ denotes a residue selected from the group comprising naphthyl, thiophenyl (thienyl), furanyl (furyl), pyrrolyl, pyrazolyl, pyranyl, triazolyl, pyridinyl, imidazolyl, indolyl, isoindolyl, indolizinyl, benzimidazolyl, benzo[b]furanyl, benzo[b]thiophenyl, benzo[d]thiazolyl, benzodiazolyl, benzotriazolyl, benzoxazolyl, benzisoxazolyl, thiazolyl, thiadiazolyl, oxazolyl, oxadiazolyl, tetrazolyl, triazinyl, isoxazolyl, pyridazinyl, pyrazinyl, pyrimidinyl, indazolyl, phthalazinyl, carbazolyl, carbolinyl, diaza-naphthyl, quinoxalinyl, quinazolinyl, quinolinyl, naphthridinyl, isoquinolinyl, indolinyl, (2,3)-dihydrobenzofuranyl, (2,3)-dihydrobenzo[d]oxazolyl, benzo[d][1,3]dioxolyl, isoindolinyl, (1,2,3,4)-tetrahydroquinolinyl and (2,3)-dihydrobenzo[b][1,4]dioxinyl, which is unsubstituted or substituted with 1, 2, 3, 4 or 5 substituents mutually independently selected from the group comprising F, Cl, Br, I, —CN, —NO₂, methyl, ethyl, n-propyl, isopropyl, n-butyl, tert-butyl, n-pentyl, ethenyl, allyl, ethynyl, propynyl, —C≡C—Si(CH₃)₃, —C≡C—Si(C₂H₅)₃, —CH₂—O—CH₃, —CH₂—O—C₂H₅, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, pyrazolyl, phenyl, —N(CH₃)₂, —N(C₂H₅)₂, —NH—CH₃, —NH—C₂H₅, —CH₂—O—C(═O)-phenyl, —C(═O)—O—CH₃, —C(═O)—O—C₂H₅, —C(═O)—O—CH(CH₃)₂, —C(═O)—O—(CH₂)₃—CH₃, —C(═O)—O—C(CH₃)₃, —C(═O)—O-phenyl, —C(═O)—H, —C(═O)—CH₃, —C(═O)—C₂H₅, —NH—C(═O)—CH₃, —NH—C(═O)—C₂H₅, —O—C(═O)-phenyl, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅, —C(═O)—NH—C(CH₃)₃, —C(═O)—N(C₂H₅)₂, —C(═O)—NH-phenyl, —C(═O)—N(CH₃)— phenyl, —C(═O)—N(C₂H₅)-phenyl, cyclopropyl, cyclobutyl, cyclopentyl, cyclohexyl, phenyl, furyl (furanyl), thiadiazolyl, thiophenyl (thienyl) and benzyl.

and M² denotes a residue selected from the group comprising naphthyl, thiophenyl (thienyl), furanyl (furyl), pyrrolyl, pyrazolyl, pyranyl, triazolyl, pyridinyl, imidazolyl, indolyl, isoindolyl, indolizinyl, benzimidazolyl, benzo[b]furanyl, benzo[b]thiophenyl, benzo[d]thiazolyl, benzodiazolyl, benzotriazolyl, benzoxazolyl, benzisoxazolyl, thiazolyl, thiadiazolyl, oxazolyl, oxadiazolyl, tetrazolyl, triazinyl, isoxazolyl, pyridazinyl, pyrazinyl, pyrimidinyl, indazolyl, phthalazinyl, carbazolyl, carbolinyl, diaza-naphthyl, quinoxalinyl, quinazolinyl, quinolinyl, naphthridinyl, isoquinolinyl, indolinyl, (2,3)-dihydrobenzofuranyl, (2,3)-dihydrobenzo[d]oxazolyl, benzo[d][1,3]dioxolyl, isoindolinyl, (1,2,3,4)-tetrahydroquinolinyl and (2,3)-dihydrobenzo[b][1,4]dioxinyl which is unsubstituted or substituted with 1, 2, 3, 4 or 5 substituents mutually independently selected from the group comprising F, Cl, Br, I, —CN, —NO₂, methyl, ethyl, n-propyl, isopropyl, n-butyl, tert-butyl, n-pentyl, ethenyl, allyl, ethynyl, propynyl, —C≡C—Si(CH₃)₃, —C≡C—Si(C₂H₅)₃, —CH₂—O—CH₃, —CH₂—O—C₂H₅, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, pyrazolyl, phenyl, —N(CH₃)₂, —N(C₂H₅)₂, —NH—CH₃, —NH—C₂H₅, —CH₂—O—C(═O)-phenyl, —C(═O)—O—CH₃, —C(═O)—O—C₂H₅, —C(═O)—O—CH(CH₃)₂, —C(═O)—O—(CH₂)₃—CH₃, —C(═O)—O—C(CH₃)₃, —C(═O)—O-phenyl, —C(═O)—H, —C(═O)—CH₃, —C(═O)—C₂H₅, —NH—C(═O)—CH₃, —NH—C(═O)—C₂H₅, —O—C(═O)-phenyl, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅, —C(═O)—NH—C(CH₃)₃, —C(═O)—N(C₂H₅)₂, —C(═O)—NH-phenyl, —C(═O)—N(CH₃)-phenyl, —C(═O)—N(C₂H₅)-phenyl, cyclopropyl, cyclobutyl, cyclopentyl, cyclohexyl, phenyl, furyl (furanyl), thiadiazolyl, thiophenyl (thienyl) and benzyl;
or M² denotes a phenyl residue which is unsubstituted or substituted with 1, 2, 3, 4 or 5 substituents mutually independently selected from the group comprising F, Cl, Br, I, —CN, —NO₂, methyl, ethyl, n-propyl, isopropyl, n-butyl, tert-butyl, n-pentyl, ethenyl, allyl, ethynyl, propynyl, —C≡C—Si(CH₃)₃, —C≡C—Si(C₂H₅)₃, —CH₂—O—CH₃, —CH₂—O—C₂H₅, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, pyrazolyl, phenyl, —N(CH₃)₂, —N(C₂H₅)₂, —NH—CH₃, —NH—C₂H₅, —CH₂—O—C(═O)-phenyl, —C(═O)—O—CH₃, —C(═O)—O—C₂H₅, —C(═O)—O—CH(CH₃)₂, —C(═O)—O—(CH₂)₃—CH₃, —C(═O)—O—C(CH₃)₃, —C(═O)—O-phenyl, —C(═O)—H, —C(═O)—CH₃, —C(═O)—C₂H₅, —NH—C(═O)—CH₃, —NH—C(═O)—C₂H₅, —O—C(═O)-phenyl, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅, —C(═O)—NH—C(CH₃)₃, —C(═O)—N(C₂H₅)₂, —C(═O)—NH-phenyl, —C(═O)—N(CH₃)-phenyl, —C(═O)—N(C₂H₅)-phenyl, cyclopropyl, cyclobutyl, cyclopentyl, cyclohexyl, phenyl, furyl (furanyl), thiadiazolyl, thiophenyl (thienyl) and benzyl;
and in each case the remaining residues have the above-mentioned meaning, in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Substituted bis(hetero)aromatic N-ethylpropiolamides of the above-mentioned general formula I are also preferred, in which R¹ and R², mutually independently, in each case denote H; F; Cl; Br; I; —NO₂; —CN; —NH₂; —OH; —SH; —O—R⁶; —S—R⁷; —NH—R⁸; —NR⁹R¹⁰; C₁₋₆-alkyl, which is unsubstituted or substituted with optionally 1, 2, 3, 4 or 5 substituents mutually independently selected from the group comprising F, Cl, Br, I, —NO₂, —CN, —OH, —SH and —NH₂; or C₃₋₇-cycloalkyl, C₅₋₆-cycloalkenyl, 5- to 7-membered heterocycloalkyl or 5- to 7-membered heterocycloalkenyl, which can be bound in each case via a C₁₋₃-alkylene-, C₂₋₃-alkenylene- or C₂₋₃-alkynylene group and/or is unsubstituted or substituted with optionally 1, 2, 3, 4 or 5 substituents mutually independently selected from the group comprising F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or a phenyl residue, which is unsubstituted or substituted with optionally 1, 2, 3, 4 or 5 substituents mutually independently selected from the group comprising F, Cl, Br, I, —CN, —NO₂, methyl, ethyl, n-propyl, isopropyl, n-butyl, tert-butyl, n-pentyl, ethenyl, allyl, ethynyl, propynyl, —C≡C—Si(CH₃)₃, —C≡C—Si(C₂H₅)₃, —CH₂—O—CH₃, —CH₂—O—C₂H₅, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —O—C(CH₃)₃, —CF₃, —CHF₂, —CH₂F, —CF₃, —O—CHF₂, —O—CH₂F, —C(═O)—CF₃, —S—CF₃, —S—CHF₂, —S—CH₂F, —S(═O)₂-phenyl, pyrazolyl, phenyl, —N(CH₃)₂, —N(C₂H₅)₂, —NH—CH₃, —NH—C₂H₅, —CH₂—H—C(═O)-phenyl, —C(═O)—O—CH₃, —C(═O)—O—C₂H₅, —C(═O)—O—CH(CH₃)₂, —C(═O)—O—(CH₂)₃—CH₃, —C(═O)—O—C(CH₃)₃, —C(═O)—O-phenyl, —C(═O)—H, —C(═O)—CH₃, —C(═O)—C₂H₅, —NH—C(═O)—CH₃, —NH—C(═O)—C₂H₅, —O—C(═O)-phenyl, —O—C(═O)—CH₃, —C(═O)—C₂H₅, —C(═O)—NH₂, —C(═O)—NH—CH₃, —C(═O)—N(CH₃)₂, —C(═O)—NH—C₂H₅, —C(═O)—NH—C(CH₃)₃, —C(═O)—N(C₂H₅)₂, —C(═O)—NH-phenyl, —C(═O)—N(CH₃)— phenyl, —C(═O)—N(C₂H₅)-phenyl, cyclopropyl, cyclobutyl, cyclopentyl, cyclohexyl, phenyl, furyl (furanyl), thiadiazolyl, thiophenyl (thienyl) and benzyl;

or R¹ and R² jointly denote an oxo group (═O).
and in each case the remaining residues have the above-mentioned meaning, in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Substituted bis(hetero)aromatic N-ethylpropiolamides of the above-mentioned general formula I are furthermore preferred, in which R³ and R⁴, mutually independently, in each case denote H; F; Cl; Br; I; —NO₂; —CN; —NH₂; —OH; —SH; —C(═O)—OH; —C(═O)—H; —NH—C(═O)—H; —O—R⁶; —S—R⁷; —NH—R⁸; —NR⁹R¹⁰; —C(═O)—R¹¹; —C(═O)—O—R¹²; —NH—C(═O)—R¹⁴; —NR¹⁵—C(═O)—R¹⁶; —C(═O)—NH₂; —C(═O)—NH—R¹⁷; —C(═O)—NR¹⁸R¹⁹; C₁₋₆-alkyl, which is unsubstituted or substituted with optionally 1, 2, 3, 4 or 5 substituents mutually independently selected from the group comprising F, Cl, Br, I, —NO₂, —CN, —OH, —SH and —NH₂; or C₃₋₇-cycloalkyl, C₅₋₆-cycloalkenyl, 5- to 7-membered heterocycloalkyl or 5- to 7-membered heterocycloalkenyl, which can be bound in each case via a C₁₋₃-alkylene-, C₂₋₃-alkenylene- or C₂₋₃-alkynylene group and/or is unsubstituted or substituted with optionally 1, 2, 3, 4 or 5 substituents mutually independently selected from the group comprising F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅, or a phenyl residue, which is unsubstituted or substituted with optionally 1, 2, 3, 4 or 5 substituents mutually independently selected from the group comprising F, Cl, Br, I, —CN, —NO₂, methyl, ethyl, n-propyl, isopropyl, n-butyl, tert-butyl, n-pentyl, ethenyl, allyl, ethynyl, propynyl, —C≡C—Si(CH₃)₃, —C≡C—Si(C₂H₅)₃, —CH₂—O—CH₃, —CH₂—O—C₂H₅, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, pyrazolyl, phenyl, —N(CH₃)₂, —N(C₂H₅)₂, —NH—CH₃, —NH—C₂H₅, —CH₂—O—C(═O)-phenyl, —C(═O)—O—CH₃, —C(═O)—O—C₂H₅, —C(═O)—O—CH(CH₃)₂, —C(═O)—O—(CH₂)₃—CH₃, —C(═O)—O—C(CH₃)₃, —C(═O)—O-phenyl, —C(═O)—H, —C(═O)—CH₃, —C(═O)—C₂H₅, —NH—C(═O)—CH₃, —NH—C(═O)—C₂H₅, —O—C(═O)-phenyl, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅, —C(═O)—NH—C(CH₃)₃, —C(═O)—N(C₂H₅)₂, —C(═O)—NH-phenyl, —C(═O)—N(CH₃)— phenyl, —C(═O)—N(C₂H₅)-phenyl, cyclopropyl, cyclobutyl, cyclopentyl, cyclohexyl, phenyl, furyl (furanyl), thiadiazolyl, thiophenyl (thienyl) and benzyl;

and in each case the remaining residues have the above-mentioned meaning, in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Substituted bis(hetero)aromatic N-ethylpropiolamides of the above-mentioned general formula I are also preferred, in which R⁵ denotes H; —C(═O)—O—R¹²; —C(═O)—NH₂; —C(═O)—NH—R¹⁷; —C(═O)—NR¹⁸R¹⁹; —S(═O)—R²⁰; —S(═O)₂—R²¹; C₁₋₆-alkyl, which is unsubstituted or substituted with optionally 1, 2, 3, 4 or 5 substituents mutually independently selected from the group comprising F, Cl, Br, I, —NO₂, —CN, —OH, —SH and —NH₂; C₃₋₇-cycloalkyl, C₅₋₆-cycloalkenyl, 5- to 7-membered heterocycloalkyl or 5- to 7-membered heterocycloalkenyl, which in each case can be bound via a C₁₋₃-alkylene-, C₂₋₃-alkenylene- or C₂₋₃-alkynylene group and/or is unsubstituted or substituted with optionally 1, 2, 3, 4 or 5 substituents mutually independently selected from the group comprising F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or a residue selected from the group comprising phenyl, naphthyl, anthracenyl, pyrrolyl, indolyl, furanyl, benzo[b]furanyl, thiophenyl, benz[b]thiophenyl, benzo[d]thiazolyl, pyrazolyl, imidazolyl, thiazolyl, thiadiazolyl, triazolyl, oxazolyl, oxadiazolyl, isoxazolyl, pyridinyl, pyridazinyl, pyrimidinyl, pyrazinyl, pyranyl, indazolyl, quinolinyl, isoquinolinyl and quinazolinyl, which in each case can be bound via a C₁₋₃-alkylene-, C₂₋₃-alkenylene- or C₂₋₃-alkynylene group and/or is unsubstituted or substituted with optionally 1, 2, 3, 4 or 5 substituents mutually independently selected from the group comprising F, Cl, Br, I, —CN, —NO₂, methyl, ethyl, n-propyl, isopropyl, n-butyl, tert-butyl, n-pentyl, ethenyl, allyl, ethynyl, propynyl, —C≡C—Si(CH₃)₃, —C≡C—Si(C₂H₅)₃, —CH₂—O—CH₃, —CH₂—O—C₂H₅, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, pyrazolyl, phenyl, —N(CH₃)₂, —N(C₂H₅)₂, —NH—CH₃, —NH—C₂H₅, —CH₂—O—C(═O)-phenyl, —C(═O)—O—CH₃, —C(═O)—O—C₂H₅, —C(═O)—O—CH(CH₃)₂, —C(═O)—O—(CH₂)₃—CH₃, —C(═O)—O—C(CH₃)₃, —C(═O)—O-phenyl, —C(═O)—H, —C(═O)—CH₃, —C(═O)—C₂H₅, —NH—C(═O)—CH₃, —NH—C(═O)—C₂H₅, —O—C(═O)-phenyl, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅, —C(═O)—NH—C(CH₃)₃, —C(═O)—N(C₂H₅)₂, —C(═O)—NH-phenyl, —C(═O)—N(CH₃)— phenyl, —C(═O)—N(C₂H₅)-phenyl, cyclopropyl, cyclobutyl, cyclopentyl, cyclohexyl, phenyl, furyl (furanyl), thiadiazolyl, thiophenyl (thienyl) and benzyl;

and, provided that M² denotes unsubstituted or substituted heteroaryl or unsubstituted or substituted naphthyl or an unsubstituted or substituted phenyl residue, which can be condensed (annelated) with unsubstituted or substituted 5- to 7-membered heterocycloalkyl or with unsubstituted or substituted C₅₋₇-cycloalkyl, R⁵ can additionally denote —C(═O)—R¹¹;
and in each case the remaining residues have the above-mentioned meaning, in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Substituted bis(hetero)aromatic N-ethylpropiolamides of the above-mentioned general formula I are furthermore preferred, in which R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³ and R²⁴, mutually independently, in each case denote C₁₋₆-alkyl, which is unsubstituted or substituted with optionally 1, 2, 3, 4 or 5 substituents mutually independently selected from the group comprising F, Cl, Br, I, —NO₂, —CN, —OH, —SH and —NH₂; C₃₋₇-cycloalkyl, C₅₋₆-cycloalkenyl, 5- to 7-membered heterocycloalkyl or 5- to 7-membered heterocycloalkenyl, which in each case can be bound via a C₁₋₃-alkylene-, C₂₋₃-alkenylene- or C₂₋₃-alkynylene group and/or is unsubstituted or substituted with optionally 1, 2, 3, 4 or 5 substituents mutually independently selected from the group comprising F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or a residue selected from the group comprising phenyl, naphthyl, anthracenyl, pyrrolyl, indolyl, furanyl, benzo[b]furanyl, thiophenyl, benz[b]thiophenyl, benzo[d]thiazolyl, pyrazolyl, imidazolyl, thiazolyl, thiadiazolyl, triazolyl, oxazolyl, oxadiazolyl, isoxazolyl, pyridinyl, pyridazinyl, pyrimidinyl, pyrazinyl, pyranyl, indazolyl, quinolinyl, isoquinolinyl and quinazolinyl, which in each case can be bound via a C₁₋₃-alkylene-, C₂₋₃-alkenylene- or C₂₋₃-alkynylene group and/or is unsubstituted or substituted with optionally 1, 2, 3, 4 or 5 substituents mutually independently selected from the group comprising F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;

and in each case the remaining residues have the above-mentioned meaning, in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Substituted bis(hetero)aromatic N-ethylpropiolamides of the above-mentioned general formula I are also preferred, in which

### I.)

M¹ denotes a phenyl residue, which is unsubstituted or substituted with 1, 2, 3, 4 or 5 substituents mutually independently selected from the group comprising F, Cl, Br, I, —CN, —NO₂, methyl, ethyl, n-propyl, isopropyl, ethenyl, allyl, ethynyl, propynyl, —CH₂—O—CH₃, —CH₂—O—C₂H₅, —OH, —SH, —NH₂, —C(═O)—OH, —S—CH₃, —S—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂, —S—CH₂F, —N(CH₃)₂, —N(C₂H₅)₂, —NH—CH₃, —NH—C₂H₅, —C(═O)—O—CH₃, —C(═O)—O—C₂H₅, —C(═O)—O—CH(CH₃)₂, —C(═O)—O—(CH₂)₃—CH₃, —C(═O)—O—C(CH₃)₃, —C(═O)—O-phenyl, —C(═O)—H, —C(═O)—CH₃, —C(═O)—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅, —C(═O)—NH—C(CH₃)₃, —C(═O)—N(C₂H₅)₂, —C(═O)—NH-phenyl, —C(═O)—N(CH₃)— phenyl, —C(═O)—N(C₂H₅)-phenyl, cyclopropyl, cyclobutyl, cyclopentyl and cyclohexyl;
and M² denotes a phenyl residue, which is substituted with 1, 2, 3, 4 or 5 substituents mutually independently selected from the group comprising F, Cl, Br, I, —CN, —NO₂, methyl, ethyl, n-propyl, isopropyl, n-butyl, isobutyl, tert-butyl, ethenyl, allyl, ethynyl, propynyl, —CH₂—O—CH₃, —CH₂—O—C₂H₅, —OH, —SH, —NH₂, —C(═O)—OH, —S—CH₃, —S—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂, —S—CH₂F, —N(CH₃)₂, —N(C₂H₅)₂, —NH—CH₃, —NH—C₂H₅, —C(═O)—O—CH₃, —C(═O)—O—C₂H₅, —C(═O)—O—CH(CH₃)₂, —C(═O)—O—(CH₂)₃—CH₃, —C(═O)—O—C(CH₃)₃, —C(═O)—O-phenyl, —C(═O)—H, —C(═O)—CH₃, —C(═O)—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅, —C(═O)—NH—C(CH₃)₃, —C(═O)—N(C₂H₅)₂, —C(═O)—NH-phenyl, —C(═O)—N(CH₃)-phenyl, —C(═O)—N(C₂H₅)-phenyl, cyclopropyl, cyclobutyl, cyclopentyl and cyclohexyl;
or M² denotes a residue selected from the group comprising indolyl, naphthyl, thiophenyl (thienyl), furanyl (furyl), pyrrolyl, pyrazolyl, triazolyl, pyridinyl, imidazolyl, thiazolyl, thiadiazolyl, oxazolyl, oxadiazolyl, tetrazolyl, triazinyl, isoxazolyl, pyridazinyl, pyrazinyl, pyrimidinyl, diaza-naphthyl, quinoxalinyl, quinazolinyl, quinolinyl, naphthridinyl, isoquinolinyl, indolinyl, (2,3)-dihydrobenzofuranyl, (2,3)-dihydrobenzo[d]oxazolyl, benzo[d][1,3]dioxolyl, isoindolinyl, (1,2,3,4)-tetrahydroquinolinyl and (2,3)-dihydrobenzo[b][1,4]dioxinyl, which is unsubstituted or substituted with 1, 2, 3, 4 or 5 substituents mutually independently selected from the group comprising F, Cl, Br, I, —CN, —NO₂, methyl, ethyl, n-propyl, isopropyl, n-butyl, isobutyl, tert-butyl, ethenyl, allyl, ethynyl, propynyl, —CH₂—O—CH₃, —CH₂—O—C₂H₅, —OH, —SH, —NH₂, —C(═O)—OH, —S—CH₃, —S—C₂H₅, —O—CH₃, —O—C₂H₅, —O—C₃H₇, —O—C(CH₃)₃, —CF₃, —CHF₂, —CH₂F, —O—CF₃, —CHF₂, —O—CH₂F, —C(═O)—CF₃, —S—CF₃, —S—CHF₂, —S—CH₂F, —N(CH₃)₂, —N(C₂H₅)₂, —NH—CH₃, —NH—C₂H₅, —C(═O)—O—CH₃, —C(═O)—O—C₂H₅, —C(═O)—O—CH(CH₃)₂, —C(═O)—O—(CH₂)₃—CH₃, —C(═O)—O—C(CH₃)₃, —C(═O)—O-phenyl, —C(═O)—H, —C(═O)—CH₃, —C(═O)—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅, —C(═O)—NH—C(CH₃)₃, —C(═O)—N(C₂H₅)₂, —C(═O)—NH-phenyl, —C(═O)—N(CH₃)-phenyl, —C(═O)—N(C₂H₅)-phenyl, cyclopropyl, cyclobutyl, cyclopentyl and cyclohexyl;

### or II.)

M¹ denotes a residue selected from the group comprising naphthyl, thiophenyl (thienyl), furanyl (furyl), pyrrolyl, pyrazolyl, triazolyl, pyridinyl, imidazolyl, indolyl, thiazolyl, thiadiazolyl, oxazolyl, oxadiazolyl, tetrazolyl, triazinyl, isoxazolyl, pyridazinyl, pyrazinyl, pyrimidinyl, diaza-naphthyl, quinoxalinyl, quinazolinyl, quinolinyl, naphthridinyl and isoquinolinyl, which is unsubstituted or substituted with 1, 2, 3, 4 or 5 substituents mutually independently selected from the group comprising F, Cl, Br, I, —CN, —NO₂, methyl, ethyl, n-propyl, isopropyl, n-butyl, isobutyl, tert-butyl, ethenyl, allyl, ethynyl, propynyl, —CH₂—O—CH₃, —CH₂—O—C₂H₅, —OH, —SH, —NH₂, —C(═O)—OH, —S—CH₃, —S—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂, —S—CH₂F, —N(CH₃)₂, —N(C₂H₅)₂, —NH—CH₃, —NH—C₂H₅, —C(═O)—O—CH₃, —C(═O)—O—C₂H₅, —C(═O)—O—CH(CH₃)₂, —C(═O)—O—(CH₂)₃—CH₃, —C(═O)—O—C(CH₃)₃, —C(═O)—O-phenyl, —C(═O)—H, —C(═O)—CH₃, —C(═O)—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅, —C(═O)—NH—C(CH₃)₃, —C(═O)—N(C₂H₅)₂, —C(═O)—NH-phenyl, —C(═O)—N(CH₃)-phenyl, —C(═O)—N(C₂H₅)-phenyl, cyclopropyl, cyclobutyl, cyclopentyl and cyclohexyl;
and M² denotes a residue selected from the group comprising indolyl, naphthyl, thiophenyl (thienyl), furanyl (furyl), pyrrolyl, pyrazolyl, triazolyl, pyridinyl, imidazolyl, thiazolyl, thiadiazolyl, oxazolyl, oxadiazolyl, tetrazolyl, triazinyl, isoxazolyl, pyridazinyl, pyrazinyl, pyrimidinyl, diaza-naphthyl, quinoxalinyl, quinazolinyl, quinolinyl, naphthridinyl, isoquinolinyl, indolinyl, (2,3)-dihydrobenzofuranyl, (2,3)-dihydrobenzo[d]oxazolyl, benzo[d][1,3]dioxolyl, isoindolinyl, (1,2,3,4)-tetrahydroquinolinyl and (2,3)-dihydrobenzo[b][1,4]dioxinyl, which is unsubstituted or substituted with 1, 2, 3, 4 or 5 substituents mutually independently selected from the group comprising F, Cl, Br, I, —CN, —NO₂, methyl, ethyl, n-propyl, isopropyl, n-butyl, isobutyl, tert-butyl, ethenyl, allyl, ethynyl, propynyl, —CH₂—O—CH₃, —CH₂—O—C₂H₅, —OH, —SH, —NH₂, —C(═O)—OH, —S—CH₃, —S—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂, —S—CH₂F, —N(CH₃)₂, —N(C₂H₅)₂, —NH—CH₃, —NH—C₂H₅, —C(═O)—O—CH₃, —C(═O)—O—C₂H₅, —C(═O)—O—CH(CH₃)₂, —C(═O)—O—(CH₂)₃—CH₃, —C(═O)—O—C(CH₃)₃, —C(═O)—O-phenyl, —C(═O)—H, —C(═O)—CH₃, —C(═O)—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅, —C(═O)—NH—C(CH₃)₃, —C(═O)—N(C₂H₅)₂, —C(═O)—NH-phenyl, —C(═O)—N(CH₃)-phenyl, —C(═O)—N(C₂H₅)-phenyl, cyclopropyl, cyclobutyl, cyclopentyl and cyclohexyl;
or M² denotes a phenyl residue, which is unsubstituted or substituted with 1, 2, 3, 4 or 5 substituents mutually independently selected from the group comprising F, Cl, Br, I, —CN, —NO₂, methyl, ethyl, n-propyl, isopropyl, n-butyl, isobutyl, tert-butyl, ethenyl, allyl, ethynyl, propynyl, —CH₂—O—CH₃, —CH₂—O—C₂H₅, —OH, —SH, —NH₂, —C(═O)—OH, —S—CH₃, —S—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂, —S—CH₂F, —N(CH₃)₂, —N(C₂H₅)₂, —NH—CH₃, —NH—C₂H₅, —C(═O)—O—CH₃, —C(═O)—O—C₂H₅, —C(═O)—O—CH(CH₃)₂, —C(═O)—O—(CH₂)₃—CH₃, —C(═O)—O—C(CH₃)₃, —C(═O)—O-phenyl, —C(═O)—H, —C(═O)—CH₃, —C(═O)—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅, —C(═O)—NH—C(CH₃)₃, —C(═O)—N(C₂H₅)₂, —C(═O)—NH-phenyl, —C(═O)—N(CH₃)-phenyl, —C(═O)—N(C₂H₅)-phenyl, cyclopropyl, cyclobutyl, cyclopentyl and cyclohexyl;
and in each case
R¹ and R², mutually independently, in each case denote H; F; Cl; Br; I; —NO₂; —CN; —OH; —SH; —O—R⁶; —S—R⁷; —NH—R⁸; —NR⁹R¹⁰; or C₁₋₆-alkyl, which is unsubstituted or substituted with optionally 1, 2, 3, 4 or 5 substituents mutually independently selected from the group comprising F, Cl, Br, I, —NO₂, —CN, —OH, —SH and —NH₂; or a phenyl residue, which is unsubstituted or substituted with 1, 2, 3, 4 or 5 substituents mutually independently selected from the group comprising 1, 2, 3, 4 or 5 substituents mutually independently selected from the group comprising F, Cl, Br, I, —CN, —NO₂, methyl, ethyl, n-propyl, isopropyl, n-butyl, tert-butyl, n-pentyl, —OH, —SH, —NH₂, —C(═O)—OH, —O—CH₃, —O—C₂H₅, —O—C₃H₇, —O—C(CH₃)₃, —CF₃, —CHF₂, —CH₂F, —O—CF₃, —O—CHF₂, —O—CH₂F, —S—CF₃, —S—CHF₂ and —S—CH₂F;
or R¹ and R² jointly denote an oxo group (═O);
R³ and R⁴, mutually independently, in each case denote H; F; Cl; Br; I; —NO₂; —CN; —OH; —SH; —C(═O)—OH; —O—R⁶; —S—R⁷; —NH—R⁸; —NR⁹R¹⁰; —C(═O)—R¹¹; —C(═O)—O—R¹²C(═O)—NH₂; —C(═O)—NH—R¹⁷; —C(═O)—NR¹⁸R¹⁹; or C₁₋₆-alkyl, which is unsubstituted or substituted with optionally 1, 2, 3, 4 or 5 substituents mutually independently selected from the group comprising F, Cl, Br, I, —NO₂, —CN, —OH, —SH and —NH₂; or a phenyl residue, which is unsubstituted or substituted with 1, 2, 3, 4 or 5 substituents mutually independently selected from the group comprising 1, 2, 3, 4 or 5 substituents mutually independently selected from the group comprising F, Cl, Br, I, —CN, —NO₂, methyl, ethyl, n-propyl, isopropyl, n-butyl, tert-butyl, n-pentyl, —OH, —SH, —NH₂, —C(═O)—OH, —O—CH₃, —O—C₂H₅, —O—C₃H₇, —O—C(CH₃)₃, —CF₃, —CHF₂, —CH₂F, —O—CF₃, —O—CHF₂, —O—CH₂F, —S—CF₃, —S—CHF₂ and —S—CH₂F;
R⁵ denotes H; —C(═O)—O—R¹²; —C(═O)—NH—R¹⁷; —C(═O)—NR¹⁸R¹⁹; —S(═O)—R²⁰; —S(═O)₂—R²¹; C₁₋₆-alkyl, which is unsubstituted or substituted with optionally 1, 2, 3, 4 or 5 substituents mutually independently selected from the group comprising F, Cl, Br, I, —NO₂, —CN, —OH, —SH and —NH₂; C₃₋₇-cycloalkyl, which is unsubstituted or substituted with optionally 1, 2, 3, 4 or 5 substituents mutually independently selected from the group comprising F, Cl, Br, I, methyl, ethyl, n-propyl, isopropyl, n-butyl, 2-butyl, isobutyl and tert-butyl; or a phenyl, benzyl or phenethyl residue, which is unsubstituted or substituted with 1, 2, 3, 4 or 5 substituents mutually independently selected from the group comprising 1, 2, 3, 4 or 5 substituents mutually independently selected from the group comprising F, Cl, Br, I, —CN, —NO₂, methyl, ethyl, n-propyl, isopropyl, n-butyl, tert-butyl, n-pentyl, —OH, —SH, —NH₂, —C(═O)—OH, —O—CH₃, —O—C₂H₅, —O—C₃H₇, —O—C(CH₃)₃, —CF₃, —CHF₂, —CH₂F, —O—CF₃, —O—CHF₂, —O—CH₂F, —S—CF₃, —S—CHF₂, —S—CH₂F, —N(CH₃)₂, —N(C₂H₅)₂, —NH—CH₃, —NH—C₂H₅, —CH₂—O—C(═O)-phenyl, —C(═O)—O—CH₃, —C(═O)—O—C₂H₅, —C(═O)—O—CH(CH₃)₂, —C(═O)—O—(CH₂)₃—CH₃, —C(═O)—O—C(CH₃)₃, —C(═O)—O-phenyl, —C(═O)—H, —C(═O)—CH₃, —C(═O)—C₂H₅, —C(═O)—NH₂, —C(═O)—NH—CH₃ and —C(═O)—N(CH₃)₂;
and, provided that M² denotes unsubstituted or substituted heteroaryl or unsubstituted or substituted naphthyl or an unsubstituted or substituted phenyl residue, which can be condensed (annelated) with unsubstituted or substituted 5- to 7-membered heterocycloalkyl or with unsubstituted or substituted C₅₋₇-cycloalkyl, R⁵ can additionally denote —C(═O)—R¹¹;
and R⁶, R⁷, R⁸, R⁹, R¹⁰, R¹¹, R¹², R¹⁷, R¹⁸, R¹⁹, R²⁰ and R²¹, mutually independently, in each case denote C₁₋₆-alkyl, which is unsubstituted or substituted with optionally 1, 2, 3, 4 or 5 substituents mutually independently selected from the group comprising F, Cl, Br, I, —NO₂, —CN, —OH, —SH and —NH₂; unsubstituted C₃₋₇-cycloalkyl; unsubstituted C₅₋₆-cycloalkenyl; unsubstituted 5- to 7-membered heterocycloalkyl and unsubstituted 5- to 7-membered heterocycloalkenyl; or a residue selected from the group comprising phenyl, benzyl, naphthyl, anthracenyl, pyrrolyl, indolyl, furanyl, benzo[b]furanyl, thiophenyl, benz[b]thiophenyl, benzo[d]thiazolyl, pyrazolyl, imidazolyl, thiazolyl, thiadiazolyl, triazolyl, oxazolyl, oxadiazolyl, isoxazolyl, pyridinyl, pyridazinyl, pyrimidinyl, pyrazinyl, pyranyl, indazolyl, quinolinyl, isoquinolinyl and quinazolinyl, which in each case is unsubstituted or substituted with optionally 1, 2, 3, 4 or 5 substituents mutually independently selected from the group comprising F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Substituted bis(hetero)aromatic N-ethylpropiolamides of the above-mentioned general formula I are particularly preferred, in which

### I.)

M¹ denotes a phenyl residue, which is unsubstituted or substituted with 1, 2, 3, 4 or 5 substituents mutually independently selected from the group comprising F, Cl, Br, I, —CN, —NO₂, methyl, ethyl, n-propyl, isopropyl, ethenyl, allyl, ethynyl, propynyl, —OH, —NH₂, —O—CH₃, —O—C₂H₅, —O—C₃H₇, —CF₃, —CHF₂, —CH₂F, —O—CF₃, —O—CHF₂, —O—CH₂F, —N(CH₃)₂, —N(C₂H₅)₂, —NH—CH₃, —NH—C₂H₅, —C(═O)—O—CH₃, —C(═O)—O—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅ and cyclopropyl;

and M² denotes a phenyl residue, which is substituted with 1, 2, 3, 4 or 5 substituents mutually independently selected from the group comprising F, Cl, Br, I, —CN, —NO₂, methyl, ethyl, n-propyl, isopropyl, n-butyl, isobutyl, tert-butyl, ethenyl, allyl, ethynyl, propynyl, —OH, —NH₂, —O—CH₃, —O—C₂H₅, —O—C₃H₇, —CF₃, —CHF₂, —CH₂F, —O—CF₃, —O—CHF₂, —O—CH₂F, —N(CH₃)₂, —N(C₂H₅)₂, —NH—CH₃, —NH—C₂H₅, —C(═O)—O—CH₃, —C(═O)—O—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅ and cyclopropyl;
or M² denotes a residue selected from the group comprising indolyl, naphthyl, thiophenyl (thienyl), furanyl (furyl), pyrrolyl, pyrazolyl, triazolyl, pyridinyl, imidazolyl, thiazolyl, thiadiazolyl, oxazolyl, oxadiazolyl, tetrazolyl, triazinyl, isoxazolyl, pyridazinyl, pyrazinyl, pyrimidinyl, diaza-naphthyl, quinoxalinyl, quinazolinyl, quinolinyl, naphthridinyl, isoquinolinyl, indolinyl, (2,3)-dihydrobenzofuranyl, (2,3)-dihydrobenzo[d]oxazolyl, benzo[d][1,3]dioxolyl, isoindolinyl, (1,2,3,4)-tetrahydroquinolinyl and (2,3)-dihydrobenzo[b][1,4]dioxinyl, which is unsubstituted or substituted with 1, 2, 3, 4 or 5 substituents mutually independently selected from the group comprising F, Cl, Br, I, —CN, —NO₂, methyl, ethyl, n-propyl, isopropyl, n-butyl, isobutyl, tert-butyl, ethenyl, allyl, ethynyl, propynyl, —OH, —NH₂, —O—CH₃, —O—C₂H₅, —O—C₃H₇, —CF₃, —CHF₂, —CH₂F, —O—CF₃, —O—CHF₂, —O—CH₂F, —N(CH₃)₂, —N(C₂H₅)₂, —NH—CH₃, —NH—C₂H₅, —C(═O)—O—CH₃, —C(═O)—O—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅ and cyclopropyl;

### or II.)

M¹ denotes a residue selected from the group comprising naphthyl, thiophenyl (thienyl), furanyl (furyl), pyrrolyl, pyrazolyl, triazolyl, pyridinyl, imidazolyl, indolyl, thiazolyl, thiadiazolyl, oxazolyl, oxadiazolyl, tetrazolyl, triazinyl, isoxazolyl, pyridazinyl, pyrazinyl, pyrimidinyl, diaza-naphthyl, quinoxalinyl, quinazolinyl, quinolinyl, naphthridinyl, isoquinolinyl, indolinyl, (2,3)-dihydrobenzofuranyl, (2,3)-dihydrobenzo[d]oxazolyl, benzo[d][1,3]dioxolyl, isoindolinyl, (1,2,3,4)-tetrahydroquinolinyl and (2,3)-dihydrobenzo[b][1,4]dioxinyl, which is unsubstituted or substituted with 1, 2, 3, 4 or 5 substituents mutually independently selected from the group comprising F, Cl, Br, I, —CN, —NO₂, methyl, ethyl, n-propyl, isopropyl, n-butyl, isobutyl, tert-butyl, ethenyl, allyl, ethynyl, propynyl, —OH, —NH₂, —O—CH₃, —O—C₂H₅, —O—C₃H₇, —CF₃, —CHF₂, —CH₂F, —O—CF₃, —O—CHF₂, —O—CH₂F, —N(CH₃)₂, —N(C₂H₅)₂, —NH—CH₃, —NH—C₂H₅, —C(═O)—O—CH₃, —C(═O)—O—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅ and cyclopropyl;
and M² denotes a residue selected from the group comprising indolyl, naphthyl, thiophenyl (thienyl), furanyl (furyl), pyrrolyl, pyrazolyl, triazolyl, pyridinyl, imidazolyl, thiazolyl, thiadiazolyl, oxazolyl, oxadiazolyl, tetrazolyl, triazinyl, isoxazolyl, pyridazinyl, pyrazinyl, pyrimidinyl, diaza-naphthyl, quinoxalinyl, quinazolinyl, quinolinyl, naphthridinyl, isoquinolinyl, indolinyl, (2,3)-dihydrobenzofuranyl, (2,3)-dihydrobenzo[d]oxazolyl, benzo[d][1,3]dioxolyl, isoindolinyl, (1,2,3,4)-tetrahydroquinolinyl and (2,3)-dihydrobenzo[b][1,4]dioxinyl, which is unsubstituted or substituted with 1, 2, 3, 4 or 5 substituents mutually independently selected from the group comprising F, Cl, Br, I, —CN, —NO₂, methyl, ethyl, n-propyl, isopropyl, n-butyl, isobutyl, tert-butyl, ethenyl, allyl, ethynyl, propynyl, —OH, —NH₂, —O—CH₃, —O—C₂H₅, —O—C₃H₇, —CF₃, —CHF₂, —CH₂F, —O—CF₃, —O—CHF₂, —O—CH₂F, —N(CH₃)₂, —N(C₂H₅)₂, —NH—CH₃, —NH—C₂H₅, —C(═O)—O—CH₃, —C(═O)—O—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅ and cyclopropyl;
or M² denotes a phenyl residue, which is unsubstituted or substituted with 1, 2, 3, 4 or 5 substituents mutually independently selected from the group comprising F, Cl, Br, I, —CN, —NO₂, methyl, ethyl, n-propyl, isopropyl, n-butyl, isobutyl, tert-butyl, ethenyl, allyl, ethynyl, propynyl, —OH, —NH₂, —O—CH₃, —O—C₂H₅, —O—C₃H₇, —CF₃, —CHF₂, —CH₂F, —O—CF₃, —O—CHF₂, —O—CH₂F, —N(CH₃)₂, —N(C₂H₅)₂, —NH—CH₃, —NH—C₂H₅, —C(═O)—O—CH₃, —C(═O)—O—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅ and cyclopropyl;
and in each case
R¹ and R², mutually independently, in each case denote H; F; Cl; Br; I; —NO₂; —CN; —OH; —SH; —O—R⁶; —S—R⁷; —NH—R⁸; —NR⁹R¹⁰; or a residue selected from the group comprising methyl, ethyl, n-propyl, isopropyl, n-butyl, tert-butyl, isobutyl, n-pentyl, —CF₃, —CF₂H, —CFH₂, —C₂F₅ and —CH₂—CF₃ or a phenyl residue, which can be unsubstituted or substituted with 1, 2, 3, 4 or 5 substituents mutually independently selected from the group comprising F, Cl, Br, I, —CN, —NO₂, methyl, ethyl, n-propyl, isopropyl, n-butyl, tert-butyl, n-pentyl, —OH, —SH, —NH₂, —O—CH₃, —O—C₂H₅, —O—C₃H₇, —O—C(CH₃)₃, —CF₃, —CHF₂, —CH₂F and —O—CF₃;
or R¹ and R² jointly denote an oxo group (═O);
R³ and R⁴, mutually independently, in each case denote H; F; Cl; Br; I; —NO₂; —CN; —OH; —SH; —C(═O)—OH; —O—R⁶; —S—R⁷; —NH—R⁸; —NR⁹R¹⁰; or a residue selected from the group comprising methyl, ethyl, n-propyl, isopropyl, n-butyl, tert-butyl, isobutyl, n-pentyl, —CF₃, —CF₂H, —CFH₂, —C₂F₅ and —CH₂—CF₃ or a phenyl residue, which can be unsubstituted or substituted with 1, 2, 3, 4 or 5 substituents mutually independently selected from the group comprising F, Cl, Br, I, —CN, —NO₂, methyl, ethyl, n-propyl, isopropyl, n-butyl, tert-butyl, n-pentyl, —OH, —SH, —NH₂, —O—CH₃, —O—C₂H₅, —O—C₃H₇, —O—C(CH₃)₃, —CF₃, —CHF₂, —CH₂F and —O—CF₃;
R⁵ denotes H; —C(═O)—O—R¹²; —S(═O)—R²⁰; —S(═O)₂—R²¹; a residue selected from the group comprising methyl, ethyl, n-propyl, isopropyl, n-butyl, tert-butyl, isobutyl, n-pentyl, —CF₃, —CF₂H, —CFH₂, —C₂F₅ and —CH₂—CF₃; a residue selected from the group comprising cyclopropyl, cyclobutyl, cyclopentyl and cyclohexyl; or a benzyl or phenethyl residue, which is unsubstituted or substituted with 1, 2, 3, 4 or 5 substituents mutually independently selected from the group comprising F, Cl, Br, I, —CN, —NO₂, methyl, ethyl, n-propyl, isopropyl, n-butyl, tert-butyl, n-pentyl, —OH, —SH, —NH₂, —O—CH₃, —O—C₂H₅, —O—C₃H₇, —O—C(CH₃)₃, —CF₃, —CHF₂, —CH₂F, —O—CF₃, —O—CHF₂, —O—CH₂F, —S—CF₃, —S—CHF₂, —S—CH₂F, —N(CH₃)₂, —N(C₂H₅)₂, —NH—CH₃ and —NH—C₂H₅;
and, provided that M² denotes unsubstituted or substituted heteroaryl or unsubstituted or substituted naphthyl or an unsubstituted or substituted phenyl residue, which can be condensed (annelated) with unsubstituted or substituted 5- to 7-membered heterocycloalkyl or with unsubstituted or substituted C₅₋₇-cycloalkyl, R⁵ can additionally denote —C(═O)—R¹¹;
and R⁶, R⁷, R⁸, R⁹, R¹⁰, R¹¹, R¹², R²⁰ and R²¹, mutually independently, in each case denote a residue selected from the group comprising methyl, ethyl, n-propyl, isopropyl, n-butyl, tert-butyl, isobutyl, n-Pentyl, —CF₃, —CF₂H, —CFH₂, —C₂F₅ and —CH₂—CF₃; a residue selected from the group comprising cyclopropyl, cyclobutyl, cyclopentyl and cyclohexyl; a residue selected from the group comprising pyrrolidinyl, piperidinyl, morpholinyl, piperazinyl, thiomorpholinyl, tetrahydropyranyl, oxetanyl, azepanyl and diazepanyl; or a residue selected from the group comprising phenyl, benzyl, naphthyl, furanyl, thiophenyl, pyrazolyl, imidazolyl, thiazolyl and thiadiazolyl, which in each case is unsubstituted or substituted with optionally 1, 2, 3, 4 or 5 substituents mutually independently selected from the group comprising F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Substituted bis(hetero)aromatic N-ethylpropiolamides of the above-mentioned general formula I are also particularly preferred, in which

### I.)

M¹ denotes a phenyl residue, which is unsubstituted or substituted with 1, 2, 3, 4 or 5 substituents mutually independently selected from the group comprising F, Cl, Br, I, —CN, —NO₂, methyl, ethyl, n-propyl, isopropyl, ethenyl, allyl, ethynyl, propynyl, —OH, —NH₂, —O—CH₃, —O—C₂H₅, —O—C₃H₇, —CF₃, —CHF₂, —CH₂F, —O—CF₃, —O—CHF₂, —O—CH₂F, —N(CH₃)₂, —N(C₂H₅)₂, —NH—CH₃, —NH—C₂H₅, —C(═O)—O—CH₃, —C(═O)—O—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅ and cyclopropyl;
and M² denotes a phenyl residue, which is substituted with 1, 2, 3, 4 or 5 substituents mutually independently selected from the group comprising F, Cl, Br, I, —CN, —NO₂, methyl, ethyl, n-propyl, isopropyl, n-butyl, Isobutyl, tert-butyl, ethenyl, allyl, ethynyl, propynyl, —OH, —NH₂, —O—CH₃, —O—C₂H₅, —O—C₃H₇, —CF₃, —CHF₂, —CH₂F, —O—CF₃, —O—CHF₂, —O—CH₂F, —N(CH₃)₂, —N(C₂H₅)₂, —NH—CH₃, —NH—C₂H₅, —C(═O)—O—CH₃, —C(═O)—O—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅ and cyclopropyl;
or M² denotes a residue selected from the group comprising indolyl, thiophenyl (thienyl), pyridinyl, thiazolyl, thiadiazolyl, pyridazinyl, pyrazinyl, pyrimidinyl, benzo[d][1,3]dioxolyl and (2,3)-dihydrobenzo[b][1,4]dioxinyl, which is unsubstituted or substituted with 1, 2, 3, 4 or 5 substituents mutually independently selected from the group comprising F, Cl, Br, I, —CN, —NO₂, methyl, ethyl, n-propyl, isopropyl, n-butyl, isobutyl, tert-butyl, ethenyl, allyl, ethynyl, propynyl, —OH, —NH₂, —O—CH₃, —O—C₂H₅, —O—C₃H₇, —CF₃, —CHF₂, —CH₂F, —O—CF₃, —O—CHF₂, —O—CH₂F, —N(CH₃)₂, —N(C₂H₅)₂, —NH—CH₃, —NH—C₂H₅, —C(═O)—O—CH₃, —C(═O)—O—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅ and cyclopropyl;

### or II.)

M¹ denotes a residue selected from the group comprising naphthyl, thiophenyl (thienyl), furanyl (furyl), pyridinyl, imidazolyl, indolyl, pyrrolyl, pyrazolyl, thiazolyl, pyridazinyl, pyrazinyl, pyrimidinyl, quinolinyl, isoquinolinyl, benzo[d][1,3]dioxolyl and (2,3)-dihydrobenzo[b][1,4]dioxinyl, which is unsubstituted or substituted with 1, 2, 3, 4 or 5 substituents mutually independently selected from the group comprising F, Cl, Br, I, —CN, —NO₂, methyl, ethyl, n-propyl, isopropyl, n-butyl, isobutyl, tert-butyl, ethenyl, allyl, ethynyl, propynyl, —OH, —NH₂, —O—CH₃, —O—C₂H₅, —O—C₃H₇, —CF₃, —CHF₂, —CH₂F, —O—CF₃, —O—CHF₂, —O—CH₂F, —N(CH₃)₂, —N(C₂H₅)₂, —NH—CH₃, —NH—C₂H₅, —C(═O)—O—CH₃, —C(═O)—O—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅ and cyclopropyl;
and M² denotes a residue selected from the group comprising indolyl, thiophenyl (thienyl), pyridinyl, thiazolyl, thiadiazolyl, pyridazinyl, pyrazinyl, pyrimidinyl, benzo[d][1,3]dioxolyl and (2,3)-dihydrobenzo[b][1,4]dioxinyl, which is unsubstituted or substituted with 1, 2, 3, 4 or 5 substituents mutually independently selected from the group comprising F, Cl, Br, I, —CN, —NO₂, methyl, ethyl, n-propyl, isopropyl, n-butyl, isobutyl, tert-butyl, ethenyl, allyl, ethynyl, propynyl, —OH, —NH₂, —O—CH₃, —O—C₂H₅, —O—C₃H₇, —CF₃, —CHF₂, —CH₂F, —O—CF₃, —O—CHF₂, —O—CH₂F, —N(CH₃)₂, —N(C₂H₅)₂, —NH—CH₃, —NH—C₂H₅, —C(═O)—O—CH₃, —C(═O)—O—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅ and cyclopropyl;
or M² denotes a phenyl residue, which is unsubstituted or substituted with 1, 2, 3, 4 or 5 substituents mutually independently selected from the group comprising F, Cl, Br, I, —CN, —NO₂, methyl, ethyl, n-propyl, isopropyl, n-butyl, isobutyl, tert-butyl, ethenyl, allyl, ethynyl, propynyl, —OH, —NH₂, —O—CH₃, —O—C₂H₅, —O—C₃H₇, —CF₃, —CHF₂, —CH₂F, —O—CF₃, —O—CHF₂, —O—CH₂F, —N(CH₃)₂, —N(C₂H₅)₂, —NH—CH₃, —NH—C₂H₅, —C(═O)—O—CH₃, —C(═O)—O—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅ and cyclopropyl;
and in each case
R¹, R², R³ and R⁴, mutually independently, in each case denote H; F; Cl; Br; —OH; —SH; —CN; —O—R⁶; —S—R⁷; —NH—R⁸; —NR⁹R¹⁰; or a residue selected from the group comprising methyl, ethyl, n-propyl, isopropyl, n-butyl, tert-butyl, isobutyl, n-pentyl, —CF₃, —CF₂H, —CFH₂, —C₂F₅ and —CH₂—CF₃ or a phenyl residue, which can be unsubstituted or substituted with 1, 2, 3, 4 or 5 substituents mutually independently selected from the group comprising F, Cl, Br, I, —CN, —NO₂, methyl, ethyl, n-propyl, isopropyl, n-butyl, tert-butyl, n-pentyl, —OH, —SH, —NH₂, —O—CH₃, —O—C₂H₅, —O—C₃H₇, —O—C(CH₃)₃, —CF₃, —CHF₂, —CH₂F and —O—CF₃;
or R¹ and R² jointly denote an oxo group (═O);
R⁵ denotes H; —C(═O)—O—R¹²; a residue selected from the group comprising methyl, ethyl, n-propyl, isopropyl, n-butyl, tert-butyl, isobutyl, n-pentyl, —CF₃, —CF₂H, —CFH₂, —C₂F₅ and —CH₂—CF₃; a residue selected from the group comprising cyclopropyl, cyclobutyl, cyclopentyl and cyclohexyl; or a benzyl or phenethyl residue, which is unsubstituted or substituted with 1, 2, 3, 4 or 5 substituents mutually independently selected from the group comprising F, Cl, Br, I, —CN, —NO₂, methyl, ethyl, n-propyl, isopropyl, n-butyl, tert-butyl, n-pentyl, —O—CH₃, —O—C₂H₅, —O—C₃H₇, —O—C(CH₃)₃ and —CF₃;
and, provided that M² denotes unsubstituted or substituted heteroaryl or unsubstituted or substituted benzo[d][1,3]dioxolyl or (2,3)-dihydrobenzo[b][1,4]dioxinyl, R⁵ can additionally denote —C(═O)—R¹¹;
and R⁶, R⁷, R⁸, R⁹, R¹⁰, R¹¹ and R¹², mutually independently, in each case denote a residue selected from the group comprising methyl, ethyl, n-propyl, isopropyl, n-butyl, tert-butyl, isobutyl, n-pentyl, —CF₃, —CF₂H, —CFH₂, —C₂F₅ and —CH₂—CF₃; or a residue selected from the group comprising phenyl and benzyl, which in each case is unsubstituted or substituted with optionally 1, 2, 3, 4 or 5 substituents mutually independently selected from the group comprising F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇ and —CF₃;
in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Substituted bis(hetero)aromatic N-ethylpropiolamides of the above-mentioned general formula I are very particularly preferred, in which

### I.)

M¹ denotes a phenyl residue, which is unsubstituted or substituted with 1, 2, 3, 4 or 5 substituents mutually independently selected from the group comprising F, Cl, Br, —CN, —NO₂, methyl, ethyl, n-propyl, isopropyl, —OH, —NH₂, —O—CH₃, —O—C₂H₅, —CF₃, —O—CF₃, —O—CHF₂, —O—CH₂F, —N(CH₃)₂, —NH—CH₃, —C(═O)—O—CH₃, —C(═O)—O—C₂H₅, —NH—C(═O)—CH₃, —O—C(═O)—CH₃, —C(═O)—N(CH₃)₂ and cyclopropyl;
and M² denotes a phenyl residue, which is substituted with 1, 2, 3, 4 or 5 substituents mutually independently selected from the group comprising F, Cl, Br, I, —CN, —OH, —NO₂, methyl, ethyl, n-propyl, isopropyl, n-butyl, isobutyl, tert-butyl, ethenyl, allyl, ethynyl, propynyl, cyclopropyl, —O—CH₃, —O—C₂H₅, —CF₃, —O—CF₃, —O—CHF₂ and —O—CH₂F;
or M² denotes an indolyl, thiophene-2-yl or thiophene-3-yl residue, which is unsubstituted or substituted with 1, 2, 3, 4 or 5 substituents mutually independently selected from the group comprising F, Cl, Br, I, —CN, —NO₂, methyl, ethyl, n-propyl, isopropyl, n-butyl, isobutyl, tert-butyl, ethenyl, allyl, ethynyl, propynyl, cyclopropyl, —OH, —O—CH₃, —O—C₂H₅, —CF₃, —O—CF₃, —O—CHF₂ and —O—CH₂F;

### or II.)

M¹ denotes a residue selected from the group comprising pyrrolyl, pyrazolyl, imidazolyl, indolyl, pyridine-2-yl, pyridine-3-yl, pyridine-4-yl, thiophene-2-yl and thiophene-3-yl, which is unsubstituted or substituted with 1, 2, 3, 4 or 5 substituents mutually independently selected from the group comprising F, Cl, Br, —CN, —NO₂, methyl, ethyl, n-propyl, isopropyl, n-butyl, isobutyl, tert-butyl, —OH, —NH₂, —O—CH₃, —O—C₂H₅, —CF₃, —O—CF₃, —O—CHF₂, —O—CH₂F, —N(CH₃)₂, —NH—CH₃, —C(═O)—O—CH₃, —C(═O)—O—C₂H₅, —NH—C(═O)—CH₃, —O—C(═O)—CH₃, —C(═O)—N(CH₃)₂ and cyclopropyl;
and M² denotes an indolyl, thiophene-2-yl or thiophene-3-yl residue, which is unsubstituted or substituted with 1, 2, 3, 4 or 5 substituents mutually independently selected from the group comprising F, Cl, Br, I, —CN, —NO₂, methyl, ethyl, n-propyl, isopropyl, n-butyl, isobutyl, tert-butyl, ethenyl, allyl, ethynyl, propynyl, cyclopropyl, —OH, —O—CH₃, —O—C₂H₅, —CF₃, —O—CF₃, —O—CHF₂ and —O—CH₂F;
or M² denotes a phenyl residue, which is unsubstituted or substituted with 1, 2, 3, 4 or 5 substituents mutually independently selected from the group comprising F, Cl, Br, I, —CN, —NO₂, —OH, methyl, ethyl, n-propyl, isopropyl, n-butyl, isobutyl, tert-butyl, ethenyl, allyl, ethynyl, propynyl, cyclopropyl, —O—CH₃, —O—C₂H₅, —CF₃, —O—CF₃, —O—CHF₂ and —O—CH₂F;
and in each case
R¹, R², R³ and R⁴, mutually independently, in each case denote H; F; Cl; —CN; —O—CH₃; —O—C₂H₅; —O—CF₃; —O—CF₂H; —O—CFH₂; —N(CH₃)₂; —N(C₂H₅)₂; phenyl; methyl; ethyl; n-propyl or isopropyl;
R⁵ denotes H; methyl; ethyl; n-propyl; isopropyl; cyclopropyl or benzyl;
and, provided that M² denotes an indolyl, thiophene-2-yl or thiophene-3-yl residue, R⁵ can additionally denote —C(═O)—CH₃, —C(═O)—C₂H₅, —C(═O)—CH(CH₃)₂, —C(═O)—C(CH₃)₃, —C(═O)-phenyl or —C(═O)-benzyl;
in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Substituted bis(hetero)aromatic N-ethylpropiolamides of the general formula Ia are also very particularly preferred,

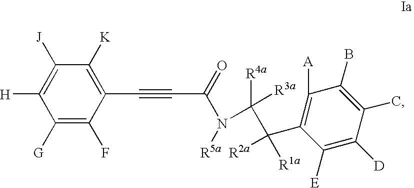

in which
A, B, C, D and E, mutually independently, in each case denote H, F, Cl, Br, —CN, —NO₂, methyl, ethyl, n-propyl, isopropyl, —OH, —NH₂, —O—CH₃, —O—C₂H₅, —CF₃, —O—CF₃, —O—CHF₂, —O—CH₂F, —N(CH₃)₂, —NH—CH₃, —C(═O)—O—CH₃, —C(═O)—O—C₂H₅, —NH—C(═O)—CH₃, —O—C(═O)—CH₃, —C(═O)—N(CH₃)₂ and cyclopropyl;
F, G, H, J and K, mutually independently, in each case denote H, F, Cl, Br, I, —CN, —NO₂, methyl, ethyl, n-propyl, isopropyl, n-butyl, isobutyl, tert-butyl, ethenyl, allyl, ethynyl, propynyl, cyclopropyl, —OH, —O—CH₃, —O—C₂H₅, —CF₃, —O—CF₃, —O—CHF₂ or —O—CH₂F, with the proviso that at least one of the substituents F, G, H, J and K is not equal to H;
R^{1a}, R^{2a}, R^{3a} and R^{4a}, mutually independently, in each case denote H; F; Cl; —CN; —O—CH₃; —O—C₂H₅; —O—CF₃; —O—CF₂H; —O—CFH₂; —N(CH₃)₂; —N(C₂H₅)₂; phenyl; methyl; ethyl; n-propyl or isopropyl;
and R^{5a} denotes H; methyl; ethyl; n-propyl; isopropyl; cyclopropyl or benzyl;
in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Substituted bis(hetero)aromatic N-ethylpropiolamides of the general formula lal are even further preferred,

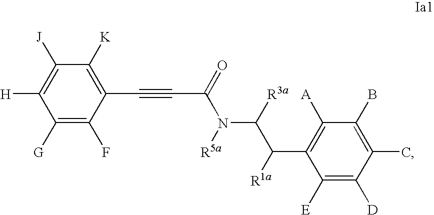

in which A, B, C, D, E, F, G, H, J, K and R^{5a} have the above-mentioned meaning,
R^{1a} denotes H; —O—CH₃; —O—C₂H₅; —O—CF₃; —O—CF₂H; —O—CFH₂; —N(CH₃)₂; —N(C₂H₅)₂; phenyl; methyl; ethyl; n-propyl or isopropyl;
and R^{3a} denotes H; —N(CH₃)₂; —N(C₂H₅)₂; phenyl; methyl; ethyl; n-propyl or isopropyl;
in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Substituted bis(hetero)aromatic N-ethylpropiolamides of the general formula Ib are also very particularly preferred,

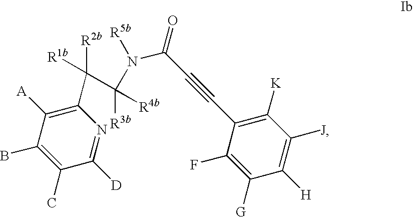

in which
A, B, C and D, mutually independently, in each case denote H, F, Cl, Br, —CN, —NO₂, methyl, ethyl, n-propyl, isopropyl, —OH, —NH₂, —O—CH₃, —O—C₂H₅, —CF₃, —O—CF₃, —O—CHF₂, —O—CH₂F, —N(CH₃)₂, —NH—CH₃, —C(═O)—O—CH₃, —C(═O)—O—C₂H₅, —NH—C(═O)—CH₃, —O—C(═O)—CH₃, —C(═O)—N(CH₃)₂ and cyclopropyl;
F, G, H, J and K, mutually independently, in each case denote H, F, Cl, Br, I, —CN, —NO₂, methyl, ethyl, n-propyl, isopropyl, n-butyl, isobutyl, tert-butyl, ethenyl, allyl, ethynyl, propynyl, cyclopropyl, —OH, —O—CH₃, —O—C₂H₅, —CF₃, —O—CF₃, —O—CHF₂ or —O—CH₂F;
R^{1b}, R^{2b}, R^{3b} and R^{4b}, mutually independently, in each case denote H; F; Cl; —CN; —O—CH₃; —O—C₂H₅; —O—CF₃; —O—CF₂H; —O—CFH₂; —N(CH₃)₂; —N(C₂H₅)₂; phenyl; methyl; ethyl; n-propyl or isopropyl;
and R^{5b} denotes H; methyl; ethyl; n-propyl; isopropyl; cyclopropyl or benzyl;
in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Substituted bis(hetero)aromatic N-ethylpropiolamides of the general formula Ib1 are even further preferred,

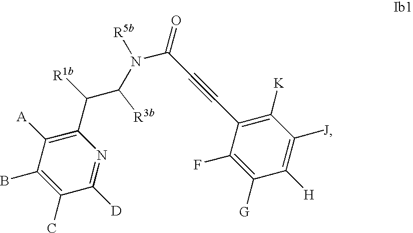

in which A, B, C, D, F, G, H, J, K and R^{5b} have the above-mentioned meaning,
R^{1b} denotes H; —O—CH₃; —O—C₂H₅; —O—CF₃; —O—CF₂H; —O—CFH₂; —N(CH₃)₂; —N(C₂H₅)₂; phenyl; methyl; ethyl; n-propyl or isopropyl;
and R^{3b} denotes H; —N(CH₃)₂; —N(C₂H₅)₂; phenyl; methyl; ethyl; n-propyl or isopropyl;
in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Substituted bis(hetero)aromatic N-ethylpropiolamides of the general formula Ic are also very particularly preferred,

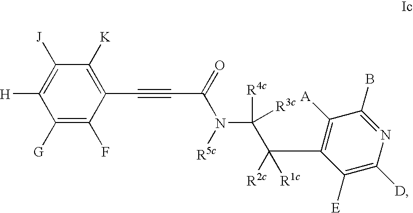

in which
A, B, D and E, mutually independently, in each case denote H, F, Cl, Br, —CN, —NO₂, methyl, ethyl, n-propyl, isopropyl, —OH, —NH₂, —O—CH₃, —O—C₂H₅, —CF₃, —O—CF₃, —O—CHF₂, —O—CH₂F, —N(CH₃)₂, —NH—CH₃, —C(═O)—O—CH₃, —C(═O)—O—C₂H₅, —NH—C(═O)—CH₃, —O—C(═O)—CH₃, —C(═O)—N(CH₃)₂ and cyclopropyl;
F, G, H, J and K, mutually independently, in each case denote H, F, Cl, Br, I, —CN, —NO₂, methyl, ethyl, n-propyl, isopropyl, n-butyl, isobutyl, tert-butyl, ethenyl, allyl, ethynyl, propynyl, cyclopropyl, —OH, —O—CH₃, —O—C₂H₅, —CF₃, —O—CF₃, —O—CHF₂ or —O—CH₂F;
R^{1c}, R^{2c}, R^{3c} and R^{4c}, mutually independently, in each case denote H; F; Cl; —CN; —O—CH₃; —O—C₂H₅; —O—CF₃; —O—CF₂H; —O—CFH₂; —N(CH₃)₂; —N(C₂H₅)₂; phenyl; methyl; ethyl; n-propyl or isopropyl;
and R^{5c} denotes H; methyl; ethyl; n-propyl; isopropyl; cyclopropyl or benzyl;
in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Substituted bis(hetero)aromatic N-ethylpropiolamides of the general formula Ic1 are even further preferred,

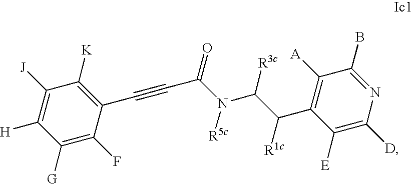

in which A, B, D, E, F, G, H, J, K and R^{5c} have the above-mentioned meaning,
R^{1c} denotes H; —O—CH₃; —O—C₂H₅; —O—CF₃; —O—CF₂H; —O—CFH₂; —N(CH₃)₂; —N(C₂H₅)₂; phenyl; methyl; ethyl; n-propyl or isopropyl;
and R^{3c} denotes H; —N(CH₃)₂; —N(C₂H₅)₂; phenyl; methyl; ethyl; n-propyl or isopropyl;
in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Substituted bis(hetero)aromatic N-ethylpropiolamides of the general formula Id are also very particularly preferred,

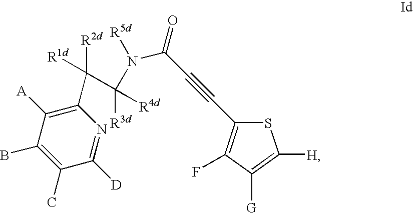

in which
A, B, C and D, mutually independently, in each case denote H, F, Cl, Br, —CN, —NO₂, methyl, ethyl, n-propyl, isopropyl, —OH, —NH₂, —O—CH₃, —O—C₂H₅, —CF₃, —O—CF₃, —O—CHF₂, —O—CH₂F, —N(CH₃)₂, —NH—CH₃, —C(═O)—O—CH₃, —C(═O)—O—C₂H₅, —NH—C(═O)—CH₃, —O—C(═O)—CH₃, —C(═O)—N(CH₃)₂ and cyclopropyl;
F, G, and H, mutually independently, in each case denote H, F, Cl, Br, I, —CN, —NO₂, methyl, ethyl, n-propyl, isopropyl, n-butyl, isobutyl, tert-butyl, ethenyl, allyl, ethynyl, propynyl, cyclopropyl, —OH, —O—CH₃, —O—C₂H₅, —CF₃, —O—CF₃, —O—CHF₂ or —O—CH₂F;
R^{1b}, R^{2d}, R^{3d} and R^{4d}, mutually independently, in each case denote H; F; Cl; —CN; —O—CH₃; —O—C₂H₅; —O—CF₃; —O—CF₂H; —O—CFH₂; —N(CH₃)₂; —N(C₂H₅)₂; phenyl; methyl; ethyl; n-propyl or isopropyl;
and R^{5d} denotes H; methyl; ethyl; n-propyl; isopropyl; cyclopropyl; benzyl; —C(═O)—CH₃, —C(═O)—C₂H₅, —C(═O)—CH(CH₃)₂, —C(═O)—C(CH₃)₃, —C(═O)-phenyl or —C(═O)-benzyl;
in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Substituted bis(hetero)aromatic N-ethylpropiolamides of the general formula Id1 are even further preferred,

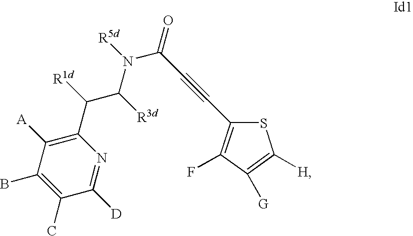

in which A, B, C, D, F, G, H and R^{5d} have the above-mentioned meaning,
R^{1d} denotes H; —O—CH₃; —O—C₂H₅; —O—CF₃; —O—CF₂H; —O—CFH₂; —N(CH₃)₂; —N(C₂H₅)₂; phenyl; methyl; ethyl; n-propyl or isopropyl;
and R^{3d} denotes H; —N(CH₃)₂; —N(C₂H₅)₂; phenyl; methyl; ethyl; n-propyl or isopropyl;
in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Substituted bis(hetero)aromatic N-ethylpropiolamides of the general formula Ie are also very particularly preferred,

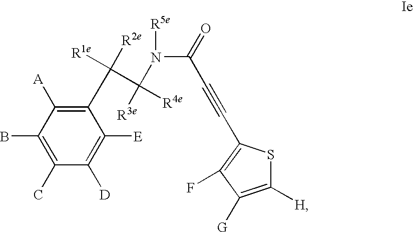

in which
A, B, C, D and E, mutually independently, in each case denote H, F, Cl, Br, —CN, —NO₂, methyl, ethyl, n-propyl, isopropyl, —OH, —NH₂, —O—CH₃, —O—C₂H₅, —CF₃, —O—CF₃, —O—CHF₂, —O—CH₂F, —N(CH₃)₂, —NH—CH₃, —C(═O)—O—CH₃, —C(═O)—O—C₂H₅, —NH—C(═O)—CH₃, —O—C(═O)—CH₃, —C(═O)—N(CH₃)₂ and cyclopropyl;
F, G, and H, mutually independently, in each case denote H, F, Cl, Br, I, —CN, —NO₂, methyl, ethyl, n-propyl, isopropyl, n-butyl, isobutyl, tert-butyl, ethenyl, allyl, ethynyl, propynyl, cyclopropyl, —OH, —O—CH₃, —O—C₂H₅, —CF₃, —O—CF₃, —O—CHF₂ or —O—CH₂F;
R^{1e}, R^{2e}, R^{3e} and R^{4e}, mutually independently, in each case denote H; F; Cl; —CN; —O—CH₃; —O—C₂H₅; —O—CF₃; —O—CF₂H; —O—CFH₂; —N(CH₃)₂; —N(C₂H₅)₂; phenyl; methyl; ethyl; n-propyl or isopropyl;
and R^{5e} denotes H; methyl; ethyl; n-propyl; isopropyl; cyclopropyl; benzyl; —C(═O)—CH₃, —C(═O)—C₂H₅, —C(═O)—CH(CH₃)₂, —C(═O)—C(CH₃)₃, —C(═O)-phenyl or —C(═O)-benzyl;
in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Substituted bis(hetero)aromatic N-ethylpropiolamides of the general formula lel are even further preferred,

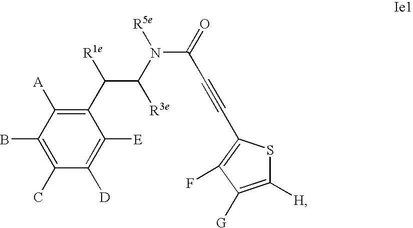

in which A, B, C, D, F, G, H and R^{5e} have the above-mentioned meaning,
R^{1e} denotes H; —O—CH₃; —O—C₂H₅; —O—CF₃; —O—CF₂H; —O—CFH₂; —N(CH₃)₂; —N(C₂H₅)₂; phenyl; methyl; ethyl; n-propyl or isopropyl;
and R^{3e} denotes H; —N(CH₃)₂; —N(C₂H₅)₂; phenyl; methyl; ethyl; n-propyl or isopropyl;
in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Substituted bis(hetero)aromatic N-ethylpropiolamides of the general formula If are also very particularly preferred,

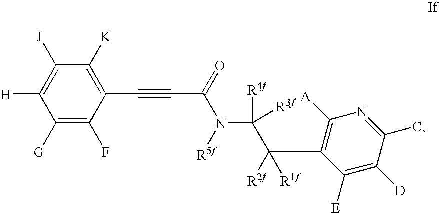

in which
A, C, D and E, mutually independently, in each case denote H, F, Cl, Br, —CN, —NO₂, methyl, ethyl, n-propyl, isopropyl, —OH, —NH₂, —O—CH₃, —O—C₂H₅, —CF₃, —O—CF₃, —O—CHF₂, —O—CH₂F, —N(CH₃)₂, —NH—CH₃, —C(═O)—O—CH₃, —C(═O)—O—C₂H₅, —NH—C(═O)—CH₃, —O—C(═O)—CH₃, —C(═O)—N(CH₃)₂ and cyclopropyl;
F, G, H, J and K, mutually independently, in each case denote H, F, Cl, Br, I, —CN, —NO₂, methyl, ethyl, n-propyl, isopropyl, n-butyl, isobutyl, tert-butyl, ethenyl, allyl, ethynyl, propynyl, cyclopropyl, —OH, —O—CH₃, —O—C₂H₅, —CF₃, —O—CF₃, —O—CHF₂ or —O—CH₂F;
R^{1f}, R^{2f}, R^{3f} and R^{4f}, mutually independently, in each case denote H; F; Cl; —CN; —O—CH₃; —O—C₂H₅; —O—CF₃; —O—CF₂H; —O—CFH₂; —N(CH₃)₂; —N(C₂H₅)₂; phenyl; methyl; ethyl; n-propyl or isopropyl;
and R^{5f} denotes H; methyl; ethyl; n-propyl; isopropyl; cyclopropyl or benzyl;
in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Substituted bis(hetero)aromatic N-ethylpropiolamides of the general formula If1 are even further preferred,

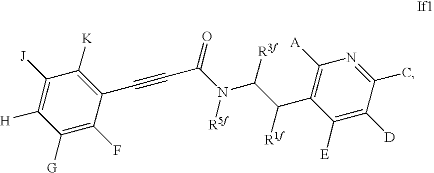

in which A, C, D, E, F, G, H, J, K and R^{5f} have the above-mentioned meaning,
R^{1f} denotes H; —O—CH₃; —O—C₂H₅; —O—CF₃; —O—CF₂H; —O—CFH₂; —N(CH₃)₂; —N(C₂H₅)₂; phenyl; methyl; ethyl; n-propyl or isopropyl;
and R^{3f} denotes H; —N(CH₃)₂; —N(C₂H₅)₂; phenyl; methyl; ethyl; n-propyl or isopropyl;
in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Substituted bis(hetero)aromatic N-ethylpropiolamides of the general formula Ia are even further preferred selected from the group comprising
[1] 3-(3-chlorophenyl)-N-methyl-N-phenethylpropiolamide,
[2] 3-(3-chlorophenyl)-N-phenethylpropiolamide,
[3] 3-(3-chlorophenyl)-N-(2-(pyridine-2-yl)ethyl)propiolamide,
[4] 3-(3-chlorophenyl)-N-(2-(pyridine-4-yl)ethyl)propiolamide,
[5] 3-(2,4-difluorophenyl)-N-(2-(pyridine-2-yl)ethyl)propiolamide,
[6] 3-(3-methoxyphenyl)-N-(2-(pyridine-2-yl)ethyl)propiolamide,
[7] 3-(4-fluoro-3-methylphenyl)-N-(2-(pyridine-2-yl)ethyl)propiolamide,
[8] 3-(2-fluorophenyl)-N-(2-(pyridine-2-yl)ethyl)propiolamide,
[9] N-(2-(pyridine-2-yl)ethyl)-3-p-tolylpropiolamide,
[10] N-(2-(pyridine-2-yl)ethyl)-3-(4-(trifluoromethyl)-phenyl)-propiolamide,
[11] 3-phenyl-N-(2-(pyridine-2-yl)ethyl)propiolamide,
[12] 3-(2,3-dimethyl-phenyl)-N-(2-(pyridine-2-yl)ethyl)propiolamide,
[13] 3-(3,5-dimethyl-phenyl)-N-(2-(pyridine-2-yl)ethyl)propiolamide,
[14] 3-(3,5-dichloro-phenyl)-N-(2-(pyridine-2-yl)ethyl)propiolamide,
[15] 3-(3-fluoro-4-methyl-phenyl)-N-(2-(pyridine-2-yl)ethyl)propiolamide,
[16] 3-(4-tert.butyl-phenyl)-N-(2-(pyridine-2-yl)ethyl)propiolamide,
[17] 3-(2,4-dichloro-phenyl)-N-(2-(pyridine-2-yl)ethyl)propiolamide,
[18] N-(2-(pyridine-2-yl)ethyl)-3-m-tolyl-propiolamide,
[19] 3-(2,4-dimethyl-phenyl)-N-(2-(pyridine-2-yl)ethyl)propiolamide,
[20] 3-(4-fluoro-phenyl)-N-(2-(pyridine-2-yl)ethyl)propiolamide,
[21] 3-(4-chloro-phenyl)-N-(2-(pyridine-2-yl)ethyl)propiolamide,
[22] 3-(2-cyano-phenyl)-N-(2-(pyridine-2-yl)ethyl)propiolamide,
[23] 3-(4-cyano-phenyl)-N-(2-(pyridine-2-yl)ethyl)propiolamide,
[24] 3-(4-methoxy-phenyl)-N-(2-(pyridine-2-yl)ethyl)propiolamide,
[25] N-(2-(pyridine-2-yl)ethyl)-3-thiophene-2-yl-propiolamide,
[26] N-benzyl-3-(2,4-difluoro-phenyl)-N-phenethylpropiolamide,
[27] N-benzyl-3-(2-fluoro-phenyl)-N-phenethylpropiolamide,
[28] N-benzyl-N-phenethyl-3-p-tolyl-propiolamide,
[29] N-benzyl-N-phenethyl-3-(4-(trifluoromethyl)-phenyl)-propiolamide,
[30] 3-(2-bromo-5-methoxy-phenyl)-N-(2-(pyridine-2-yl)ethyl)propiolamide,
[31] N-(2-(pyridine-2-yl)ethyl)-3-o-tolyl-propiolamide,
[32] N-(2-(pyridine-2-yl)ethyl)-3-(2-(trifluoromethyl)-phenyl)-propiolamide,
[33] 3-(3-bromo-4-methoxy-phenyl)-N-(2-(pyridine-2-yl)ethyl)propiolamide,
[34] N-benzyl-N-phenethyl-3-phenyl-propiolamide,
[35] N-benzyl-3-(2,3-dimethyl-phenyl)-N-phenethyl-propiolamide,
[36] N-benzyl-3-(3,5-dichloro-phenyl)-N-phenethyl-propiolamide,
[37] N-benzyl-3-(2,4-dichloro-phenyl)-N-phenethyl-propiolamide,
[38] 3-(1H-indol-5-yl)-N-(2-(pyridine-2-yl)ethyl)propiolamide,
[39] N-benzyl-3-(1H-indol-5-yl)-N-phenethylpropiolamide,
[40] N-(3,4-dimethoxyphenethyl)-3-(4-fluoro-3-methylphenyl)-N-methylpropiolamide,
[41] 3-(4-fluoro-3-methylphenyl)-N-methyl-N-phenethylpropiolamide,
[42] 3-(2-fluorophenyl)-N-methyl-N-phenethylpropiolamide,
[43] N-(3,4-dimethoxyphenethyl)-N-methyl-3-p-tolylpropiolamide,
[44] N-methyl-N-phenethyl-3-p-tolylpropiolamide,
[45] 3-(2,4-dichlorophenyl)-N-methyl-N-phenethylpropiolamide,
[46] N-methyl-N-phenethyl-3-m-tolylpropiolamide,
[47] 3-(2,4-dimethylphenyl)-N-methyl-N-phenethylpropiolamide,
[48] N-(3,4-dimethoxyphenethyl)-3-(4-fluorophenyl)-N-methylpropiolamide,
[49] N-methyl-N-phenethyl-3-(3-(trifluoromethyl)phenyl)propiolamide,
[50] N-(3,4-dimethoxyphenethyl)-N-methyl-3-o-tolylpropiolamide,
[51] N-methyl-N-phenethyl-3-o-tolylpropiolamide,
[52] N-(3,4-dimethoxyphenethyl)-N-methyl-3-(2-(trifluoromethyl)phenyl)propiolamide,
[53] N-methyl-N-phenethyl-3-(2-(trifluoromethyl)phenyl)propiolamide,
[54] 3-(2-cyanophenyl)-N-(3,4-dimethoxyphenethyl)-N-methylpropiolamide,
[55] 3-(2-cyanophenyl)-N-methyl-N-phenethylpropiolamide,
[56] 3-(4-fluoro-3-methylphenyl)-N-(2-(thiophene-2-yl)ethyl)propiolamide,
[57] N-(2-(1H-indol-3-yl)ethyl)-3-m-tolylpropiolamide,
[58] N-(3-chlorophenethyl)-3-(3-methoxyphenyl)propiolamide,
[59] 3-(3-methoxyphenyl)-N-(4-methylphenethyl)propiolamide,
[60] N-(2,2-diphenylethyl)-3-(3-methoxyphenyl)propiolamide,
[61] N-(4-fluorophenethyl)-3-(3-methoxyphenyl)propiolamide,
[62] N-(3-chlorophenethyl)-3-(4-fluoro-3-methylphenyl)propiolamide,
[63] 3-(4-fluoro-3-methylphenyl)-N-(4-methylphenethyl)propiolamide,
[47] N-(2,2-diphenylethyl)-3-(4-fluoro-3-methylphenyl)propiolamide,
[69] N-(3-chlorophenethyl)-3-m-tolylpropiolamide,
[70] N-(4-methylphenethyl)-3-m-tolylpropiolamide,
[71] N-(2,2-diphenylethyl)-3-m-tolylpropiolamide,
[72] 3-(4-fluorophenyl)-N-phenethylpropiolamide,
[73] 3-(thiophene-2-yl)-N-(2-(thiophene-2-yl)ethyl)propiolamide,
[74] N-benzyl-3-(4-fluorophenyl)-N-phenethylpropiolamide,
[75] N-benzyl-3-(4-tert-butylphenyl)-N-phenethylpropiolamide,
[76] N-benzyl-N-phenethyl-3-(2-(trifluoromethyl)phenyl)propiolamide,
[77] N-benzyl-N-phenethyl-3-(thiophene-2-yl)propiolamide,
[78] 3-(2,4-difluorophenyl)-N-methyl-N-phenethylpropiolamide,
[79] N-(3,4-dimethoxyphenethyl)-3-(3-methoxyphenyl)-N-methylpropiolamide,
[80] 3-(3-methoxyphenyl)-N-methyl-N-phenethylpropiolamide,
[81] 3-(3-methoxyphenyl)-N-phenethylpropiolamide,
[82] 3-(4-fluoro-3-methylphenyl)-N-phenethylpropiolamide,
[84] N-phenethyl-3-m-tolylpropiolamide,
[85] N-phenethyl-3-(thiophene-2-yl)propiolamide,
[86] 3-(3-cyanophenyl)-N-phenethylpropiolamide,
[87] N-(2-(1H-indol-3-yl)ethyl)-3-(3-methoxyphenyl)propiolamide,
[88] N-(2-(1H-indol-3-yl)ethyl)-3-(4-fluoro-3-methylphenyl)propiolamide,
[89] N-(3-chlorophenethyl)-3-(4-fluorophenyl)propiolamide,
[90] 3-(4-fluorophenyl)-N-(4-methylphenethyl)propiolamide,
[91] N-(2,2-diphenylethyl)-3-(4-fluorophenyl)propiolamide,
[92] N-(4-fluorophenethyl)-3-(4-fluorophenyl)propiolamide,
[93] N-(3-chlorophenethyl)-3-(3-chlorophenyl)propiolamide,
[94] N-(3-chlorophenethyl)-3-(thiophene-2-yl)propiolamide,
[95] N-(4-methylphenethyl)-3-(thiophene-2-yl)propiolamide,
[96] N-(2,2-diphenylethyl)-3-(thiophene-2-yl)propiolamide,
[97] 3-(3-cyanophenyl)-N-(4-methylphenethyl)propiolamide,
[98] 3-(3-cyanophenyl)-N-(2,2-diphenylethyl)propiolamide,
[99] 3-(3-cyanophenyl)-N-(4-fluorophenethyl)propiolamide,
[100] N-(3,4-dichlorophenethyl)-3-(4-fluoro-3-methylphenyl)propiolamide,
[101] N-(3,4-dichlorophenethyl)-3-phenylpropiolamide,
[102] N-(3,4-dichlorophenethyl)-3-m-tolylpropiolamide,
[103] N-(3,4-dichlorophenethyl)-3-(thiophene-2-yl)propiolamide,
[104] 3-(3-cyanophenyl)-N-(3,4-dichlorophenethyl)propiolamide,
[105] N-(2-(1H-indol-3-yl)ethyl)-3-(2,4-difluorophenyl)propiolamide,
[106] N-(2-(1H-indol-3-yl)ethyl)-3-(2-fluorophenyl)propiolamide,
[107] N-(2-(1H-indol-3-yl)ethyl)-3-p-tolylpropiolamide,
[108] N-(2-(1H-indol-3-yl)ethyl)-3-(2,4-dichlorophenyl)propiolamide,
[109] N-(2-(1H-indol-3-yl)ethyl)-3-(2,4-dimethylphenyl)propiolamide,
[110] N-(2-(1H-indol-3-yl)ethyl)-3-(4-tert-butylphenyl)propiolamide,
[111] N-(2-(1H-indol-3-yl)ethyl)-3-(3,4-dimethylphenyl)propiolamide,
[112] N-(2-(1H-indol-3-yl)ethyl)-3-(2-bromo-5-methoxyphenyl)propiolamide,
[113] N-(2-(1H-indol-3-yl)ethyl)-3-o-tolylpropiolamide,
[114] N-(2-(1H-indol-3-yl)ethyl)-3-(2-(trifluoromethyl)phenyl)propiolamide,
[115] N-(2-(1H-indol-3-yl)ethyl)-3-(2-cyanophenyl)propiolamide,
[116] N-(2-(1H-indol-3-yl)ethyl)-3-(3,5-dichlorophenyl)propiolamide,
[117] 3-(2,4-difluorophenyl)-N-(2-(thiophene-2-yl)ethyl)propiolamide,
[118] 3-(2-fluorophenyl)-N-(2-(thiophene-2-yl)ethyl)propiolamide,
[119] N-(2-(thiophene-2-yl)ethyl)-3-p-tolylpropiolamide,
[120] N-(2-(thiophene-2-yl)ethyl)-3-(4-(trifluoromethyl)phenyl)propiolamide,
[121] 3-(2,3-dimethylphenyl)-N-(2-(thiophene-2-yl)ethyl)propiolamide,
[122] 3-(3,5-dichlorophenyl)-N-(2-(thiophene-2-yl)ethyl)propiolamide,
[123] 3-(2,4-dichlorophenyl)-N-(2-(thiophene-2-yl)ethyl)propiolamide,
[124] 3-(3-fluoro-4-methylphenyl)-N-(2-(thiophene-2-yl)ethyl)propiolamide,
[125] 3-(4-tert-butylphenyl)-N-(2-(thiophene-2-yl)ethyl)propiolamide,
[126] 3-(3,4-dimethylphenyl)-N-(2-(thiophene-2-yl)ethyl)propiolamide,
[127] 3-(2-bromo-5-methoxyphenyl)-N-(2-(thiophene-2-yl)ethyl)propiolamide,
[128] N-(2-(thiophene-2-yl)ethyl)-3-o-tolylpropiolamide,
[129] N-(2-(thiophene-2-yl)ethyl)-3-(2-(trifluoromethyl)phenyl)propiolamide,
[130] 3-(2-cyanophenyl)-N-(2-(thiophene-2-yl)ethyl)propiolamide,
[131] 3-(4-cyanophenyl)-N-(2-(thiophene-2-yl)ethyl)propiolamide,
[132] N-(2-methoxyphenethyl)-3-(3-methoxyphenyl)propiolamide,
[133] N-(4-chlorophenethyl)-3-(3-methoxyphenyl)propiolamide,
[134] 3-(4-fluoro-3-methylphenyl)-N-(2-methoxyphenethyl)propiolamide,
[135] N-(4-chlorophenethyl)-3-(4-fluoro-3-methylphenyl)propiolamide,
[138] N-(2-methoxyphenethyl)-3-m-tolylpropiolamide,
[139] N-(4-chlorophenethyl)-3-m-tolylpropiolamide,
[140] N-(4-chlorophenethyl)-3-(4-fluorophenyl)propiolamide,
[141] 3-(3,5-dichlorophenyl)-N-(2-methoxyphenethyl)propiolamide,
[142] 3-(2,4-dichlorophenyl)-N-(2-methoxyphenethyl)propiolamide,
[143] 3-(2,4-dimethylphenyl)-N-phenethylpropiolamide,
[144] N-(3-chlorophenethyl)-3-(2,4-difluorophenyl)propiolamide,
[145] 3-(2,4-difluorophenyl)-N-(4-methylphenethyl)propiolamide,
[146] 3-(2,4-difluorophenyl)-N-(2,2-diphenylethyl)propiolamide,
[147] 3-(2,4-difluorophenyl)-N-(4-fluorophenethyl)propiolamide,
[148] N-(3-chlorophenethyl)-3-(2-fluorophenyl)propiolamide,
[149] 3-(2-fluorophenyl)-N-(4-methylphenethyl)propiolamide,
[150] N-(2,2-diphenylethyl)-3-(2-fluorophenyl)propiolamide,
[151] N-(4-fluorophenethyl)-3-(2-fluorophenyl)propiolamide,
[152] N-(3-chlorophenethyl)-3-p-tolylpropiolamide,
[153] N-(4-methylphenethyl)-3-p-tolylpropiolamide,
[154] N-(2,2-diphenylethyl)-3-p-tolylpropiolamide,
[155] N-(3-chlorophenethyl)-3-(4-(trifluoromethyl)phenyl)propiolamide,
[156] N-(4-methylphenethyl)-3-(4-(trifluoromethyl)phenyl)propiolamide,
[157] N-(2,2-diphenylethyl)-3-(4-(trifluoromethyl)phenyl)propiolamide,
[158] N-(4-fluorophenethyl)-3-(4-(trifluoromethyl)phenyl)propiolamide,
[159] N-(3,4-dichlorophenethyl)-3-(4-(trifluoromethyl)phenyl)propiolamide,
[160] N-(3-chlorophenethyl)-3-(2,3-dimethylphenyl)propiolamide,
[161] 3-(2,3-dimethylphenyl)-N-(4-methylphenethyl)propiolamide,
[162] 3-(2,3-dimethylphenyl)-N-(2,2-diphenylethyl)propiolamide,
[163] 3-(2,3-dimethylphenyl)-N-(4-fluorophenethyl)propiolamide,
[164] N-(3,4-dichlorophenethyl)-3-(2,3-dimethylphenyl)propiolamide,
[165] N-(3-chlorophenethyl)-3-(3,5-dimethylphenyl)propiolamide,
[166] 3-(3,5-dimethylphenyl)-N-(4-methylphenethyl)propiolamide,
[167] 3-(3,5-dimethylphenyl)-N-(2,2-diphenylethyl)propiolamide,
[168] 3-(4-hydroxy-3-methylphenyl)-N-(4-methylphenethyl)propiolamide,
[169] N-(2,2-diphenylethyl)-3-(4-hydroxy-3-methylphenyl)propiolamide,
[170] 3-(1H-indol-5-yl)-N-(4-methylphenethyl)propiolamide,
[171] N-(2,2-diphenylethyl)-3-(1H-indol-5-yl)propiolamide,
[172] N-(4-fluorophenethyl)-3-(1H-indol-5-yl)propiolamide,
[173] N-(3,4-dichlorophenethyl)-3-(1H-indol-5-yl)propiolamide,
[174] N-(3-chlorophenethyl)-3-(3,5-dichlorophenyl)propiolamide,
[175] 3-(3,5-dichlorophenyl)-N-(4-methylphenethyl)propiolamide,
[176] 3-(3,5-dichlorophenyl)-N-(2,2-diphenylethyl)propiolamide,
[177] 3-(3,5-dichlorophenyl)-N-(4-fluorophenethyl)propiolamide,
[178] N-(3,4-dichlorophenethyl)-3-(3,5-dichlorophenyl)propiolamide,
[179] 3-(2,4-dichlorophenyl)-N-(4-methylphenethyl)propiolamide,
[180] 3-(2,4-dichlorophenyl)-N-(4-fluorophenethyl)propiolamide,
[181] N-(3,4-dichlorophenethyl)-3-(2,4-dichlorophenyl)propiolamide,
[182] N-(3-chlorophenethyl)-3-o-tolylpropiolamide,
[183] N-(4-methylphenethyl)-3-o-tolylpropiolamide,
[184] N-(2,2-diphenylethyl)-3-o-tolylpropiolamide,
[185] N-(4-fluorophenethyl)-3-o-tolylpropiolamide,
[186] N-(3,4-dichlorophenethyl)-3-o-tolylpropiolamide,
[187] N-(3,4-dichlorophenethyl)-3-(4-hydroxy-3-methylphenyl)propiolamide,
[188] 3-(3-chlorophenyl)-N-(2-methoxyphenethyl)propiolamide,
[189] N-(2-methoxyphenethyl)-3-(thiophene-2-yl)propiolamide,
[190] N-(4-chlorophenethyl)-3-(thiophene-2-yl)propiolamide,
[191] 3-(3-cyanophenyl)-N-(2-methoxyphenethyl)propiolamide,
[192] N-(4-chlorophenethyl)-3-(3-cyanophenyl)propiolamide,
[193] 3-(2,4-difluorophenyl)-N-(2-methoxyphenethyl)propiolamide,
[194] 3-(2-fluorophenyl)-N-(2-methoxyphenethyl)propiolamide,
[195] N-(2-methoxyphenethyl)-3-(4-(trifluoromethyl)phenyl)propiolamide,
[196] 3-(3-fluoro-4-methylphenyl)-N-phenethylpropiolamide,
[197] 3-(4-tert-butylphenyl)-N-phenethylpropiolamide,
[198] 3-(3,4-dimethylphenyl)-N-phenethylpropiolamide,
[199] 3-(2,4-difluorophenyl)-N-phenethylpropiolamide,
[200] N-phenethyl-3-p-tolylpropiolamide,
[201] N-phenethyl-3-(4-(trifluoromethyl)phenyl)propiolamide,
[202] 3-(2,3-dimethylphenyl)-N-phenethylpropiolamide,
[203] 3-(3,5-dimethylphenyl)-N-phenethylpropiolamide,
[204] 3-(1H-indol-5-yl)-N-phenethylpropiolamide,
[205] 3-(2,3-dimethylphenyl)-N-(2-methoxyphenethyl)propiolamide,
[206] 3-(3,5-dimethylphenyl)-N-(2-methoxyphenethyl)propiolamide,
[207] 3-(3,5-dichlorophenyl)-N-phenethylpropiolamide,
[208] N-(3,4-dichlorophenethyl)-3-(2,4-difluorophenyl)propiolamide,
[209] N-(3,4-dichlorophenethyl)-3-(2-fluorophenyl)propiolamide,
[210] N-(4-fluorophenethyl)-3-p-tolylpropiolamide,
[211] N-(3,4-dichlorophenethyl)-3-p-tolylpropiolamide,
[212] 3-(3,5-dimethylphenyl)-N-(4-fluorophenethyl)propiolamide,
[213] N-(3-chlorophenethyl)-3-(2,4-dichlorophenyl)propiolamide,
[214] 3-(2,4-dichlorophenyl)-N-(2,2-diphenylethyl)propiolamide,
[215] 3-(3-chlorophenyl)-N-(2-fluorophenethyl)propiolamide,
[216] 3-(3-chlorophenyl)-N-(3-fluorophenethyl)propiolamide,
[217] 3-(3-chlorophenyl)-N-(4-fluorophenethyl)propiolamide,
[218] 3-(3-chlorophenyl)-N-(2-fluorophenethyl)-N-methylpropiolamide,
[219] 3-(3-chlorophenyl)-N-(3-(trifluoromethyl)phenethyl)propiolamide,
[220] 3-(3-chlorophenyl)-N-(4-fluorophenethyl)-N-methylpropiolamide,
[221] 3-(3-chlorophenyl)-N-(3-fluorophenethyl)-N-methylpropiolamide,
[222] 3-(3-chlorophenyl)-N-methyl-N-(3-(trifluoromethyl)phenethyl)propiolamide,
[223] 3-(3-chlorophenyl)-N-(2-methylphenethyl)propiolamide,
[224] 3-(3-chlorophenyl)-N-(3-methylphenethyl)propiolamide,
[225] 3-(3-chlorophenyl)-N-(4-methylphenethyl)propiolamide,
[226] 3-(3-chlorophenyl)-N-methyl-N-(2-methylphenethyl)propiolamide,
[227] 3-(3-chlorophenyl)-N-methyl-N-(3-methylphenethyl)propiolamide,
[228] 3-(3-chlorophenyl)-N-methyl-N-(4-methylphenethyl)propiolamide,
[229] 3-(3-chlorophenyl)-N-(2-(dimethylamino)-2-phenylethyl)propiolamide,
[230] N-(2-(1H-pyrrol-1-yl)ethyl)-3-(3-chlorophenyl)propiolamide,
[231] N-(2-(1H-pyrazol-1-yl)ethyl)-3-(3-chlorophenyl)propiolamide,
[232] N-(2-(1H-imidazol-1-yl)ethyl)-3-(3-chlorophenyl)propiolamide,
[233] N-(2-(1H-pyrrol-1-yl)ethyl)-3-(3-chlorophenyl)-N-methylpropiolamide,
[234] N-(2-(1H-pyrazol-1-yl)ethyl)-3-(3-chlorophenyl)-N-methylpropiolamide,
[235] 3-(3-chlorophenyl)-N-(2-(pyridine-3-yl)ethyl)propiolamide,
[236] 3-(3-chlorophenyl)-N-methyl-N-(2-(pyridine-2-yl)ethyl)propiolamide,
[237] 3-(3-chlorophenyl)-N-ethyl-N-phenethylpropiolamide,
[238] 3-(3-chlorophenyl)-N-isopropyl-N-phenethylpropiolamide,
[239] 3-(3-chlorophenyl)-N-cyclopropyl-N-phenethylpropiolamide,
[240] 3-(3-chlorophenyl)-N-(2-methoxyphenethyl)-N-methylpropiolamide,
[241] 3-(3-chlorophenyl)-N-methyl-N-(1-phenylpropane-2-yl)propiolamide,
[242] 3-(3-chlorophenyl)-N-(1-phenylpropane-2-yl)propiolamide,
[243] 3-(3-chlorophenyl)-N-methyl-N-(2-(pyridine-3-yl)ethyl)propiolamide,
[244] 3-(3-chlorophenyl)-N-methyl-N-(2-(pyridine-4-yl)ethyl)propiolamide,
[245] 3-(3-chlorophenyl)-N-methyl-N-(2-(thiophene-2-yl)ethyl)propiolamide,
[246] 3-(3-chlorophenyl)-N-(1-(pyridine-3-yl)propane-2-yl)propiolamide,
[247] 3-(3-chlorophenyl)-N-methyl-N-(1-(pyridine-3-yl)propane-2-yl)propiolamide,
[248] 3-(3-chlorophenyl)-N-(2-(2-methylpyridine-3-yl)ethyl)propiolamide,
[249] 3-(3-chlorophenyl)-N-methyl-N-(2-(2-methylpyridine-3-yl)ethyl)propiolamide,
[250] 3-(3-chlorophenyl)-N-(2-(trifluoromethyl)phenethyl)propiolamide,
[251] 3-(3-chlorophenyl)-N-methyl-N-(2-(trifluormethyl)phenethyl)propiolamide,
[252] 3-(3-chlorophenyl)-N-(4-(trifluoromethyl)phenethyl)propiolamide and [253] 3-(3-chlorophenyl)-N-methyl-N-(4-(trifluoromethyl)phenethyl)propiolamide;
   in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Substituted bis(hetero)aromatic N-ethylpropiolamides of the above-mentioned general formula I are also particularly preferred, which, after 60 minutes of incubation in 450 μg protein from pig brain homogenate at a temperature between 20° C. and 25° C. in a concentration of less than 2000 nM, preferably of less than 1000 nM, particularly preferably of less than 700 nM, very particularly preferably of less than 100 nM, even more preferably of less than 30 nM, bring about a 50-percent displacement of [³H]-2-methyl-6-(3-methoxyphenyl)-ethynylpyridine which is present in a concentration of 5 nM.

Thereby, the determination of the displacement of [³H]-2-methyl-6-(3-methoxyphenyl)-ethynylpyridine is performed as described in the section Pharmacological Methods, I. Method for determining the inhibition of [³H]-MPEP-bonding in the mGluR5 receptor bonding assay.

A further subject matter of the present invention is a method for producing compounds of the general formula I indicated above according to which at least one compound of the general formula II,

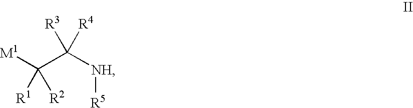

in which R¹, R², R³, R⁴, R⁵ and M¹ have the above-mentioned meaning, is transferred by conversion with at least one compound of the general formula M²-C≡C(═O)—OH, in which M² has the above-mentioned meaning, optionally in a reaction medium, optionally in the presence of at least one suitable coupling agent, optionally in the presence of at least one base, preferably at a temperature of −70° C. to 100° C., or by conversion with at least one compound of the general formula M²-C≡C(═O)—X, in which M² has the above-mentioned meaning and X denotes a leaving group, preferably a halogen residue, particularly preferably a chlorine or bromine residue, in a reaction medium, optionally in the presence of at least one base, preferably at a temperature of −70° C. to 100° C., into at least one corresponding compound of the general formula I, optionally in the form of a corresponding salt,

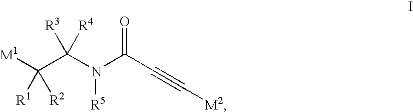

in which R¹, R², R³, R⁴, R⁵, M¹ and M² have the above-mentioned meaning, and this is optionally purified and/or isolated;
or at least one compound of the general formula II is transferred by conversion with propiolic acid [HC≡C≡C(═O)—OH] optionally in a reaction medium, optionally in the presence of at least one suitable coupling agent, optionally in the presence of at least one base, preferably at a temperature of −70° C. to 100° C., or by conversion with at least one compound of the general formula HC≡C—C(═O)—X, in which X denotes a leaving group, preferably a halogen residue, particularly preferably a chlorine or bromine residue, in a reaction medium, optionally in the presence of at least one base, preferably at a temperature of −70° C. to 100° C., into at least one corresponding compound of the general formula III, optionally in the form of a corresponding salt,

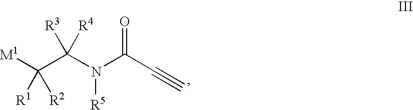

in which R¹, R², R³, R⁴, R⁵, M¹ and M² have the above-mentioned meaning, and this is optionally purified and/or isolated,
and at least one compound of the general formula III is transferred into at least one corresponding compound of the general formula I, optionally in the form of a corresponding salt by conversion with at least one compound of the general formula M²-X, in which M² has the above-mentioned meaning and X denotes a leaving group, preferably a halogen residue or a sulphonic acid ester, particularly preferably iodine, bromine or triflate, optionally in a reaction medium, optionally in the presence of at least one catalyst, preferably in the presence of at least one palladium catalyst selected from the group comprising palladium chloride [PdCl₂], palladium acetate [Pd(OAc)₂], tetrakistriphenylphosphinepalladium [Pd(PPh₃)₄], bistriphenylphosphinepalladium dichloride [Pd(PPh₃)₂Cl₂] and bistriphenylphosphinepalladium acetate [Pd(PPh₃)₂(OAc)₂], optionally in the presence of at least one ligand, preferably in the presence of at least one ligand selected from the group comprising triphenylphosphine, triphenylarsine and tri-2-furyl-phosphine, optionally in the presence of at least one inorganic salt, preferably in the presence of at least one inorganic salt selected from the group comprising lithium chloride and zinc chlorid, optionally in the presence of at least one copper salt, preferably in the presence of copper iodide, optionally in the presence of at least one organic or inorganic base, preferably in the presence of at least one base selected from the group comprising triethylamine, [1,4]-diazabicyclo-[2.2.2]-octane, diisopropylamine, diisopropylethylamine, potassium carbonate and sodium hydrogencarbonate, preferably at a temperature between −70° C. and 300° C., and this is optionally purified and/or isolated.

A method according to the invention for producing substituted bis(hetero)aromatic N-ethylpropiolamides of the above-mentioned general formula I is also indicated in following Diagram 1.

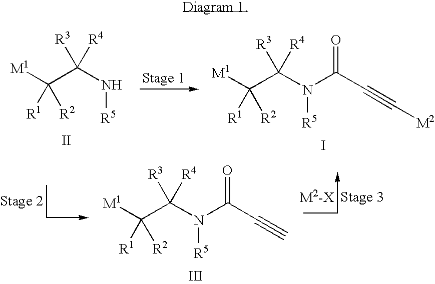

In stage 1, compounds of the above-mentioned general formula II are converted with carboxylic acids of the above-mentioned general formula M²-C≡C—C(═O)—OH in a reaction medium, preferably selected from the group comprising diethylether, tetrahydrofuran, acetonitrile, methanol, ethanol, (1,2)-dichloroethane, dimethylformamide, dichloromethane and corresponding mixtures, optionally in the presence of at least one coupling agent, preferably selected from the group comprising 1-benzotriazolyloxy-tris-(dimethylamino)-phosphonium hexafluorophosphate (BOP), dicyclohexylcarbodiimide (DCC), N′-(3-dimethylaminopropyl)-N-ethylcarbodiimide (EDCl), PL-EDC (polymer-bound N-benzyl-3-((ethylimino)methylenamino)-N,N-dimethylpropane-1-aminium chloride), 1,1′-carbonyl-diimidazol (CDI), N-[(dimethyamino)-1H-1,2,3-triazolo[4,5-b]pyridino-1-ylmethylene]-N-methylmethanaminium hexafluorophosphate N-oxide (HATU), O -(benzotriazol-1-yl)-N,N,N′,N′-tetramethyluroniom hexafluorophosphate (HBTU), O -(benzotriazl-1-yl)-N,N,N′,N′-tetramethyluronium-tetrafluoroborate (TBTU), 1-hydroxy-7-azabenzotriazol (HOAt) and polymer-bound carbodiimide resin (PS-carbodiimid resin, PSii), particularly preferably in the presence of at least one coupling agent selected from the group comprising TBTU, EDCl and PL-EDC, optionally in the presence of at least one organic base, preferably selected from the group comprising triethylamine, pyridine, dimethylaminopyridine, N-methylmorpholine, [1,4]-diazabicyclo-[2.2.2]-octane (DABCO), 1,8-diazabicyclo[5.4.0]undec-7-en (DBU), 1,5-diazabicyclo[4.3.0]non-5-en (DBN) and diisopropylethylamine, preferably in the presence of diisopropylethylamine or triethylamine, preferably at temperatures of −70° C. to 250° C. to yield compounds of the general formula I.

Alternatively, compounds of the above-mentioned general formula II are converted with carboxylic acid derivatives of the above-mentioned general formula M²-C≡C—C(═O)—X, in which X denotes a leaving group, preferably a halogen residue, particularly preferably chlorine or bromine, in a reaction medium, preferably selected from the group comprising diethylether, tetrahydrofuran, acetonitrile, methanol, ethanol, dimethylformamide, dichloromethane, 1,2-dichloroethane and corresponding mixtures, optionally in the presence of an organic or inorganic base, preferably selected from the group comprising triethylamine, dimethylaminopyridine, pyridine and diisopropylamine, at temperatures of −70° C. to 250° C. to yield compounds of the general formula I.

In stage 2, compounds of the above-mentioned general formula II are converted with propiolic acid H—C≡C—C(═O)—OH or with carboxylic acid derivatives of the general formula H—C≡C—C(═O)—X, in which X denotes a leaving group, preferably a halogen residue, particularly preferably chlorine or bromine, as described in Diagram 1, Stage 1, to yield compounds of the general formula III.

In stage 3, compounds of the above-mentioned general formula III are converted with compounds of the general formula M²-X, in which M² has the above-mentioned meaning and X denotes a leaving group, preferably a halogen residue or a sulphonic acid ester, particularly preferably iodine, bromine or triflate, in a reaction medium, preferably in a reaction medium selected from the group comprising methanol, ethylacetate, ethanol, isopropanol, n-butanol, diethylether, dioxane, tetrahydrofuran, chloroform, dichloromethane, dimethylformamide, acetonitrile, pyridine, dimethylsulphoxide, water, toluol and corresponding mixtures, preferably in dimethylformamide, water, ethylacetate, tetrahydrofuran and corresponding mixtures, optionally in the presence of at least one catalyst, preferably in the presence of a palladium catalyst selected from the group comprising palladium chloride [PdCl₂], palladium acetate [Pd(OAc)₂], tetrakistriphenylphosphine palladium [Pd(PPh₃)₄], bistriphenylphosphinepalladium dichloride [Pd(PPh₃)₂Cl₂] and bistriphenylphosphinepalladium acetate [Pd(PPh₃)₂(OAc)₂], preferably in the presence of Pd(PPh₃)₄, Pd(PPh₃)₂Cl₂ and Pd(PPh₃)₂(OAc)₂, optionally in the presence of at least one ligand, preferably in the presence of at least one ligand selected from the group comprising triphenylphosphine, triphenylarsine and tri-2-furyl-phosphine, preferably in the presence of triphenylphosphine, optionally in the presence of at least one inorganic salt, preferably in the presence of at least one inorganic salt selected from the group comprising lithium chloride and zinc chlorid, optionally in the presence of at least one copper salt, preferably in the presence of copper iodide, optionally in the presence of at least one organic or inorganic base, preferably in the presence of at least one base selected from the group comprising triethylamine, [1,4]-diazabicyclo-[2.2.2]-octane, diisopropylamine, diisopropylethylamine, potassium carbonate and sodium hydrogencarbonate, preferably at a temperature between −70° C. and 250° C. to yield a compound of the general formula I. Compounds of the general formula M²-I or M²-Br are particularly preferably converted with compounds of the general formula III in a reaction medium selected from the group comprising dimethylformamide or ethylacetate in the presence of Pd(PPh₃)₂Cl₂, copper(I) iodide and a base selected from the group comprising diisopropylamine or triethylamine.

The compounds of the above-mentioned general formula II, and of the general formulae M²-C≡C—C(═O)—OH, M²-C≡C—C(═O)—X, M²-X and H—C≡C—C(═O)—X, are in each case commercially available on the market and/or can be produced according to the conventional methods known to the person skilled in the art.

The conversions described above can in each case be performed under normal conditions familiar to the person skilled in the art, for example, in terms of pressure or the sequence of the addition of components. The optimum performance of the method according to the respective conditions can optionally be determined by the person skilled in the art by simple preliminary tests.

The intermediate and end products obtained according to the conversions described above can in each case, if desired and/or necessary, be purified and/or isolated according to conventional methods known to the person skilled in the art. Suitable purification methods are, for example, extraction methods and chromatographic methods such as column chromatography or preparative chromatography.

All of the method steps described above and in each case also the purification and/or isolation of intermediate or end products can partially or entirely be performed under an inert gas atmosphere, preferably under a nitrogen atmosphere.

In so far as the substituted bis(hetero)aromatic N-ethylpropiolamides according to the invention of the above-mentioned general formulae I, Ia, Ib, Ic, Id, Ie and If, referred to below as substituted bis(hetero)aromatic N-ethylpropiolamides of the general formula I, can be obtained after their production in the form of a mixture of the stereoisomers thereof, preferably in the form of the racemates thereof or other mixtures of the various enantiomers and/or diastereomers thereof, these can be separated and optionally isolated according to conventional methods known to the person skilled in the art. Chromatographic separating methods, in particular liquid chromatography methods under normal pressure or under increased pressure, preferably MPLC and HPLC methods, and methods of fractionated crystallisation are cited by way of example. Therein, in particular individual enantiomers, e.g. diastereomeric salts formed by means of HPLC on the chiral phase or by means of crystallisation with chiral acids such as (+) tartaric acid, (−) tartaric acid or (+) 10-camphor sulphonic acid, can be separated from one another.

The substituted bis(hetero)aromatic N-ethylpropiolamides according to the invention of the above-mentioned genera formula I and optionally in each case corresponding stereoisomers can be obtained according to conventional methods known to the person skilled in the art in the form of corresponding salts, preferably in the form of corresponding hydrochlorides, particularly in the form of corresponding physiologically acceptable salts, whereby the medicament according to the invention can have one or more salts or several of these compounds.

The respective salts of the substituted bis(hetero)aromatic N-ethylpropiolamides according to the invention of the above-mentioned general formula I and corresponding stereoisomers can, for example, be obtained by conversion with one or more inorganic acids and/or one or more organic acids. Suitable acids can be preferably selected from the group comprising perchloric acid, hydrochloric acid, hydrobromic acid, sulphuric acid, methanesulphonic acid, formic acid, acetic acid, oxalic acid, succinic acid, tartaric acid, mandelic acid, fumaric acid, lactic acid, citric acid, glutamic acid, saccharin acid, cyclohexanesulphonamide acid, aspartame, monomethylsebacic acid, 5-oxo-proline, hexane-1-sulphonic acid, nicotinic acid, 2-aminobenzoic acid, 3-aminobenzoic acid or 4-aminobenzoic acid, 2,4,6-trimethylbenzoic acid, α-lipoic acid, acetylglycine, hippuric acid, phosphoric acid, maleic acid, malonic acid and asparaginic acid.

The substituted bis(hetero)aromatic N-ethylpropiolamides according to the invention of the above-mentioned genera formula I and optionally corresponding stereoisomers and in each case their physiologically acceptable salts can be obtained according to conventional methods known to the person skilled in the art also in the form of the solvates thereof, in particular in the form of the hydrates thereof.

It has been surprisingly found that the substituted bis(hetero)aromatic N-ethylpropiolamides according to the invention of the above-mentioned general formula I are suitable for mGluR5 receptor regulation and can therefore be used in particular as active pharmaceutical ingredients in medicaments for the prevention and/or treatment of disorders or illnesses related to these receptors or processes.

The substituted bis(hetero)aromatic N-ethylpropiolamides according to the invention of the above-mentioned general formula I and optionally corresponding stereoisomers and in each case the corresponding salts and solvates appear to be toxicologically safe and are therefore suitable as active pharmaceutical ingredients in medicaments.

A further subject matter of the present invention is therefore a medicament containing at least one substituted bis(hetero)aromatic N-ethylpropiolamide according to the invention of the above-mentioned general formula I, in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates, and optionally one or more pharmaceutically acceptable auxiliary substances.

A further subject matter of the present invention is therefore a medicament containing at least one compound selected from the group comprising
[65] N-(3-chlorophenethyl)-3-phenylpropiolamide,
[66] N-(4-methylphenethyl)-3-phenylpropiolamide,
[67] N-(2,2-diphenylethyl)-3-phenylpropiolamide,
[68] N-(4-fluorophenethyl)-3-phenylpropiolamide,
[83] N-phenethyl-3-phenylpropiolamide,
[136] N-(2-methoxyphenethyl)-3-phenylpropiolamide and
[137] N-(4-chlorophenethyl)-3-phenylpropiolamide,
   in each case optionally in the form of a corresponding salt, or in each case in the form of a corresponding solvate, and optionally one or more physiologically acceptable auxiliary substances.

The medicament according to the invention is suitable for mGluR5 receptor regulation, in particular for inhibition of the mGluR5 receptor.

The medicament according to the invention is particularly suitable for the prevention and/or treatment of disorders and/or illnesses which are at least partially mediated by mGluR5 receptors.

The medicament according to the invention is therefore particularly preferably suitable for the treatment and/or prevention of pain, preferably of pain selected from the group consisting of acute pain, chronic pain, neuropathic pain and visceral pain; migraine; depression; neurodegenerative diseases, preferably selected from the group consisting of multiple sclerosis, Alzheimer's disease, Parkinson's disease and Huntington's chorea; cognitive dysfunction, preferably cognitive deficiency states, particularly preferably Attention Deficit Disorder (ADD); anxiety states; panic attacks; epilepsy; coughing; urinary incontinence; diarrhoea; pruritus; schizophrenia; cerebral ischaemia; muscle spasms; cramps; lung illnesses, preferably selected from the group comprising asthma and pseudo-croup; regurgitation (vomiting); stroke; dyskinesia; retinopathy; listlessness; laryngitis; disorders of food intake, preferably selected from the group consisting of bulimia, cachexia, anorexia and obesity; dependency on alcohol; dependency on medicines; dependency on drugs, preferably dependency on nicotine and/or cocaine; alcohol abuse; abuse of medication; drug abuse; preferably nicotine and/or cocaine abuse; withdrawal symptoms associated with dependency on alcohol, medications and/or drugs (in particular nicotine and/or cocaine); development of tolerance to medications, preferably to natural or synthetic opioids; stomach-esophagus-reflux-syndrome; gastroesophagal reflux; irritable bowel syndrome; for diuresis; for antinatriuresis; for influencing the cardiovascular system; for increasing vigilance; for increasing libido; for modulating locomotor activity or for local anaesthesia.

The medicament according to the invention is very particularly preferably suitable for the prevention of pain, preferably of pain selected from the group consisting of acute pain, chronic pain, neuropathic pain and visceral pain; anxiety states; panic attacks; dependency on alcohol; dependency on medicines; disorders of food intake, preferably selected from the group consisting of bulimia, cachexia, anorexia and obesity; dependency on drugs, preferably dependency on nicotine and/or cocaine; alcohol abuse; abuse of medication; drug abuse; preferably nicotine and/or cocaine abuse; withdrawal symptoms associated with dependency on alcohol, medications and/or drugs (in particular nicotine and/or cocaine); development of tolerance to medications and/or drugs, preferably to natural or synthetic opioids; stomach-esophagus-reflux-syndrome; gastroesophagal reflux and irritable bowel syndrome.

The medicament according to the invention is even more preferably suitable for the prevention and/or the treatment of pain, preferably of pain selected from the group consisting of acute pain, chronic pain, neuropathic pain and visceral pain; anxiety states and panic attacks.

The medicament according to the invention is most preferably suitable for the prevention and/or the treatment of pain, preferably of acute pain, chronic pain, neuropathic pain or visceral pain.

A further subject matter of the present invention is the use of at least one substituted bis(hetero)aromatic N-ethylpropiolamide according to the invention of the above-mentioned general formula I, in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of a corresponding salt, or in each case in the form of a corresponding solvate, and optionally one or more pharmaceutically acceptable auxiliary substances for the production of a medicament for mGluR5 receptor regulation, preferably for inhibition of the mGluR5 receptor.

The use of at least one substituted bis(hetero)aromatic N-ethylpropiolamide according to the invention of the above-mentioned general formula I is preferred, in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of a corresponding salt or in each case in the form of a corresponding solvate, and optionally one or more pharmaceutically acceptable auxiliary substances for the production of a medicament for the prevention and/or treatment of disorders and/or illnesses which are at least partially mediated by mGluR5 receptors.

The use of at least one substituted bis(hetero)aromatic N-ethylpropiolamide according to the invention of the above-mentioned general formula I is particularly preferred, in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of a corresponding salt, or in each case in the form of a corresponding solvate, and optionally one or more pharmaceutically acceptable auxiliary substances for the production of a medicament for the prevention and/or treatment of pain, preferably of pain selected from the group consisting of acute pain, chronic pain, neuropathic pain and visceral pain; migraine; depression; neurodegenerative diseases, preferably selected from the group consisting of multiple sclerosis, Alzheimer's disease, Parkinson's disease and Huntington's chorea; cognitive dysfunction, preferably cognitive deficiency states, particularly preferably Attention Deficit Disorder (ADD); anxiety states; panic attacks; epilepsy; coughing; urinary incontinence; diarrhoea; pruritus; schizophrenia; cerebral ischaemia; muscle spasms; cramps; lung illnesses, preferably selected from the group comprising asthma and pseudo-croup; regurgitation (vomiting); stroke; dyskinesia; retinopathy; listlessness; laryngitis; disorders of food intake, preferably selected from the group consisting of bulimia, cachexia, anorexia and obesity; dependency on alcohol; dependency on medicines; dependency on drugs, preferably dependency on nicotine and/or cocaine; alcohol abuse; abuse of medication; drug abuse; preferably nicotine and/or cocaine abuse; withdrawal symptoms associated with dependency on alcohol, medications and/or drugs (in particular nicotine and/or cocaine); development of tolerance to medications, preferably to natural or synthetic opioids; stomach-esophagus-reflux-syndrome; gastroesophagal reflux; irritable bowel syndrome; for diuresis; for antinatriuresis; for influencing the cardiovascular system; for increasing vigilance; for increasing libido; for modulating locomotor activity or for local anaesthesia.

The use of at least one substituted bis(hetero)aromatic N-ethylpropiolamide according to the invention of the above-mentioned general formula I is very particularly preferred, in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of a corresponding salt or in each case in the form of a corresponding solvate, and optionally one or more pharmaceutically acceptable auxiliary substances for the production of a medicament for the prevention and/or treatment of pain, preferably of pain selected from the group consisting of acute pain, chronic pain, neuropathic pain and visceral pain; anxiety states; panic attacks; dependency on alcohol; dependency on medicines; disorders of food intake, preferably selected from the group consisting of bulimia, cachexia, anorexia and obesity; dependency on drugs, preferably dependency on nicotine and/or cocaine; alcohol abuse; abuse of medication; drug abuse; preferably nicotine and/or cocaine abuse; withdrawal symptoms associated with dependency on alcohol, medications and/or drugs (in particular nicotine and/or cocaine); development of tolerance to medications and/or drugs, particularly to natural or synthetic opioids; stomach-esophagus-reflux-syndrome; gastroesophagal reflux and irritable bowel syndrome.

The use of at least one substituted bis(hetero)aromatic N-ethylpropiolamide according to the invention of the above-mentioned general formula I is even more preferred, in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of a corresponding salt or in each case in the form of a corresponding solvate, and optionally one or more pharmaceutically acceptable auxiliary substances for the production of a medicament for the prevention and/or treatment of pain, preferably of pain selected from the group consisting of acute pain, chronic pain, neuropathic pain and visceral pain; anxiety states and panic attacks.

The medicament according to the invention is suitable for administration to adults and childrens including infants.

The medicament according to the invention may be formulated as a liquid, semisolid or solid dosage form, for example in the form of solutions for injection, drops, succi, syrups, sprays, suspensions, tablets, patches, capsules, dressings, suppositories, ointments, creams, lotions, gels, emulsions, aerosols or in multiparticulate form, for example, in the form of pellets or granules, optionally pressed into tablets, packaged in capsules or suspended in a liquid, and may also be administered as such.

In addition to at least one substituted bis(hetero)aromatic N-ethylpropiolamide according to the invention of the above-mentioned general formula I, optionally in the form of one of the pure stereoisomers thereof, in particular enantiomers or diastereomers, the racemate thereof or in the form of mixtures of the stereoisomers, in particular the enantiomers or diastereomers, in any desired mixing ratio, or optionally in the form of a corresponding salt or in each case in the form of a corresponding solvate, the medicament according to the invention conventionally contains further physiologically acceptable pharmaceutical auxiliary substances, which are preferably selected from the group consisting of matrix materials, fillers, solvents, diluents, surface-active substances, dyes, preservatives, disintegrants, slip agents, lubricants, aromas and binders.

Selection of the physiologically acceptable auxiliary substances and the quantities thereof which are to be used depends upon whether the medicament is to be administered orally, subcutaneously, parenterally, intravenously, intraperitoneally, intradermally, intramuscularly, intranasally, buccally, rectally or topically, for example onto infections of the skin, mucous membranes or eyes. Preparations in the form of tablets, coated tablets, capsules, granules, pellets, drops, succi and syrups are preferred for oral administration, while solutions, suspensions, readily reconstitutible dried preparations and sprays are preferred for parenteral, topical and inhalatory administration.

The substituted bis(hetero)aromatic N-ethylpropiolamides used in the medicament according to the invention of the above-mentioned general formula I in a depot in dissolved form or in a dressing, optionally with the addition of skin penetration promoters, are suitable percutaneous administration preparations.

Orally or percutaneously administrable formulations may also release the respective substituted bis(hetero)aromatic N-ethylpropiolamides of the above-mentioned general formula I in a delayed manner.

Production of the medicaments according to the invention proceeds with the assistance of conventional means, devices, methods and processes well known from the prior art, such as are described for example in “Remington's Pharmaceutical Sciences”, ed. A. R. Gennaro, 17th ed., Mack Publishing Company, Easton, Pa. (1985), in particular in part 8, chapters 76 to 93. The corresponding description is hereby introduced as a reference and is deemed to be part of the disclosure.

The quantity of the respective substituted bis(hetero)aromatic N-ethylpropiolamide of the above-mentioned general formula I to be administered to the patient may vary and is for example dependent on the weight or age of the patient and on the mode of administration, the indication and the severity of the complaint. Conventionally, 0.05 to 100 mg/kg, preferably 0.05 to 10 mg/kg of patient body weight of at least one such compound are administered.

### Pharmacological Methods:

I. Method for Determining the Inhibition of the [³H]-MPEP Bond in the mGluR5 Receptor Bond Assay

Pig brain homogenate is produced by homogenisation (Polytron PT 3000, Kinematica AG, 10,000 rpm for 90 seconds) of pig brain halves without medulla, cerebellum and pons in buffer pH 8.0 (30 mM Hepes, Sigma, order no. H3375+1 tablet complete to 100 ml, Roche Diagnostics, order no. 1836145) in the ratio 1:20 (brain weight/volume) and differential centrifugation at 900×g and 40,000×g. In each case, 450 μg protein from brain homogenate is incubated with 5 nM ³[H]-MPEP (Tocris, order no. R1212) (MPEP=2-methyl-6-(3-methoxyphenyl)-ethynylpyridine) in 250 μl incubation batches in 96 well microtitration plates and the compounds to be tested (10 μM in the test) in buffer (as above) at room temperature for 60 min.

Thereafter, the batches are filtered with the help of a Brandel Cell Harvester (Brandel, TYP Robotic 9600) on unifilter plates with glass fibre filter mats (Perkin Elmer, order no. 6005177) and subsequently washed with buffer (as above) 3 times with in each case 250 μl per sample. The filter plates are subsequently dried for 60 min at 55° C. 30 μL Ultima Gold™ scintillator (Packard BioScience, order no. 6013159) is subsequently added per well and the samples are measured after 3 hours on the β-counter (Mikrobeta, Perkin Elmer). The unspecific bond is determined by addition of 10 μM MPEP (Tocris, order no. 1212).

II. Method for Determining the Ca²⁺ Influx in the mGluR5 Receptor Assay

An agonistic and/or antagonistic effect of substances can be determined on the mGluR5 receptor of the rat species with the following assay. According to this assay, the intracellular Ca²⁺ release is quantified after activation of the mGluR5 receptor with the help of a Ca²⁺-sensitive dye (type Fluo-4, Molecular Probes Europe BV, Leiden Netherlands) in the FlexStation (Molecular Devices, Sunnyvale, USA).

### Preparation of Cortical Neurons:

Cortical neurons are prepared under sterile conditions from postnatal rats (P2-6). To this end, the cortex is removed and transferred directly into collagenase solution (PAA Laboratories GmbH, Cölbe, Germany) and incubated for 45 minutes in a heated separator (37° C., 300 rpm). The collagenase solution is subsequently removed and culture medium is added to the tissue.

### Culture Medium (100 ml):

Neurobasal medium (Gibco Invitrogen GmbH, Karlsruhe, Germany)

### 2 mM L-glutamine (Sigma, Taufkirchen, Germany)

1 vol-% antibiotic/antimycotic solution (PAA Laboratories GmbH, Cölbe, Germany)
15 ng/ml NGF (Gibco Invitrogen GmbH, Karlsruhe, Germany)

### 1 ml B27 Supplement (Gibco Invitrogen GmbH, Karlsruhe, Germany)

1 ml ITS Supplement (Sigma, Taufkirchen, Germany)

The cells are separated by resuspension and centrifuged after addition of 15 ml neurobasal medium through a 70 μm filter insert (BD Biosciences, Heidelberg, Germany). The resultant cell pellet is received in culture medium. The cells are subsequently plated out on poly-D-lysine-coated, black 96-hole-plates with a clear base (BD Biosciences, Heidelberg, Germany), which were previously coated with laminin (2 μg/cm², Gibco Invitrogen GmbH, Karlsruhe, Germany). The cell density is 15,000 cells/hole. The cells are incubated at 37° C. and 5% CO₂ and a change of medium is performed on the 2^{nd} or 3^{rd} day after preparation. Depending on cell growth, the functional investigation can be performed on the 3^{rd}-7^{th} day after preparation.

### Description of the Functional Ca²⁺ Influx Assay

20,000 CHO-hmGluR5 cells/well (Euroscreen, Gosselies, Belgium) are pipetted into 96 well plates (BD Biosciences, Heidelberg, Germany, Ref 356640, clear bottom, 96 well, Poly-D-Lysine) and incubated overnight in HBSS buffer (Gibco No. 14025-050) with the following additions: 10% FCS (GIBCO, 10270-106) and doxycycline (BD Biosciences Clontech 631311600 ng/ml).

For the functional investigation, the cells were loaded with 2 μM fluo-4 and 0.01 Vol % Pluronic F127 (Molecular Probes Europe BV, Leiden Netherlands) in HBSS buffer (Hank's buffered saline solution, Gibco Invitrogen GmbH, Karlsruhe, Germany) with probenicide (Sigma P8761, 0.69 mg/ml) for 30 min at 37° C. The cells are then washed 3 times with washing buffer (HBSS buffer, Gibco No. 14025-050, with probenicide (Sigma P8761, 0.69 mg/ml) and subsequently received with the same buffer ad 100 μl. After 15 min., the plates are transferred into a Fluorometric Imaging Plate Reader (FLIPR, Molecular Devices, Sunnyvale, Calif.) for the determination of Ca²⁺ measurements in the presence of DHPG ((S)-3,5-dihydroxyphenylglycine, Tocris Biotrend Chemikalien GmbH, Cologne, Germany, final DHPG concentration: 10 μM) and in the presence or absence of test substances.

In this case, the Ca²⁺-dependent fluorescence is measured before and after addition of test substances. Quantification is performed by measurement of the maximum fluorescence intensity over time.

After recording the fluorescence base line for 10 sec., 50 μl test substance solution (various test substance concentrations in HBSS buffer with 1% DMSO and 0.02% Tween 20, Sigma) is added and the fluorescence signal is measured for 6 min. 50 μl DHPG solution ((S)-3,5-dihydroxyphenylglycine, Tocris Biotrend Chemikalien GmbH, Cologne, Germany, final DHPG concentration: 10 μM) is subsequently added and the inflow of Ca²⁺ is simultaneously measured for 60 sec. The final DMSO concentration is 0.25% and the final Tween 20 content is 0.005%. The data are analysed with Microsoft Excel and GraphPad Prism. The dose-effect curves are calculated with non-linear regression and IC₅₀ values determined. Each data point is determined 3 times and IC₅₀ values are averaged from a minimum of 2 independent measurements.

Ki values are calculated according to the following formula: Ki=IC50/(1+(AG_{Conc.}/EC50)).

AG_{Conc.}=10 μM; EC50 corresponds to the DHPG concentration which is required for half the maximum inflow of Ca²⁺.

### III. Formaline Test in Rats:

The formaline test (Dubuisson, D. and Dennis, S. G., 1977, Pain, 4, 161-174) represents a model for acute and chronic pain. A biphasic nociceptive reaction, which is recorded by observation of three clearly differentiable behavioural patterns, is induced by a single formaline injection into the dorsal side of a rear paw in freely mobile test animals. The reaction has two phases: Phase 1=Immediate reaction (duration up to 10 min; paw shaking, licking), Phase 2=Late reaction (after a rest phase; likewise, paw shaking, licking; duration up to 60 min). The 1^{st} phase reflects a direct stimulation of the peripheral nocisensors with high spinal nociceptive input or glutamate release (acute pain phase); the 2^{nd} phase reflects a spinal and peripheral hypersensitisation (chronic pain phase). In the investigations presented here, the chronic pain component (phase 2) was evaluated.

Formaline with a volume of 50 μl and a concentration of 5% is administered subcutaneously into the dorsal side of the right rear paw of each animal. The substances to be tested are administered 30 min before the formaline injection orally (p.o.), intravenously (i.v.) or intraperitoneally (i.p.). The specific changes in behaviour such as lifting and shaking the paw, shifts in weight of the animal as well as biting and licking reactions are observed and registered in the period of observation from 21 to 27 min after formaline injection. The various forms of behaviour are summarised in the so-called pain rate (PR), which, relative to the sub-intervals of 3 min, represents the calculation of an average nociception reaction. The calculation of PR is performed on the basis of a numerical weighting (=in each case factor 1, 2, 3) of the observed forms of behaviour (corresponding behavioural score 1, 2, 3) and is calculated with the following formula:

PR=[(T₀×0)+(T₁×1)+(T₂×2)+(T₃×3)]/180

whereby T₀, T₁, T₂, and T₃ in each case corresponds to the time in seconds in which the animal demonstrates modes of behaviour 0, 1, 2 or 3. The group size is 10 animals (n=10).

The following examples serve to explain the invention and do not restrict the general concept of the invention.

### EXAMPLES

The yields of the produced compounds are not optimised.

All temperatures are uncorrected.

The chemicals and solvents used were commercially acquired from the normal suppliers (Acros, Avocado, Aldrich, Bachem, Fluka, Lancaster, Maybridge, Merck, Sigma, TCl, etc.) or synthesised.

Silica gel 60 (0.040-0.063 mm) from E. Merck, Darmstadt was used as the stationary phase for the column chromathography.

The thin layer chromatographic tests were carried out with HPTLC ready plates, silica gel 60 F 254, from E. Merck, Darmstadt.

The mixture ratios of solvents, mobile solvents or for chromatographic investigations are always indicated in volume/volume.

Analysis was performed by mass spectroscopy and NMR, unless indicated otherwise.

### Abbreviations:

aq. aqueous
Brine saturated aqueous NaCl solution
CDl 1,1′-carbonyl-diimidazol
CHCl₃ chloroform
DCE 1,2-dichloroethane
DCM dichloromethane
DIC N,N′-diisopropylcarbodiimide

### DMF N,N-dimethylformamide

EA ethylacetate
HOBt 1-hydroxy-benzotriazol
sol solution
M molar
MeCN acetonitrile
MeOH methanol
PL-EDC a polymer-bound carbodiimide with following structure:

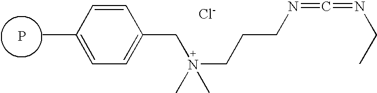

Loading: 1.4 mmol/g
Particle size: 300-500 μm
   PS-carbodiimide a polymer-bound carbodiimide with following structure:

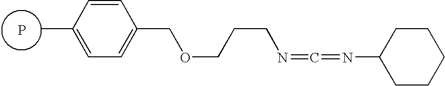

Loading: 0.9-1.4 mmol/g
Particle size: 75-150 μm
   RT room temperature
   CC column chromatography

### Example 1

### Synthesis of 3-(3-chlorophenyl)-N-methyl-N-phenethylpropiolamide

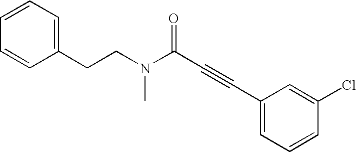

3.5 g (≅5.1 mmol) PL-EDC was added to a solution of 254 μl (1.75 mmol) N-methyl-2-phenylethylamine and 451 mg (2.5 mmol) 3-(3-chlorophenyl)-propiolic acid in DCM (35 ml) and the reaction solution was shaken for 4 h at RT. The resin was subsequently filtered off and washed with DCM and MeOH. The filtrate was concentrated in a vacuum and CC (DCM) was performed with the residue, whereby 222 mg (0.75 mmol, 43%) 3-(3-chlorophenyl)-N-methyl-N-phenethylpropiolamide was obtained.

MS: [MH+] 297.1

The synthesis of examples 2-4 (Table 1) was performed according to the method described for Example 1.

TABLE 1

| | | |
|---|---|---|
| 2 | 3-(3-chlorophenyl)-N-phenethylpropiolamide | 284.1 |
| 3 | 3-(3-chlorophenyl)-N-(2-(pyridine-2- | 285.1 |
| | yl)ethyl)propiolamide | |
| 4 | 3-(3-chlorophenyl)-N-(2-(pyridine-4- | 285.1 |
| | yl)ethyl)propiolamide | |

General Synthesis Instructions for the Conversion of Primary and Secondary Amines with Aromatically Substituted Propiolic Acids (AAV 1)

A solution of CDI (105 μmol, 1.05 equivalents) in DCM (1.05 ml) was added to a solution of the respective aromatically substituted propiolic acid (100 μmol) in DCM (2 ml). The reaction solution was stirred for 1 h at 20° C. gerührt and a solution of the respective primary or secondary amine (100 μmol, 1.0 equivalents) in DCM (1 ml) was subsequently added. Stirring was subsequently performed for a further 16 h at RT. Water (3 ml) was then added to the reaction mixture and the phases were separated. The organic phase was washed with water (3 ml) and with brine (3 ml), dried over MgSO₄ and filtered. After removal of the solvent in a vacuum, the respective target compound was isolated from the residue by means of preparative HPLC.

The synthesis of examples 5 to 214 (Table 2) was performed according to the general method described for conversion of primary and secondary amines with aromatically substituted propiolic acids. Therein, it is apparent to the person skilled in the art which starting compounds and intermediate products were used in each case.

TABLE 2

| | | |
|---|---|---|
| 5 | 3-(2,4-difluorophenyl)-N-(2-(pyridine-2-yl)ethyl)propiolamide | 287.1 |
| 6 | 3-(3-methoxyphenyl)-N-(2-(pyridine-2-yl)ethyl)propiolamide | 281.1 |
| 7 | 3-(4-fluoro-3-methylphenyl)-N-(2-(pyridine-2-yl)ethyl)propiolamide | 283.1 |
| 8 | 3-(2-fluorophenyl)-N-(2-(pyridine-2-yl)ethyl)propiolamide | 269.1 |
| 9 | N-(2-(pyridine-2-yl)ethyl)-3-p-tolylpropiolamide | 265.1 |
| 10 | N-(2-(pyridine-2-yl)ethyl)-3-(4-(trifluoromethyl)-phenyl)-propiolamide | 319.1 |
| 11 | 3-phenyl-N-(2-(pyridine-2-yl)ethyl)propiolamide | 251.1 |
| 12 | 3-(2,3-dimethyl-phenyl)-N-(2-(pyridine-2-yl)ethyl)propiolamide | 279.1 |
| 13 | 3-(3,5-dimethyl-phenyl)-N-(2-(pyridine-2-yl)ethyl)propiolamide | 279.1 |
| 14 | 3-(3,5-dichloro-phenyl)-N-(2-(pyridine-2-yl)ethyl)propiolamide | 319.0 |
| 15 | 3-(3-fluoro-4-methyl-phenyl)-N-(2-(pyridine-2-yl)ethyl)propiolamide | 283.1 |
| 16 | 3-(4-tert.butyl-phenyl)-N-(2-(pyridine-2-yl)ethyl)propiolamide | 307.2 |
| 17 | 3-(2,4-dichloro-phenyl)-N-(2-(pyridine-2-yl)ethyl)propiolamide | 319.0 |
| 18 | N-(2-(pyridine-2-yl)ethyl)-3-m-tolyl-propiolamide | 265.1 |
| 19 | 3-(2,4-dimethyl-phenyl)-N-(2-(pyridine-2-yl)ethyl)propiolamide | 279.1 |
| 20 | 3-(4-fluoro-phenyl)-N-(2-(pyridine-2-yl)ethyl)propiolamide | 269.1 |
| 21 | 3-(4-chloro-phenyl)-N-(2-(pyridine-2-yl)ethyl)propiolamide | 285.1 |
| 22 | 3-(2-cyano-phenyl)-N-(2-(pyridine-2-yl)ethyl)propiolamide | 276.1 |
| 23 | 3-(4-cyano-phenyl)-N-(2-(pyridine-2-yl)ethyl)propiolamide | 276.1 |
| 24 | 3-(4-methoxy-phenyl)-N-(2-(pyridine-2-yl)ethyl)propiolamide | 281.1 |
| 25 | N-(2-(pyridine-2-yl)ethyl)-3-thiophene-2-yl-propiolamide | 257.1 |
| 26 | N-benzyl-3-(2,4-difluoro-phenyl)-N-phenethylpropiolamide | 376.1 |
| 27 | N-benzyl-3-(2-fluoro-phenyl)-N-phenethylpropiolamide | 358.2 |
| 28 | N-benzyl-N-phenethyl-3-p-tolyl-propiolamide | 354.2 |
| 29 | N-benzyl-N-phenethyl-3-(4-(trifluoromethyl)-phenyl)-propiolamide | 408.1 |
| 30 | 3-(2-bromo-5-methoxy-phenyl)-N-(2-(pyridine-2-yl)ethyl)propiolamide | 359.0 |
| 31 | N-(2-(pyridine-2-yl)ethyl)-3-o-tolyl-propiolamide | 265.1 |
| 32 | N-(2-(pyridine-2-yl)ethyl)-3-(2-(trifluoromethyl)-phenyl)-propiolamide | 319.1 |
| 33 | 3-(3-bromo-4-methoxy-phenyl)-N-(2-(pyridine-2-yl)ethyl)propiolamide | 359.0 |
| 34 | N-benzyl-N-phenethyl-3-phenyl-propiolamide | 340.2 |
| 35 | N-benzyl-3-(2,3-dimethyl-phenyl)-N-phenethyl-propiolamide | 368.2 |
| 36 | N-benzyl-3-(3,5-dichloro-phenyl)-N-phenethyl-propiolamide | 408.1 |
| 37 | N-benzyl-3-(2,4-dichloro-phenyl)-N-phenethyl-propiolamide | 408.1 |
| 38 | 3-(1H-indol-5-yl)-N-(2-(pyridine-2-yl)ethyl)propiolamide | 290.1 |
| 39 | N-benzyl-3-(1H-indol-5-yl)-N-phenethylpropiolamide | 379.2 |
| 40 | N-(3,4-dimethoxyphenethyl)-3-(4-fluoro-3-methylphenyl)-N-methylpropiolamide | 356.2 |
| 41 | 3-(4-fluoro-3-methylphenyl)-N-methyl-N-phenethylpropiolamide | 296.1 |
| 42 | 3-(2-fluorophenyl)-N-methyl-N-phenethylpropiolamide | 282.1 |
| 43 | N-(3,4-dimethoxyphenethyl)-N-methyl-3-p-tolylpropiolamide | 338.2 |
| 44 | N-methyl-N-phenethyl-3-p-tolylpropiolamide | 278.1 |
| 45 | 3-(2,4-dichlorophenyl)-N-methyl-N-phenethylpropiolamide | 332.1 |
| 46 | N-methyl-N-phenethyl-3-m-tolylpropiolamide | 278.1 |
| 47 | 3-(2,4-dimethylphenyl)-N-methyl-N-phenethylpropiolamide | 292.2 |
| 48 | N-(3,4-dimethoxyphenethyl)-3-(4-fluorophenyl)-N- | 342.1 |
| | methylpropiolamide | |
| 49 | N-methyl-N-phenethyl-3-(3-(trifluoromethyl)phenyl)propiolamide | 332.1 |
| 50 | N-(3,4-dimethoxyphenethyl)-N-methyl-3-o-tolylpropiolamide | 338.2 |
| 51 | N-methyl-N-phenethyl-3-o-tolylpropiolamide | 278.1 |
| 52 | N-(3,4-dimethoxyphenethyl)-N-methyl-3-(2- | 392.1 |
| | (trifluoromethyl)phenyl)propiolamide | |
| 53 | N-methyl-N-phenethyl-3-(2-(trifluoromethyl)phenyl)propiolamide | 332.1 |
| 54 | 3-(2-cyanophenyl)-N-(3,4-dimethoxyphenethyl)-N- | 349.1 |
| | methylpropiolamide | |
| 55 | 3-(2-cyanophenyl)-N-methyl-N-phenethylpropiolamide | 289.1 |
| 56 | 3-(4-fluoro-3-methylphenyl)-N-(2-(thiophene-2-yl)ethyl)propiolamide | 288.1 |
| 57 | N-(2-(1H-indol-3-yl)ethyl)-3-m-tolylpropiolamide | 303.1 |
| 58 | N-(3-chlorophenethyl)-3-(3-methoxyphenyl)propiolamide | 314.1 |
| 59 | 3-(3-methoxyphenyl)-N-(4-methylphenethyl)propiolamide | 294.1 |
| 60 | N-(2,2-diphenylethyl)-3-(3-methoxyphenyl)propiolamide | 356.2 |
| 61 | N-(4-fluorophenethyl)-3-(3-methoxyphenyl)propiolamide | 298.1 |
| 62 | N-(3-chlorophenethyl)-3-(4-fluoro-3-methylphenyl)propiolamide | 316.1 |
| 63 | 3-(4-fluoro-3-methylphenyl)-N-(4-methylphenethyl)propiolamide | 296.1 |
| 64 | N-(2,2-diphenylethyl)-3-(4-fluoro-3-methylphenyl)propiolamide | 358.2 |
| 65 | N-(3-chlorophenethyl)-3-phenylpropiolamide | 284.1 |
| 66 | N-(4-methylphenethyl)-3-phenylpropiolamide | 264.1 |
| 67 | N-(2,2-diphenylethyl)-3-phenylpropiolamide | 326.1 |
| 68 | N-(4-fluorophenethyl)-3-phenylpropiolamide | 268.1 |
| 69 | N-(3-chlorophenethyl)-3-m-tolylpropiolamide | 298.1 |
| 70 | N-(4-methylphenethyl)-3-m-tolylpropiolamide | 278.1 |
| 71 | N-(2,2-diphenylethyl)-3-m-tolylpropiolamide | 340.2 |
| 72 | 3-(4-fluorophenyl)-N-phenethylpropiolamide | 268.1 |
| 73 | 3-(thiophene-2-yl)-N-(2-(thiophene-2-yl)ethyl)propiolamide | 262.0 |
| 74 | N-benzyl-3-(4-fluorophenyl)-N-phenethylpropiolamide | 358.2 |
| 75 | N-benzyl-3-(4-tert-butylphenyl)-N-phenethylpropiolamide | 396.2 |
| 76 | N-benzyl-N-phenethyl-3-(2-(trifluoromethyl)phenyl)propiolamide | 408.1 |
| 77 | N-benzyl-N-phenethyl-3-(thiophene-2-yl)propiolamide | 346.1 |
| 78 | 3-(2,4-difluorophenyl)-N-methyl-N-phenethylpropiolamide | 300.1 |
| 79 | N-(3,4-dimethoxyphenethyl)-3-(3-methoxyphenyl)-N- | 354.2 |
| | methylpropiolamide | |
| 80 | 3-(3-methoxyphenyl)-N-methyl-N-phenethylpropiolamide | 294.1 |
| 81 | 3-(3-methoxyphenyl)-N-phenethylpropiolamide | 280.1 |
| 82 | 3-(4-fluoro-3-methylphenyl)-N-phenethylpropiolamide | 282.1 |
| 83 | N-phenethyl-3-phenylpropiolamide | 250.1 |
| 84 | N-phenethyl-3-m-tolylpropiolamide | 264.1 |
| 85 | N-phenethyl-3-(thiophene-2-yl)propiolamide | 256.1 |
| 86 | 3-(3-cyanophenyl)-N-phenethylpropiolamide | 275.1 |
| 87 | N-(2-(1H-indol-3-yl)ethyl)-3-(3-methoxyphenyl)propiolamide | 319.1 |
| 88 | N-(2-(1H-indol-3-yl)ethyl)-3-(4-fluoro-3-methylphenyl)propiolamide | 321.1 |
| 89 | N-(3-chlorophenethyl)-3-(4-fluorophenyl)propiolamide | 302.1 |
| 90 | 3-(4-fluorophenyl)-N-(4-methylphenethyl)propiolamide | 282.1 |
| 91 | N-(2,2-diphenylethyl)-3-(4-fluorophenyl)propiolamide | 344.1 |
| 92 | N-(4-fluorophenethyl)-3-(4-fluorophenyl)propiolamide | 286.1 |
| 93 | N-(3-chlorophenethyl)-3-(3-chlorophenyl)propiolamide | 318.0 |
| 94 | N-(3-chlorophenethyl)-3-(thiophene-2-yl)propiolamide | 290.0 |
| 95 | N-(4-methylphenethyl)-3-(thiophene-2-yl)propiolamide | 270.1 |
| 96 | N-(2,2-diphenylethyl)-3-(thiophene-2-yl)propiolamide | 332.1 |
| 97 | 3-(3-cyanophenyl)-N-(4-methylphenethyl)propiolamide | 289.1 |
| 98 | 3-(3-cyanophenyl)-N-(2,2-diphenylethyl)propiolamide | 351.1 |
| 99 | 3-(3-cyanophenyl)-N-(4-fluorophenethyl)propiolamide | 293.1 |
| 100 | N-(3,4-dichlorophenethyl)-3-(4-fluoro-3-methylphenyl)propiolamide | 350.0 |
| 101 | N-(3,4-dichlorophenethyl)-3-phenylpropiolamide | 318.0 |
| 102 | N-(3,4-dichlorophenethyl)-3-m-tolylpropiolamide | 332.1 |
| 103 | N-(3,4-dichlorophenethyl)-3-(thiophene-2-yl)propiolamide | 324.0 |
| 104 | 3-(3-cyanophenyl)-N-(3,4-dichlorophenethyl)propiolamide | 343.0 |
| 105 | N-(2-(1H-indol-3-yl)ethyl)-3-(2,4-difluorophenyl)propiolamide | 325.1 |
| 106 | N-(2-(1H-indol-3-yl)ethyl)-3-(2-fluorophenyl)propiolamide | 307.1 |
| 107 | N-(2-(1H-indol-3-yl)ethyl)-3-p-tolylpropiolamide | 303.1 |
| 108 | N-(2-(1H-indol-3-yl)ethyl)-3-(2,4-dichlorophenyl)propiolamide | 357.0 |
| 109 | N-(2-(1H-indol-3-yl)ethyl)-3-(2,4-dimethylphenyl)propiolamide | 317.2 |
| 110 | N-(2-(1H-indol-3-yl)ethyl)-3-(4-tert-butylphenyl)propiolamide | 345.2 |
| 111 | N-(2-(1H-indol-3-yl)ethyl)-3-(3,4-dimethylphenyl)propiolamide | 317.2 |
| 112 | N-(2-(1H-indol-3-yl)ethyl)-3-(2-bromo-5- | 397.0 |
| | methoxyphenyl)propiolamide | |
| 113 | N-(2-(1H-indol-3-yl)ethyl)-3-o-tolylpropiolamide | 303.1 |
| 114 | N-(2-(1H-indol-3-yl)ethyl)-3-(2-(trifluoromethyl)phenyl)propiolamide | 357.1 |
| 115 | N-(2-(1H-indol-3-yl)ethyl)-3-(2-cyanophenyl)propiolamide | 314.1 |
| 116 | N-(2-(1H-indol-3-yl)ethyl)-3-(3,5-dichlorophenyl)propiolamide | 357.0 |
| 117 | 3-(2,4-difluorophenyl)-N-(2-(thiophene-2-yl)ethyl)propiolamide | 292.1 |
| 118 | 3-(2-fluorophenyl)-N-(2-(thiophene-2-yl)ethyl)propiolamide | 274.1 |
| 119 | N-(2-(thiophene-2-yl)ethyl)-3-p-tolylpropiolamide | 270.1 |
| 120 | N-(2-(thiophene-2-yl)ethyl)-3-(4- | 324.1 |
| | (trifluoromethyl)phenyl)propiolamide | |
| 121 | 3-(2,3-dimethylphenyl)-N-(2-(thiophene-2-yl)ethyl)propiolamide | 284.1 |
| 122 | 3-(3,5-dichlorophenyl)-N-(2-(thiophene-2-yl)ethyl)propiolamide | 324.0 |
| 123 | 3-(2,4-dichlorophenyl)-N-(2-(thiophene-2-yl)ethyl)propiolamide | 324.0 |
| 124 | 3-(3-fluoro-4-methylphenyl)-N-(2-(thiophene-2-yl)ethyl)propiolamide | 288.1 |
| 125 | 3-(4-tert-butylphenyl)-N-(2-(thiophene-2-yl)ethyl)propiolamide | 312.1 |
| 126 | 3-(3,4-dimethylphenyl)-N-(2-(thiophene-2-yl)ethyl)propiolamide | 284.1 |
| 127 | 3-(2-bromo-5-methoxyphenyl)-N-(2-(thiophene-2- | 364.0 |
| | yl)ethyl)propiolamide | |
| 128 | N-(2-(thiophene-2-yl)ethyl)-3-o-tolylpropiolamide | 270.1 |
| 129 | N-(2-(thiophene-2-yl)ethyl)-3-(2- | 324.1 |
| | (trifluoromethyl)phenyl)propiolamide | |
| 130 | 3-(2-cyanophenyl)-N-(2-(thiophene-2-yl)ethyl)propiolamide | 281.1 |
| 131 | 3-(4-cyanophenyl)-N-(2-(thiophene-2-yl)ethyl)propiolamide | 281.1 |
| 132 | N-(2-methoxyphenethyl)-3-(3-methoxyphenyl)propiolamide | 310.1 |
| 133 | N-(4-chlorophenethyl)-3-(3-methoxyphenyl)propiolamide | 314.1 |
| 134 | 3-(4-fluoro-3-methylphenyl)-N-(2-methoxyphenethyl)propiolamide | 312.1 |
| 135 | N-(4-chlorophenethyl)-3-(4-fluoro-3-methylphenyl)propiolamide | 316.1 |
| 136 | N-(2-methoxyphenethyl)-3-phenylpropiolamide | 280.1 |
| 137 | N-(4-chlorophenethyl)-3-phenylpropiolamide | 284.1 |
| 138 | N-(2-methoxyphenethyl)-3-m-tolylpropiolamide | 294.1 |
| 139 | N-(4-chlorophenethyl)-3-m-tolylpropiolamide | 298.1 |
| 140 | N-(4-chlorophenethyl)-3-(4-fluorophenyl)propiolamide | 302.1 |
| 141 | 3-(3,5-dichlorophenyl)-N-(2-methoxyphenethyl)propiolamide | 348.0 |
| 142 | 3-(2,4-dichlorophenyl)-N-(2-methoxyphenethyl)propiolamide | 348.0 |
| 143 | 3-(2,4-dimethylphenyl)-N-phenethylpropiolamide | 278.1 |
| 144 | N-(3-chlorophenethyl)-3-(2,4-difluorophenyl)propiolamide | 320.1 |
| 145 | 3-(2,4-difluorophenyl)-N-(4-methylphenethyl)propiolamide | 300.1 |
| 146 | 3-(2,4-difluorophenyl)-N-(2,2-diphenylethyl)propiolamide | 362.1 |
| 147 | 3-(2,4-difluorophenyl)-N-(4-fluorophenethyl)propiolamide | 304.1 |
| 148 | N-(3-chlorophenethyl)-3-(2-fluorophenyl)propiolamide | 302.1 |
| 149 | 3-(2-fluorophenyl)-N-(4-methylphenethyl)propiolamide | 282.1 |
| 150 | N-(2,2-diphenylethyl)-3-(2-fluorophenyl)propiolamide | 344.1 |
| 151 | N-(4-fluorophenethyl)-3-(2-fluorophenyl)propiolamide | 286.1 |
| 152 | N-(3-chlorophenethyl)-3-p-tolylpropiolamide | 298.1 |
| 153 | N-(4-methylphenethyl)-3-p-tolylpropiolamide | 278.1 |
| 154 | N-(2,2-diphenylethyl)-3-p-tolylpropiolamide | 340.2 |
| 155 | N-(3-chlorophenethyl)-3-(4-(trifluoromethyl)phenyl)propiolamide | 352.1 |
| 156 | N-(4-methylphenethyl)-3-(4-(trifluoromethyl)phenyl)propiolamide | 332.1 |
| 157 | N-(2,2-diphenylethyl)-3-(4-(trifluoromethyl)phenyl)propiolamide | 394.1 |
| 158 | N-(4-fluorophenethyl)-3-(4-(trifluoromethyl)phenyl)propiolamide | 336.1 |
| 159 | N-(3,4-dichlorophenethyl)-3-(4-(trifluoromethyl)phenyl)propiolamide | 386.0 |
| 160 | N-(3-chlorophenethyl)-3-(2,3-dimethylphenyl)propiolamide | 312.1 |
| 161 | 3-(2,3-dimethylphenyl)-N-(4-methylphenethyl)propiolamide | 292.2 |
| 162 | 3-(2,3-dimethylphenyl)-N-(2,2-diphenylethyl)propiolamide | 354.2 |
| 163 | 3-(2,3-dimethylphenyl)-N-(4-fluorophenethyl)propiolamide | 296.1 |
| 164 | N-(3,4-dichlorophenethyl)-3-(2,3-dimethylphenyl)propiolamide | 346.1 |
| 165 | N-(3-chlorophenethyl)-3-(3,5-dimethylphenyl)propiolamide | 312.1 |
| 166 | 3-(3,5-dimethylphenyl)-N-(4-methylphenethyl)propiolamide | 292.2 |
| 167 | 3-(3,5-dimethylphenyl)-N-(2,2-diphenylethyl)propiolamide | 354.2 |
| 168 | 3-(4-hydroxy-3-methylphenyl)-N-(4-methylphenethyl)propiolamide | 294.1 |
| 169 | N-(2,2-diphenylethyl)-3-(4-hydroxy-3-methylphenyl)propiolamide | 356.2 |
| 170 | 3-(1H-indol-5-yl)-N-(4-methylphenethyl)propiolamide | 303.1 |
| 171 | N-(2,2-diphenylethyl)-3-(1H-indol-5-yl)propiolamide | 365.2 |
| 172 | N-(4-fluorophenethyl)-3-(1H-indol-5-yl)propiolamide | 307.1 |
| 173 | N-(3,4-dichlorophenethyl)-3-(1H-indol-5-yl)propiolamide | 357.0 |
| 174 | N-(3-chlorophenethyl)-3-(3,5-dichlorophenyl)propiolamide | 352.0 |
| 175 | 3-(3,5-dichlorophenyl)-N-(4-methylphenethyl)propiolamide | 332.1 |
| 176 | 3-(3,5-dichlorophenyl)-N-(2,2-diphenylethyl)propiolamide | 394.1 |
| 177 | 3-(3,5-dichlorophenyl)-N-(4-fluorophenethyl)propiolamide | 336.0 |
| 178 | N-(3,4-dichlorophenethyl)-3-(3,5-dichlorophenyl)propiolamide | 386.0 |
| 179 | 3-(2,4-dichlorophenyl)-N-(4-methylphenethyl)propiolamide | 332.1 |
| 180 | 3-(2,4-dichlorophenyl)-N-(4-fluorophenethyl)propiolamide | 336.0 |
| 181 | N-(3,4-dichlorophenethyl)-3-(2,4-dichlorophenyl)propiolamide | 386.0 |
| 182 | N-(3-chlorophenethyl)-3-o-tolylpropiolamide | 298.1 |
| 183 | N-(4-methylphenethyl)-3-o-tolylpropiolamide | 278.1 |
| 184 | N-(2,2-diphenylethyl)-3-o-tolylpropiolamide | 340.2 |
| 185 | N-(4-fluorophenethyl)-3-o-tolylpropiolamide | 282.1 |
| 186 | N-(3,4-dichlorophenethyl)-3-o-tolylpropiolamide | 332.1 |
| 187 | N-(3,4-dichlorophenethyl)-3-(4-hydroxy-3- | 348.0 |
| | methylphenyl)propiolamide | |
| 188 | 3-(3-chlorophenyl)-N-(2-methoxyphenethyl)propiolamide | 314.1 |
| 189 | N-(2-methoxyphenethyl)-3-(thiophene-2-yl)propiolamide | 286.1 |
| 190 | N-(4-chlorophenethyl)-3-(thiophene-2-yl)propiolamide | 290.0 |
| 191 | 3-(3-cyanophenyl)-N-(2-methoxyphenethyl)propiolamide | 305.1 |
| 192 | N-(4-chlorophenethyl)-3-(3-cyanophenyl)propiolamide | 309.1 |
| 193 | 3-(2,4-difluorophenyl)-N-(2-methoxyphenethyl)propiolamide | 316.1 |
| 194 | 3-(2-fluorophenyl)-N-(2-methoxyphenethyl)propiolamide | 298.1 |
| 195 | N-(2-methoxyphenethyl)-3-(4-(trifluoromethyl)phenyl)propiolamide | 348.1 |
| 196 | 3-(3-fluoro-4-methylphenyl)-N-phenethylpropiolamide | 282.1 |
| 197 | 3-(4-tert-butylphenyl)-N-phenethylpropiolamide | 306.2 |
| 198 | 3-(3,4-dimethylphenyl)-N-phenethylpropiolamide | 278.1 |
| 199 | 3-(2,4-difluorophenyl)-N-phenethylpropiolamide | 286.1 |
| 200 | N-phenethyl-3-p-tolylpropiolamide | 264.1 |
| 201 | N-phenethyl-3-(4-(trifluoromethyl)phenyl)propiolamide | 318.1 |
| 202 | 3-(2,3-dimethylphenyl)-N-phenethylpropiolamide | 278.1 |
| 203 | 3-(3,5-dimethylphenyl)-N-phenethylpropiolamide | 278.1 |
| 204 | 3-(1H-indol-5-yl)-N-phenethylpropiolamide | 289.1 |
| 205 | 3-(2,3-dimethylphenyl)-N-(2-methoxyphenethyl)propiolamide | 308.2 |
| 206 | 3-(3,5-dimethylphenyl)-N-(2-methoxyphenethyl)propiolamide | 308.2 |
| 207 | 3-(3,5-dichlorophenyl)-N-phenethylpropiolamide | 318.0 |
| 208 | N-(3,4-dichlorophenethyl)-3-(2,4-difluorophenyl)propiolamide | 354.0 |
| 209 | N-(3,4-dichlorophenethyl)-3-(2-fluorophenyl)propiolamide | 336.0 |
| 210 | N-(4-fluorophenethyl)-3-p-tolylpropiolamide | 282.1 |
| 211 | N-(3,4-dichlorophenethyl)-3-p-tolylpropiolamide | 332.1 |
| 212 | 3-(3,5-dimethylphenyl)-N-(4-fluorophenethyl)propiolamide | 296.1 |
| 213 | N-(3-chlorophenethyl)-3-(2,4-dichlorophenyl)propiolamide | 352.0 |
| 214 | 3-(2,4-dichlorophenyl)-N-(2,2-diphenylethyl)propiolamide | 394.1 |

### Example 215

### Synthesis of 3-(3-chlorophenyl)-N-(2-fluorophenethyl)propiolamide

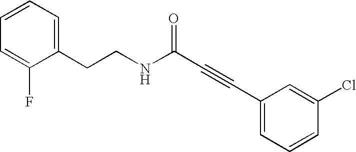

1.0 g (5.54 mmol) 3-(3-chlorophenyl)-propiolic acid, 698 mg (5.54 mmol) DIC and 748 mg (5.54 mmol) HOBt were consecutively added to a solution of 770 mg (5.54 mmol) 2-fluorophenethylamine in DMF (10 ml). Stirring was subsequently performed for 72 h at RT. After 1 M aq. NaHCO₃ sol. was added and dilution with EA, the phases were separated. The organic phase was dried over Na₂SO₄, filtered and concentrated in a vacuum. CC (EA/hexane 1:2) was performed with the residue, whereby 863 mg (2.86 mmol, 52%) 3-(3-chlorophenyl)-N-(2-fluorophenethyl)propiolamide was obtained.

### Example 218

### Synthesis of 3-(3-chlorophenyl)-N-(2-fluorophenethyl)-N-methylpropiolamide

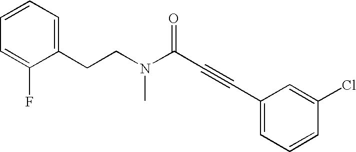

0.1 g sodium hydride (60% in mineral oil, 2.5 mmol) was added to a solution of 620 mg (2.1 mmol) 3-(3-chlorophenyl)-N-(2-fluorophenethyl)-propiolamide (Example 215) in MeCN (20 ml) and the reaction solution was stirred for 1 h at RT. 255 μl (4.2 mmol) iodomethan was subsequently added and stirring was performed for a further 16 h at RT. After quenching with MeOH (1 ml), the reaction solution was concentrated in a vacuum. The residue was received in DCM and washed in each case twice with water and brine. The organic phase was dried over Na₂SO₄, filtered and concentrated in a vacuum. In this process, 609 mg (1.9 mmol, 94%) 3-(3-chlorophenyl)-N-(2-fluorophenethyl)-N-methylpropiolamid was obtained.

### Example 235

### Synthesis of 3-(3-chlorophenyl)-N-(2-(pyridine-3-yl)ethyl)propiolamide

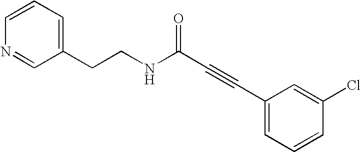

3.1 g (≅3.5 mmol) PS-carbodiimide was added to a solution of 244 mg (2.0 mmol) 2-(pyridine-3-yl)ethylamine and 542 mg (3.0 mmol) 3-(3-chlorophenyl)-propiolic acid in DCM (50 ml) and the reaction solution was shaken for 16 h at RT. The resin was subsequently filtered off and rinsed with DCM and MeOH. The filtrate was concentrated in a vacuum and CC(CHCl₃/MeOH 40:1) was performed with the residue, whereby 447 mg (1.6 mmol, 80%) 3-(3-chlorophenyl)-N-(2-(pyridine-3-yl)ethyl)propiolamide was obtained.

### Example 237

### Synthesis of 3-(3-chlorophenyl)-N-ethyl-N-phenethylpropiolamide

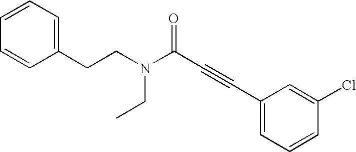

170 mg sodium hydride (60% in mineral oil, 4.23 mmol) and 379 μl (5.08 mmol) ethylbromide were consecutively added to a solution of 240 mg (0.85 mmol) 3-(3-chlorophenyl)-N-methyl-N-phenethylpropiolamide (Example 1) in MeCN (10 ml). The reaction solution was subsequently stirred for 16 h at 40° C. Thereafter, the solution was concentrated in a vacuum and the residue was received with DCM. This solution was washed with water and brine and the organic phase was dried over MgSO₄, filtered and concentrated in a vacuum. CC (hexane/EA 4:1) was performed with the residue, whereby 119 mg (0.38 mmol, 45%) 3-(3-chlorophenyl)-N-ethyl-N-phenethylpropiolamide was obtained.

The synthesis of examples 216, 217, 219, 223-225 and 229-232 was performed in accordance with the method described for Example 215.

The synthesis of examples 220-222, 226-228, 233, 234, 238, 241, 247, 249, 251 and 253 was performed in accordance with the method described for Example 218 for methylating propiolamides. Therein, it is apparent to the person skilled in the art which previously described propiolamide was used in each case.

The synthesis of examples 236, 239, 240, 242-246, 248, 250 and 252 was performed in accordance with the method described for Example 235.

TABLE 3

| | | Mass |
|---|---|---|
| Ex. | Name | [MH+] |
| 215 | 3-(3-chlorophenyl)-N-(2-fluorophenethyl)propiolamide | 302.1 |
| 216 | 3-(3-chlorophenyl)-N-(3-fluorophenethyl)propiolamide | 302.1 |
| 217 | 3-(3-chlorophenyl)-N-(4-fluorophenethyl)propiolamide | 302.1 |
| 218 | 3-(3-chlorophenyl)-N-(2-fluorophenethyl)-N-methylpropiolamide | 316.1 |
| 219 | 3-(3-chlorophenyl)-N-(3-(trifluoromethyl)phenethyl)propiolamide | 352.1 |
| 220 | 3-(3-chlorophenyl)-N-(4-fluorophenethyl)-N-methylpropiolamide | 316.1 |
| 221 | 3-(3-chlorophenyl)-N-(3-fluorophenethyl)-N-methylpropiolamide | 316.1 |
| 222 | 3-(3-chlorophenyl)-N-methyl-N-(3- | 366.1 |
| | (trifluoromethyl)phenethyl)propiolamide | |
| 223 | 3-(3-chlorophenyl)-N-(2-methylphenethyl)propiolamide | 298.1 |
| 224 | 3-(3-chlorophenyl)-N-(3-methylphenethyl)propiolamide | 298.1 |
| 225 | 3-(3-chlorophenyl)-N-(4-methylphenethyl)propiolamide | 298.1 |
| 226 | 3-(3-chlorophenyl)-N-methyl-N-(2-methylphenethyl)propiolamide | 312.1 |
| 227 | 3-(3-chlorophenyl)-N-methyl-N-(3-methylphenethyl)propiolamide | 312.1 |
| 228 | 3-(3-chlorophenyl)-N-methyl-N-(4-methylphenethyl)propiolamide | 312.1 |
| 229 | 3-(3-chlorophenyl)-N-(2-(dimethylamino)-2-phenylethyl)propiolamide | 327.1 |
| 230 | N-(2-(1H-pyrrol-1-yl)ethyl)-3-(3-chlorophenyl)propiolamide | 273.1 |
| 231 | N-(2-(1H-pyrazol-1-yl)ethyl)-3-(3-chlorophenyl)propiolamide | 274.1 |
| 232 | N-(2-(1H-imidazol-1-yl)ethyl)-3-(3-chlorophenyl)propiolamide | 274.1 |
| 233 | N-(2-(1H-pyrrol-1-yl)ethyl)-3-(3-chlorophenyl)-N-methylpropiolamide | 287.1 |
| 234 | N-(2-(1H-pyrazol-1-yl)ethyl)-3-(3-chlorophenyl)-N-methylpropiolamide | 288.1 |
| 235 | 3-(3-chlorophenyl)-N-(2-(pyridine-3-yl)ethyl)propiolamide | 285.1 |
| 236 | 3-(3-chlorophenyl)-N-methyl-N-(2-(pyridine-2-yl)ethyl)propiolamide | 299.1 |
| 237 | 3-(3-chlorophenyl)-N-ethyl-N-phenethylpropiolamide | 312.1 |
| 238 | 3-(3-chlorophenyl)-N-isopropyl-N-phenethylpropiolamide | 326.1 |
| 239 | 3-(3-chlorophenyl)-N-cyclopropyl-N-phenethylpropiolamide | 324.1 |
| 240 | 3-(3-chlorophenyl)-N-(2-methoxyphenethyl)-N-methylpropiolamide | 328.1 |
| 241 | 3-(3-chlorophenyl)-N-methyl-N-(1-phenylpropane-2-yl)propiolamide | 312.1 |
| 242 | 3-(3-chlorophenyl)-N-(1-phenylpropane-2-yl)propiolamide | 298.1 |
| 243 | 3-(3-chlorophenyl)-N-methyl-N-(2-(pyridine-3-yl)ethyl)propiolamide | 299.1 |
| 244 | 3-(3-chlorophenyl)-N-methyl-N-(2-(pyridine-4-yl)ethyl)propiolamide | 299.1 |
| 245 | 3-(3-chlorophenyl)-N-methyl-N-(2-(thiophene-2-yl)ethyl)propiolamide | 304.0 |
| 246 | 3-(3-chlorophenyl)-N-(1-(pyridine-3-yl)propane-2-yl)propiolamide | 299.1 |
| 247 | 3-(3-chlorophenyl)-N-methyl-N-(1-(pyridine-3-yl)propane-2- | 313.1 |
| | yl)propiolamide | |
| 248 | 3-(3-chlorophenyl)-N-(2-(2-methylpyridine-3-yl)ethyl)propiolamide | 299.1 |
| 249 | 3-(3-chlorophenyl)-N-methyl-N-(2-(2-methylpyridine-3- | 313.1 |
| | yl)ethyl)propiolamide | |
| 250 | 3-(3-chlorophenyl)-N-(2-(trifluoromethyl)phenethyl)propiolamide | 352.1 |
| 251 | 3-(3-chlorophenyl)-N-methyl-N-(2- | 366.1 |
| | (trifluormethyl)phenethyl)propiolamide | |
| 252 | 3-(3-chlorophenyl)-N-(4-(trifluoromethyl)phenethyl)propiolamide | 352.1 |
| 253 | 3-(3-chlorophenyl)-N-methyl-N-(4- | 366.1 |
| | (trifluoromethyl)phenethyl)propiolamide | |

### Pharmacological Data:

1. The affinity of the substituted bis(hetero)aromatic N-ethylpropiolamides according to the invention of the general formula I to the mGluR5 receptor was determined as described above (method I).

The substituted bis(hetero)aromatic N-ethylpropiolamides according to the invention exhibit an outstanding affinity to the mGluR5 receptor.

The pharmacological data of substituted bis(hetero)aromatic N-ethylpropiolamides are reproduced in the following table 4:

TABLE 4

| | mGluR5 receptor (pig) | IC₅₀ |
|---|---|---|
| | (10 μM) | mGluR5 receptor (pig) |
| Ex. | Inhibition (%) | [³H]-MPEP bond [μM] |
| 1 | | 0.012 |
| 2 | | 0.044 |
| 3 | | 0.034 |
| 4 | | 2.000 |
| 6 | 82 | |
| 7 | 82 | |
| 8 | 70 | |
| 11 | 81 | |
| 18 | 90 | |
| 20 | 54 | |
| 25 | 68 | |
| 38 | 36 | |
| 41 | 92 | |
| 42 | 90 | |
| 46 | 97 | |
| 47 | 51 | |
| 49 | 108 | |
| 55 | 40 | |
| 56 | 54 | |
| 57 | 91 | |
| 58 | 91 | |
| 59 | 57 | |
| 61 | 85 | |
| 62 | 75 | |
| 65 | 97 | |
| 66 | 64 | |
| 68 | 90 | |
| 69 | 107 | |
| 70 | 89 | |
| 71 | 57 | |
| 72 | 57 | |
| 73 | 76 | |
| 78 | 69 | |
| 80 | 70 | |
| 81 | 79 | |
| 82 | 69 | |
| 83 | 91 | |
| 84 | 89 | |
| 85 | 79 | |
| 86 | 86 | |
| 87 | 42 | |
| 89 | 64 | |
| 92 | 42 | |
| 93 | 89 | |
| 94 | 84 | |
| 95 | 75 | |
| 96 | 31 | |
| 97 | 96 | |
| 98 | 40 | |
| 99 | 89 | |
| 101 | 89 | |
| 102 | 105 | |
| 103 | 88 | |
| 104 | 84 | |
| 117 | 33 | |
| 118 | 67 | |
| 132 | 83 | |
| 133 | 65 | |
| 134 | 77 | |
| 135 | 44 | |
| 136 | 110 | |
| 137 | 67 | |
| 138 | 97 | |
| 139 | 91 | |
| 140 | 53 | |
| 148 | 79 | |
| 149 | 57 | |
| 151 | 66 | |
| 189 | 85 | |
| 190 | 65 | |
| 191 | 96 | |
| 192 | 91 | |
| 193 | 31 | |
| 194 | 81 | |
| 215 | | 0.009 |
| 216 | | 0.012 |
| 217 | | 0.140 |
| 218 | | 0.016 |
| 219 | | 0.450 |
| 220 | | 0.009 |
| 221 | | 0.008 |
| 222 | | 0.075 |
| 223 | | 0.013 |
| 224 | | 0.040 |
| 225 | | 0.210 |
| 226 | | 0.008 |
| 227 | | 0.015 |
| 228 | | 0.280 |
| 230 | | 0.190 |
| 231 | | 0.140 |
| 233 | | 0.031 |
| 234 | | 0.051 |
| 235 | | 0.007 |
| 236 | | 0.029 |
| 237 | | 0.044 |
| 238 | | 0.070 |
| 239 | | 0.280 |
| 240 | | 0.012 |
| 241 | | 0.013 |
| 242 | | 0.002 |
| 243 | | 0.012 |
| 244 | | 0.023 |
| 245 | | 0.011 |
| 246 | | 0.010 |
| 247 | | 0.140 |
| 248 | | 0.260 |
| 249 | | 0.120 |
| 250 | | 0.033 |
| 251 | | 0.028 |

2. The substituted bis(hetero)aromatic N-ethylpropiolamides according to the invention of the general formula I also exhibit an outstanding effect in the formaline test in rats (Method 3), as shown in Example 1, as a result of whose i. v. administration at 21.5 mg/kg a 60% inhibition of the pain reaction is achieved.

3. The affinity of the substituted bis(hetero)aromatic N-ethylpropiolamides according to the invention of the general formula I to the mGluR5 receptor was also determined as described above in Method II (Table 5).

TABLE 5

| | Kᵢ mGluR5 receptor |
|---|---|
| Ex. | (human) Ca²⁺ influx [nM] |
| 1 | 0.0016 |
| 2 | 0.0069 |
| 3 | 0.0062 |
| 4 | 1.4468 |
| 235 | 0.0139 |
| 242 | 0.0056 |
| 243 | 0.2074 |

1. A substituted bis(hetero)aromatic N-ethylpropiolamides of the formula I, 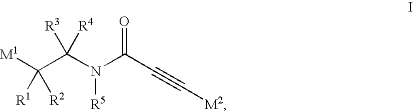
in which
I.)
M¹ denotes phenyl, which can be unsubstituted or substituted with 1, 2, 3, 4 or 5 substituents mutually independently selected from the group consisting of F, Cl, Br, I, —CN, —NO₂, —OH, —SH, —NH₂, —C(═O)—OH, methyl, ethyl, n-propyl, isopropyl, n-butyl, isobutyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, —S(═O)₂—C₁₋₅-alkyl, —S(═O)—C₁₋₅-alkyl, —NH—C₁₋₅-alkyl, —N(C₁₋₅-alkyl)₂, —C(═O)—O—C₁₋₅-alkyl, —C(═O)—O-phenyl, —C(═O)—H; —C(═O)—C₁₋₅-alkyl, —CH₂—O—C(═O)-phenyl, —O—C(═O)-phenyl, —O—C(═O)—C₁₋₅-alkyl, —NH—C(═O)—C₁₋₅-alkyl, —C(═O)—NH₂, —C(═O)—NH—C₁₋₅-alkyl, —C(═O)—N(C₁₋₅-alkyl)₂, —C(═O)—N(C₁₋₅-alkyl)(phenyl), —C(═O)—NH-phenyl, cyclopropyl, cyclobutyl, cyclopentyl, cyclohexyl, pyrazolyl, phenyl, furyl (furanyl), thiazolyl, thiadiazolyl, thiophenyl (thienyl), benzyl and phenethyl, whereby the above-mentioned C₁₋₅-alkyl residues can in each case be linear or branched and the cyclic substituents or the cyclic residues of these substituents themselves can be substituted with optionally 1, 2, 3, 4 or 5 substituents mutually independently selected from the group consisting of F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂ and —S—CH₂F;
and M² denotes phenyl, which is substituted with 1, 2, 3, 4 or 5 substituents mutually independently selected from the group consisting of F, Cl, Br, I, —CN, —NO₂, —OH, —SH, —NH₂, —C(═O)—OH, methyl, ethyl, n-propyl, isopropyl, n-butyl, isobutyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, —S(═O)₂—C₁₋₅-alkyl, —S(═O)—C₁₋₅-alkyl, —NH—C₁₋₅-alkyl, —N(C₁₋₅alkyl)₂, —C(═O)—O—C₁₋₅-alkyl, —C(═O)—O-phenyl, —C(═O)—H; —C(═O)—C₁₋₅-alkyl, —CH₂—O—C(═O)—phenyl, —O—C(═O)-phenyl, —O—C(═O)—C₁₋₅-alkyl, —NH—C(═O)—C₁₋₅-alkyl, —C(═O)—NH₂, —C(═O)—NH—C₁₋₅-alkyl, —C(═O)—N(C₁₋₅-alkyl)₂, —C(═O)—N(C₁₋₅-alkyl)(phenyl), —C(═O)—NH-phenyl, cyclopropyl, cyclobutyl, cyclopentyl, cyclohexyl, pyrazolyl, phenyl, furyl (furanyl), thiazolyl, thiadiazolyl, thiophenyl (thienyl), benzyl and phenethyl, whereby the above-mentioned C₁₋₅-alkyl residues can in each case be linear or branched and the cyclic substituents or the cyclic residues of these substituents themselves can be substituted with optionally 1, 2, 3, 4 or 5 substituents mutually independently selected from the group consisting of F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂ and —S—CH₂F;
or M² denotes unsubstituted or substituted heteroaryl or unsubstituted or substituted naphthyl or denotes an unsubstituted or substituted phenyl residue, which can be condensed (annelated) with unsubstituted or substituted 5- to 7-membered heterocycloalkyl or with unsubstituted or substituted C₅₋₇-cycloalkyl;
or II.)
M¹ denotes unsubstituted or substituted heteroaryl or unsubstituted or substituted naphthyl or an unsubstituted or substituted phenyl residue, which can be condensed (annelated) with unsubstituted or substituted 5- to 7-membered heterocycloalkyl or with unsubstituted or substituted C₅₋₇-cycloalkyl;
and M² denotes unsubstituted or substituted heteroaryl or unsubstituted or substituted naphthyl or an unsubstituted or substituted phenyl residue, which can be condensed (annelated) with unsubstituted or substituted 5- to 7-membered heterocycloalkyl or with unsubstituted or substituted C₅₋₇-cycloalkyl;
or M² denotes phenyl, which can be unsubstituted or substituted with 1, 2, 3, 4 or 5 substituents mutually independently selected from the group consisting of F. Cl, Br, I, —CN, —NO₂, —OH, —SH, —NH₂, —C(═O)—OH, methyl, ethyl, n-propyl, isopropyl, n-butyl, isobutyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, —S(═O)₂—C₁₋₅-alkyl, —S(═O)—C₁₋₅-alkyl, —NH—C₁₋₅-alkyl, —N(C₁₋₅alkyl)₂, —C(═O)—O—C₁₋₅-alkyl, —C(═O)—O-phenyl, —C(═O)—H; —C(═O)—C₁₋₅-alkyl, —CH₂—O—C(═O)-phenyl, —O—C(═O)-phenyl, —O—C(═O)—C₁₋₅-alkyl, —NH—C(═O)—C₁₋₅-alkyl, —C(═O)—NH₂, —C(═O)—NH—C₁₋₅-alkyl, —C(═O)—N(C₁₋₅-alkyl)₂, —C(═O)—N(C₁₋₅-alkyl)(phenyl), —C(═O)—NH-phenyl, cyclopropyl, cyclobutyl, cyclopentyl, cyclohexyl, pyrazolyl, phenyl, furyl (furanyl), thiazolyl, thiadiazolyl, thiophenyl (thienyl), benzyl and phenethyl, whereby the above-mentioned C₁₋₅-alkyl residues can in each case be linear or branched and the cyclic substituents or the cyclic residues of these substituents themselves can be substituted with optionally 1, 2, 3, 4 or 5 substituents mutually independently selected from the group consisting of F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂ and —S—CH₂F;
and in each case
R¹ and R², mutually independently, in each case denote H; F; Cl; Br; I; —NO₂; —CN; —NH₂; —OH; —SH; —O—R⁶; —S—R⁷; —NH—R⁸; —NR⁹R¹⁰; unsubstituted or substituted alkyl, alkenyl or alkynyl; unsubstituted or substituted heteroalkyl, heteroalkenyl or heteroalkynyl; unsubstituted or substituted cycloalkyl or cycloalkenyl; unsubstituted or substituted heterocycloalkyl or heterocycloalkenyl; unsubstituted or substituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or substituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; or unsubstituted or substituted -(heteroalkylene)-heterocycloalkyl, -(heteroalkenylene)-heterocycloalkyl, -(heteroalkylene)-heterocycloalkenyl or -(heteroalkenylene)-heterocycloalkenyl; or aryl;
or R¹ and R² jointly denote an oxo group (═O);
R³ and R⁴, mutually independently, in each case denote H; F; Cl; Br; I; —NO₂; —CN; —NH₂; —OH; —SH; —C(═O)—OH; —NH—C(═O)—H; —O—R⁶; —S—R⁷; —NH—R⁸; —NR⁹R¹⁰; —C(═O)—R¹¹; —O—C(═O)—R¹³; —NH—C(═O)—R¹⁴; —NR¹⁵—C(═O)—R¹⁶; —C(═O)—NH₂; —C(═O)—NH—R¹⁷; —C(═O)—NR¹⁸R¹⁹; —S(═O)—R²⁰; —S(═O)₂—R²¹; —NH—S(═O)₂—R²²; —NR²³—S(═O)₂—R²⁴; unsubstituted or substituted alkyl, alkenyl or alkynyl; unsubstituted or substituted heteroalkyl, heteroalkenyl or heteroalkynyl; unsubstituted or substituted cycloalkyl or cycloalkenyl; unsubstituted or substituted heterocycloalkyl or heterocycloalkenyl; unsubstituted or substituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or substituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; or unsubstituted or substituted -(heteroalkylene)-heterocycloalkyl, -(heteroalkenylene)-heterocycloalkyl, -(heteroalkylene)-heterocycloalkenyl or -(heteroalkenylene)-heterocycloalkenyl; or aryl;
R⁵ denotes H; —C(═O)—O—R¹²; —C(═O)—NH₂; —C(═O)—NH—R¹⁷; —C(═O)—NR¹⁸R¹⁹; —S(═O)—R²⁰; —S(═O)₂—R²¹; unsubstituted or substituted alkyl, alkenyl or alkynyl; unsubstituted or substituted heteroalkyl, heteroalkenyl or heteroalkynyl; unsubstituted or substituted cycloalkyl or cycloalkenyl; unsubstituted or substituted heterocycloalkyl or heterocycloalkenyl; unsubstituted or substituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or substituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; unsubstituted or substituted -(heteroalkylene)-heterocycloalkyl, -(heteroalkenylene)-heterocycloalkyl, -(heteroalkylene)-heterocycloalkenyl or -(heteroalkenylene)-heterocycloalkenyl; unsubstituted or substituted aryl; unsubstituted or substituted heteroaryl; unsubstituted or substituted -(alkylene)-aryl, whereby aryl can be unsubstituted or substituted with 1, 2, 3, 4 or 5 substituents mutually independently selected from the group consisting of F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, —S(═O)₂—C₁₋₅-alkyl, —S(═O)—C₁₋₅-alkyl, —NH—C₁₋₅-alkyl, N(C₁₋₅alkyl)₂, —C(═O)—O—C₁₋₅-alkyl, —C(═O)—O-phenyl, —C(═O)—H, —C(═O)—C₁₋₅-alkyl, —CH₂—O—C(═O)-phenyl, —O—C(═O)-phenyl, —O—C(═O)—C₁₋₅-alkyl, —NH—C(═O)—C₁₋₅-alkyl, —C(═O)—NH₂, —C(═O)—NH—C₁₋₅-alkyl, —C(═O)—N(C₁₋₅-alkyl)₂, —C(═O)—N(C₁₋₅-alkyl)(phenyl), —C(═O)—NH-phenyl, cyclopropyl, cyclobutyl, cyclopentyl, cyclohexyl, pyrazolyl, phenyl, furyl (furanyl), thiazolyl, thiadiazolyl, thiophenyl (thienyl), benzyl and phenethyl; -(alkenylene)-aryl, -(alkynylene)-aryl, -(heteroalkylene)-aryl or -(heteroalkenylene)-aryl; or unsubstituted or substituted -(alkylene)-heteroaryl, -(alkenylene)-heteroaryl, -(alkynylene)-heteroaryl, -(heteroalkylene)-heteroaryl or -(heteroalkenylene)-heteroaryl;
and, provided that M² denotes unsubstituted or substituted heteroaryl or unsubstituted or substituted naphthyl or an unsubstituted or substituted phenyl residue, which can be condensed (annelated) with unsubstituted or substituted 5- to 7-membered heterocycloalkyl or with unsubstituted or substituted C₅₋₇-cycloalkyl, R⁵ additionally can denote —C(═O)—R¹¹;
and R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³ and R²⁴, mutually independently, in each case denote unsubstituted or substituted alkyl, alkenyl or alkynyl; unsubstituted or substituted heteroalkyl, heteroalkenyl or heteroalkynyl; unsubstituted or substituted cycloalkyl or cycloalkenyl; unsubstituted or substituted heterocycloalkyl or heterocycloalkenyl; unsubstituted or substituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or substituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; unsubstituted or substituted -(heteroalkylene)-heterocycloalkyl, -(heteroalkenylene)-heterocycloalkyl, -(heteroalkylene)-heterocycloalkenyl; or -(heteroalkenylene)-heterocycloalkenyl; unsubstituted or substituted aryl; unsubstituted or substituted heteroaryl; unsubstituted or substituted -(alkylene)-aryl, -(alkenylene)-aryl, -(alkynylene)-aryl, -(heteroalkylene)-aryl or -(heteroalkenylene)-aryl; or unsubstituted or substituted -(alkylene)-heteroaryl, -(alkenylene)-heteroaryl, -(alkynylene)-heteroaryl, -(heteroalkylene)-heteroaryl or -(heteroalkenylene)-heteroaryl;
in each case optionally in the form of one of the pure stereoisomers thereof, the racemates thereof or in the form of a mixture of stereoisomers in any desired mixing ratio, or in each case in the form of a corresponding salts or in each case in the form of a corresponding solvates.

2. A compound according to claim 1, wherein
I.)
M¹ denotes phenyl, which can be unsubstituted or substituted with 1, 2, 3, 4 or 5 substituents mutually independently selected from the group consisting of F, Cl, Br, I, —CN, —NO₂, —OH, —SH, —NH₂, —C(═O)—OH, methyl, ethyl, n-propyl, isopropyl, n-butyl, isobutyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, —S(═O)₂—C₁₋₅-alkyl, —S(═O)—C₁₋₅-alkyl, —NH—C₁₋₅-alkyl, —N(C₁₋₅-alkyl)₂, —C(═O)—O—C₁₋₅-alkyl, —C(═O)—O-phenyl, —C(═O)—H; —C(═O)—C₁₋₅-alkyl, —CH₂—O—C(═O)-phenyl, —O—C(═O)-phenyl, —O—C(═O)—C₁₋₅-alkyl, —NH—C(═O)—C₁₋₅-alkyl, —C(═O)—NH₂, —C(═O)—NH—C₁₋₅-alkyl, —C(═O)—N(C₁₋₅-alkyl)₂, —C(═O)—N(C₁₋₅-alkyl)(phenyl), —C(═O)—NH-phenyl, cyclopropyl, cyclobutyl, cyclopentyl, cyclohexyl, pyrazolyl, phenyl, furyl (furanyl), thiazolyl, thiadiazolyl, thiophenyl (thienyl), benzyl and phenethyl, whereby the above-mentioned C₁₋₅-alkyl residues can in each case be linear or branched and the cyclic substituents or the cyclic residues of these substituents themselves can be substituted with optionally 1, 2, 3, 4 or 5 substituents mutually independently selected from the group consisting of F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂ and —S—CH₂F;
and M² denotes phenyl, which is substituted with 1, 2, 3, 4 or 5 substituents mutually independently selected from the group consisting of F, Cl, Br, I, —CN, —NO₂, —OH, —SH, —NH₂, —C(═O)—OH, methyl, ethyl, n-propyl, isopropyl, n-butyl, isobutyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, —S(═O)₂—C₁₋₅-alkyl, —S(═O)—C₁₋₅-alkyl, —NH—C₁₋₅-alkyl, —N(C₁₋₅alkyl)₂, —C(═O)—O—C₁₋₅-alkyl, —C(═O)—O-phenyl, —C(═O)—H; —C(═O)—C₁₋₅-alkyl, —CH₂—O—C(═O)-phenyl, —O—C(═O)-phenyl, —O—C(═O)—C₁₋₅-alkyl, —NH—C(═O)—C₁₋₅-alkyl, —C(—O)—NH₂, —C(═O)—NH—C₁₋₅-alkyl, —C(═O)—N(C₁₋₅-alkyl)₂, —C(═O)—N(C₁₋₅-alkyl)(phenyl), —C(═O)—NH-phenyl, cyclopropyl, cyclobutyl, cyclopentyl, cyclohexyl, pyrazolyl, phenyl, furyl (furanyl), thiazolyl, thiadiazolyl, thiophenyl (thienyl), benzyl and phenethyl, whereby the above-mentioned C₁₋₅-alkyl residues can in each case be linear or branched and the cyclic substituents or the cyclic residues of these substituents themselves can be substituted with optionally 1, 2, 3, 4 or 5 substituents mutually independently selected from the group consisting of F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂ and —S—CH₂F;
or M² denotes unsubstituted or substituted heteroaryl or unsubstituted or substituted naphthyl or denotes an unsubstituted or substituted phenyl residue, which can be condensed (annelated) with unsubstituted or substituted 5- to 7-membered heterocycloalkyl or with unsubstituted or substituted C₅₋₇-cycloalkyl;
or II.)
M¹ denotes unsubstituted or substituted heteroaryl or unsubstituted or substituted naphthyl or an unsubstituted or substituted phenyl residue, which can be condensed (annelated) with unsubstituted or substituted 5- to 7-membered heterocycloalkyl or with unsubstituted or substituted C₅₋₇-cycloalkyl;
and M² denotes unsubstituted or substituted heteroaryl or unsubstituted or substituted naphthyl or an unsubstituted or substituted phenyl residue, which can be condensed (annelated) with unsubstituted or substituted 5- to 7-membered heterocycloalkyl or with unsubstituted or substituted C₅₋₇-cycloalkyl;
or M² denotes phenyl, which can be unsubstituted or substituted with 1, 2, 3, 4 or 5 substituents mutually independently selected from the group consisting of F, Cl, Br, I, —CN, —NO₂, —OH, —SH, —NH₂, —C(═O)—OH, methyl, ethyl, n-propyl, isopropyl, n-butyl, isobutyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, —S(═O)₂—C₁₋₅-alkyl, —S(═O)—C₁₋₅-alkyl, —NH—C₁₋₅-alkyl, —N(C₁₋₅alkyl)₂, —C(═O)—O—C₁₋₅-alkyl, —C(═O)—O-phenyl, —C(═O)—H; —C(—O)—C₁₋₅-alkyl, —CH₂—O—C(═O)-phenyl, —O—C(═O)-phenyl, —O—C(═O)—C₁₋₅-alkyl, —NH—C(═O)—C₁₋₅-alkyl, —C(═O)—NH₂, —C(═O)—NH—C₁₋₅-alkyl, —C(═O)—N(C₁₋₅-alkyl)₂, —C(═O)—N(C₁₋₅-alkyl)(phenyl), —C(═O)—NH-phenyl, cyclopropyl, cyclobutyl, cyclopentyl, cyclohexyl, pyrazolyl, phenyl, furyl (furanyl), thiazolyl, thiadiazolyl, thiophenyl (thienyl), benzyl and phenethyl, whereby the above-mentioned C₁₋₅-alkyl residues can in each case be linear or branched and the cyclic substituents or the cyclic residues of these substituents themselves can be substituted with optionally 1, 2, 3, 4 or 5 substituents mutually independently selected from the group consisting of F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂ and —S—CH₂F;
and in each case
R¹ and R², mutually independently, in each case denote H; F; Cl; Br; I; —NO₂; —CN; —NH₂; —OH; —SH; —O—R⁶; —S—R⁷; —NH—R⁸; —NR⁹R¹⁰; unsubstituted or substituted alkyl, alkenyl or alkynyl; unsubstituted or substituted heteroalkyl, heteroalkenyl or heteroalkynyl; unsubstituted or substituted cycloalkyl or cycloalkenyl; unsubstituted or substituted heterocycloalkyl or heterocycloalkenyl; unsubstituted or substituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or substituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; or unsubstituted or substituted -(heteroalkylene)-heterocycloalkyl, -(heteroalkenylene)-heterocycloalkyl, -(heteroalkylene)-heterocycloalkenyl or -(heteroalkenylene)-heterocycloalkenyl; or aryl;
or R¹ and R² jointly denote an oxo group (═O);
R³ and R⁴, mutually independently, in each case denote H; F; Cl; Br; I; —NO₂; —CN; —NH₂; —OH; —SH; —C(═O)—OH; —C(═O)—H; —NH—C(═O)—H; —O—R⁶; —S—R⁷; —NH—R⁸; —NR⁹R¹⁰; —C(═O)—R¹¹; —O—C(═O)—R¹³; —NH—C(═O)—R¹⁴; —NR¹⁵—C(═O)—R¹⁶; —C(═O)—NH₂; —C(═O)—NH—R¹⁷; —C(═O)—NR¹⁸R¹⁹; —S(═O)—R²⁰; —S(═O)₂—R²¹; —NH—S(═O)₂—R²²; —NR²³—S(═O)₂—R²⁴; unsubstituted or substituted alkyl, alkenyl or alkynyl; unsubstituted or substituted heteroalkyl, heteroalkenyl or heteroalkynyl; unsubstituted or substituted cycloalkyl or cycloalkenyl; unsubstituted or substituted heterocycloalkyl or heterocycloalkenyl; unsubstituted or substituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or substituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; or unsubstituted or substituted -(heteroalkylene)-heterocycloalkyl, -(heteroalkenylene)-heterocycloalkyl, -(heteroalkylene)-heterocycloalkenyl or -(heteroalkenylene)-heterocycloalkenyl; or aryl;
R⁵ denotes H; —C(═O)—O—R¹²; —C(═O)—NH₂; —C(═O)—NH—R¹⁷; —C(═O)—NR¹⁸R¹⁹; —S(═O)—R²⁰; —S(═O)₂—R²¹; unsubstituted or substituted alkyl, alkenyl or alkynyl; unsubstituted or substituted heteroalkyl, heteroalkenyl or heteroalkynyl; unsubstituted or substituted cycloalkyl or cycloalkenyl; unsubstituted or substituted heterocycloalkyl or heterocycloalkenyl; unsubstituted or substituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or substituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; unsubstituted or substituted -(heteroalkylene)-heterocycloalkyl, -(heteroalkenylene)-heterocycloalkyl, -(heteroalkylene)-heterocycloalkenyl or -(heteroalkenylene)-heterocycloalkenyl; unsubstituted or substituted aryl; unsubstituted or substituted heteroaryl; unsubstituted or substituted -(alkylene)-aryl, whereby aryl can be unsubstituted or substituted with 1, 2, 3, 4 or 5 substituents mutually independently selected from the group consisting of F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, —S(═O)₂—C₁₋₅-alkyl, —S(═O)—C₁₋₅-alkyl, —NH—C₁₋₅-alkyl, N(C₁₋₅alkyl)₂, —C(═O)—O—C₁₋₅-alkyl, —C(═O)—O-phenyl, —C(═O)—H, —C(═O)—C₁₋₅-alkyl, —CH₂—O—C(═O)-phenyl, —O—C(═O)-phenyl, —O—C(═O)—C₁₋₅-alkyl, —NH—C(═O)—C₁₋₅-alkyl, —C(═O)—NH₂, —C(═O)—NH—C₁₋₅-alkyl, —C(═O)—N(C₁₋₅-alkyl)₂, —C(═O)—N(C₁₋₅-alkyl)(phenyl), —C(═O)—NH-phenyl, cyclopropyl, cyclobutyl, cyclopentyl, cyclohexyl, pyrazolyl, phenyl, furyl (furanyl), thiazolyl, thiadiazolyl, thiophenyl (thienyl), benzyl and phenethyl; -(alkenylene)-aryl, -(alkynylene)-aryl, -(heteroalkylene)-aryl or -(heteroalkenylene)-aryl; or unsubstituted or substituted -(alkylene)-heteroaryl, -(alkenylene)-heteroaryl, -(alkynylene)-heteroaryl, -(heteroalkylene)-heteroaryl or -(heteroalkenylene)-heteroaryl;
and, provided that M² denotes unsubstituted or substituted heteroaryl or unsubstituted or substituted naphthyl or an unsubstituted or substituted phenyl residue, which can be condensed (annelated) with unsubstituted or substituted 5- to 7-membered heterocycloalkyl or with unsubstituted or substituted C₅₋₇-cycloalkyl, R⁵ additionally can denote —C(═O)—R¹¹;
and R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³ and R²⁴, mutually independently, in each case denote unsubstituted or substituted alkyl, alkenyl or alkynyl; unsubstituted or substituted heteroalkyl, heteroalkenyl or heteroalkynyl; unsubstituted or substituted cycloalkyl or cycloalkenyl; unsubstituted or substituted heterocycloalkyl or heterocycloalkenyl; unsubstituted or substituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or substituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; unsubstituted or substituted -(heteroalkylene)-heterocycloalkyl, -(heteroalkenylene)-heterocycloalkyl, -(heteroalkylene)-heterocycloalkenyl; or -(heteroalkenylene)-heterocycloalkenyl; unsubstituted or substituted aryl; unsubstituted or substituted heteroaryl; unsubstituted or substituted -(alkylene)-aryl, -(alkenylene)-aryl, -(alkynylene)-aryl, -(heteroalkylene)-aryl or -(heteroalkenylene)-aryl; or unsubstituted or substituted -(alkylene)-heteroaryl, -(alkenylene)-heteroaryl, -(alkynylene)-heteroaryl, -(heteroalkylene)-heteroaryl or -(heteroalkenylene)-heteroaryl;
whereby
the above-mentioned alkyl residues are in each case branched or straight-chained and have 1, 2, 3, 4, 5, 6, 7, 8, 9, 10, 11 or 12 carbon atoms as chain members;
the above-mentioned alkenyl residues are in each case branched or straight-chained and have 2, 3, 4, 5, 6, 7, 8, 9, 10, 11 or 12 carbon atoms as chain members;
the above-mentioned alkynyl residues are in each case branched or straight-chained and have 2, 3, 4, 5, 6, 7, 8, 9, 10, 11 or 12 carbon atoms as chain members;
the above-mentioned heteroalkyl residues, heteroalkenyl residues and heteroalkynyl residues are in each case 2-, 3-, 4-, 5-, 6-, 7-, 8-, 9-, 10-, 11- or 12-membered;
the above-mentioned heteroalkyl residues, heteroalkenyl residues and heteroalkynyl residues in each case have optionally 1, 2 or 3 heteroatom(s), mutually independently, selected from the group consisting of oxygen, sulphur and nitrogen as the chain member(s);
the above-mentioned alkyl residues, alkenyl residues, alkynyl residues, heteroalkyl residues, heteroalkenyl residues and heteroalkynyl residues can in each case be substituted with optionally 1, 2, 3, 4 or 5 substituents mutually independently selected from the group consisting of F, Cl, Br, I, —NO₂, —CN, —OH, —SH, —NH₂, —N(C₁₋₅-alkyl)₂, —N(C₁₋₅-alkyl)(phenyl), —N(C₁₋₅-alkyl)(CH₂-phenyl), —N(C₁₋₅-alkyl)(CH₂—CH₂-phenyl), —C(═O)—H, —C(═O)—C₁₋₅-alkyl, —C(═O)-phenyl, —C(═S)—C₁₋₅-alkyl, —C(═S)-phenyl, —C(═O)—O—C₁₋₅-alkyl, —C(═O)—O-phenyl, —C(═O)—NH₂, —C(═O)—NH—C₁₋₅-alkyl, —C(═O)—N(C₁₋₅-alkyl)₂, —S(═O)—C₁₋₅-alkyl, —S(═O)-phenyl, —S(═O)₂—C₁₋₅-alkyl, —S(═O)₂-phenyl, —S(═O)₂—NH₂ and —SO₃H, whereby the phenyl residues can be substituted with 1, 2, 3, 4 or 5 substituents mutually independently selected from the group consisting of F, Cl, Br, I, —CN, —CF₃, —OH, —NH₂, —O—CF₃, —SH, —O—CH₃, —O—C₂H₅, —O—C₃H₇, methyl, ethyl, n-propyl, isopropyl, n-butyl, 2-butyl, isobutyl and tert-butyl;
unless indicated otherwise, the above-mentioned cycloalkyl residues in each case have 3, 4, 5, 6, 7, 8 or 9 carbon atoms as ring members;
the above-mentioned cycloalkenyl residues in each case have 3, 4, 5, 6, 7, 8 or 9 carbon atoms as ring members;
unless indicated otherwise, the above-mentioned heterocycloalkyl residues are in each case 3-, 4-, 5-, 6-, 7-, 8- or 9-membered;
the above-mentioned heterocycloalkenyl residues are in each case 4-, 5-, 6-, 7-, 8- or 9-membered;
the above-mentioned heterocycloalkyl residues and heterocycloalkenyl residues in each case have optionally 1, 2 or 3 heteroatom(s), mutually independently, selected from the group consisting of oxygen, sulphur and nitrogen (NH) as the ring member(s);
the above-mentioned cycloalkyl residues, heterocycloalkyl residues, cycloalkenyl residues or heterocycloalkenyl residues can in each case be substituted with optionally 1, 2, 3, 4 or 5 substituents mutually independently selected from the group consisting of F, Cl, Br, I, —CN, —CF₃, —OH, —NH₂, —O—CF₃, —SH, —O—C₁₋₅-alkyl, —O-phenyl, —O—CH₂-phenyl, —(CH₂)—O—C₁₋₅-alkyl, —S—C₁₋₅-alkyl, —S-phenyl, —S—CH₂-phenyl, —C₁₋₅-alkyl, —C₂₋₅-alkenyl, C₂₋₅-alkynyl, —C≡C—Si(CH₃)₃, —C≡C—Si(C₂H₅)₃, —C(═O)—O—C₁₋₅-alkyl, —C(═O)—CF₃, —S(═O)₂—C₁₋₅-alkyl, —S(═O)—C₁₋₅-alkyl, —S(═O)₂-phenyl, oxo (═O), thioxo (═S), —N(C₁₋₅-alkyl)₂, —N(H)(C₁₋₅-alkyl), —NO₂, —S—CF₃, —C(═O)—OH, —NH—C(═O)—C₁₋₅-alkyl, —C(═O)—H, —C(═O)—C₁₋₅-alkyl, —C(═O)—NH₂, —C(═O)—N(C₁₋₅-alkyl)₂, —C(═O)—N(H)(C₁₋₅-alkyl) and phenyl, whereby the phenyl residues can respectively be unsubstituted or substituted with 1, 2, 3, 4 or 5 substituents mutually independently selected from the group consisting of F, Cl, Br, I, —CN, —CF₃, —OH, —NH₂, —O—CF₃, —SH, —O—C₁₋₅-alkyl, —O-phenyl, —O—CH₂-phenyl, —(CH₂)—O—C₁₋₅-alkyl, —S—C₁₋₅-alkyl, —S-phenyl, —S—CH₂-phenyl, —C₁₋₅-alkyl, —C₂₋₅-alkenyl, —C₂₋₅-alkynyl, —C≡C—Si(CH₃)₃, —C≡C—Si(C₂H₅)₃, —C(═O)—O—C₁₋₅-alkyl and —C(═O)—CF₃, whereby the above-mentioned phenyl residues can be substituted with 1, 2, 3, 4 or 5 substituents mutually independently selected from the group consisting of F, Cl, Br, I, —CN, —CF₃, —OH, —NH₂, —O—CF₃, —SH, —O—CH₃, —O—C₂H₅, —O—C₃H₇, methyl, ethyl, n-propyl, isopropyl, n-butyl, 2-butyl, isobutyl and tert-butyl;
the above-mentioned alkylene residues are in each case branched or straight-chained and have 1, 2, 3, 4, 5, 6, 7, 8, 9, 10, 11 or 12 carbon atoms as chain members;
the above-mentioned alkenylene residues are in each case branched or straight-chained and have 2, 3, 4, 5, 6, 7, 8, 9, 10, 11 or 12 carbon atoms as chain members;
the above-mentioned alkynylene residues are in each case branched or straight-chained and have 2, 3, 4, 5, 6, 7, 8, 9, 10, 11 or 12 carbon atoms as chain members;
the above-mentioned heteroalkylene residues and heteroalkenylene residues are in each case 2-, 3-, 4-, 5-, 6-, 7-, 8-, 9-, 10-, 11- or 12-membered;
the above-mentioned heteroalkylene and heteroalkenylene groups have in each case optionally 1, 2 or 3 heteroatom(s), mutually independently, selected from the group consisting of oxygen, sulphur and nitrogen (NH) as the chain member(s);
the above-mentioned alkylene, alkenylene, alkynylene, heteroalkylene or heteroalkenylene group can in each case be unsubstituted or substituted with optionally 1, 2, 3, 4 or 5 substituents mutually independently selected from the group consisting of phenyl, F, Cl, Br, I, —NO₂, —CN, —OH, —O-phenyl, —O—CH₂-phenyl, —SH, —S-phenyl, —S—CH₂-phenyl, NH₂, —N(C₁₋₅-alkyl)₂, —NH-phenyl, —N(C₁₋₅-alkyl)(phenyl), —N(C₁₋₅-alkyl)(CH₂-phenyl), —N(C₁₋₅-alkyl)(CH₂—CH₂-phenyl), —C(═O)—H, —C(═O)—C₁₋₅-alkyl, —C(═O)-phenyl, —C(═S)—C₁₋₅-alkyl, —C(═S)-phenyl, —C(═O)—OH, —C(═O)—O—C₁₋₅-alkyl, —C(═O)—O-phenyl, —C(═O)—NH₂, —C(═O)—NH—C₁₋₅-alkyl, —C(═O)—N(C₁₋₅-alkyl)₂, —S(═O)—C₁₋₅-alkyl, —S(═O)-phenyl, —S(═O)₂—C₁₋₅-alkyl, —S(═O)₂-phenyl, —S(═O)₂—NH₂ and —SO₃H, whereby the phenyl residues can be substituted with 1, 2, 3, 4 or 5 substituents mutually independently selected from the group consisting of F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂ and —S—CH₂F;
the above-mentioned aryl residues are mono- or bicyclic and have 6, 10 or 14 carbon atoms;
the above-mentioned heteroaryl residues are mono-, bi- or tricyclic and 5-, 6-, 7-, 8-, 9-, 10-, 11-, 12-, 13- or 14-membered;
the above-mentioned 5- to 14-membered heteroaryl residues have optionally 1, 2, 3, 4 or 5 heteroatom(s), mutually independently, selected from the group consisting of oxygen, sulphur and nitrogen (NH) as the ring member(s);
and, unless indicated otherwise, the above-mentioned naphthyl residues, aryl residues and heteroaryl residues can in each case be substituted with optionally 1, 2, 3, 4 or 5 substituents mutually independently selected from the group consisting of F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, —S(═O)₂—C₁₋₅ alkyl, —S(═O)—C₁₋₅-alkyl, —NH—C₁₋₅-alkyl, N(C₁₋₅-alkyl)₂, —C(═O)—O—C₁₋₅-alkyl, —C(═O)—O-phenyl, —C(═O)—H, —C(═O)—C₁₋₅-alkyl, —CH₂—O—C(═O)-phenyl, —O—C(═O)-phenyl, —O—C(═O)—C₁₋₅-alkyl, —NH—C(═O)—C₁₋₅-alkyl, —C(═O)—NH₂, —C(═O)—NH—C₁₋₅-alkyl, —C(═O)—N(C₁₋₅-alkyl)₂, —C(═O)—N(C₁₋₅-alkyl)(phenyl), —C(═O)—NH-phenyl, cyclopropyl, cyclobutyl, cyclopentyl, cyclohexyl, pyrazolyl, phenyl, furyl (furanyl), thiazolyl, thiadiazolyl, thiophenyl (thienyl), benzyl and phenethyl, whereby the cyclic substituents or the cyclic residues of these substituents themselves can be substituted with optionally 1, 2, 3, 4 or 5 substituents mutually independently selected from the group consisting of F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂ and —S—CH₂F;
in each case optionally in the form of one of the pure stereoisomers thereof, the racemates thereof or in the form of a mixture of stereoisomers in any desired mixing ratio, or in each case in the form of a corresponding salts or in each case in the form of a corresponding solvates.

3. A compound according to claim 1, wherein
M¹ denotes a phenyl residue, which is unsubstituted or substituted with 1, 2, 3, 4 or 5 substituents mutually independently selected from the group consisting of F, Cl, Br, I, —CN, —NO₂, methyl, ethyl, n-propyl, isopropyl, ethenyl, allyl, ethynyl, propynyl, —C≡C—Si(CH₃)₃, —C≡C—Si(C₂H₅)₃, —CH₂—O—CH₃, —CH₂—O—C₂H₅, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, pyrazolyl, phenyl, —N(CH₃)₂, —N(C₂H₅)₂, —NH—CH₃, —NH—C₂H₅, —CH₂—O—C(═O)-phenyl, —C(═O)—O—CH₃, —C(═O)—O—C₂H₅, —C(═O)—O—CH(CH₃)₂, —C(═O)—O—(CH₂)₃—CH₃, —C(═O)—O—C(CH₃)₃, —C(═O)—O-phenyl, —C(═O)—H, —C(═O)—CH₃, —C(═O)—C₂H₅, —NH—C(═O)—CH₃, —NH—C(═O)—C₂H₅, —O—C(═O)-phenyl, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅, —C(═O)—NH—C(CH₃)₃, —C(═O)—N(C₂H₅)₂, —C(═O)—NH-phenyl, —C(═O)—N(CH₃)-phenyl, —C(═O)—N(C₂H₅)-phenyl, cyclopropyl, cyclobutyl, cyclopentyl, cyclohexyl, phenyl, furyl (furanyl), thiadiazolyl, thiophenyl (thienyl) and benzyl;
and M² denotes a phenyl residue, which is substituted with 1, 2, 3, 4 or 5 substituents mutually independently selected from the group consisting of F, Cl, Br, I, —CN, —NO₂, methyl, ethyl, n-propyl, isopropyl, n-butyl, tert-butyl, n-pentyl, ethenyl, allyl, ethynyl, propynyl, —C≡C—Si(CH₃)₃, —C≡C—Si(C₂H₅)₃, —CH₂—O—CH₃, —CH₂—O—C₂H₅, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, pyrazolyl, phenyl, —N(CH₃)₂, —N(C₂H₅)₂, —NH—CH₃, —NH—C₂H₅, —CH₂—O—C(═O)-phenyl, —C(═O)—O—CH₃, —C(═O)—O—C₂H₅, —C(═O)—O—CH(CH₃)₂, —C(═O)—O—(CH₂)₃—CH₃, —C(═O)—O—C(CH₃)₃, —C(═O)—O-phenyl, —C(═O)—H, —C(═O)—CH₃, —C(═O)—C₂H₅, —NH—C(═O)—CH₃, —NH—C(═O)—C₂H₅, —O—C(═O)-phenyl, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅, —C(═O)—NH—C(CH₃)₃, —C(═O)—N(C₂H₅)₂, —C(═O)—NH-phenyl, —C(═O)—N(CH₃)-phenyl, —C(═O)—N(C₂H₅)-phenyl, cyclopropyl, cyclobutyl, cyclopentyl, cyclohexyl, phenyl, furyl (furanyl), thiadiazolyl, thiophenyl (thienyl) and benzyl;
or M² denotes a residue selected from the group c of naphthyl, thiophenyl (thienyl), furanyl (furyl), pyrrolyl, pyrazolyl, pyranyl, triazolyl, pyridinyl, imidazolyl, indolyl, isoindolyl, indolizinyl, benzimidazolyl, benzo[b]furanyl, benzo[b]thiophenyl, benzo[d]thiazolyl, benzodiazolyl, benzotriazolyl, benzoxazolyl, benzisoxazolyl, thiazolyl, thiadiazolyl, oxazolyl, oxadiazolyl, tetrazolyl, triazinyl, isoxazolyl, pyridazinyl, pyrazinyl, pyrimidinyl, indazolyl, phthalazinyl, carbazolyl, carbolinyl, diaza-naphthyl, quinoxalinyl, quinazolinyl, quinolinyl, naphthridinyl, isoquinolinyl, indolinyl, (2,3)-dihydrobenzofuranyl, (2,3)-dihydrobenzo[d]oxazolyl, benzo[d][1,3]dioxolyl, isoindolinyl, (1,2,3,4)-tetrahydroquinolinyl and (2,3)-dihydrobenzo[b][1,4]dioxinyl which is unsubstituted or substituted with 1, 2, 3, 4 or 5 substituents mutually independently selected from the group consisting of F, Cl, Br, I, —CN, —NO₂, methyl, ethyl, n-propyl, isopropyl, n-butyl, tert-butyl, n-pentyl, ethenyl, allyl, ethynyl, propynyl, —C≡C—Si(CH₃)₃, —C≡C—Si(C₂H₅)₃, —CH₂—O—CH₃, —CH₂—O—C₂H₅, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, pyrazolyl, phenyl, —N(CH₃)₂, —N(C₂H₅)₂, —NH—CH₃, —NH—C₂H₅, —CH₂—O—C(═O)-phenyl, —C(═O)—O—CH₃, —C(═O)—O—C₂H₅, —C(═O)—O—CH(CH₃)₂, —C(═O)—O—(CH₂)₃—CH₃, —C(═O)—O—C(CH₃)₃, —C(═O)—O-phenyl, —C(═O)—H, —C(═O)—CH₃, —C(═O)—C₂H₅, —NH—C(═O)—CH₃, —NH—C(═O)—C₂H₅, —O—C(═O)-phenyl, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅, —C(═O)—NH—C(CH₃)₃, —C(═O)—N(C₂H₅)₂, —C(═O)—NH-phenyl, —C(═O)—N(CH₃)-phenyl, —C(═O)—N(C₂H₅)-phenyl, cyclopropyl, cyclobutyl, cyclopentyl, cyclohexyl, phenyl, furyl (furanyl), thiadiazolyl, thiophenyl (thienyl) and benzyl.

4. A compound according to claim 1, wherein
M¹ denotes a residue selected from the group consisting of naphthyl, thiophenyl (thienyl), furanyl (furyl), pyrrolyl, pyrazolyl, pyranyl, triazolyl, pyridinyl, imidazolyl, indolyl, isoindolyl, indolizinyl, benzimidazolyl, benzo[b]furanyl, benzo[b]thiophenyl, benzo[d]thiazolyl, benzodiazolyl, benzotriazolyl, benzoxazolyl, benzisoxazolyl, thiazolyl, thiadiazolyl, oxazolyl, oxadiazolyl, tetrazolyl, triazinyl, isoxazolyl, pyridazinyl, pyrazinyl, pyrimidinyl, indazolyl, phthalazinyl, carbazolyl, carbolinyl, diaza-naphthyl, quinoxalinyl, quinazolinyl, quinolinyl, naphthridinyl, isoquinolinyl, indolinyl, (2,3)-dihydrobenzofuranyl, (2,3)-dihydrobenzo[d]oxazolyl, benzo[d][1,3]dioxolyl, isoindolinyl, (1,2,3,4)-tetrahydroquinolinyl and (2,3)-dihydrobenzo[b][1,4]dioxinyl, which is unsubstituted or substituted with 1, 2, 3, 4 or 5 substituents mutually independently selected from the group consisting of F. Cl, Br, I, —CN, —NO₂, methyl, ethyl, n-propyl, isopropyl, n-butyl, tert-butyl, n-pentyl, ethenyl, allyl, ethynyl, propynyl, —C≡C—Si(CH₃)₃, C≡C—Si(C₂H₅)₃, —CH₂—O—CH₃, —CH₂—O—C₂H₅, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, pyrazolyl, phenyl, —N(CH₃)₂, —N(C₂H₅)₂, —NH—CH₃, —NH—C₂H₅, —CH₂—O—C(═O)-phenyl, —C(═O)—O—CH₃, —C(═O)—O—C₂H₅, —C(═O)—O—CH(CH₃)₂, —C(═O)—O—(CH₂)₃—CH₃, —C(═O)—O—C(CH₃)₃, —C(═O)—O-phenyl, —C(═O)—H, —C(═O)—CH₃, —C(═O)—C₂H₅, —NH—C(═O)—CH₃, —NH—C(═O)—C₂H₅, —O—C(═O)-phenyl, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅, —C(═O)—NH—C(CH₃)₃, —C(═O)—N(C₂H₅)₂, —C(═O)—NH-phenyl, —C(═O)—N(CH₃)-phenyl, —C(═O)—N(C₂H₅)-phenyl, cyclopropyl, cyclobutyl, cyclopentyl, cyclohexyl, phenyl, furyl (furanyl), thiadiazolyl, thiophenyl (thienyl) and benzyl,
and M² denotes a residue selected from the group consisting of naphthyl, thiophenyl (thienyl), furanyl (furyl), pyrrolyl, pyrazolyl, pyranyl, triazolyl, pyridinyl, imidazolyl, indolyl, isoindolyl, indolizinyl, benzimidazolyl, benzo[b]furanyl, benzo[b]thiophenyl, benzo[d]thiazolyl, benzodiazolyl, benzotriazolyl, benzoxazolyl, benzisoxazolyl, thiazolyl, thiadiazolyl, oxazolyl, oxadiazolyl, tetrazolyl, triazinyl, isoxazolyl, pyridazinyl, pyrazinyl, pyrimidinyl, indazolyl, phthalazinyl, carbazolyl, carbolinyl, diaza-naphthyl, quinoxalinyl, quinazolinyl, quinolinyl, naphthridinyl, isoquinolinyl, indolinyl, (2,3)-dihydrobenzofuranyl, (2,3)-dihydrobenzo[d]oxazolyl, benzo[d][1,3]dioxolyl, isoindolinyl, (1,2,3,4)-tetrahydroquinolinyl and (2,3)-dihydrobenzo[b][1,4]dioxinyl which is unsubstituted or substituted with 1, 2, 3, 4 or 5 substituents mutually independently selected from the group consisting of F, Cl, Br, I, —CN, —NO₂, methyl, ethyl, n-propyl, isopropyl, n-butyl, tert-butyl, n-pentyl, ethenyl, allyl, ethynyl, propynyl, —C≡C—Si(CH₃)₃, —C≡C—Si(C₂H₅)₃, —CH₂—O—CH₃, —CH₂—O—C₂H₅, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, pyrazolyl, phenyl, —N(CH₃)₂, —N(C₂H₅)₂, —NH—CH₃, —NH—C₂H₅, —CH₂—O—C(═O)-phenyl, —C(═O)—O—CH₃, —C(═O)—O—C₂H₅, —C(═O)—O—CH(CH₃)₂, —C(═O)—O—(CH₂)₃—CH₃, —C(═O)—O—C(CH₃)₃, —C(═O)—O-phenyl, —C(═O)—H, —C(═O)—CH₃, —C(═O)—C₂H₅, —NH—C(═O)—CH₃, —NH—C(═O)—C₂H₅, —O—C(═O)-phenyl, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅, —C(═O)—NH—C(CH₃)₃, —C(═O)—N(C₂H₅)₂, —C(═O)—NH-phenyl, —C(═O)—N(CH₃)-phenyl, —C(═O)—N(C₂H₅)-phenyl, cyclopropyl, cyclobutyl, cyclopentyl, cyclohexyl, phenyl, furyl (furanyl), thiadiazolyl, thiophenyl (thienyl) and benzyl;
or M² denotes a phenyl residue which is unsubstituted or substituted with 1, 2, 3, 4 or 5 substituents mutually independently selected from the group consisting of F, Cl, Br, I, —CN, —NO₂, methyl, ethyl, n-propyl, isopropyl, n-butyl, tert-butyl, n-pentyl, ethenyl, allyl, ethynyl, propynyl, —C≡C—Si(CH₃)₃, —C≡C—Si(C₂H₅)₃, —CH₂—O—CH₃, —CH₂—O—C₂H₅, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, pyrazolyl, phenyl, —N(CH₃)₂, —N(C₂H₅)₂, —NH—CH₃, —NH—C₂H₅, —CH₂—O—C(═O)-phenyl, —C(═O)—O—CH₃, —C(═O)—O—C₂H₅, —C(═O)—O—CH(CH₃)₂, —C(═O)—O—(CH₂)₃—CH₃, —C(═O)—O—C(CH₃)₃, —C(═O)—O-phenyl, —C(═O)—H, —C(═O)—CH₃, —C(═O)—C₂H₅, —NH—C(═O)—CH₃, —NH—C(═O)—C₂H₅, —O—C(═O)-phenyl, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅, —C(═O)—NH—C(CH₃)₃, —C(═O)—N(C₂H₅)₂, —C(═O)—NH-phenyl, —C(═O)—N(CH₃)-phenyl, —C(═O)—N(C₂H₅)-phenyl, cyclopropyl, cyclobutyl, cyclopentyl, cyclohexyl, phenyl, furyl (furanyl), thiadiazolyl, thiophenyl (thienyl) and benzyl.

5. A compound according to one or more of claim 4, wherein
R¹ and R², mutually independently, in each case denote H; F; Cl; Br; I; —NO₂; —CN; —NH₂; —OH; —SH; —O—R⁶; —S—R⁷; —NH—R⁸; —NR⁹R¹⁰; C₁₋₆-alkyl, which is unsubstituted or substituted with optionally 1, 2, 3, 4 or 5 substituents mutually independently selected from the group o consisting of F, Cl, Br, I, —NO₂, —CN, —OH, —SH and —NH₂; or C₃₋₇-cycloalkyl, C₅₋₆-cycloalkenyl, 5- to 7-membered heterocycloalkyl or 5- to 7-membered heterocycloalkenyl, which can be bound in each case via a C₁₋₃-alkylene-, C₂₋₃-alkenylene- or C₂₋₃-alkynylene group and/or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅;
or a phenyl residue, which is unsubstituted or substituted with optionally 1, 2, 3, 4 or 5 substituents mutually independently selected from the group consisting of F, Cl, Br, I, —CN, —NO₂, methyl, ethyl, n-propyl, isopropyl, n-butyl, tert-butyl, n-pentyl, ethenyl, allyl, ethynyl, propynyl, —C≡C—Si(CH₃)₃, —C≡C—Si(C₂H₅)₃, —CH₂—O—CH₃, —CH₂—O—C₂H₅, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, pyrazolyl, phenyl, —N(CH₃)₂, —N(C₂H₅)₂, —NH—CH₃, —NH—C₂H₅, —CH₂—O—C(═O)-phenyl, —C((═O)—O—CH₃, —C(═O)—O—C₂H₅, —C(═O)—O—CH(CH₃)₂, —C(═O)—O—(CH₂)₃—CH₃, —C(═O)—O—C(CH₃)₃, —C(═O)—O-phenyl, —C(═O)—H, —C(═O)—CH₃, —C(═O)—C₂H₅, —NH—C(═O)—CH₃, —NH—C(═O)—C₂H₅, —O—C(═O)-phenyl, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅, —C(═O)—NH—C(CH₃)₃, —C(═O)—N(C₂H₅)₂, —C(═O)—NH-phenyl, —C(═O)—N(CH₃)-phenyl, —C(═O)—N(C₂H₅)-phenyl, cyclopropyl, cyclobutyl, cyclopentyl, cyclohexyl, phenyl, furyl (furanyl), thiadiazolyl, thiophenyl (thienyl) and benzyl;
or R¹ and R² jointly denote an oxo group (═O).

6. A compound according to claim 1, wherein
R³ and R⁴, mutually independently, in each case denote H; F; Cl; Br; I; —NO₂; —CN; —NH₂; —OH; —SH; —C(═O)—H; —NH—C(═O)—H; —O—R⁶; —S—R⁷; —NH—R⁸; —NR⁹R¹⁰; —C(═O)—R¹¹; —C(═O)—O—R¹²; —NH—C(═O)—R¹⁴; —NR¹⁵—C(═O)—R¹⁶; —C(═O)—NH₂; —C(═O)—NH—R¹⁷; —C(═O)—NR¹⁸R¹⁹; C₁₋₆-alkyl, which is unsubstituted or substituted with optionally 1, 2, 3, 4 or 5 substituents mutually independently selected from the group consisting of F, Cl, Br, I, —NO₂, —CN, —OH, —SH and —NH₂; or C₃₋₇-cycloalkyl, C₅₋₆-cycloalkenyl, 5- to 7-membered heterocycloalkyl or 5- to 7-membered heterocycloalkenyl, which can be bound in each case via a C₁₋₃-alkylene-, C₂₋₃-alkenylene- or C₂₋₃-alkynylene group and/or is unsubstituted or substituted with optionally 1, 2, 3, 4 or 5 substituents mutually independently selected from the group consisting, of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅, or a phenyl residue, which is unsubstituted or substituted with optionally 1, 2, 3, 4 or 5 substituents mutually independently selected from the group consisting of F, Cl, Br, I, —CN, —NO₂, methyl, ethyl, n-propyl, isopropyl, n-butyl, tert-butyl, n-pentyl, ethenyl, allyl, ethynyl, propynyl, —C≡C—Si(CH₃)₃, —C≡C—Si(C₂H₅)₃, —CH₂—O—CH₃, —CH₂—O—C₂H₅, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, pyrazolyl, phenyl, —N(CH₃)₂, —N(C₂H₅)₂, —NH—CH₃, —NH—C₂H₅, —CH₂—O—C(═O)-phenyl, —C(═O)—O—CH₃, —C(═O)—O—C₂H₅, —C(═O)—O—CH(CH₃)₂, —C(═O)—O—(CH₂)₃—CH₃, —C(═O)—O—C(CH₃)₃, —C(═O)—O-phenyl, —C(═O)—H, —C(═O)—CH₃, —C(═O)—C₂H₅, —NH—C(═O)—CH₃, —NH—C(═O)—C₂H₅, —O—C(═O)-phenyl, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅, —C(═O)—NH—C(CH₃)₃, —C(═O)—N(C₂H₅)₂, —C(═O)—NH-phenyl, —C(═O)—N(CH₃)— phenyl, —C(═O)—N(C₂H₅)-phenyl, cyclopropyl, cyclobutyl, cyclopentyl, cyclohexyl, phenyl, furyl (furanyl), thiadiazolyl, thiophenyl (thienyl) and benzyl.

7. A compound according to claim 1, wherein
R⁵ denotes H; —C(═O)—O—R¹²; —C(═O)—NH₂; —C(═O)—NH—R¹⁷; —C(═O)—NR¹⁸R¹⁹; —S(═O)—R²⁰; —S(═O)₂—R²¹; C₁₋₆-alkyl, which is unsubstituted or substituted with optionally 1, 2, 3, 4 or 5 substituents mutually independently selected from the group consisting of F, Cl, Br, I, —NO₂, —CN, —OH, —SH and —NH₂; C₃₋₇-cycloalkyl, C₅₋₆-cycloalkenyl, 5- to 7-membered heterocycloalkyl or 5- to 7-membered heterocycloalkenyl, which in each case can be bound via a C₁₋₃-alkylene-, C₂₋₃-alkenylene- or C₂₋₃-alkynylene group and/or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or a residue selected from the group consisting of phenyl, naphthyl, anthracenyl, pyrrolyl, indolyl, furanyl, benzo[b]furanyl, thiophenyl, benz[b]thiophenyl, benzo[d]thiazolyl, pyrazolyl, imidazolyl, thiazolyl, thiadiazolyl, triazolyl, oxazolyl, oxadiazolyl, isoxazolyl, pyridinyl, pyridazinyl, pyrimidinyl, pyrazinyl, pyranyl, indazolyl, quinolinyl, isoquinolinyl and quinazolinyl, which in each case can be bound via a C₁₋₃-alkylene-, C₂₋₃-alkenylene- or C₂₋₃-alkynylene group and/or is unsubstituted or substituted with optionally 1, 2, 3, 4 or 5 substituents mutually independently selected from the group consisting of F, Cl, Br, I, —CN, —NO₂, methyl, ethyl, n-propyl, isopropyl, n-butyl, tert-butyl, n-pentyl, ethenyl, allyl, ethynyl, propynyl, —C≡C—Si(CH₃)₃, —C≡C—Si(C₂H₅)₃, —CH₂—O—CH₃, —CH₂—O—C₂H₅, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, pyrazolyl, phenyl, —N(CH₃)₂, —N(C₂H₅)₂, —NH—CH₃, —NH—C₂H₅, —CH₂—O—C(═O)-phenyl, —C(═O)—O—CH₃, —C(═O)—O—C₂H₅, —C(═O)—O—CH(CH₃)₂, —C(═O)—O—(CH₂)₃—CH₃, —C(═O)—O—C(CH₃)₃, —C(═O)—O-phenyl, —C(═O)—H, —C(═O)—CH₃, —C(═O)—C₂H₅, —NH—C(═O)—CH₃, —NH—C(═O)—C₂H₅, —O—C(═O)-phenyl, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅, —C(═O)—NH—C(CH₃)₃, —C(═O)—N(C₂H₅)₂, —C(═O)—NH-phenyl, —C(═O)—N(CH₃)-phenyl, —C(═O)—N(C₂H₅)-phenyl, cyclopropyl, cyclobutyl, cyclopentyl, cyclohexyl, phenyl, furyl (furanyl), thiadiazolyl, thiophenyl (thienyl) and benzyl;
and, provided that M² denotes unsubstituted or substituted heteroaryl or unsubstituted or substituted naphthyl or an unsubstituted or substituted phenyl residue, which can be condensed (annelated) with unsubstituted or substituted 5- to 7-membered heterocycloalkyl or with unsubstituted or substituted C₅₋₇-cycloalkyl, R⁵ can additionally denote —C(═O)—R¹¹.

8. A compound according to claim 1, wherein
R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³ and R²⁴, mutually independently, in each case denote C₁₋₆-alkyl, which is unsubstituted or substituted with optionally 1, 2, 3, 4 or 5 substituents mutually independently selected from the group consisting of F, Cl, Br, I, —NO₂, —CN, —OH, —SH and —NH₂; C₃₋₇-cycloalkyl, C₅₋₆-cycloalkenyl, 5- to 7-membered heterocycloalkyl or 5- to 7-membered heterocycloalkenyl, which in each case can be bound via a C₁₋₃-alkylene-, C₂₋₃-alkenylene- or C₂₋₃-alkynylene group and/or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or a residue selected from the group consisting of phenyl, naphthyl, anthracenyl, pyrrolyl, indolyl, furanyl, benzo[b]furanyl, thiophenyl, benz[b]thiophenyl, benzo[d]thiazolyl, pyrazolyl, imidazolyl, thiazolyl, thiadiazolyl, triazolyl, oxazolyl, oxadiazolyl, isoxazolyl, pyridinyl, pyridazinyl, pyrimidinyl, pyrazinyl, pyranyl, indazolyl, quinolinyl, isoquinolinyl and quinazolinyl, which in each case can be bound via a C₁₋₃-alkylene-, C₂₋₃-alkenylene- or C₂₋₃-alkynylene group and/or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅.

9. A compound according to claim 1, wherein
I.)
M¹ denotes a phenyl residue, which is unsubstituted or substituted with 1, 2, 3, 4 or 5 substituents mutually independently selected from the group consisting f F, Cl, Br, I, —CN, —NO₂, methyl, ethyl, n-propyl, isopropyl, ethenyl, allyl, ethynyl, propynyl, —CH₂—O—CH₃, —CH₂—O—C₂H₅, —OH, —SH, —NH₂, —C(═O)—OH, —S—CH₃, —S—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂, —S—CH₂F, —N(CH₃)₂, —N(C₂H₅)₂, —NH—CH₃, —NH—C₂H₅, —C(═O)—O—CH₃, —C(═O)—O—C₂H₅, —C(═O)—O—CH(CH₃)₂, —C(═O)—O—(CH₂)₃—CH₃, —C(═O)—O—C(CH₃)₃, —C(═O)—O-phenyl, —C(═O)—H, —C(═O)—CH₃, —C(═O)—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅, —C(═O)—NH—C(CH₃)₃, —C(═O)—N(C₂H₅)₂, —C(═O)—NH-phenyl, —C(═O)—N(CH₃)-phenyl, —C(═O)—N(C₂H₅)-phenyl, cyclopropyl, cyclobutyl, cyclopentyl and cyclohexyl;
and M² denotes a phenyl residue, which is substituted with 1, 2, 3, 4 or 5 substituents mutually independently selected from the group consisting of F, Cl, Br, I, —CN, —NO₂, methyl, ethyl, n-propyl, isopropyl, n-butyl, isobutyl, tert-butyl, ethenyl, allyl, ethynyl, propynyl, —CH₂—O—CH₃, —CH₂—O—C₂H₅, —OH, —SH, —NH₂, —C(═O)—OH, —S—CH₃, —S—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂, —S—CH₂F, —N(CH₃)₂, —N(C₂H₅)₂, —NH—CH₃, —NH—C₂H₅, —C(═O)—O—CH₃, —C(═O)—O—C₂H₅, —C(═O)—O—CH(CH₃)₂, —C(═O)—O—(CH₂)₃—CH₃, —C(═O)—O—C(CH₃)₃, —C(═O)—O-phenyl, —C(═O)—H, —C(═O)—CH₃, —C(═O)—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅, —C(═O)—NH—C(CH₃)₃, —C(═O)—N(C₂H₅)₂, —C(═O)—NH-phenyl, —C(═O)—N(CH₃)-phenyl, —C(═O)—N(C₂H₅)-phenyl, cyclopropyl, cyclobutyl, cyclopentyl and cyclohexyl;
or M² denotes a residue selected from the group consisting of indolyl, naphthyl, thiophenyl (thienyl), furanyl (furyl), pyrrolyl, pyrazolyl, triazolyl, pyridinyl, imidazolyl, thiazolyl, thiadiazolyl, oxazolyl, oxadiazolyl, tetrazolyl, triazinyl, isoxazolyl, pyridazinyl, pyrazinyl, pyrimidinyl, diaza-naphthyl, quinoxalinyl, quinazolinyl, quinolinyl, naphthridinyl, isoquinolinyl, indolinyl, (2,3)-dihydrobenzofuranyl, (2,3)-dihydrobenzo[d]oxazolyl, benzo[d][1,3]dioxolyl, isoindolinyl, (1,2,3,4)-tetrahydroquinolinyl and (2,3)-dihydrobenzo[b][1,4]dioxinyl, which is unsubstituted or substituted with 1, 2, 3, 4 or 5 substituents mutually independently selected from the group consisting of F, Cl, Br, I, —CN, —NO₂, methyl, ethyl, n-propyl, isopropyl, n-butyl, isobutyl, tert-butyl, ethenyl, allyl, ethynyl, propynyl, —CH₂—O—CH₃, —CH₂—O—C₂H₅, —OH, —SH, —NH₂, —C(═O)—OH, —S—CH₃, —S—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂, —S—CH₂F, —N(CH₃)₂, —N(C₂H₅)₂, —NH—CH₃, —NH—C₂H₅, —C(═O)—O—CH₃, —C(═O)—O—C₂H₅, —C(═O)—O—CH(CH₃)₂, —C(═O)—O—(CH₂)₃—CH₃, —C(═O)—O—C(CH₃)₃, —C(═O)—O-phenyl, —C(═O)—H, —C(═O)—CH₃, —C(═O)—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅, —C(═O)—NH—C(CH₃)₃, —C(═O)—N(C₂H₅)₂, —C(═O)—NH-phenyl, —C(═O)—N(CH₃)-phenyl, —C(═O)—N(C₂H₅)-phenyl, cyclopropyl, cyclobutyl, cyclopentyl and cyclohexyl;
or II.)
M¹ denotes a residue selected from the group consisting of naphthyl, thiophenyl (thienyl), furanyl (furyl), pyrrolyl, pyrazolyl, triazolyl, pyridinyl, imidazolyl, indolyl, thiazolyl, thiadiazolyl, oxazolyl, oxadiazolyl, tetrazolyl, triazinyl, isoxazolyl, pyridazinyl, pyrazinyl, pyrimidinyl, diaza-naphthyl, quinoxalinyl, quinazolinyl, quinolinyl, naphthridinyl and isoquinolinyl, which is unsubstituted or substituted with 1, 2, 3, 4 or 5 substituents mutually independently selected from the group consisting of F, Cl, Br, I, —CN, —NO₂, methyl, ethyl, n-propyl, isopropyl, n-butyl, isobutyl, tert-butyl, ethenyl, allyl, ethynyl, propynyl, —CH₂—O—CH₃, —CH₂—O—C₂H₅, —OH, —SH, —NH₂, —C(═O)—OH, —S—CH₃, —S—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂, —S—CH₂F, —N(CH₃)₂, —N(C₂H₅)₂, —NH—CH₃, —NH—C₂H₅, —C(═O)—O—CH₃, —C(═O)—O—C₂H₅, —C(═O)—O—CH(CH₃)₂, —C(═O)—O—(CH₂)₃—CH₃, —C(═O)—O—C(CH₃)₃, —C(═O)—O-phenyl, —C(═O)—H, —C(═O)—CH₃, —C(═O)—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅, —C(═O)—NH—C(CH₃)₃, —C(═O)—N(C₂H₅)₂, —C(═O)—NH-phenyl, —C(═O)—N(CH₃)-phenyl, —C(═O)—N(C₂H₅)-phenyl, cyclopropyl, cyclobutyl, cyclopentyl and cyclohexyl;
and M² denotes a residue selected from the group consisting of indolyl, naphthyl, thiophenyl (thienyl), furanyl (furyl), pyrrolyl, pyrazolyl, triazolyl, pyridinyl, imidazolyl, thiazolyl, thiadiazolyl, oxazolyl, oxadiazolyl, tetrazolyl, triazinyl, isoxazolyl, pyridazinyl, pyrazinyl, pyrimidinyl, diaza-naphthyl, quinoxalinyl, quinazolinyl, quinolinyl, naphthridinyl, isoquinolinyl, indolinyl, (2,3)-dihydrobenzofuranyl, (2,3)-dihydrobenzo[d]oxazolyl, benzo[d][1,3]dioxolyl, isoindolinyl, (1,2,3,4)-tetrahydroquinolinyl and (2,3)-dihydrobenzo[b][1,4]dioxinyl, which is unsubstituted or substituted with 1, 2, 3, 4 or 5 substituents mutually independently selected from the group consisting of F, Cl, Br, I, —CN, —NO₂, methyl, ethyl, n-propyl, isopropyl, n-butyl, isobutyl, tert-butyl, ethenyl, allyl, ethynyl, propynyl, —CH₂—O—CH₃, —CH₂—O—C₂H₅, —OH, —SH, —NH₂, —C(═O)—OH, —S—CH₃, —S—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂, —S—CH₂F, —N(CH₃)₂, —N(C₂H₅)₂, —NH—CH₃, —NH—C₂H₅, —C(═O)—O—CH₃, —C(═O)—O—C₂H₅, —C(═O)—O—CH(CH₃)₂, —C(═O)—O—(CH₂)₃—CH₃, —C(═O)—O—C(CH₃)₃, —C(═O)—O-phenyl, —C(═O)—H, —C(═O)—CH₃, —C(═O)—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅, —C(═O)—NH—C(CH₃)₃, —C(═O)—N(C₂H₅)₂, —C(═O)—NH-phenyl, —C(═O)—N(CH₃)-phenyl, —C(═O)—N(C₂H₅)-phenyl, cyclopropyl, cyclobutyl, cyclopentyl and cyclohexyl;
or M² denotes a phenyl residue, which is unsubstituted or substituted with 1, 2, 3, 4 or 5 substituents mutually independently selected from the group consisting of F. Cl, Br, I, —CN, —NO₂, methyl, ethyl, n-propyl, isopropyl, n-butyl, isobutyl, tert-butyl, ethenyl, allyl, ethynyl, propynyl, —CH₂—O—CH₃, —CH₂—O—C₂H₅, —OH, —SH, —NH₂, —C(═O)—OH, —S—CH₃, —S—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂, —S—CH₂F, —N(CH₃)₂, —N(C₂H₅)₂, —NH—CH₃, —NH—C₂H₅, —C(═O)—O—CH₃, —C(═O)—O—C₂H₅, —C(═O)—O—CH(CH₃)₂, —C(═O)—O—(CH₂)₃—CH₃, —C(═O)—O—C(CH₃)₃, —C(═O)—O-phenyl, —C(═O)—H, —C(═O)—CH₃, —C(═O)—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅, —C(═O)—NH—C(CH₃)₃, —C(═O)—N(C₂H₅)₂, —C(═O)—NH-phenyl, —C(═O)—N(CH₃)-phenyl, —C(═O)—N(C₂H₅)-phenyl, cyclopropyl, cyclobutyl, cyclopentyl and cyclohexyl;
and in each case
R¹ and R², mutually independently, in each case denote H; F; Cl; Br; I; —NO₂; —CN; —OH; —SH; —O—R⁶; —S—R⁷; —NH—R⁸; —NR⁹R¹⁰; or C₁₋₆-alkyl, which is unsubstituted or substituted with optionally 1, 2, 3, 4 or 5 substituents mutually independently selected from the group consisting of F, Cl, Br, I, —NO₂, —CN, —OH, —SH and —NH₂; or a phenyl residue, which is unsubstituted or substituted with 1, 2, 3, 4 or 5 substituents mutually independently selected from the group consisting of 1, 2, 3, 4 or 5 substituents mutually independently selected from the group consisting of F, Cl, Br, I, —CN, —NO₂, methyl, ethyl, n-propyl, isopropyl, n-butyl, tert-butyl, n-pentyl, —OH, —SH, —NH₂, —C(═O)—OH, —O—CH₃, —O—C₂H₅, —O—C₃H₇, —O—C(CH₃)₃, —CF₃, —CHF₂, —CH₂F, —O—CF₃, —O—CHF₂, —O—CH₂F, —S—CF₃, —S—CHF₂ and —S—CH₂F;
or R¹ and R² jointly denote an oxo group (═O);
R³ and R⁴, mutually independently, in each case denote H; F; Cl; Br; I; —NO₂; —CN; —OH; —SH; —O—R⁶; —S—R⁷; —NH—R⁸; —NR⁹R¹⁰; —C(═O)—R¹¹; —C(═O)—NH₂; —C(═O)—NH—R¹⁷; —C(═O)—NR¹⁸R¹⁹; or C₁₋₆-alkyl, which is unsubstituted or substituted with optionally 1, 2, 3, 4 or 5 substituents mutually independently selected from the group consisting of F, Cl, Br, I, —NO₂, —CN, —OH, —SH and —NH₂; or a phenyl residue, which is unsubstituted or substituted with 1, 2, 3, 4 or 5 substituents mutually independently selected from the group consisting of 1, 2, 3, 4 or 5 substituents mutually independently selected from the group consisting of F, Cl, Br, I, —CN, —NO₂, methyl, ethyl, n-propyl, isopropyl, n-butyl, tert-butyl, n-pentyl, —OH, —SH, —NH₂, —C(═O)—OH, —O—CH₃, —O—C₂H₅, —O—C₃H₇, —O—C(CH₃)₃, —CF₃, —CHF₂, —CH₂F, —O—CF₃, —O—CHF₂, —O—CH₂F, —S—CF₃, —S—CHF₂ and —S—CH₂F;
R⁵ denotes H; —C(═O)—O—R¹²; —C(═O)—NH—R¹⁷; —C(═O)—NR¹⁸R¹⁹; —S(═O)—R²⁰; —S(═O)₂—R²¹; C₁₋₆-alkyl, which is unsubstituted or substituted with optionally 1, 2, 3, 4 or 5 substituents mutually independently selected from the group consisting of F, Cl, Br, I, —NO₂, —CN, —OH, —SH and —NH₂; C₃₋₇-cycloalkyl, which is unsubstituted or substituted with optionally 1, 2, 3, 4 or 5 substituents mutually independently selected from the group consisting of F, Cl, Br, I, methyl, ethyl, n-propyl, isopropyl, n-butyl, 2-butyl, isobutyl and tert-butyl; or a phenyl, benzyl or phenethyl residue, which is unsubstituted or substituted with 1, 2, 3, 4 or 5 substituents mutually independently selected from the group consisting of 1, 2, 3, 4 or 5 substituents mutually independently selected from the group consisting of F, Cl, Br, I, —CN, —NO₂, methyl, ethyl, n-propyl, isopropyl, n-butyl, tert-butyl, n-pentyl, —OH, —SH, —NH₂, —C(═O)—OH, —O—CH₃, —O—C₂H₅, —O—C₃H₇, —O—C(CH₃)₃, —CF₃, —CHF₂, —CH₂F, —O—CF₃, —O—CHF₂, —O—CH₂F, —S—CF₃, —S—CHF₂, —S—CH₂F, —N(CH₃)₂, —N(C₂H₅)₂, —NH—CH₃, —NH—C₂H₅, —CH₂—O—C(═O)-phenyl, —C(═O)—O—CH₃, —C(═O)—O—C₂H₅, —C(═O)—O—CH(CH₃)₂, —C(═O)—O—(CH₂)₃—CH₃, —C(═O)—O—C(CH₃)₃, —C(═O)—O-phenyl, —C(═O)—H, —C(═O)—CH₃, —C(═O)—C₂H₅, —C(═O)—NH₂, —C(═O)—NH—CH₃ and —C(═O)—N(CH₃)₂;
and, provided that M² denotes unsubstituted or substituted heteroaryl or unsubstituted or substituted naphthyl or an unsubstituted or substituted phenyl residue, which can be condensed (annelated) with unsubstituted or substituted 5- to 7-membered heterocycloalkyl or with unsubstituted or substituted C₅₋₇-cycloalkyl, R⁵ can additionally denote —C(═O)—R¹¹;
and R⁶, R⁷, R⁸, R⁹, R¹⁰, R¹¹, R¹², R¹⁷, R¹⁸, R¹⁹, R²⁰ and R²¹, mutually independently, in each case denote C₁₋₆-alkyl, which is unsubstituted or substituted with optionally 1, 2, 3, 4 or 5 substituents mutually independently selected from the group consisting of F, Cl, Br, I, —NO₂, —CN, —OH, —SH and —NH₂; unsubstituted C₃₋₇-cycloalkyl; unsubstituted C₅₋₆-cycloalkenyl; unsubstituted 5- to 7-membered heterocycloalkyl and unsubstituted 5 to 7-membered heterocycloalkenyl; or a residue selected from the group consisting of phenyl, benzyl, naphthyl, anthracenyl, pyrrolyl, indolyl, furanyl, benzo[b]furanyl, thiophenyl, benz[b]thiophenyl, benzo[d]thiazolyl, pyrazolyl, imidazolyl, thiazolyl, thiadiazolyl, triazolyl, oxazolyl, oxadiazolyl, isoxazolyl, pyridinyl, pyridazinyl, pyrimidinyl, pyrazinyl, pyranyl, indazolyl, quinolinyl, isoquinolinyl and quinazolinyl, which in each case is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
in each case optionally in the form of one of the pure stereoisomers thereof, the racemates thereof or in the form of a mixture of stereoisomers in any desired mixing ratio, or in each case in the form of a corresponding salts or in each case in the form of a corresponding solvates.

10. A compound according to claim 1, wherein
I.)
M¹ denotes a phenyl residue, which is unsubstituted or substituted with 1, 2, 3, 4 or 5 substituents mutually independently selected from the group consisting of F, Cl, Br, I, —CN, —NO₂, methyl, ethyl, n-propyl, isopropyl, ethenyl, allyl, ethynyl, propynyl, —OH, —NH₂, —O—CH₃, —O—C₂H₅, —O—C₃H₇, —CF₃, —CHF₂, —CH₂F, —O—CF₃, —O—CHF₂, —O—CH₂F, —N(CH₃)₂, —N(C₂H₅)₂, —NH—CH₃, —NH—C₂H₅, —C(═O)—O—CH₃, —C(═O)—O—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅ and cyclopropyl;
and M² denotes a phenyl residue, which is substituted with 1, 2, 3, 4 or 5 substituents selected from the group comp consisting of F, Cl, Br, I, —CN, —NO₂, methyl, ethyl, n-propyl, isopropyl, n-butyl, isobutyl, tert-butyl, ethenyl, allyl, ethynyl, propynyl, —OH, —NH₂, —O—CH₃, —O—C₂H₅, —O—C₃H₇, —CF₃, —CHF₂, —CH₂F, —O—CF₃, —O—CHF₂, —O—CH₂F, —N(CH₃)₂, —N(C₂H₅)₂, —NH—CH₃, —NH—C₂H₅, —C(═O)—O—CH₃, —C(═O)—O—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅ and cyclopropyl;
or M² denotes a residue selected from the group consisting of indolyl, naphthyl, thiophenyl (thienyl), furanyl (furyl), pyrrolyl, pyrazolyl, triazolyl, pyridinyl, imidazolyl, thiazolyl, thiadiazolyl, oxazolyl, oxadiazolyl, tetrazolyl, triazinyl, isoxazolyl, pyridazinyl, pyrazinyl, pyrimidinyl, diaza-naphthyl, quinoxalinyl, quinazolinyl, quinolinyl, naphthridinyl, isoquinolinyl, indolinyl, (2,3)-dihydrobenzofuranyl, (2,3)-dihydrobenzo[d]oxazolyl, benzo[d][1,3]dioxolyl, isoindolinyl, (1,2,3,4)-tetrahydroquinolinyl and (2,3)-dihydrobenzo[b][1,4] dioxinyl, which is unsubstituted or substituted with 1, 2, 3, 4 or 5 substituents selected from the group consisting of F, Cl, Br, I, —CN, —NO₂, methyl, ethyl, n-propyl, isopropyl, n-butyl, isobutyl, tert-butyl, ethenyl, allyl, ethynyl, propynyl, —OH, —NH₂, —O—CH₃, —O—C₂H₅, —O—C₃H₇, —CF₃, —CHF₂, —CH₂F, —O—CF₃, —O—CHF₂, —O—CH₂F, —N(CH₃)₂, —N(C₂H₅)₂, —NH—CH₃, —NH—C₂H₅, —C(═O)—O—CH₃, —C(═O)—O—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅ and cyclopropyl;
or II.)
M¹ denotes a residue selected from the group consisting of naphthyl, thiophenyl (thienyl), furanyl (furyl), pyrrolyl, pyrazolyl, triazolyl, pyridinyl, imidazolyl, indolyl, thiazolyl, thiadiazolyl, oxazolyl, oxadiazolyl, tetrazolyl, triazinyl, isoxazolyl, pyridazinyl, pyrazinyl, pyrimidinyl, diaza-naphthyl, quinoxalinyl, quinazolinyl, quinolinyl, naphthridinyl, isoquinolinyl, indolinyl, (2,3)-dihydrobenzofuranyl, (2,3)-dihydrobenzo[d]oxazolyl, benzo[d][1,3]dioxolyl, isoindolinyl, (1,2,3,4)-tetrahydroquinolinyl and (2,3)-dihydrobenzo[b][1,4]dioxinyl, which is unsubstituted or substituted with 1, 2, 3, 4 or 5 substituents mutually independently selected from the group consisting of F, Cl, Br, I, —CN, —NO₂, methyl, ethyl, n-propyl, isopropyl, n-butyl, isobutyl, tert-butyl, ethenyl, allyl, ethynyl, propynyl, —OH, —NH₂, —O—CH₃, —O—C₂H₅, —O—C₃H₇, —CF₃, —CHF₂, —CH₂F, —O—CF₃, —O—CHF₂, —O—CH₂F, —N(CH₃)₂, —N(C₂H₅)₂, —NH—CH₃, —NH—C₂H₅, —C(═O)—O—CH₃, —C(═O)—O—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅ and cyclopropyl;
and M² denotes a residue selected from the group consisting of indolyl, naphthyl, thiophenyl (thienyl), furanyl (furyl), pyrrolyl, pyrazolyl, triazolyl, pyridinyl, imidazolyl, thiazolyl, thiadiazolyl, oxazolyl, oxadiazolyl, tetrazolyl, triazinyl, isoxazolyl, pyridazinyl, pyrazinyl, pyrimidinyl, diaza-naphthyl, quinoxalinyl, quinazolinyl, quinolinyl, naphthridinyl, isoquinolinyl, indolinyl, (2,3)-dihydrobenzofuranyl, (2,3)-dihydrobenzo[d]oxazolyl, benzo[d][1,3]dioxolyl, isoindolinyl, (1,2,3,4)-tetrahydroquinolinyl and (2,3)-dihydrobenzo[b][1,4]dioxinyl, which is unsubstituted or substituted with 1, 2, 3, 4 or 5 substituents mutually independently selected from the group consisting of F, Cl, Br, I, —CN, —NO₂, methyl, ethyl, n-propyl, isopropyl, n-butyl, isobutyl, tert-butyl, ethenyl, allyl, ethynyl, propynyl, —OH, —NH₂, —O—CH₃, —O—C₂H₅, —O—C₃H₇, —CF₃, —CHF₂, —CH₂F, —O—CF₃, —O—CHF₂, —O—CH₂F, —N(CH₃)₂, —N(C₂H₅)₂, —NH—CH₃, —NH—C₂H₅, —C(═O)—O—CH₃, —C(═O)—O—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅ and cyclopropyl;
or M² denotes a phenyl residue, which is unsubstituted or substituted with 1, 2, 3, 4 or 5 substituents mutually independently selected from the group consisting of F, Cl, Br, I, —CN, —NO₂, methyl, ethyl, n-propyl, isopropyl, n-butyl, isobutyl, tert-butyl, ethenyl, allyl, ethynyl, propynyl, —OH, —NH₂, —O—CH₃, —O—C₂H₅, —O—C₃H₇, —CF₃, —CHF₂, —CH₂F, —O—CF₃, —O—CHF₂, —O—CH₂F, —N(CH₃)₂, —N(C₂H₅)₂, —NH—CH₃, —NH—C₂H₅, —C(═O)—O—CH₃, —C(═O)—O—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅ and cyclopropyl;
and in each case
R¹ and R², mutually independently, in each case denote H; F; Cl; Br; I; —NO₂; —CN; —OH; —SH; —O—R⁶; —S—R⁷; —NH—R⁸; —NR⁹R¹⁰; or a residue selected from the group consisting of methyl, ethyl, n-propyl, isopropyl, n-butyl, tert-butyl, isobutyl, n-pentyl, —CF₃, —CF₂H, —CFH₂, —C₂F₅ and —CH₂—CF₃ or a phenyl residue, which can be unsubstituted or substituted with 1, 2, 3, 4 or 5 substituents mutually independently selected from the group consisting of F, Cl, Br, I, —CN, —NO₂, methyl, ethyl, n-propyl, isopropyl, n-butyl, tert-butyl, n-pentyl, —OH, —SH, —NH₂, —O—CH₃, —O—C₂H₅, —O—C₃H₇, —O—C(CH₃)₃, —CF₃, —CHF₂, —CH₂F and —O—CF₃;
or R¹ and R² jointly denote an oxo group (═O);
R³ and R⁴, mutually independently, in each case denote H; F; Cl; Br; I; —NO₂; —CN; —OH; —SH; —O—R⁶; —S—R⁷; —NH—R⁸; —NR⁹R¹⁰; or a residue selected from the group consisting of methyl, ethyl, n-propyl, isopropyl, n-butyl, tert-butyl, isobutyl, n-pentyl, —CF₃, —CF₂H, —CFH₂, —C₂F₅ and —CH₂—CF₃ or a phenyl residue, which can be unsubstituted or substituted with 1, 2, 3, 4 or 5 substituents mutually independently selected from the group consisting of F, Cl, Br, I, —CN, —NO₂, methyl, ethyl, n-propyl, isopropyl, n-butyl, tert-butyl, n-pentyl, —OH, —SH, —NH₂, —O—CH₃, —O—C₂H₅, —O—C₃H₇, —O—C(CH₃)₃, —CF₃, —CHF₂, —CH₂F and —O—CF₃;
R⁵ denotes H; —C(═O)—O—R¹²; —S(═O)—R²⁰; —S(═O)₂—R²¹; a residue selected from the group consisting of methyl, ethyl, n-propyl, isopropyl, n-butyl, tert-butyl, isobutyl, n-pentyl, —CF₃, —CF₂H, —CFH₂, —C₂F₅ and —CH₂—CF₃; a residue selected from the group consisting of cyclopropyl, cyclobutyl, cyclopentyl and cyclohexyl; or a benzyl or phenethyl residue, which is unsubstituted or substituted with 1, 2, 3, 4 or 5 substituents mutually independently selected from the group consisting of F, Cl, Br, I, —CN, —NO₂, methyl, ethyl, n-propyl, isopropyl, n-butyl, tert-butyl, n-pentyl, —OH, —SH, —NH₂, —O—CH₃, —O—C₂H₅, —O—C₃H₇, —O—C(CH₃)₃, —CF₃, —CHF₂, —CH₂F, —O—CF₃, —O—CHF₂, —O—CH₂F, —S—CF₃, —S—CHF₂, —S—CH₂F, —N(CH₃)₂, —N(C₂H₅)₂, —NH—CH₃ and —NH—C₂H₅;
and, provided that M² denotes unsubstituted or substituted heteroaryl or unsubstituted or substituted naphthyl or an unsubstituted or substituted phenyl residue, which can be condensed (annelated) with unsubstituted or substituted 5- to 7-membered heterocycloalkyl or with unsubstituted or substituted C₅₋₇-cycloalkyl, R⁵ can additionally denote —C(═O)—R¹¹;
and R⁶, R⁷, R⁸, R⁹, R¹⁰, R¹¹, R¹², R²⁰ and R²¹, mutually independently, in each case denote a residue selected from the group consisting of methyl, ethyl, n-propyl, isopropyl, n-butyl, tert-butyl, isobutyl, n-pentyl, —CF₃, —CF₂H, —CFH₂, —C₂F₅ and —CH₂—CF₃; a residue selected from the group consisting of cyclopropyl, cyclobutyl, cyclopentyl and cyclohexyl; a residue selected from the group consisting of pyrrolidinyl, piperidinyl, morpholinyl, piperazinyl, thiomorpholinyl, tetrahydropyranyl, oxetanyl, azepanyl and diazepanyl; or a residue selected from the group consisting of phenyl, benzyl, naphthyl, furanyl, thiophenyl, pyrazolyl, imidazolyl, thiazolyl and thiadiazolyl, which in each case is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
in each case optionally in the form of one of the pure stereoisomers thereof, the racemates thereof or in the form of a mixture of stereoisomers in any desired mixing ratio, or in each case in the form of a corresponding salts or in each case in the form of a corresponding solvates.

11. A compound according to claim 1, wherein
I.)
M¹ denotes a phenyl residue, which is unsubstituted or substituted with 1, 2, 3, 4 or 5 substituents mutually independently selected from the group consisting of F, Cl, Br, I, —CN, —NO₂, methyl, ethyl, n-propyl, isopropyl, ethenyl, allyl, ethynyl, propynyl, —OH, —NH₂, —O—CH₃, —O—C₂H₅, —O—C₃H₇, —CF₃, —CHF₂, —CH₂F, —O—CF₃, —O—CHF₂, —O—CH₂F, —N(CH₃)₂, —N(C₂H₅)₂, —NH—CH₃, —NH—C₂H₅, —C(═O)—O—CH₃, —C(═O)—O—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅ and cyclopropyl;
and M² denotes a phenyl residue, which is substituted with 1, 2, 3, 4 or 5 substituents mutually independently selected from the group consisting of F, Cl, Br, I, —CN, —NO₂, methyl, ethyl, n-propyl, isopropyl, n-butyl, isobutyl, tert-butyl, ethenyl, allyl, ethynyl, propynyl, —OH, —NH₂, —O—CH₃, —O—C₂H₅, —O—C₃H₇, —CF₃, —CHF₂, —CH₂F, —O—CF₃, —O—CHF₂, —O—CH₂F, —N(CH₃)₂, —N(C₂H₅)₂, —NH—CH₃, —NH—C₂H₅, —C(═O)—O—CH₃, —C(═O)—O—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅ and cyclopropyl;
or M² denotes a residue selected from the group consisting of indolyl, thiophenyl (thienyl), pyridinyl, thiazolyl, thiadiazolyl, pyridazinyl, pyrazinyl, pyrimidinyl, benzo[d][1,3]dioxolyl and (2,3)-dihydrobenzo[b][1,4]dioxinyl, which is unsubstituted or substituted with 1, 2, 3, 4 or 5 substituents mutually independently selected from the group consisting of F, Cl, Br, I, —CN, —NO₂, methyl, ethyl, n-propyl, isopropyl, n-butyl, isobutyl, tert-butyl, ethenyl, allyl, ethynyl, propynyl, —OH, —NH₂, —O—CH₃, —O—C₂H₅, —O—C₃H₇, —CF₃, —CHF₂, —CH₂F, —O—CF₃, —O—CHF₂, —O—CH₂F, —N(CH₃)₂, —N(C₂H₅)₂, —NH—CH₃, —NH—C₂H₅, —C(═O)—O—CH₃, —C(═O)—O—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅ and cyclopropyl;
or II.)
M¹ denotes a residue selected from the group consisting of naphthyl, thiophenyl (thienyl), furanyl (furyl), pyridinyl, pyrrolyl, pyrazolyl, imidazolyl, indolyl, thiazolyl, pyridazinyl, pyrazinyl, pyrimidinyl, quinolinyl, isoquinolinyl, benzo[d][1,3]dioxolyl and (2,3)-dihydrobenzo[b][1,4]dioxinyl, which is unsubstituted or substituted with 1, 2, 3, 4 or 5 substituents mutually independently selected from the group consisting of F, Cl, Br, I, —CN, —NO₂, methyl, ethyl, n-propyl, isopropyl, n-butyl, isobutyl, tert-butyl, ethenyl, allyl, ethynyl, propynyl, —OH, —NH₂, —O—CH₃, —O—C₂H₅, —O—C₃H₇, —CF₃, —CHF₂, —CH₂F, —O—CF₃, —O—CHF₂, —O—CH₂F, —N(CH₃)₂, —N(C₂H₅)₂, —NH—CH₃, —NH—C₂H₅, —C(═O)—O—CH₃, —C(═O)—O—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅ and cyclopropyl;
and M² denotes a residue selected from the group consisting of indolyl, thiophenyl (thienyl), pyridinyl, thiazolyl, thiadiazolyl, pyridazinyl, pyrazinyl, pyrimidinyl, benzo[d][1,3]dioxolyl and (2,3)-dihydrobenzo[b][1,4]dioxinyl, which is unsubstituted or substituted with 1, 2, 3, 4 or 5 substituents mutually independently selected from the group consisting of F Cl, Br, I, —CN, —NO₂, methyl, ethyl, n-propyl, isopropyl, n-butyl, isobutyl, tert-butyl, ethenyl, allyl, ethynyl, propynyl, —OH, —NH₂, —O—CH₃, —O—C₂H₅, —O—C₃H₇, —CF₃, —CHF₂, —CH₂F, —O—CF₃, —O—CHF₂, —O—CH₂F, —N(CH₃)₂, —N(C₂H₅)₂, —NH—CH₃, —NH—C₂H₅, —C(═O)—O—CH₃, —C(═O)—O—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅ and cyclopropyl;
or M² denotes a phenyl residue, which is unsubstituted or substituted with 1, 2, 3, 4 or 5 substituents mutually independently selected from the group consisting of F, Cl, Br, I, —CN, —NO₂, methyl, ethyl, n-propyl, isopropyl, n-butyl, isobutyl, tert-butyl, ethenyl, allyl, ethynyl, propynyl, —OH, —NH₂, —O—CH₃, —O—C₂H₅, —O—C₃H₇, —CF₃, —CHF₂, —CH₂F, —O—CF₃, —O—CHF₂, —O—CH₂F, —N(CH₃)₂, —N(C₂H₅)₂, —NH—CH₃, —NH—C₂H₅, —C(═O)—O—CH₃, —C(═O)—O—C₂H₅, —NH—C(═O)—CH₃, —O—C(═O)—CH₃, —O—C(═O)—C₂H₅, —C(═O)—NH₂, —C(═O)—NH—CH₃, —C(═O)—N(CH₃)₂, —C(═O)—NH—C₂H₅ and cyclopropyl;
and in each case
R¹, R², R³ and R⁴, mutually independently, in each case denote H; F; Cl; Br; —OH; —SH; —CN; —O—R⁶; —S—R⁷; —NH—R⁸; —NR⁹R¹⁰; or a residue selected from the group consisting of methyl, ethyl, n-propyl, isopropyl, n-butyl, tert-butyl, isobutyl, n-pentyl, —CF₃, —CF₂H, —CFH₂, —C₂F₅ and —CH₂—CF₃ or a phenyl residue, which can be unsubstituted or substituted with 1, 2, 3, 4 or 5 substituents mutually independently selected from the group consisting of F, Cl, Br, I, —CN, —NO₂, methyl, ethyl, n-propyl, isopropyl, n-butyl, tert-butyl, n-pentyl, —OH, —SH, —NH₂, —O—CH₃, —O—C₂H₅, —O—C₃H₇, —O—C(CH₃)₃, —CF₃, —CHF₂, —CH₂F and —O—CF₃;
or R¹ and R² jointly denote an oxo group (═O);
R⁵ denotes H; —C(═O)—O—R¹²; a residue selected from the group consisting of methyl, ethyl, n-propyl, isopropyl, n-butyl, tert-butyl, isobutyl, n-pentyl, —CF₃, —CF₂H, —CFH₂, —C₂F₅ and —CH₂—CF₃; a residue selected from the group consisting of cyclopropyl, cyclobutyl, cyclopentyl and cyclohexyl; or a benzyl or phenethyl residue, which is unsubstituted or substituted with 1, 2, 3, 4 or 5 substituents mutually independently selected from the group consisting of F, Cl, Br, I, —CN, —NO₂, methyl, ethyl, n-propyl, isopropyl, n-butyl, tert-butyl, n-pentyl, —O—CH₃, —O—C₂H₅, —O—C₃H₇, —O—C(CH₃)₃ and —CF₃;
and, provided that M² denotes unsubstituted or substituted heteroaryl or unsubstituted or substituted benzo[d][1,3]dioxolyl or (2,3)-dihydrobenzo[b][1,4]dioxinyl, R⁵ can additionally denote —C(═O)—R¹¹;
and R⁶, R⁷, R⁸, R⁹, R¹⁰, R¹¹ and R¹², mutually independently, in each case denote a residue selected from the group consisting of methyl, ethyl, n-propyl, isopropyl, n-butyl, tert-butyl, isobutyl, n-pentyl, —CF₃, —CF₂H, —CFH₂, —C₂F₅ and —CH₂—CF₃; or a residue selected from the group consisting of phenyl and benzyl, which in each case is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇ and —CF₃;
in each case optionally in the form of one of the pure stereoisomers thereof, the racemates thereof or in the form of a mixture of stereoisomers in any desired mixing ratio, or in each case in the form of a corresponding salts or in each case in the form of a corresponding solvates.

12. A compound according to one or more of claim 1, wherein
I.)
M¹ denotes a phenyl residue, which is unsubstituted or substituted with 1, 2, 3, 4 or 5 substituents mutually independently selected from the group consisting of F, Cl, Br, —CN, —NO₂, methyl, ethyl, n-propyl, isopropyl, —OH, —NH₂, —O—CH₃, —O—C₂H₅, —CF₃, —O—CF₃, —O—CHF₂, —O—CH₂F, —N(CH₃)₂, —NH—CH₃, —C(═O)—O—CH₃, —C(═O)—O—C₂H₅, —NH—C(═O)—CH₃, —O—C(═O)—CH₃, —C(═O)—N(CH₃)₂ and cyclopropyl;
and M² denotes a phenyl residue, which is substituted with 1, 2, 3, 4 or 5 substituents mutually independently selected from the group consisting of F, Cl, Br, I, —CN, —OH, —NO₂, methyl, ethyl, n-propyl, isopropyl, n-butyl, isobutyl, tert-butyl, ethenyl, allyl, ethynyl, propynyl, cyclopropyl, —O—CH₃, —O—C₂H₅, —CF₃, —O—CF₃, —O—CHF₂ and —O—CH₂F;
or M² denotes an indolyl, thiophene-2-yl or thiophene-3-yl residue, which is unsubstituted or substituted with 1, 2, 3, 4 or 5 substituents mutually independently selected from the group consisting of F, Cl, Br, I, —CN, —NO₂, methyl, ethyl, n-propyl, isopropyl, n-butyl, isobutyl, tert-butyl, ethenyl, allyl, ethynyl, propynyl, cyclopropyl, —OH, —O—CH₃, —O—C₂H₅, —CF₃, —O—CF₃, —O—CHF₂ and —O—CH₂F;
or II.)
M¹ denotes a residue selected from the group consisting of pyrrolyl, pyrazolyl, imidazolyl, indolyl, pyridine-2-yl, pyridine-3-yl, pyridine-4-yl, thiophene-2-yl and thiophene-3-yl, which is unsubstituted or substituted with 1, 2, 3, 4 or 5 substituents mutually independently selected from the group consisting of F, Cl, Br, —CN, —NO₂, methyl, ethyl, n-propyl, isopropyl, n-butyl, isobutyl, tert-butyl, —OH, —NH₂, —O—CH₃, O—C₂H₅, —CF₃, —O—CF₃, —O—CHF₂, —O—CH₂F, —N(CH₃)₂, —NH—CH₃, —C(═O)—O—CH₃, —C(═O)—O—C₂H₅, —NH—C(═O)—CH₃, —O—C(═O)—CH₃, —C(═O)—N(CH₃)₂ and cyclopropyl;
and M² denotes an indolyl, thiophene-2-yl or thiophene-3-yl residue, which is unsubstituted or substituted with 1, 2, 3, 4 or 5 substituents mutually independently selected from the group consisting of F, Cl, Br, I, —CN, —NO₂, methyl, ethyl, n-propyl, isopropyl, n-butyl, isobutyl, tert-butyl, ethenyl, allyl, ethynyl, propynyl, cyclopropyl, —OH, —O—CH₃, —O—C₂H₅, —CF₃, —O—CF₃, —O—CHF₂ and —O—CH₂F;
or M² denotes a phenyl residue, which is unsubstituted or substituted with 1, 2, 3, 4 or 5 substituents mutually independently selected from the group consisting of F, Cl, Br, I, —CN, —NO₂, methyl, ethyl, n-propyl, isopropyl, n-butyl, isobutyl, tert-butyl, ethenyl, allyl, ethynyl, —OH, propynyl, cyclopropyl, —O—CH₃, —O—C₂H₅, —CF₃, —O—CF₃, —O—CHF₂ and —O—CH₂F;
and in each case
R¹, R², R³ and R⁴, mutually independently, in each case denote H; F; Cl; —CN; —O—CH₃; —O—C₂H₅; —O—CF₃; —O—CF₂H; —O—CFH₂; —N(CH₃)₂; —N(C₂H₅)₂; phenyl; methyl; ethyl; n-propyl or isopropyl;
R⁵ denotes H; methyl; ethyl; n-propyl; isopropyl; cyclopropyl or benzyl;
and, provided that M² denotes an indolyl, thiophene-2-yl or thiophene-3-yl residue, R⁵ can additionally denote —C(═O)—CH₃, —C(═O)—C₂H₅, —C(═O)—CH(CH₃)₂, —C(═O)—C(CH₃)₃, —C(═O)-phenyl or —C(═O)-benzyl;
in each case optionally in the form of one of the pure stereoisomers thereof, the racemates thereof or in the form of a mixture of stereoisomers in any desired mixing ratio, or in each case in the form of a corresponding salts or in each case in the form of a corresponding solvates.

13. A compound according to claim 1, which has the formula Ia: 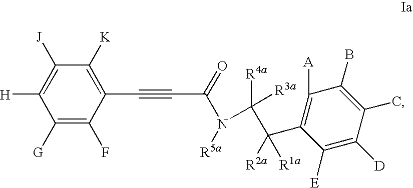
in which
A, B, C, D and E, mutually independently, in each case denote H, F, Cl, Br, —CN, —NO₂, methyl, ethyl, n-propyl, isopropyl, —OH, —NH₂, —O—CH₃, —O—C₂H₅, —CF₃, —O—CF₃, —O—CHF₂, —O—CH₂F, —N(CH₃)₂, —NH—CH₃, —C(═O)—O—CH₃, —C(═O)—O—C₂H₅, —NH—C(═O)—CH₃, —O—C(═O)—CH₃, —C(═O)—N(CH₃)₂ or cyclopropyl;
F, G, H, J and K, mutually independently, in each case denote H, F, Cl, Br, I, —CN, —NO₂, methyl, ethyl, n-propyl, isopropyl, n-butyl, isobutyl, tert-butyl, ethenyl, allyl, ethynyl, propynyl, cyclopropyl, —OH, —O—CH₃, —O—C₂H₅, —CF₃, —O—CF₃, —O—CHF₂ or —O—CH₂F, with the proviso that at least one of the substituents F, G, H, J and K is not equal to H;
R^{1a}, R^{2a}, R^{3a} and R^{4a}, mutually independently, in each case denote H; F; Cl; —CN; —O—CH₃; —O—C₂H₅; —O—CF₃; —O—CF₂H; —N(CH₃)₂; —N(C₂H₅)₂; phenyl; —O—CFH₂; methyl; ethyl; n-propyl or isopropyl;
and R^{5a} denotes H; methyl; ethyl; n-propyl; isopropyl; cyclopropyl or benzyl;
in each case optionally in the form of one of the pure stereoisomers thereof, the racemates thereof or in the form of a mixture of stereoisomers in any desired mixing ratio, or in each case in the form of a corresponding salts or in each case in the form of a corresponding solvates.

14. A compound according to claim 1, which has the Ib: 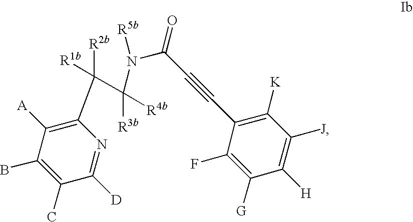
in which
A, B, C and D, mutually independently, in each case denote H, F, Cl, Br, —CN, —NO₂, methyl, ethyl, n-propyl, isopropyl, —OH, —NH₂, —O—CH₃, —O—C₂H₅, —CF₃, —O—CF₃, —O—CHF₂, —O—CH₂F, —N(CH₃)₂, —NH—CH₃, —C(═O)—O—CH₃, —C(═O)—O—C₂H₅, —NH—C(═O)—CH₃, —O—C(═O)—CH₃, —C(═O)—N(CH₃)₂ andor cyclopropyl;
F, G, H, J and K, mutually independently, in each case denote H, F, Cl, Br, I, —CN, —NO₂, methyl, ethyl, n-propyl, isopropyl, n-butyl, isobutyl, tert-butyl, ethenyl, allyl, ethynyl, propynyl, cyclopropyl, —OH, —O—CH₃, —O—C₂H₅, —CF₃, —O—CF₃, —O—CHF₂ or —O—CH₂F;
R^{1b}, R^{2b}, R^{3b} and R^{4b}, mutually independently, in each case denote H; F; Cl; —CN; —O—CH₃; —O—C₂H₅; —O—CF₃; —O—CF₂H; —N(CH₃)₂; —N(C₂H₅)₂; phenyl; —O—CFH₂; methyl; ethyl; n-propyl or isopropyl;
and R^{5b} denotes H; methyl; ethyl; n-propyl; isopropyl; cyclopropyl or benzyl;
in each case optionally in the form of one of the pure stereoisomers thereof, the racemates thereof or in the form of a mixture of stereoisomers in any desired mixing ratio, or in each case in the form of a corresponding salts or in each case in the form of a corresponding solvates.

15. A compound according to claim 1, which has the formula Ic: 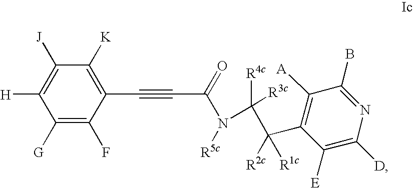
in which
A, B, D and E, mutually independently, in each case denote H, F, Cl, Br, —CN, —NO₂, methyl, ethyl, n-propyl, isopropyl, —OH, —NH₂, —O—CH₃, —O—C₂H₅, —CF₃, —O—CF₃, —O—CHF₂, —O—CH₂F, —N(CH₃)₂, —NH—CH₃, —C(═O)—O—CH₃, —C(═O)—O—C₂H₅, —NH—C(═O)—CH₃, —O—C(═O)—CH₃, —C(═O)—N(CH₃)₂ or cyclopropyl;
F, G, H, J and K, mutually independently, in each case denote H, F, Cl, Br, I, —CN, —NO₂, methyl, ethyl, n-propyl, isopropyl, n-butyl, isobutyl, tert-butyl, ethenyl, allyl, ethynyl, propynyl, cyclopropyl, —OH, —O—CH₃, —O—C₂H₅, —CF₃, —O—CF₃, —O—CHF₂ or —O—CH₂F;
R^{1c}, R^{2c}, R^{3c} and R^{4c}, mutually independently, in each case denote H; F; Cl; —CN; —O—CH₃; —O—C₂H₅; —O—CF₃; —O—CF₂H; —N(CH₃)₂; —N(C₂H₅)₂; phenyl; —O—CFH₂; methyl; ethyl; n-propyl or isopropyl;
and R^{5c} denotes H; methyl; ethyl; n-propyl; isopropyl; cyclopropyl or benzyl;
in each case optionally in the form of one of the pure stereoisomers thereof, the racemates thereof or in the form of a mixture of stereoisomers in any desired mixing ratio, or in each case in the form of a corresponding salts or in each case in the form of a corresponding solvates.

16. A compound according to claim 1, which has the formula Id: 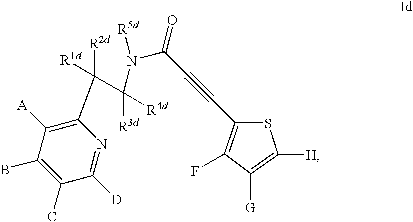
in which
A, B, C and D, mutually independently, in each case denote H, F, Cl, Br, —CN, —NO₂, methyl, ethyl, n-propyl, isopropyl, —OH, —NH₂, —O—CH₃, —O—C₂H₅, —CF₃, —O—CF₃, —O—CHF₂, —O—CH₂F, —N(CH₃)₂, —NH—CH₃, —C(═O)—O—CH₃, —C(═O)—O—C₂H₅, —NH—C(═O)—CH₃, —O—C(═O)—CH₃, —C(═O)—N(CH₃)₂ or cyclopropyl;
F, G, and H, mutually independently, in each case denote H, F, Cl, Br, I, —CN, —NO₂, methyl, ethyl, n-propyl, isopropyl, n-butyl, isobutyl, tert-butyl, ethenyl, allyl, ethynyl, propynyl, cyclopropyl, —OH, —O—CH₃, —O—C₂H₅, —CF₃, —O—CF₃, —O—CHF₂ or —O—CH₂F;
R^{1b}, R^{2d}, R^{3d} and R^{4d}, mutually independently, in each case denote H; F; Cl; —CN; —O—CH₃; —O—C₂H₅; —O—CF₃; —O—CF₂H; —O—CFH₂; —N(CH₃)₂; —N(C₂H₅)₂; phenyl; methyl; ethyl;
n-propyl or isopropyl;
and R^{5d} denotes H; methyl; ethyl; n-propyl; isopropyl; cyclopropyl; benzyl; —C(═O)—CH₃, —C(═O)—C₂H₅, —C(═O)—CH(CH₃)₂, —C(═O)—C(CH₃)₃, —C(═O)-phenyl or —C(═O)-benzyl;
in each case optionally in the form of one of the pure stereoisomers thereof, the racemates thereof or in the form of a mixture of stereoisomers in any desired mixing ratio, or in each case in the form of a corresponding salts or in each case in the form of a corresponding solvates.

17. A compound according to claim 1, which has the formula Ie: 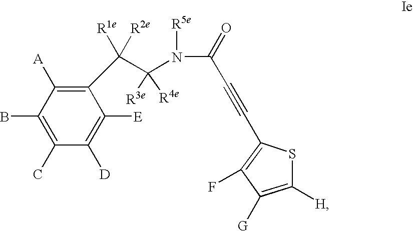
in which
A, B, C, D and E, mutually independently, in each case denote H, F, Cl, Br, —CN, —NO₂, methyl, ethyl, n-propyl, isopropyl, —OH, —NH₂, —O—CH₃, —O—C₂H₅, —CF₃, —O—CF₃, —O—CHF₂, —O—CH₂F, —N(CH₃)₂, —NH—CH₃, —C(═O)—O—CH₃, —C(═O)—O—C₂H₅, —NH—C(═O)—CH₃, —O—C(═O)—CH₃, —C(═O)—N(CH₃)₂ or cyclopropyl;
F, G, and H, mutually independently, in each case denote H, F, Cl, Br, I, —CN, —NO₂, methyl, ethyl, n-propyl, isopropyl, n-butyl, isobutyl, tert-butyl, ethenyl, allyl, ethynyl, propynyl, cyclopropyl, —OH, —O—CH₃, —O—C₂H₅, —CF₃, —O—CF₃, —O—CHF₂ or —O—CH₂F;
R^{1e}, R^{2e}, R^{3e} and R^{4e}, mutually independently, in each case denote H; F; Cl; —CN; —O—CH₃; —O—C₂H₅; —O—CF₃; —O—CF₂H; —O—CFH₂; —N(CH₃)₂; —N(C₂H₅)₂; phenyl; methyl; ethyl; n-propyl or isopropyl;
and R^{5e} denotes H; methyl; ethyl; n-propyl; isopropyl; cyclopropyl; benzyl; —C(═O)—CH₃, —C(═O)—C₂H₅, —C(═O)—CH(CH₃)₂, —C(═O)—C(CH₃)₃, —C(═O)-phenyl or —C(═O)-benzyl;
in each case optionally in the form of one of the pure stereoisomers thereof, the racemates thereof or in the form of a mixture of stereoisomers in any desired mixing ratio, or in each case in the form of a corresponding salts or in each case in the form of a corresponding solvates.

18. A compound according to claim 1, which has the formula If: 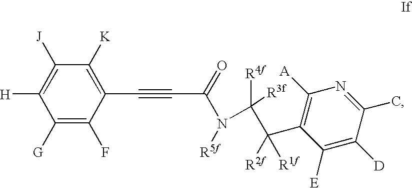
in which
A, C, D and E, mutually independently, in each case denote H, F, Cl, Br, —CN, —NO₂, methyl, ethyl, n-propyl, isopropyl, —OH, —NH₂, —O—CH₃, —O—C₂H₅, —CF₃, —O—CF₃, —O—CHF₂, —O—CH₂F, —N(CH₃)₂, —NH—CH₃, —C(═O)—O—CH₃, —C(═O)—O—C₂H₅, —NH—C(═O)—CH₃, —O—C(═O)—CH₃, —C(═O)—N(CH₃)₂ or cyclopropyl;
F, G, H, J and K, mutually independently, in each case denote H, F, Cl, Br, I, —CN, —NO₂, methyl, ethyl, n-propyl, isopropyl, n-butyl, isobutyl, tert-butyl, ethenyl, allyl, ethynyl, propynyl, cyclopropyl, —OH, —O—CH₃, —O—C₂H₅, —CF₃, —O—CF₃, —O—CHF₂ or —O—CH₂F;
R^{1f}, R^{2f}, R^{3f} and R^{4f}, mutually independently, in each case denote H; F; Cl; —CN; —O—CH₃; —O—C₂H₅; —O—CF₃; —O—CF₂H; —O—CFH₂; —N(CH₃)₂; —N(C₂H₅)₂; phenyl; methyl; ethyl; n-propyl or isopropyl;
and R^{5f} denotes H; methyl; ethyl; n-propyl; isopropyl; cyclopropyl or benzyl;
in each case optionally in the form of one of the pure stereoisomers thereof, the racemates thereof or in the form of a mixture of stereoisomers in any desired mixing ratio, or in each case in the form of a corresponding salts or in each case in the form of a corresponding solvates.

19. A compound according to claim 1, which is selected from the group of:
[1] 3-(3-chlorophenyl)-N-methyl-N-phenethylpropiolamide,
[2] 3-(3-chlorophenyl)-N-phenethylpropiolamide,
[3] 3-(3-chlorophenyl)-N-(2-(pyridine-2-yl)ethyl)propiolamide,
[4] 3-(3-chlorophenyl)-N-(2-(pyridine-4-yl)ethyl)propiolamide,
[5] 3-(2,4-difluorophenyl)-N-(2-(pyridine-2-yl)ethyl)propiolamide,
[6] 3-(3-methoxyphenyl)-N-(2-(pyridine-2-yl)ethyl)propiolamide,
[7] 3-(4-fluoro-3-methylphenyl)-N-(2-(pyridine-2-yl)ethyl)propiolamide,
[8] 3-(2-fluorophenyl)-N-(2-(pyridine-2-yl)ethyl)propiolamide,
[9] N-(2-(pyridine-2-yl)ethyl)-3-p-tolylpropiolamide,
[10] N-(2-(pyridine-2-yl)ethyl)-3-(4-(trifluoromethyl)-phenyl)-propiolamide,
[11] 3-phenyl-N-(2-(pyridine-2-yl)ethyl)propiolamide,
[12] 3-(2,3-dimethyl-phenyl)-N-(2-(pyridine-2-yl)ethyl)propiolamide,
[13] 3-(3,5-dimethyl-phenyl)-N-(2-(pyridine-2-yl)ethyl)propiolamide,
[14] 3-(3,5-dichloro-phenyl)-N-(2-(pyridine-2-yl)ethyl)propiolamide,
[15] 3-(3-fluoro-4-methyl-phenyl)-N-(2-(pyridine-2-yl)ethyl)propiolamide,
[16] 3-(4-tert.butyl-phenyl)-N-(2-(pyridine-2-yl)ethyl)propiolamide,
[17] 3-(2,4-dichloro-phenyl)-N-(2-(pyridine-2-yl)ethyl)propiolamide,
[18] N-(2-(pyridine-2-yl)ethyl)-3-m-tolyl-propiolamide,
[19] 3-(2,4-dimethyl-phenyl)-N-(2-(pyridine-2-yl)ethyl)propiolamide,
[20] 3-(4-fluoro-phenyl)-N-(2-(pyridine-2-yl)ethyl)propiolamide,
[21] 3-(4-chloro-phenyl)-N-(2-(pyridine-2-yl)ethyl)propiolamide,
[22] 3-(2-cyano-phenyl)-N-(2-(pyridine-2-yl)ethyl)propiolamide,
[23] 3-(4-cyano-phenyl)-N-(2-(pyridine-2-yl)ethyl)propiolamide,
[24] 3-(4-methoxy-phenyl)-N-(2-(pyridine-2-yl)ethyl)propiolamide,
[25] N-(2-(pyridine-2-yl)ethyl)-3-thiophene-2-yl-propiolamide,
[26] N-benzyl-3-(2,4-difluoro-phenyl)-N-phenethylpropiolamide,
[27] N-benzyl-3-(2-fluoro-phenyl)-N-phenethylpropiolamide,
[28] N-benzyl-N-phenethyl-3-p-tolyl-propiolamide,
[29] N-benzyl-N-phenethyl-3-(4-(trifluoromethyl)-phenyl)-propiolamide,
[30] 3-(2-bromo-5-methoxy-phenyl)-N-(2-(pyridine-2-yl)ethyl)propiolamide,
[31] N-(2-(pyridine-2-yl)ethyl)-3-o-tolyl-propiolamide,
[32] N-(2-(pyridine-2-yl)ethyl)-3-(2-(trifluoromethyl)-phenyl)-propiolamide,
[33] 3-(3-bromo-4-methoxy-phenyl)-N-(2-(pyridine-2-yl)ethyl)propiolamide,
[34] N-benzyl-N-phenethyl-3-phenyl-propiolamide,
[35] N-benzyl-3-(2,3-dimethyl-phenyl)-N-phenethyl-propiolamide,
[36] N-benzyl-3-(3,5-dichloro-phenyl)-N-phenethyl-propiolamide,
[37] N-benzyl-3-(2,4-dichloro-phenyl)-N-phenethyl-propiolamide,
[38] 3-(1H-indol-5-yl)-N-(2-(pyridine-2-yl)ethyl)propiolamide,
[39] N-benzyl-3-(1H-indol-5-yl)-N-phenethylpropiolamide,
[40] N-(3,4-dimethoxyphenethyl)-3-(4-fluoro-3-methylphenyl)-N-methylpropiolamide,
[41] 3-(4-fluoro-3-methylphenyl)-N-methyl-N-phenethylpropiolamide,
[42] 3-(2-fluorophenyl)-N-methyl-N-phenethylpropiolamide,
[43] N-(3,4-dimethoxyphenethyl)-N-methyl-3-p-tolylpropiolamide,
[44] N-methyl-N-phenethyl-3-p-tolylpropiolamide,
[45] 3-(2,4-dichlorophenyl)-N-methyl-N-phenethylpropiolamide,
[46] N-methyl-N-phenethyl-3-m-tolylpropiolamide,
[47] 3-(2,4-dimethylphenyl)-N-methyl-N-phenethylpropiolamide,
[48] N-(3,4-dimethoxyphenethyl)-3-(4-fluorophenyl)-N-methylpropiolamide,
[49] N-methyl-N-phenethyl-3-(3-(trifluoromethyl)phenyl)propiolamide,
[50] N-(3,4-dimethoxyphenethyl)-N-methyl-3-o-tolylpropiolamide,
[51] N-methyl-N-phenethyl-3-o-tolylpropiolamide,
[52] N-(3,4-dimethoxyphenethyl)-N-methyl-3-(2-(trifluoromethyl)phenyl)propiolamide,
[53] N-methyl-N-phenethyl-3-(2-(trifluoromethyl)phenyl)propiolamide,
[54] 3-(2-cyanophenyl)-N-(3,4-dimethoxyphenethyl)-N-methylpropiolamide,
[55] 3-(2-cyanophenyl)-N-methyl-N-phenethylpropiolamide,
[56] 3-(4-fluoro-3-methylphenyl)-N-(2-(thiophene-2-yl)ethyl)propiolamide,
[57] N-(2-(1H-indol-3-yl)ethyl)-3-m-tolylpropiolamide,
[58] N-(3-chlorophenethyl)-3-(3-methoxyphenyl)propiolamide,
[59] 3-(3-methoxyphenyl)-N-(4-methylphenethyl)propiolamide,
[60] N-(2,2-diphenylethyl)-3-(3-methoxyphenyl)propiolamide,
[61] N-(4-fluorophenethyl)-3-(3-methoxyphenyl)propiolamide,
[62] N-(3-chlorophenethyl)-3-(4-fluoro-3-methylphenyl)propiolamide,
[63] 3-(4-fluoro-3-methylphenyl)-N-(4-methylphenethyl)propiolamide,
[64] N-(2,2-diphenylethyl)-3-(4-fluoro-3-methylphenyl)propiolamide,
[69] N-(3-chlorophenethyl)-3-m-tolylpropiolamide,
[70] N-(4-methylphenethyl)-3-m-tolylpropiolamide,
[71] N-(2,2-diphenylethyl)-3-m-tolylpropiolamide,
[72] 3-(4-fluorophenyl)-N-phenethylpropiolamide,
[73] 3-(thiophene-2-yl)-N-(2-(thiophene-2-yl)ethyl)propiolamide,
[74] N-benzyl-3-(4-fluorophenyl)-N-phenethylpropiolamide,
[75] N-benzyl-3-(4-tert-butylphenyl)-N-phenethylpropiolamide,
[76] N-benzyl-N-phenethyl-3-(2-(trifluoromethyl)phenyl)propiolamide,
[77] N-benzyl-N-phenethyl-3-(thiophene-2-yl)propiolamide,
[78] 3-(2,4-difluorophenyl)-N-methyl-N-phenethylpropiolamide,
[79] N-(3,4-dimethoxyphenethyl)-3-(3-methoxyphenyl)-N-methylpropiolamide,
[80] 3-(3-methoxyphenyl)-N-methyl-N-phenethylpropiolamide,
[81] 3-(3-methoxyphenyl)-N-phenethylpropiolamide,
[82] 3-(4-fluoro-3-methylphenyl)-N-phenethylpropiolamide,
[84] N-phenethyl-3-m-tolylpropiolamide,
[85] N-phenethyl-3-(thiophene-2-yl)propiolamide,
[86] 3-(3-cyanophenyl)-N-phenethylpropiolamide,
[87] N-(2-(1H-indol-3-yl)ethyl)-3-(3-methoxyphenyl)propiolamide,
[88] N-(2-(1H-indol-3-yl)ethyl)-3-(4-fluoro-3-methylphenyl)propiolamide,
[89] N-(3-chlorophenethyl)-3-(4-fluorophenyl)propiolamide,
[90] 3-(4-fluorophenyl)-N-(4-methylphenethyl)propiolamide,
[91] N-(2,2-diphenylethyl)-3-(4-fluorophenyl)propiolamide,
[92] N-(4-fluorophenethyl)-3-(4-fluorophenyl)propiolamide,
[93] N-(3-chlorophenethyl)-3-(3-chlorophenyl)propiolamide,
[94] N-(3-chlorophenethyl)-3-(thiophene-2-yl)propiolamide,
[95] N-(4-methylphenethyl)-3-(thiophene-2-yl)propiolamide,
[96] N-(2,2-diphenylethyl)-3-(thiophene-2-yl)propiolamide,
[97] 3-(3-cyanophenyl)-N-(4-methylphenethyl)propiolamide,
[98] 3-(3-cyanophenyl)-N-(2,2-diphenylethyl)propiolamide,
[99] 3-(3-cyanophenyl)-N-(4-fluorophenethyl)propiolamide,
[100] N-(3,4-dichlorophenethyl)-3-(4-fluoro-3-methylphenyl)propiolamide,
[101] N-(3,4-dichlorophenethyl)-3-phenylpropiolamide,
[102] N-(3,4-dichlorophenethyl)-3-m-tolylpropiolamide,
[103] N-(3,4-dichlorophenethyl)-3-(thiophene-2-yl)propiolamide,
[104] 3-(3-cyanophenyl)-N-(3,4-dichlorophenethyl)propiolamide,
[105] N-(2-(1H-indol-3-yl)ethyl)-3-(2,4-difluorophenyl)propiolamide,
[106] N-(2-(1H-indol-3-yl)ethyl)-3-(2-fluorophenyl)propiolamide,
[107] N-(2-(1H-indol-3-yl)ethyl)-3-p-tolylpropiolamide,
[108] N-(2-(1H-indol-3-yl)ethyl)-3-(2,4-dichlorophenyl)propiolamide,
[109] N-(2-(1H-indol-3-yl)ethyl)-3-(2,4-dimethylphenyl)propiolamide,
[110] N-(2-(1H-indol-3-yl)ethyl)-3-(4-tert-butylphenyl)propiolamide,
[111] N-(2-(1H-indol-3-yl)ethyl)-3-(3,4-dimethylphenyl)propiolamide,
[112] N-(2-(1H-indol-3-yl)ethyl)-3-(2-bromo-5-methoxyphenyl)propiolamide,
[113] N-(2-(1H-indol-3-yl)ethyl)-3-o-tolylpropiolamide,
[114] N-(2-(1H-indol-3-yl)ethyl)-3-(2-(trifluoromethyl)phenyl)propiolamide,
[115] N-(2-(1H-indol-3-yl)ethyl)-3-(2-cyanophenyl)propiolamide,
[116] N-(2-(1H-indol-3-yl)ethyl)-3-(3,5-dichlorophenyl)propiolamide,
[117] 3-(2,4-difluorophenyl)-N-(2-(thiophene-2-yl)ethyl)propiolamide,
[118] 3-(2-fluorophenyl)-N-(2-(thiophene-2-yl)ethyl)propiolamide,
[119] N-(2-(thiophene-2-yl)ethyl)-3-p-tolylpropiolamide,
[120] N-(2-(thiophene-2-yl)ethyl)-3-(4-(trifluoromethyl)phenyl)propiolamide,
[121] 3-(2,3-dimethylphenyl)-N-(2-(thiophene-2-yl)ethyl)propiolamide,
[122] 3-(3,5-dichlorophenyl)-N-(2-(thiophene-2-yl)ethyl)propiolamide,
[123] 3-(2,4-dichlorophenyl)-N-(2-(thiophene-2-yl)ethyl)propiolamide,
[124] 3-(3-fluoro-4-methylphenyl)-N-(2-(thiophene-2-yl)ethyl)propiolamide,
[125] 3-(4-tert-butylphenyl)-N-(2-(thiophene-2-yl)ethyl)propiolamide,
[126] 3-(3,4-dimethylphenyl)-N-(2-(thiophene-2-yl)ethyl)propiolamide,
[127] 3-(2-bromo-5-methoxyphenyl)-N-(2-(thiophene-2-yl)ethyl)propiolamide,
[128] N-(2-(thiophene-2-yl)ethyl)-3-o-tolylpropiolamide,
[129] N-(2-(thiophene-2-yl)ethyl)-3-(2-(trifluoromethyl)phenyl)propiolamide,
[130] 3-(2-cyanophenyl)-N-(2-(thiophene-2-yl)ethyl)propiolamide,
[131] 3-(4-cyanophenyl)-N-(2-(thiophene-2-yl)ethyl)propiolamide,
[132] N-(2-methoxyphenethyl)-3-(3-methoxyphenyl)propiolamide,
[133] N-(4-chlorophenethyl)-3-(3-methoxyphenyl)propiolamide,
[134] 3-(4-fluoro-3-methylphenyl)-N-(2-methoxyphenethyl)propiolamide,
[135] N-(4-chlorophenethyl)-3-(4-fluoro-3-methylphenyl)propiolamide,
[138] N-(2-methoxyphenethyl)-3-m-tolylpropiolamide,
[139] N-(4-chlorophenethyl)-3-m-tolylpropiolamide,
[140] N-(4-chlorophenethyl)-3-(4-fluorophenyl)propiolamide,
[141] 3-(3,5-dichlorophenyl)-N-(2-methoxyphenethyl)propiolamide,
[142] 3-(2,4-dichlorophenyl)-N-(2-methoxyphenethyl)propiolamide,
[143] 3-(2,4-dimethylphenyl)-N-phenethylpropiolamide,
[144] N-(3-chlorophenethyl)-3-(2,4-difluorophenyl)propiolamide,
[145] 3-(2,4-difluorophenyl)-N-(4-methylphenethyl)propiolamide,
[146] 3-(2,4-difluorophenyl)-N-(2,2-diphenylethyl)propiolamide,
[147] 3-(2,4-difluorophenyl)-N-(4-fluorophenethyl)propiolamide,
[148] N-(3-chlorophenethyl)-3-(2-fluorophenyl)propiolamide,
[149] 3-(2-fluorophenyl)-N-(4-methylphenethyl)propiolamide,
[150] N-(2,2-diphenylethyl)-3-(2-fluorophenyl)propiolamide,
[151] N-(4-fluorophenethyl)-3-(2-fluorophenyl)propiolamide,
[152] N-(3-chlorophenethyl)-3-p-tolylpropiolamide,
[153] N-(4-methylphenethyl)-3-p-tolylpropiolamide,
[154] N-(2,2-diphenylethyl)-3-p-tolylpropiolamide,
[155] N-(3-chlorophenethyl)-3-(4-(trifluoromethyl)phenyl)propiolamide,
[156] N-(4-methylphenethyl)-3-(4-(trifluoromethyl)phenyl)propiolamide,
[157] N-(2,2-diphenylethyl)-3-(4-(trifluoromethyl)phenyl)propiolamide,
[158] N-(4-fluorophenethyl)-3-(4-(trifluoromethyl)phenyl)propiolamide,
[159] N-(3,4-dichlorophenethyl)-3-(4-(trifluoromethyl)phenyl)propiolamide,
[160] N-(3-chlorophenethyl)-3-(2,3-dimethylphenyl)propiolamide,
[161] 3-(2,3-dimethylphenyl)-N-(4-methylphenethyl)propiolamide,
[162] 3-(2,3-dimethylphenyl)-N-(2,2-diphenylethyl)propiolamide,
[163] 3-(2,3-dimethylphenyl)-N-(4-fluorophenethyl)propiolamide,
[164] N-(3,4-dichlorophenethyl)-3-(2,3-dimethylphenyl)propiolamide,
[165] N-(3-chlorophenethyl)-3-(3,5-dimethylphenyl)propiolamide,
[166] 3-(3,5-dimethylphenyl)-N-(4-methylphenethyl)propiolamide,
[167] 3-(3,5-dimethylphenyl)-N-(2,2-diphenylethyl)propiolamide,
[168] 3-(4-hydroxy-3-methylphenyl)-N-(4-methylphenethyl)propiolamide,
[169] N-(2,2-diphenylethyl)-3-(4-hydroxy-3-methylphenyl)propiolamide,
[170] 3-(1H-indol-5-yl)-N-(4-methylphenethyl)propiolamide,
[171] N-(2,2-diphenylethyl)-3-(1H-indol-5-yl)propiolamide,
[172] N-(4-fluorophenethyl)-3-(1H-indol-5-yl)propiolamide,
[173] N-(3,4-dichlorophenethyl)-3-(1H-indol-5-yl)propiolamide,
[174] N-(3-chlorophenethyl)-3-(3,5-dichlorophenyl)propiolamide,
[175] 3-(3,5-dichlorophenyl)-N-(4-methylphenethyl)propiolamide,
[176] 3-(3,5-dichlorophenyl)-N-(2,2-diphenylethyl)propiolamide,
[177] 3-(3,5-dichlorophenyl)-N-(4-fluorophenethyl)propiolamide,
[178] N-(3,4-dichlorophenethyl)-3-(3,5-dichlorophenyl)propiolamide,
[179] 3-(2,4-dichlorophenyl)-N-(4-methylphenethyl)propiolamide,
[180] 3-(2,4-dichlorophenyl)-N-(4-fluorophenethyl)propiolamide,
[181] N-(3,4-dichlorophenethyl)-3-(2,4-dichlorophenyl)propiolamide,
[182] N-(3-chlorophenethyl)-3-o-tolylpropiolamide,
[183] N-(4-methylphenethyl)-3-o-tolylpropiolamide,
[184] N-(2,2-diphenylethyl)-3-o-tolylpropiolamide,
[185] N-(4-fluorophenethyl)-3-o-tolylpropiolamide,
[186] N-(3,4-dichlorophenethyl)-3-o-tolylpropiolamide,
[187] N-(3,4-dichlorophenethyl)-3-(4-hydroxy-3-methylphenyl)propiolamide,
[188] 3-(3-chlorophenyl)-N-(2-methoxyphenethyl)propiolamide,
[189] N-(2-methoxyphenethyl)-3-(thiophene-2-yl)propiolamide,
[190] N-(4-chlorophenethyl)-3-(thiophene-2-yl)propiolamide,
[191] 3-(3-cyanophenyl)-N-(2-methoxyphenethyl)propiolamide,
[192] N-(4-chlorophenethyl)-3-(3-cyanophenyl)propiolamide,
[193] 3-(2,4-difluorophenyl)-N-(2-methoxyphenethyl)propiolamide,
[194] 3-(2-fluorophenyl)-N-(2-methoxyphenethyl)propiolamide,
[195] N-(2-methoxyphenethyl)-3-(4-(trifluoromethyl)phenyl)propiolamide,
[196] 3-(3-fluoro-4-methylphenyl)-N-phenethylpropiolamide,
[197] 3-(4-tert-butylphenyl)-N-phenethylpropiolamide,
[198] 3-(3,4-dimethylphenyl)-N-phenethylpropiolamide,
[199] 3-(2,4-difluorophenyl)-N-phenethylpropiolamide,
[200] N-phenethyl-3-p-tolylpropiolamide,
[201] N-phenethyl-3-(4-(trifluoromethyl)phenyl)propiolamide,
[202] 3-(2,3-dimethylphenyl)-N-phenethylpropiolamide,
[203] 3-(3,5-dimethylphenyl)-N-phenethylpropiolamide,
[204] 3-(1H-indol-5-yl)-N-phenethylpropiolamide,
[205] 3-(2,3-dimethylphenyl)-N-(2-methoxyphenethyl)propiolamide,
[206] 3-(3,5-dimethylphenyl)-N-(2-methoxyphenethyl)propiolamide,
[207] 3-(3,5-dichlorophenyl)-N-phenethylpropiolamide,
[208] N-(3,4-dichlorophenethyl)-3-(2,4-difluorophenyl)propiolamide,
[209] N-(3,4-dichlorophenethyl)-3-(2-fluorophenyl)propiolamide,
[210] N-(4-fluorophenethyl)-3-p-tolylpropiolamide,
[211] N-(3,4-dichlorophenethyl)-3-p-tolylpropiolamide,
[212] 3-(3,5-dimethylphenyl)-N-(4-fluorophenethyl)propiolamide,
[213] N-(3-chlorophenethyl)-3-(2,4-dichlorophenyl)propiolamide,
[214] 3-(2,4-dichlorophenyl)-N-(2,2-diphenylethyl)propiolamide,
[215] 3-(3-chlorophenyl)-N-(2-fluorophenethyl)propiolamide,
[216] 3-(3-chlorophenyl)-N-(3-fluorophenethyl)propiolamide,
[217] 3-(3-chlorophenyl)-N-(4-fluorophenethyl)propiolamide,
[218] 3-(3-chlorophenyl)-N-(2-fluorophenethyl)-N-methylpropiolamide,
[219] 3-(3-chlorophenyl)-N-(3-(trifluoromethyl)phenethyl)propiolamide,
[220] 3-(3-chlorophenyl)-N-(4-fluorophenethyl)-N-methylpropiolamide,
[221] 3-(3-chlorophenyl)-N-(3-fluorophenethyl)-N-methylpropiolamide,
[222] 3-(3-chlorophenyl)-N-methyl-N-(3-(trifluoromethyl)phenethyl)propiolamide,
[223] 3-(3-chlorophenyl)-N-(2-methylphenethyl)propiolamide,
[224] 3-(3-chlorophenyl)-N-(3-methylphenethyl)propiolamide,
[225] 3-(3-chlorophenyl)-N-(4-methylphenethyl)propiolamide,
[226] 3-(3-chlorophenyl)-N-methyl-N-(2-methylphenethyl)propiolamide,
[227] 3-(3-chlorophenyl)-N-methyl-N-(3-methylphenethyl)propiolamide,
[228] 3-(3-chlorophenyl)-N-methyl-N-(4-methylphenethyl)propiolamide,
[229] 3-(3-chlorophenyl)-N-(2-(dimethylamino)-2-phenylethyl)propiolamide,
[230] N-(2-(1H-pyrrol-1-yl)ethyl)-3-(3-chlorophenyl)propiolamide,
[231] N-(2-(1H-pyrazol-1-yl)ethyl)-3-(3-chlorophenyl)propiolamide,
[232] N-(2-(1H-imidazol-1-yl)ethyl)-3-(3-chlorophenyl)propiolamide,
[233] N-(2-(1H-pyrrol-1-yl)ethyl)-3-(3-chlorophenyl)-N-methylpropiolamide,
[234] N-(2-(1H-pyrazol-1-yl)ethyl)-3-(3-chlorophenyl)-N-methylpropiolamide,
[235] 3-(3-chlorophenyl)-N-(2-(pyridine-3-yl)ethyl)propiolamide,
[236] 3-(3-chlorophenyl)-N-methyl-N-(2-(pyridine-2-yl)ethyl)propiolamide,
[237] 3-(3-chlorophenyl)-N-ethyl-N-phenethylpropiolamide,
[238] 3-(3-chlorophenyl)-N-isopropyl-N-phenethylpropiolamide,
[239] 3-(3-chlorophenyl)-N-cyclopropyl-N-phenethylpropiolamide,
[240] 3-(3-chlorophenyl)-N-(2-methoxyphenethyl)-N-methylpropiolamide,
[241] 3-(3-chlorophenyl)-N-methyl-N-(1-phenylpropane-2-yl)propiolamide,
[242] 3-(3-chlorophenyl)-N-(1-phenylpropane-2-yl)propiolamide,
[243] 3-(3-chlorophenyl)-N-methyl-N-(2-(pyridine-3-yl)ethyl)propiolamide,
[244] 3-(3-chlorophenyl)-N-methyl-N-(2-(pyridine-4-yl)ethyl)propiolamide,
[245] 3-(3-chlorophenyl)-N-methyl-N-(2-(thiophene-2-yl)ethyl)propiolamide,
[246] 3-(3-chlorophenyl)-N-(1-(pyridine-3-yl)propane-2-yl)propiolamide,
[247] 3-(3-chlorophenyl)-N-methyl-N-(1-(pyridine-3-yl)propane-2-yl)propiolamide,
[248] 3-(3-chlorophenyl)-N-(2-(2-methylpyridine-3-yl)ethyl)propiolamide,
[249] 3-(3-chlorophenyl)-N-methyl-N-(2-(2-methylpyridine-3-yl)ethyl)propiolamide,
[250] 3-(3-chlorophenyl)-N-(2-(trifluoromethyl)phenethyl)propiolamide,
[251] 3-(3-chlorophenyl)-N-methyl-N-(2-(trifluormethyl)phenethyl)propiolamide,
[252] 3-(3-chlorophenyl)-N-(4-(trifluoromethyl)phenethyl)propiolamide and
[253] 3-(3-chlorophenyl)-N-methyl-N-(4-(trifluoromethyl)phenethyl)propiolamide;
in each case optionally in the form of one of the pure stereoisomers thereof, the racemates thereof or in the form of a mixture of stereoisomers in any desired mixing ratio, or in each case in the form of a corresponding salts or in each case in the form of a corresponding solvates.

20. A compound according to claim 1, which, after 60 minutes of incubation in 450 μg protein from pig brain homogenate at a temperature between 20° C. and 25° C. in a concentration of less than 2000 nM, brings about a 50-percent displacement of [³H]-2-methyl-6-(3-methoxyphenyl)-ethynylpyridine which is present in a concentration of 5 nM.

21. A method for producing a compound of the formula I according to claim 1, wherein at least one compound of the formula II, 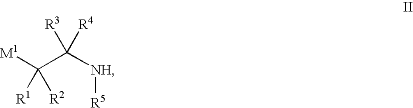
in which R¹, R², R³, R⁴, R⁵ and M¹ have the meaning according to claim 1, is transferred by conversion with at least one compound of the formula M²-C≡C≡C(═O)—OH, in which M² has the meaning according to claim 1, optionally in a reaction medium, optionally in the presence of at least one suitable coupling agent, optionally in the presence of at least one base, or by conversion with at least one compound of the formula M²-C≡C≡C(═O)—X, in which M² has the above-mentioned meaning and X denotes a leaving group in a reaction medium, optionally in the presence of at least one base, into at least one corresponding compound of the formula I, optionally in the form of a corresponding salt, 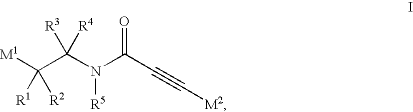
in which R¹, R², R³, R⁴, R⁵, M¹ and M² have the above-mentioned meaning, and this is optionally purified and/or isolated;
or at least one compound of the formula II is transferred by conversion with propiolic acid [HC≡C—C(═O)—OH] optionally in a reaction medium, optionally in the presence of at least one suitable coupling agent, optionally in the presence of at least one base, or by conversion with at least one compound of the general formula HC≡C—C(═O)—X, in which X denotes a leaving group, in a reaction medium, optionally in the presence of at least one base, into at least one corresponding compound of the formula III, optionally in the form of a corresponding salt, 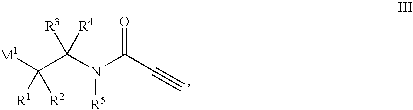
in which R¹, R², R³, R⁴, R⁵, M¹ and M² have the above-mentioned meaning, and this is optionally purified and/or isolated,
and at least one compound of the formula III is transferred into at least one corresponding compound of the formula I, optionally in the form of a corresponding salt by conversion with at least one compound of the formula M²-X, in which M² has the meaning according to claim 1 and X denotes a leaving group, optionally in a reaction medium, optionally in the presence of at least one catalyst, optionally in the presence of at least one ligand, optionally in the presence of at least one inorganic salt, optionally in the presence of at least one copper salt, optionally in the presence of at least one organic or inorganic base, and this is optionally purified and/or isolated.

22. A pharmaceutical composition comprising at least one compound according to claim 1 and optionally one or more physiologically acceptable auxiliary substances.

23. Pharmaceutical composition according to claim 22, wherein the at least one compound is selected from the group consisting of:
[65] N-(3-chlorophenethyl)-3-phenylpropiolamide,
[66] N-(4-methylphenethyl)-3-phenylpropiolamide,
[67] N-(2,2-diphenylethyl)-3-phenylpropiolamide,
[68] N-(4-fluorophenethyl)-3-phenylpropiolamide,
[83] N-phenethyl-3-phenylpropiolamide,
[136] N-(2-methoxyphenethyl)-3-phenylpropiolamide and
[137] N-(4-chlorophenethyl)-3-phenylpropiolamide,
in each case optionally in the form of a corresponding salt, or in each case in the form of a corresponding solvate

24. (canceled)

25. (canceled)

26. (canceled)

27. A method for regulating the mGluR5 receptor said method comprising administering to a patient in need of such regulating an effective amount therefor of at least one compound according to claim 1.

28. A method for preventing or treating a disorder or illness that is at least partially mediated by mGluR5 receptors, said method comprising administering to a patient in need of such preventing or treating an effective amount therefor of at least one compound according to claim 1.

29. The method according to claim 28, wherein the illness or disorder is selected from the group consisting of pain; migraine; depression; neurodegenerative diseases cognitive dysfunction; anxiety states; panic attacks; epilepsy; coughing; urinary incontinence; diarrhoea; pruritus; schizophrenia; cerebral ischaemia; muscle spasms; cramps; lung illnesses; regurgitation (vomiting); stroke; dyskinesia; retinopathy; listlessness; laryngitis; disorders of food intake; dependency on alcohol; dependency on medicines; dependency on drugs; alcohol abuse; abuse of medication; drug abuse; withdrawal symptoms associated with dependency on alcohol, medications and/or drugs; development of tolerance to medications; stomach-esophagus-reflux-syndrome; gastroesophagal reflux; irritable bowel syndrome; diuresis; antinatriuresis; disorders of the cardiovascular system; reduced vigilance; reduced libido; and disorders relating to locomotor activity.

30. The method according to claim 28, wherein the illness or disorder is selected from the group consisting of pain selected from the group consisting of acute pain, chronic pain, neuropathic pain and visceral pain; anxiety states; panic attacks; dependency on alcohol; dependency on medicines; cognitive dysfunction; disorders of food intake; alcohol abuse; abuse of medication; drug abuse; withdrawal symptoms associated with dependency on alcohol, medications and/or drugs; development of tolerance to medications and/or drugs; stomach-esophagus-reflux-syndrome; gastroesophagal reflux and irritable bowel syndrome.

31. The method according to claim 28, wherein the illness or disorder is pain selected from the group consisting of acute pain, chronic pain, neuropathic pain and visceral pain.

32. The method according to claim 28, wherein the illness or disorder is selected from the group consisting of anxiety states and panic attacks.

33. A method of providing local anaesthesia to a patient in need thereof, said method comprising locally administering to said patient at least one compound according to claim 1.
